(19) United States
(12) Patent Application Publication
(10) Pub. No.: US 2012/0115869 A1
(43) Pub. Date: May 10, 2012

(54) TETRAZOLE DERIVATIVES

(75) Inventors: Stefano Crosignani, St. Genis-Pouilly (FR); Christophe Cleva, La Tour (FR); Anna Quattropani, Geneva (CH); Gwenaelle Desforges, Annemasse (FR); Agnès Bombrun, Chambesy (CH)

(73) Assignee: MERCK SERONO S.A., COINSINS, VAUD (CH)

(21) Appl. No.: 13/383,848

(22) PCT Filed: Jul. 14, 2010
(86) PCT No.: PCT/EP10/60151

§ 371 Date: Jan. 13, 2012

Related U.S. Application Data
(60) Provisional application No. 61/229,369, filed on Jul. 29, 2009.

(30) Foreign Application Priority Data
Jul. 15, 2009 (EP) 09165566.2

(51) Int. Cl. A61K 31/41; C07D 403/12; C07D 405/12; C07D 409/12; C07D 413/12; C07D 417/12; C07D 401/12; A61K 31/4439; A61K 31/4709; A61K 31/5377; A61P 37/08; A61P 11/06; A61P 27/02; A61P 27/00; A61K 31/422; A61K 31/428; A61K 31/4245; A61K 31/427; A61K 31/4155; A61P 17/00; A61P 29/00; C07D 257/04

(52) U.S. Cl. 514/236.2; 548/253; 514/381; 514/382; 548/125; 546/268.4; 514/340; 546/175; 514/314; 544/132; 548/247; 514/378; 548/180; 514/367; 514/364; 548/203; 514/365

(57) ABSTRACT

The present invention relates to compounds of formula (I) for use as pharmaceutical active compounds, as well as pharmaceutical formulations containing the same, for the treatment of allergic diseases.

The present invention relates to compounds of formula (I) for use as pharmaceutical active compounds, as well as pharmaceutical formulations containing such compounds. Specifically, the invention relates to tetrazole derivatives of Formula (I):

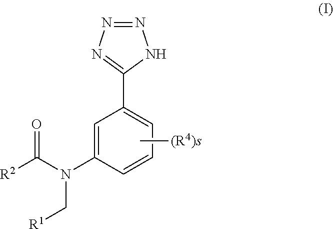

R¹ is —(CH₂)ₙ—Ar, —(CH₂)ₙHet, —(CH₂)ₚ—(CHR⁸)ₘ—(CH₂)_{q}—Ar, or —(CH₂)ₚ—(CHR⁸)ₘ—(CH₂)_{q}—Het,
R² is A, Het, Ar, or a cycloalkyl having 1 to 8 carbon atoms,

### R⁴ is H, Hal, A, CN, OA, CF₃, OCF₃,

n is 0, 1, 2, 3, or 4
p, q are 0, 1, 2 or 3
m is 0, 1 or 2
s 1, 2 or 3
R⁸ denotes a group selected from an alkyl having 1 to 8 carbon atoms, —CH₂F, —CF₃, OR³, N(R³)₂, —CH₂OCH₃, —CH₂OCF₃, —CH₂CONH₂, or CN.
A is branched or linear alkyl having 1 to 12 C-atoms, wherein one or more, preferably 1 to 7 H-atoms may be replaced by Hal, OR³, CN, N(R³)₂, CON(R³)₂, Ar or Het and wherein one or more, preferably 1 to 7 non-adjacent CH₂-groups may be replaced by O, NR³ or S and/or by —CH═CH— or —C≡C— groups, or denotes cycloalkyl or cycloalkylalkylen having 3 to 7 ring C atoms.
R³ denotes H or A

### Hal is F, Cl, Br or I,

Ar denotes a monocyclic or fused bicyclic, unsaturated or aromatic carbocyclic ring having 6 to 14 carbon atoms which may be unsubstituted or monosubstituted, disubstituted or trisubstituted by Hal, A, CH₂OA, —CH₂OR³, OR³, CF₃, OCF₃, N(R³)₂, NO₂, CN, NR³COA, NR³SO₂A, COR³, SO₂N(R³)₂, SOA, SO₂A, SOAr, SO₂Ar, SOHet, SO₂Het, Ar′, Het, or by —CH═CH—R³ or —C≡C—R³.
Het denotes a monocyclic or bicyclic, saturated, unsaturated or aromatic heterocyclic ring, having 1 to 4 N, O and/or S atoms, which may be unsubstituted or monosubstituted, disubstituted or trisubstituted by Hal, A, OR³, —(CH₂)OR³, CF₃, OCF₃, N(R³)₂, NO₂, CN, NR³COA, NR³SO₂A, COR³, SO₂N(R³)₂, SOA, SO₂A, SOAr, SO₂Ar, SOHet, SO₂Het, Ar, Het′, or by —CH═CH—R³ or —C≡C—R³.
Ar′ denotes a monocyclic or bicyclic, unsaturated or aromatic carbocyclic ring having 6 to 14 carbon atoms which may be unsubstituted or monosubstituted, disubstituted or trisubstituted by Hal, A, —(CH₂)OR³, —OR³, —CF₃, —OCF₃.
Het′ denotes a monocyclic or bicyclic, saturated, unsaturated or aromatic heterocyclic ring, having 1 to 4 N, O and/or S atoms, which may be unsubstituted or monosubstituted, disubstituted or trisubstituted by Hal, A, CH₂OR³, OR³, CF₃, OCF₃.

As well as pharmaceutically usable derivatives, enantiomers, diastereoisomers, tautomers, salts, solvates and mixtures thereof in all ratios.

Said derivatives are useful for the treatment and/or prevention of allergic diseases and inflammatory dermatoses. Specifically, the present invention is related to the use of tetrazole derivatives for the modulation of CRTH2 activity.

The present invention furthermore relates to methods of the preparation of tetrazole derivatives.

The present invention also relates to a kit or a set consisting of separate packs of
(b) (a) an effective amount of a compound according to formula (I) and/or pharmaceutically usable derivatives, tautomers, salts, solvates and stereoisomers thereof, including mixtures thereof in all ratios,
   and
(c) an effective amount of a further medicament active ingredient.

Prostaglandin D2 (PGD2) has long been associated with inflammatory and atopic conditions, specifically allergic diseases such as asthma, rhinitis and atopic dermatitis (Lewis et al. (1982) J. Immunol. 129, 1627). PGD2 belongs to a class of compounds derived from the 20-carbon fatty acid skeleton of arachidonic acid. In response to an antigen challenge, PGD2 is released in large amounts into the airway as well as to the skin during an acute allergic response. The DP receptor, which is a member of the G-protein coupled receptor (GPCR) subfamily, has long been thought to be the only receptor of PGD2. DP's role in allergic asthma has been demonstrated with DP deficient mice (Matsuoka et al. (2000) Science 287, 2013-2017). However, despite intense interest in the role of PGD2 in the inflammatory response, a direct link between DP receptor activation and PGD2-stimulated eosinophil migration has not been established (Woodward et al. (1990) Invest. Ophthalomol. Vis. Sci. 31, 138-146; Woodward et al. (1993) Eur. J. Pharmacol. 230, 327-333).

More recently, another G-protein coupled receptor, referred to as “Chemoattractant Receptor-Homologous molecule expressed on T-Helper 2 cells” (CRTH2) (Nagata et al. (1999) J. Immunol. 162, 1278-1286, Hirai et al. (2001) J. Exp. Med. 193, 255-261) has recently been identified as a receptor for PGD2 and this discovery has begun to shed light on the mechanism of action of PGD2. CRTH2, which is also referred to as DP2, GPR44 or DLIR, shows little structural similarity with the DP receptor and other prostanoid receptors. However, CRTH2 possesses similar affinity for PGD2. Among peripheral blood T lymphocytes, human CRTH2 is selectively expressed on Th2 cells and is highly expressed on cell types associated with allergic inflammation such as eosinophils, basophiles and Th2 cells. In addition, CRTH2 mediates PGD2 dependent cell migration of blood eosinophils and basophiles. Furthermore, increased numbers of circulating T cells expressing CRTH2 have been correlated with the severity of atopic dermatitis (Cosmi et al. (2000) Eur. J. Immunol. 30, 2972-2979). The interaction of CRTH2 with PGD2 plays a critical role in the allergen-induced recruitment of Th2 cells in the target tissues of allergic inflammation. Compounds that inhibit the binding of CRTH2 and PGD2 should therefore be useful for the treatment of allergic diseases.

Allergic disease, like asthma, and inflammatory dermatoses represent a major class of complex, and typically chronic, inflammatory diseases that currently affect about 10% of the population and that number appears to be increasing (Bush, R. K., Georgitis J. W., Handbook of asthma and rhinitis 1st ed. (1997), Abingdon: Blackwell Science. 270). Atopic dermatitis is a chronic skin disease, wherein the skin becomes extremely itchy. It accounts for 10 to 20 percent of all visits to dermatologists. The increasing incidence of allergic diseases and inflammatory dermatoses worldwide underscores the need for new therapies to effectively treat or prevent these diseases. Currently, numerous classes of pharmaceutical agents are widely used to treat these diseases, for example, antihistamines, decongestants, anticholinergics, methylxanthines, cromolyns, corticosteroids, and leukotriene modulators. However, the usefulness of these agents is often limited by side effects and low efficacy.

The invention further provides a pharmaceutical composition comprising a compound of Formula (I), together with a pharmaceutically acceptable excipient or carrier.

The invention further relates to the use of compounds of Formula (I) for the preparation of a medicament for the treatment and/or prevention of diseases selected from allergic diseases such as allergic asthma, allergic rhinitis, allergic conjunctivitis, and inflammatory dermatoses such as atopic dermatitis, contact hypersensitivity, allergic contact dermatitis, chronic urticaria/chronic idiopathic/autoimmune urticaria, drug-induced exanthems (e.g. toxic epidermal necrolysis or Lyell's syndrome/Stevens-Johnson syndrome/drug hypersensitivity syndrome), photodermatosis or polymorphous light eruption (e.g. photo-irritant contact dermatitis, photoallergic contact dermatitis, chronic actinic dermatitis), and myositis, neurodegenerative disorders such as neuropatic pain, and other diseases with an inflammatory component such as rheumatoid arthritis, multiple sclerosis, osteoarthritis, and inflammatory bowel disease (IBD) and other diseases and disorders associated with CTRH2 activity. Specifically the present invention is related to the use of compounds of Formula (I) for the modulation of CRTH2 activity.

The invention further relates to a method for treating and/or preventing a patient suffering from a disease selected from allergic diseases such as allergic asthma, allergic rhinitis, allergic conjunctivitis, and inflammatory dermatoses such as atopic dermatitis, contact hypersensitivity, allergic contact dermatitis, chronic urticaria/chronic idiopathic/autoimmune urticaria, drug-induced exanthems (e.g. toxic epidermal necrolysis or Lyell's syndrome/Stevens-Johnson syndrome/drug hypersensitivity syndrome), photodermatosis or polymorphous light eruption (e.g. photo-irritant contact dermatitis, photoallergic contact dermatitis, chronic actinic dermatitis), and myositis, neurodegenerative disorders such as neuropatic pain and other diseases with an inflammatory component such as rheumatoid arthritis, multiple sclerosis, osteoarthritis, and inflammatory bowel disease (IBD) and other diseases and disorders associated with CTRH2 activity, by administering a compound according to Formula (I).

The invention further relates to the use of compounds of Formula (I) for the preparation of a pharmaceutical composition.

The invention finally relates to novel compounds of Formula (I) as well as to methods to synthesize compounds of Formula (I).

In a preferred embodiment, the present invention provides compounds of formula (I) wherein R² is a branched or linear alkyl having 1 to 6 carbon atoms or a group —(CH₂)_{q}—R⁷ wherein R⁷ is —CH₂F, —CF₃, —CH₂OCH₃, —CH₂OCF₃, —CH₂CONH₂, or CN, and wherein q is 0, 1 or 2.

In another preferred embodiment, the present invention provides compounds of formula (I) wherein R¹ is a group Ar or Het,

In another preferred embodiment, the present invention provides compounds of formula (I) wherein R⁴ is H, Hal, —CN, —CF₃, —CH₂F, OR³, or —OCF₃.

In a preferred embodiment, the present invention provides compounds of Formula (Ia)

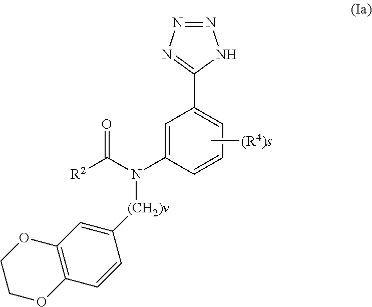

Wherein R², R⁴, S are as defined above and v is 1, 2, 3 or 4.

In another preferred embodiment, the present invention provides compounds of Formula (Ib)

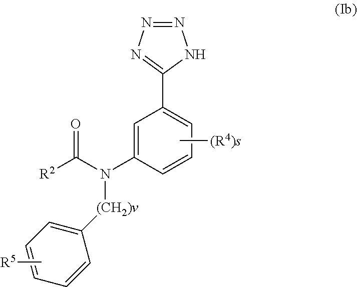

wherein R², R⁴, and S are as defined above, v is 1, 2, 3 or 4, and
wherein R⁵ denotes H or a group selected from Hal, —OCF₃, —OCH₃, —CF₃, —(CH₂)_{T}—C≡C—R⁶, —(CH₂)_{T}—CH═CH—R⁶, or SO₂(C₁-C₆alkyl),

Wherein R⁶ is H, a linear or branched C₁-C₆ alkyl, or a group selected from —CH₂F, —CF₃, —CH₂OCH₃, —CH₂OCF₃, —CH₂CONH₂, CN, Ar or Het, and wherein

T is 0, 1, 2 or 3, preferably T is 0.

In another preferred embodiment, the present invention provides compounds of Formula (Ic)

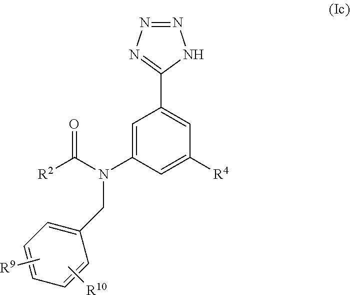

Wherein

R² is as defined above, preferrably, R² denotes a branched or linear C₁-C₆-alkyl, wherein 1 H atom may be replaced by a phenyl group.

R⁴ is as defined above. Preferably, R⁴ is Hal, and most preferably, R⁴ is F.

R⁹ denotes H, Hal, CF₃, OCF₃, SO₂(C₁-C₆)alkyl,

R¹⁰ denotes H, Hal, preferrably Cl.

In another preferred embodiment, the present invention provides compounds of Formula (Ic) wherein R² denotes ethyl, butyl, or a benzyl group,

R⁹ denotes H or Cl, and

R¹⁰ denotes CF₃, OCF₃, or SO₂CH₃.

The present invention also encompasses tautomers (IA) and (IB) of compounds of Formula (I) and related formulae (Ia), (Ib) and (Ic):

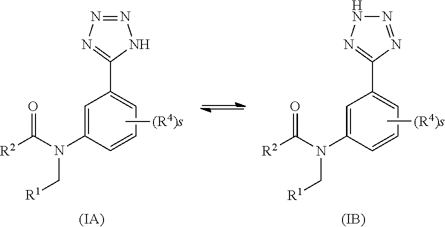

As well as pharmaceutically usable derivatives, enantiomers, diastereoisomers, salts, solvates and mixtures thereof in all ratios.

Generally, compounds of Formula (I), wherein R¹, R², R⁴ are defined as above, can be obtained from a compound of Formula (II) as outlined in Scheme 1.

The first step consists in the reaction of a compound of Formula (II), wherein R⁴ is defined as above, with a compound of Formula R¹—CHO. The reaction is performed using conditions known to those skilled in the art for performing a reductive amination, such as but not limited to using NaBH₃CN, NaBH(OAc)₃ or polymer-supported cyanoborohydride reagents in the presence or absence of a suitable acid such as AcOH, in a suitable solvent such as but not limited to THF, dioxane, DMF, DMSO, preferably DMF, at a temperature between 20° C. to 100° C., preferably at 25° C., for a few hours, e.g. one hour to 48 h. Alternatively, the reaction can be carried out in two steps, the first consisting in the condensation of a compound of Formula (II), wherein R⁴ is defined as above, with a compound of Formula R¹—CHO in the presence or absence of a suitable catalyst such as p-toluenesulfonic acid, in a suitable solvent such as toluene or benzene, preferably toluene, at a temperature between 20° C. to 100° C., preferably at 110° C., for a few hours, e.g. one hour to 48 h, in the presence of a system suitable for removing water from the reaction (such as a Dean-Stark apparatus), followed by treatment with a suitable reducing agent, such as NaBH₄, NaBH₃CN, NaBH(OAc)₃ or hydrogen gas in the presence of a suitable catalyst, such as Pd/C or PtO₂, in the presence or absence of a suitable acid such as AcOH, in a suitable solvent such as but not limited to MeOH, MeOH/DCM, THF, dioxane, DMF, preferably a mixture of MeOH and DCM, at a temperature between 20° C. to 100° C., preferably at 25° C., for a few hours, e.g. one hour to 48 h.

Conversion of compounds of Formula (III) to give compounds of Formula (I) can be achieved using conditions and methods well known to those skilled in the art for the preparation of amides from a carboxylic acid derivative (e.g. acyl chloride) with aryl amines, in the presence of bases such as TEA, DIEA, NMM, polymer-supported morpholine, in a suitable solvent such as DCM, THF or DMF, at a temperature rising from 20° C. to 100° C., preferably at 50° C., for a few hours, e.g. one hour to 24 h. Alternatively, an the aryl amines of Formula (II) can be treated with a carboxylic acid, with standard coupling agents, such as but not limited to 1-alkyl-2-chloropyridinium salt or preferably polymer-supported 1-alkyl-2-chloropyridinium salt (polymer-supported Mukaiyama's reagent), 1-methyl-2-chloropyridinium iodide (Mukaiyama's reagent), a carbodiimide (such as DCC, DIC, EDC) and HOBt, HATU, TBTU, PyBOP® and other such reagents well known to those skilled in the art, in the presence or absence of bases such as TEA, DIEA, NMM, polymer-supported morpholine, in a suitable solvent such as DCM, THF or DMF, at a temperature between 20° C. to 50° C., preferably at room temperature, for a few hours, e.g. one hour to 24 h.

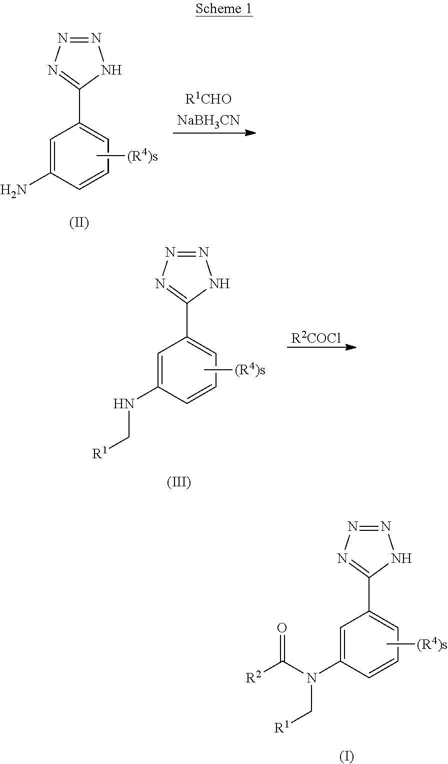

Alternatively, compounds of Formula (I), wherein R¹, R², R⁴ are defined as above, can be obtained from a compound of Formula (IV) as outlined in Scheme 2.

Compounds of Formula (IV) wherein R⁴ is defined as above, can be converted into the corresponding compounds of Formula (V), wherein R¹, R⁴ are defined as above, by treatment with a compound of Formula R¹—CHO, in the presence or absence of a suitable catalyst such as p-toluenesulfonic acid, in a suitable solvent such as toluene or benzene, preferably toluene, at a temperature between 20° C. to 100° C., preferably at 110° C., for a few hours, e.g. one hour to 48 h, in the presence of a system suitable for removing water from the reaction (such as a Dean-Stark apparatus). followed by treatment with a suitable reducing agent, such as NaBH₄, NaBH₃CN, NaBH(OAc)₃, or hydrogen gas in the presence of a suitable catalyst, such as Pd/C or PtO₂, in the presence or absence of a suitable acid such as AcOH, in a suitable solvent such as but not limited to MeOH, MeOH/DCM, THF, dioxane, preferably a mixture of MeOH and DCM, at a temperature between 20° C. to 100° C., preferably at 25° C., for a few hours, e.g. one hour to 48 h. Alternatively the reaction can be carried out in one step using conditions known to those skilled in the art for performing a reductive amination, such as but not limited to using NaBH₃CN, NaBH(OAc)₃, polymer-supported cyanoborohydride reagents in the presence or absence of a suitable acid such as AcOH, in a suitable solvent such as but not limited to THF, dioxane, DMF, DMSO, preferably DMF, at a temperature between 20° C. to 100° C., preferably at 25° C., for a few hours, e.g. one hour to 48 h.

Conversion of compounds of Formula (V) to give compounds of Formula (VI) can be achieved using conditions and methods well known to those skilled in the art for the preparation of amides from a carboxylic acid derivative (e.g. acyl chloride) with aryl amines, in the presence of bases such as TEA, DIEA, NMM, polymer-supported morpholine, in a suitable solvent such as DCM, THF or DMF, at a temperature rising from 20° C. to 100° C., preferably at 50° C., for a few hours, e.g. one hour to 24 h. Alternatively, the aryl amines of Formula (II) can be treated with a carboxylic acid, with standard coupling agents, such as but not limited to 1-alkyl-2-chloropyridinium salt or preferably polymer-supported 1-alkyl-2-chloropyridinium salt (polymer-supported Mukaiyama's reagent), 1-methyl-2-chloropyridinium iodide (Mukaiyama's reagent), a carbodiimide (such as DCC, DIC, EDC) and HOBt, HATU, TBTU, PyBOP® and other such reagents well known to those skilled in the art, in the presence or absence of bases such as TEA, DIEA, NMM, polymer-supported morpholine, in a suitable solvent such as DCM, THF or DMF, at a temperature between 20° C. to 50° C., preferably at room temperature, for a few hours, e.g. one hour to 24 h.

Finally, conversion of the aryl nitriles of Formula (VI) into compounds of Formula (I) can be achieved by treatment with a suitable azide, such as but not limited to sodium azide or TMS-azide, in the presence of a suitable catalyst such as Bu₂SnO or Cu₂O, in the presence of a suitable solvent such as toluene, methanol or DMF, preferably toluene when using Bu₂SnO or a 1:10 mixture of MeOH/DMF when using Cu₂O, at a temperature between 20° C. to 110° C., preferably at 80-110° C., for a few hours, e.g. one hour to 48 h.

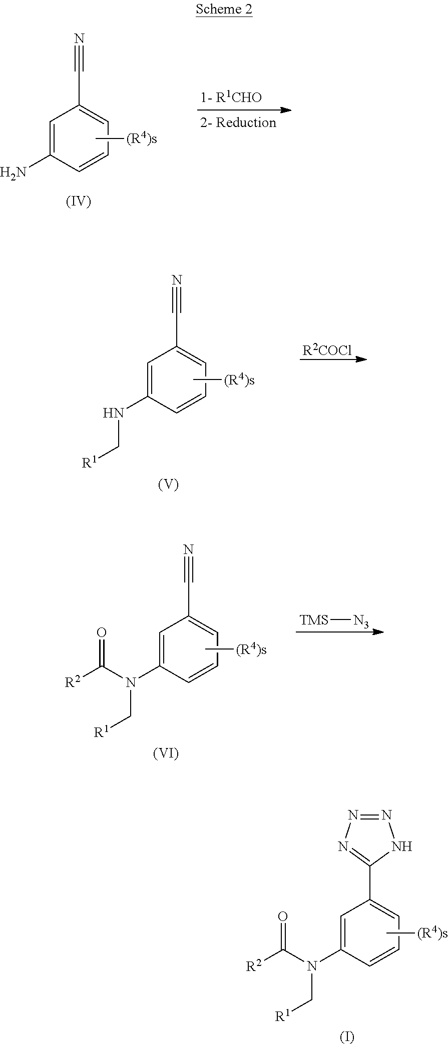

Alternatively, the compounds of Formula (VI) can be prepared as depicted in Scheme 3. Conversion of compounds of Formula (IV), wherein R⁴ is defined as above, to give compounds of Formula (VII), wherein R², R⁴ are defined as above, can be achieved using conditions and methods well known to those skilled in the art for the preparation of amides from a carboxylic acid derivative (e.g. acyl chloride) with aryl amines, in the presence of bases such as TEA, DIEA, NMM in a suitable solvent such as DCM, THF or DMF, at a temperature rising from 20° C. to 100° C., preferably at 50° C., for a few hours, e.g. one hour to 24 h. Alternatively, the aryl amines of Formula (IV) can be treated with a carboxylic acid, with standard coupling agents, such as but not limited to 1-alkyl-2-chloropyridinium salt or preferably polymer-supported 1-alkyl-2-chloropyridinium salt (polymer-supported Mukaiyama's reagent), 1-methyl-2-chloropyridinium iodide (Mukaiyama's reagent), a carbodiimide (such as DCC, DIC, EDC) and HOBt, HATU, TBTU, PyBOP® and other such reagents well known to those skilled in the art, in the presence or absence of bases such as TEA, DIEA, NMM in a suitable solvent such as DCM, THF or DMF, at a temperature between 20° C. to 50° C., preferably at room temperature, for a few hours, e.g. one hour to 24 h.

The compounds of Formula (VII), wherein R², R⁴ are defined as above, thus obtained can be converted into compounds of Formula (VI), wherein R¹, R², R⁴ are defined as above, by treatment with a compound of Formula R¹(CH₂)—X, wherein R¹ is defined as above and X is a suitable leaving group, such as but not limited to Cl, Br, I, OMs, OTf and others known to those skilled in the art. The reaction is performed in the presence of a suitable base, such as but not limited to K₂CO₃, Na₂CO₃, NaHCO₃, NaOH, KOH, KOtBu, NaH, LDA, LiHMDS, BuLi, preferably NaH, in the presence of absence of NaI or KI (in catalytic or stoichiometric amount) in a suitable solvent such as but not limited to THF, dioxane, DMF, DMSO, preferably DMF, at a temperature between −80° C. to 160° C., preferably at −10° C. to 25° C., for a few hours, e.g. one hour to 48 h.

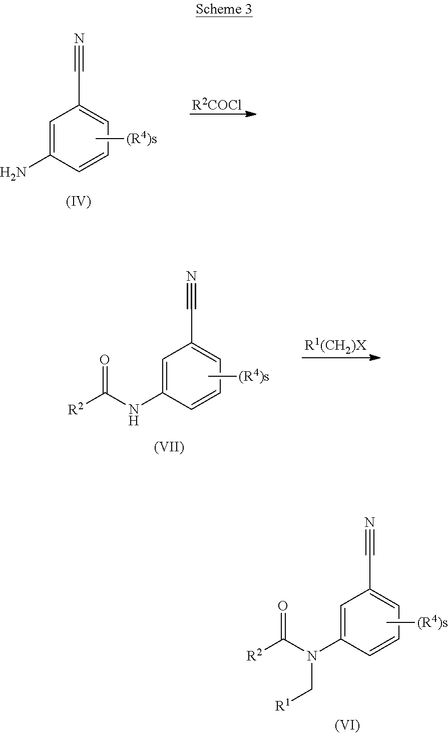

Alternatively, the compounds of Formula (VIb), wherein R² and R⁴ are as defined above, and R⁵ is Ar or Het can be prepared as depicted in Scheme 4. Compounds of Formula VIa, wherein R² and R⁴ are as defined above and R¹ is a substituted or unsubstituted iodophenyl group (such as but not limited to 3-iodophenyl or 4-iodophenyl), can be obtained as described above and depicted in Scheme 3. Conversion of these compounds of Formula (VIa) to the compounds of Formula (VIb) can be achieved by reaction with an appropriate aryl boronic acid, aryl boronic ester, heteroaryl boronic acid or heteroaryl boronic ester R⁵B(OR)₂. The reaction is performed in the presence of a suitable catalyst, such as but not limited to PdCl₂(PPh₃)₂, Pd(PPh₃)₄ and other known to those skilled in the art, in the presence of a suitable base such as but not limited to CsF, Cs₂CO₃, K₂CO₃, in the presence of absence of additional ligands, such as but not limited to PPh₃, X-Phos, S-Phos, in a suitable solvent such as a mixture dioxane, toluene, acetone, water or mixtures in variable proportions of the aforementioned solvents, at a temperature between 20° C. and 180° C., preferably between 80° C. and 150° C., with or without microwave irradiation, or under other conditions known to those skilled in the art for performing a Suzuki coupling reaction.

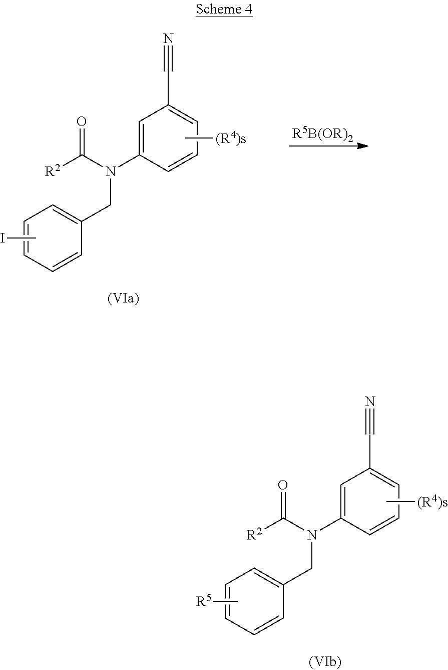

“cycloalkyl” denotes a monovalent saturated carbocyclic ring having 3 to 7 carbon atoms. Preferred cycloalkyl groups are cyclopropyl, cyclobutyl, cyclopentyl and cyclohexyl.

“cycloalkylen” denotes a divalent saturated carbocyclic ring having 3 to 7 carbon atoms.

“cycloalkylalkylen” denotes a carbon chain having 1 to 6 carbon atoms wherein 1 H atom is substituted by a cycloalkyl group.

In the compounds of Formula (I), wherein a substituent occurs more than once, such as R⁴, each of them has the meaning hereby defined, independently from one anothers.

The group A preferably denotes a branched or linear alkyl having 1 to 6 C-atoms, wherein one or more, preferably 1 to 7 H-atoms may be replaced by Hal, OR³, CN, or N(R³)₂ and wherein one or more, preferably 1 to 7 non-adjacent CH₂-groups may be replaced by O, NR³ or S and/or by —CH═CH— or —C≡C— groups.

Alternatively, A denotes a linear or branched alkyl having 1 to 6 carbon atoms, wherein 1 H atom may be replaced by Ar, preferably a phenyl group, and wherein 1 to 3 CH₂ groups may be replaced by —O—.

R¹ preferably denotes —(CH₂)ₙ—Ar, or —(CH₂)ₙHet wherein n is as defined above. More preferably, R¹ is —(CH₂)ₙ—Ar, or —(CH₂)ₙHet, wherein n is 0 or 1, and most preferably, R¹ is —(CH₂)ₙ—Ar, or —(CH₂)ₙHet wherein n is 0.

R¹ is preferably selected from the following groups:

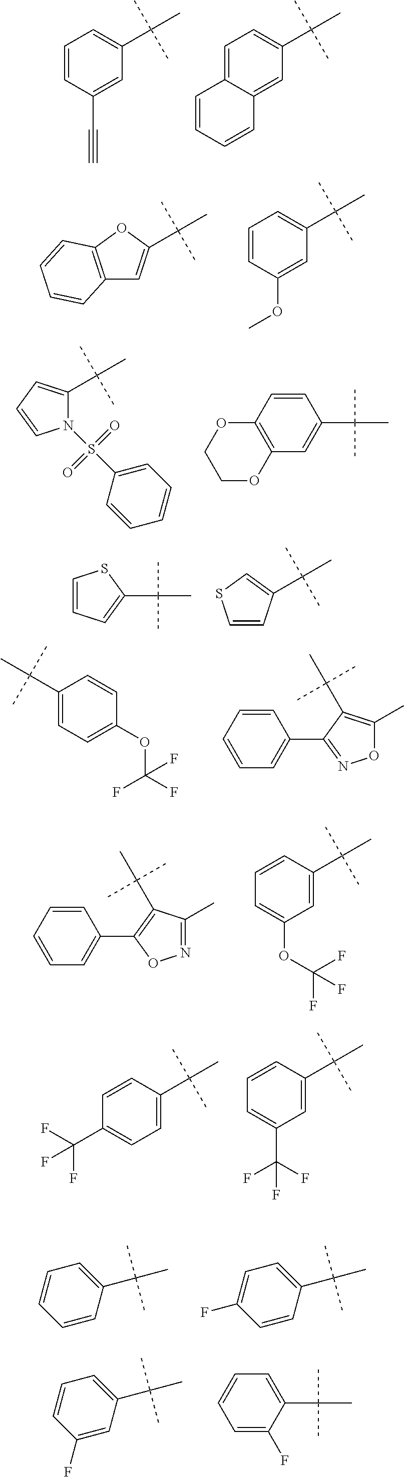

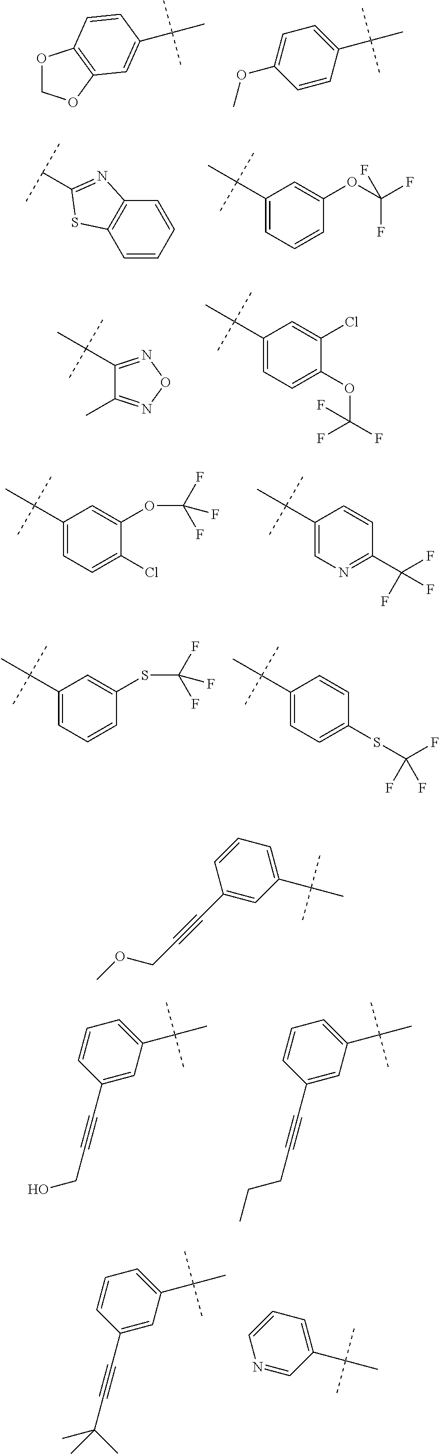

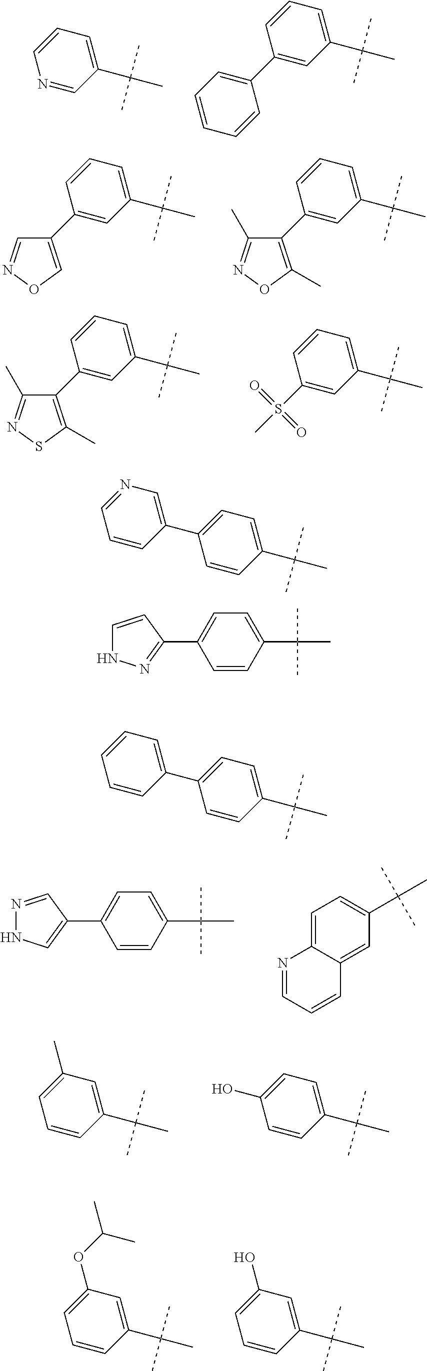

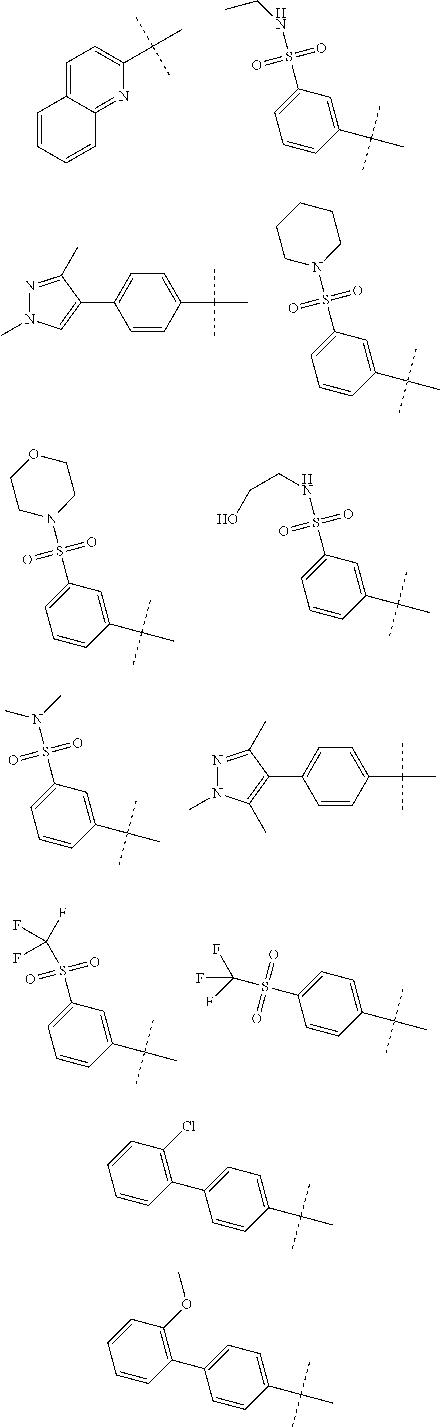

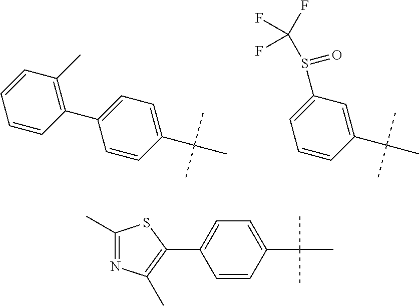

R² are preferably linear or branched alkyl having 1 to 8 carbon atoms. More preferably, R² denotes methyl, ethyl, propyl, iso-propyl, n-butyl, iso-butyl, tert-butyl, pentyl or iso-pentyl. In a more preferred embodiment, R² denotes a linear alkyl having 3 to 8 carbon atoms. Alternatively R² denotes Het. When R² is Het, it is preferably selected from pyridine, morpholine, pyran, dihydro- or tetrahydro-pyrane,

R⁴ preferably denotes H, CH₃ or Hal. Most preferably R⁴ is H, F or Cl.

Ar preferably denotes a monocyclic or fused bicyclic, unsaturated or aromatic carbocyclic ring having 6 to 14 carbon atoms which may be unsubstituted or monosubstituted, disubstituted by Hal, —CH₂OR³, —OR³, —CF₃, —OCF₃, —CN, —(CH₂)_{T}—C≡C—R⁶, —(CH₂)_{T}—CH═CH—R⁶, Ar′ or Het,

wherein R⁶ and T are as defined above.

More preferably Ar denotes the following group:

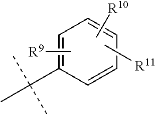

Wherein R⁹, R¹⁰ and R¹¹, are independently selected from H, Hal, —CH₂OR³, —OR³, —CF₃, —OCF₃, —CN, —(CH₂)_{T}—C≡C—R⁶, —(CH₂)_{T}—CH═CH—R⁶, Ar′, Het or SO₂(C₁-C₆)alkyl,

Whereby R³, R⁶ and T are as above defined.

Most preferably, Ar denotes one of the following groups:

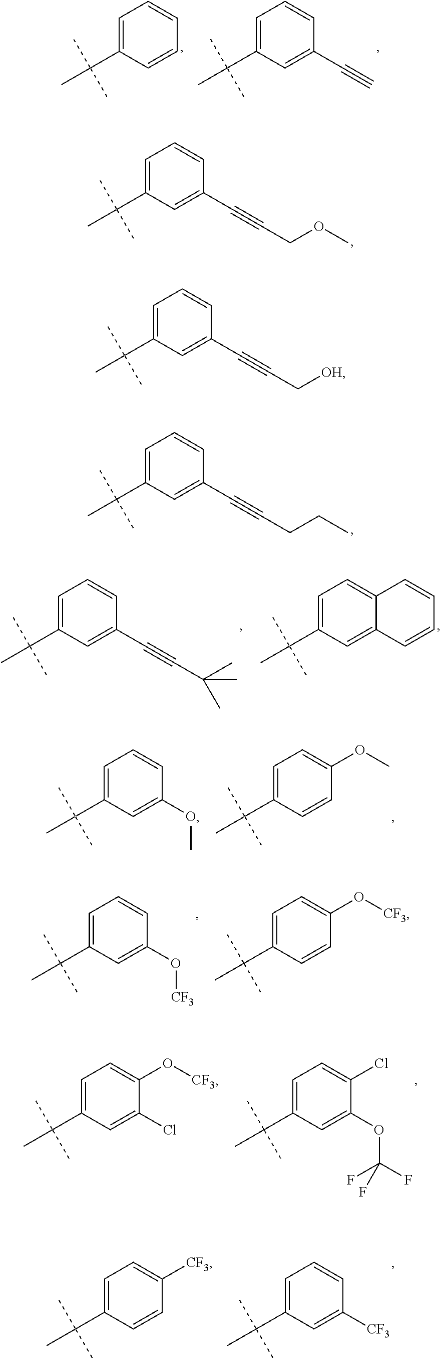

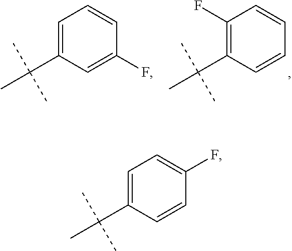

Ar′ preferably denotes a phenyl group unsubstituted or substituted with 1 or 2 groups selected from Hal, A and an alkyl having 1 to 6 carbon atoms.

Het preferably denotes a monocyclic or bicyclic, saturated, unsaturated or aromatic heterocyclic ring, having 1 to 2 N atoms and/or 1 O or S atom, which may be unsubstituted, monosubstituted, or disubstituted by Hal, —CH₂OR³, —OR³, —CF₃, —OCF₃, —CN, —(CH₂)_{T}—C≡C—R⁶, —(CH₂)_{T}—CH═CH—R⁶, Ar or Het′,

wherein R⁶ and T are as defined above.

More preferably, Het denotes, not withstanding further substitutions, for example, 2- or 3-furyl, 2- or 3-thienyl, 1-, 2- or 3-pyrrolyl, 1-, 2-, 4- or 5-imidazolyl, 1-, 3-, 4- or 5-pyrazolyl, 2-, 4- or 5-oxazolyl, 3-, 4- or 5-isoxazolyl, 2-, 4- or 5-thiazolyl, 3-, 4- or 5-isothiazolyl, 2-, 3- or 4-pyridyl, 2-, 4-, 5- or 6-pyrimidinyl, furthermore preferably 1,2,3-triazol-1-, -4- or -5-yl, 1,2,4-triazol-1-, -3- or -5-yl, 1- or 5-tetrazolyl, 1,2,3-oxadiazol-4- or -5-yl, 1,2,4-oxadiazol-3- or -5-yl, 1,3,4-thiadiazol-2- or -5-yl, 1,2,4-thiadiazol-3- or -5-yl, 1,2,3-thiadiazol-4- or -5-yl, 3- or 4-pyridazinyl, pyrazinyl, 1-, 2-, 3-, 4-, 5-, 6- or 7-indolyl, indazolyl, 4- or 5-isoindolyl, 1-, 2-, 4- or 5-benzimidazolyl, 1-, 3-, 4-, 5-, 6- or 7-benzopyrazolyl, 2-, 4-, 5-, 6- or 7-benzoxazolyl, 3-, 4-, 5-, 6- or 7-benzisoxazolyl, 2-, 4-, 5-, 6- or 7-benzothiazolyl, 2-, 4-, 5-, 6- or 7-benzisothiazolyl, 4-, 5-, 6- or 7-benz-2,1,3-oxadiazolyl, 2-, 3-, 4-, 5-, 6-, 7- or 8-quinolyl, 1-, 3-, 4-, 5-, 6-, 7- or 8-isoquinolyl, 3-, 4-, 5-, 6-, 7- or 8-cinnolinyl, 2-, 4-, 5-, 6-, 7- or 8-quinazolinyl, 5- or 6-quinoxalinyl, 2-, 3-, 5-, 6-, 7- or 8-2H-benzo-1,4-oxazinyl, furthermore preferably 1,3-benzodioxol-5-yl, 1,4-benzodioxane-6-yl, 2,1,3-benzothiadiazol-4- or -5-yl or 2,1,3-benzoxadiazol-5-yl.

The heterocyclic radicals may also be partially or fully hydrogenated.

Het can thus also denote, for example, 2,3-dihydro-2-, -3-, -4- or -5-furyl, 2,5-dihydro-2-, -3-, -4- or -5-furyl, tetrahydro-2- or -3-furyl, 1,3-dioxolan-4-yl, tetrahydro-2- or -3-thienyl, 2,3-dihydro-1-, -2-, -3-, -4- or -5-pyrrolyl, 2,5-dihydro-1-, -2-, -3-, -4- or -5-pyrrolyl, 1-, 2- or 3-pyrrolidinyl, tetrahydro-1-, -2- or -4-imidazolyl, 2,3-dihydro-1-, -2-, -3-, -4- or -5-pyrazolyl, tetrahydro-1-, -3- or -4-pyrazolyl, 1,4-dihydro-1-, -2-, -3- or -4-pyridyl, 1,2,3,4-tetrahydro-1-, -2-, -3-, -4-, -5- or -6-pyridyl, 1-, 2-, 3- or 4-piperidinyl, 2-, 3- or 4-morpholinyl, tetrahydro-2-, -3- or -4-pyranyl, 1,4-dioxaneyl, 1,3-dioxane-2-, -4- or -5-yl, hexahydro-1-, -3- or -4-pyridazinyl, hexahydro-1-, -2-, -4- or -5-pyrimidinyl, 1-, 2- or 3-piperazinyl, 1,2,3,4-tetrahydro-1-, -2-, -3-, -4-, -5-, -6-, -7- or -8-quinolyl, 1,2,3,4-tetrahydro-1-, -2-, -3-, -4-, -5-, -6-, -7- or -8-isoquinolyl, 2-, 3-, 5-, 6-, 7- or 8-3,4-dihydro-2H-benzo-1,4-oxazinyl, furthermore preferably 2,3-methylenedioxyphenyl, 3,4-methylenedioxyphenyl, 2,3-ethylenedioxyphenyl, 3,4-ethylenedioxyphenyl, 3,4-(difluoromethylenedioxy)phenyl, 2,3-dihydrobenzofuran-5- or -6-yl, 2,3-(2-oxomethylenedioxy)phenyl or also 3,4-dihydro-2H-1,5-benzodioxepin-6- or -7-yl, furthermore preferably 2,3-dihydrobenzofuranyl or 2,3-dihydro-2-oxofuranyl.

The more preferred Het groups are selected from the following groups:

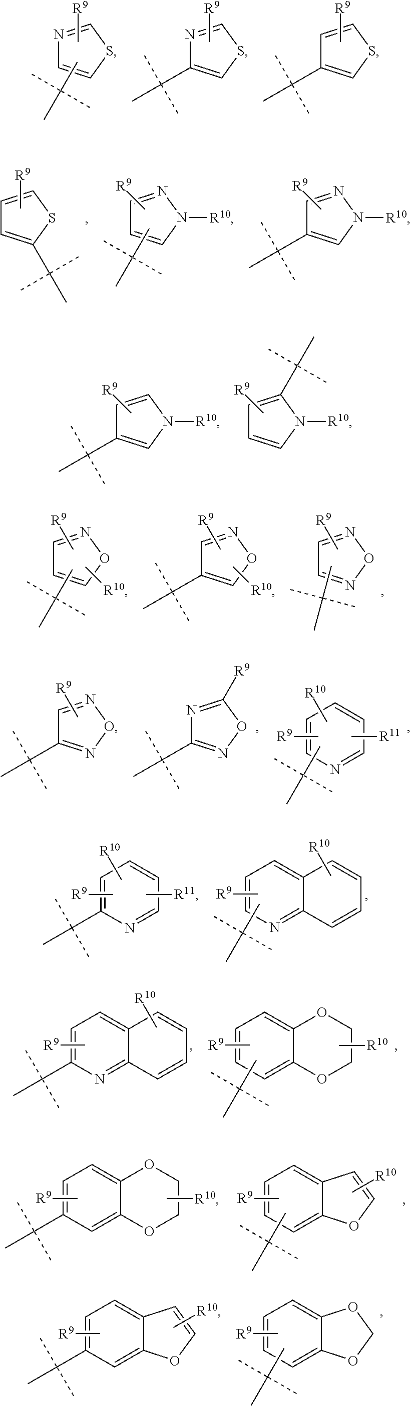

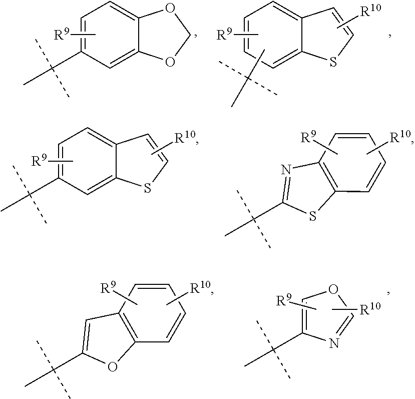

Wherein R⁹, R¹⁰ and R¹¹ are independently selected from H, A, Hal, linear or branched alkyl having 1 to 6 carbon atoms, Ar, OR³, CN, CF₃, and OCF₃, SO₂Ar, SO₂Het, SO₂(C₁-C₆)alkyl whereby R³ is as defined above.

Most preferably, Het is selected from the following groups:

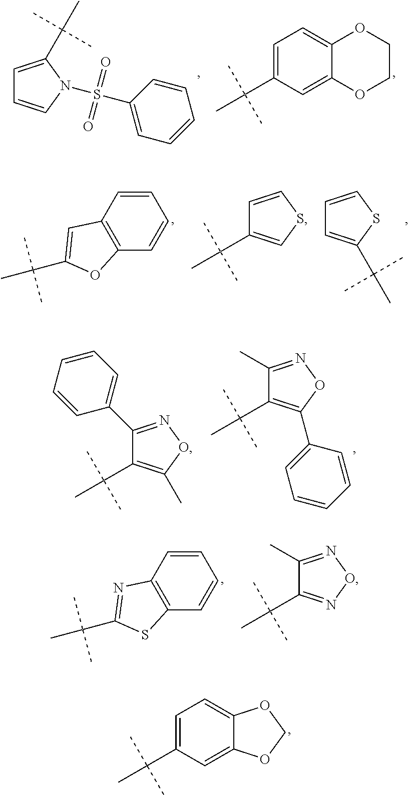

Preferred compounds of the present invention are selected from the following group:

| EX. | Formula |
|---|---|
| 1 | 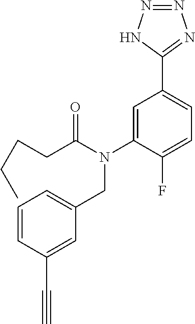 |
| 2 | 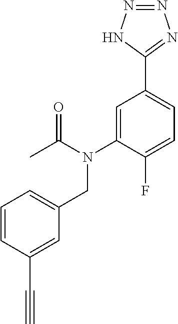 |
| 3 | 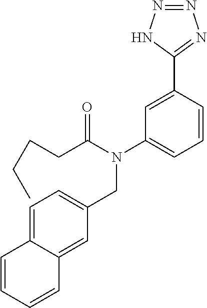 |
| 4 | 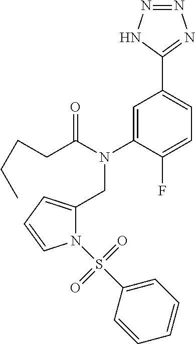 |
| 5 | 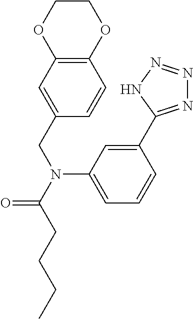 |
| 6 | 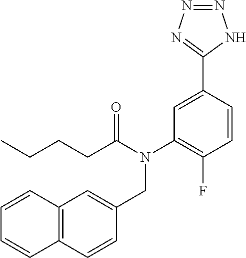 |
| 7 | 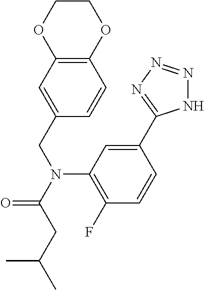 |
| 8 | 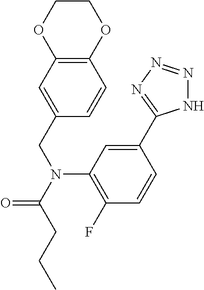 |
| 9 | 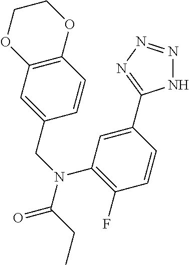 |
| 10 | 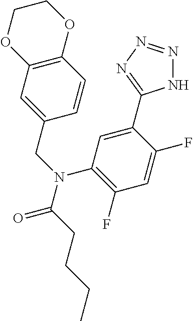 |
| 11 | 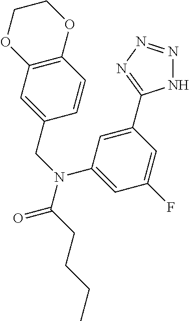 |
| 12 | 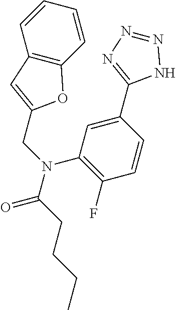 |
| 13 | 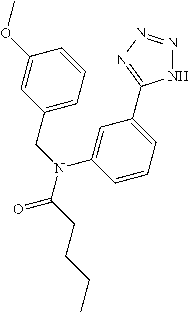 |
| 14 | 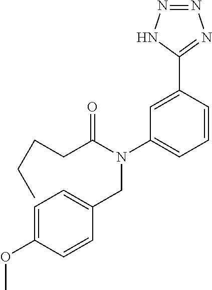 |
| 15 | 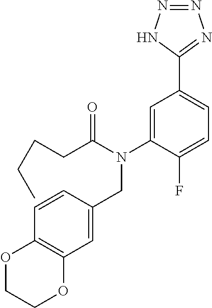 |
| 16 | 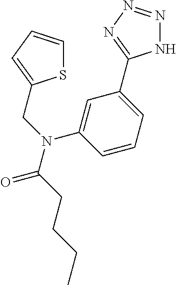 |
| 17 | 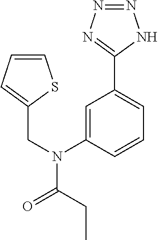 |
| 18 | 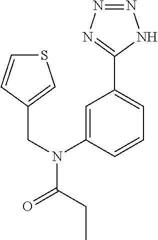 |
| 19 | 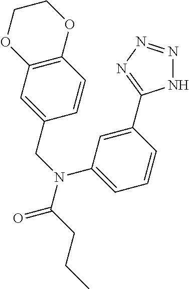 |
| 20 | 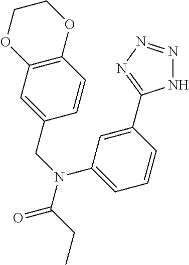 |
| 21 | 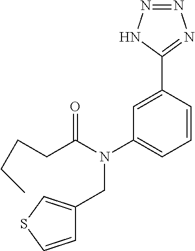 |
| 22 | 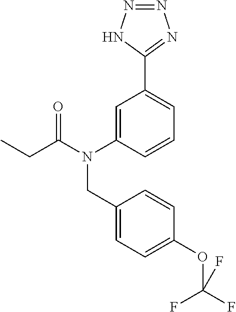 |
| 23 | 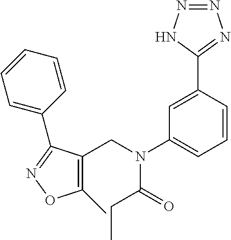 |
| 24 | 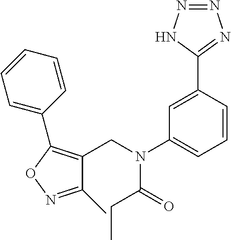 |
| 25 | 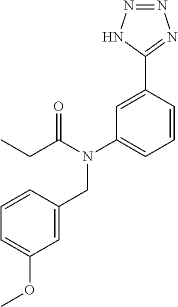 |
| 26 | 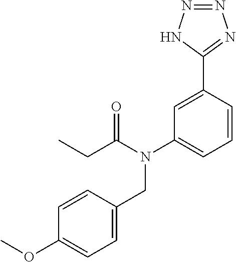 |
| 27 | 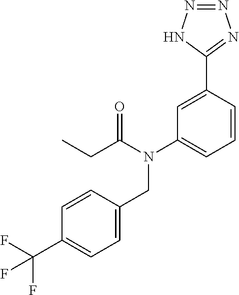 |
| 28 | 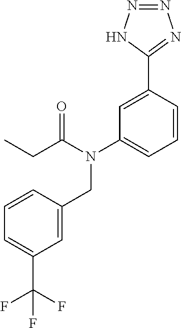 |
| 29 | 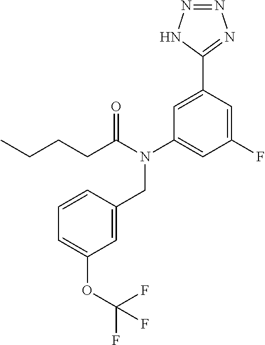 |
| 30 | 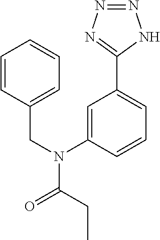 |
| 31 | 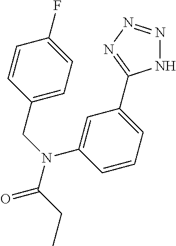 |
| 32 | 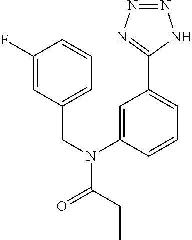 |
| 33 | 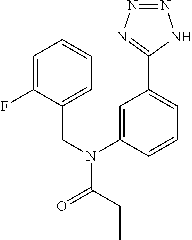 |
| 34 | 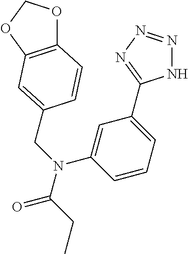 |
| 35 | 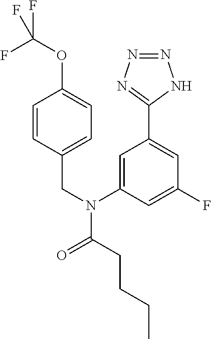 |
| 36 | 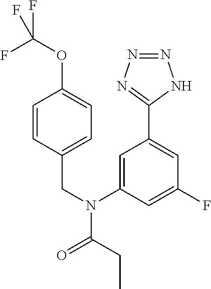 |
| 37 | 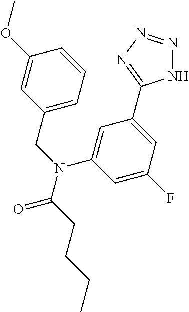 |
| 38 | 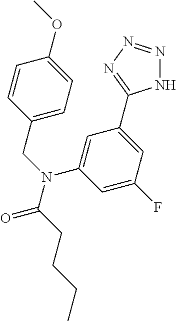 |
| 39 | 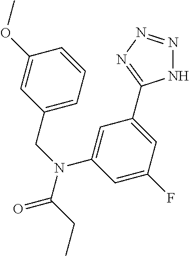 |
| 40 | 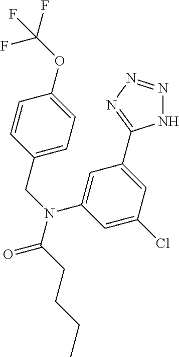 |
| 41 | 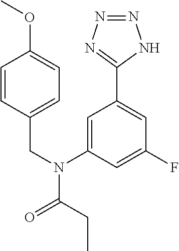 |
| 42 | 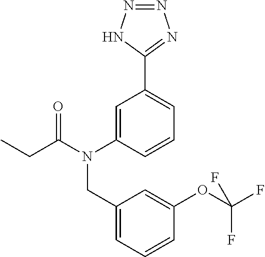 |
| 43 | 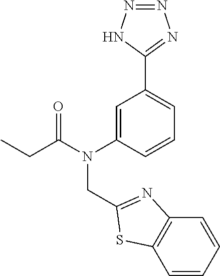 |
| 44 | 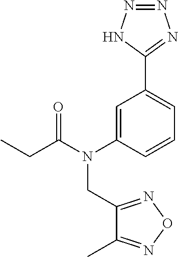 |
| 45 | 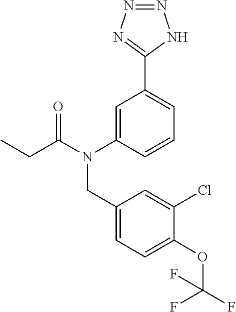 |
| 46 | 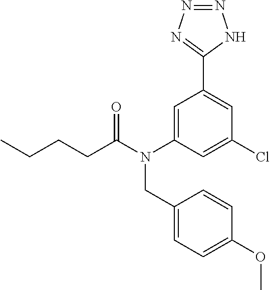 |
| 47 | 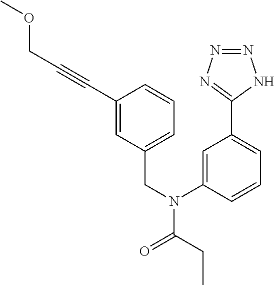 |
| 48 | 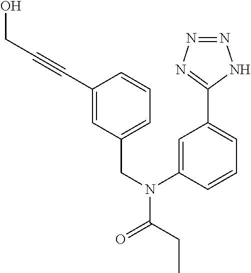 |
| 49 | 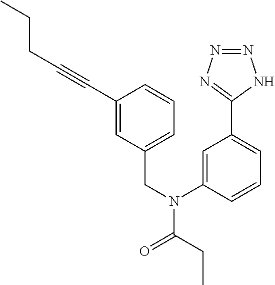 |
| 50 | 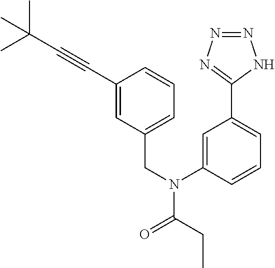 |
| 51 | 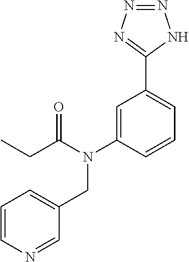 |
| 52 | 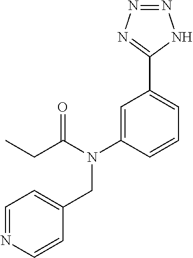 |
| 53 | 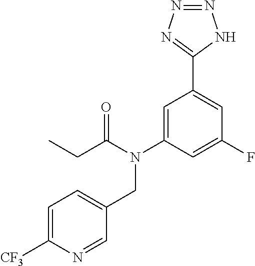 |
| 54 | 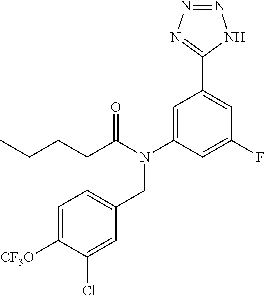 |
| 55 | 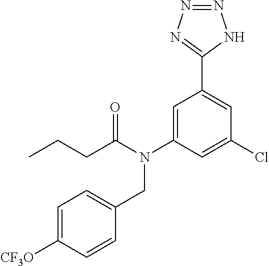 |
| 56 | 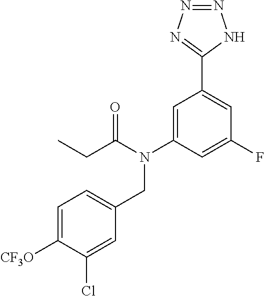 |
| 57 | 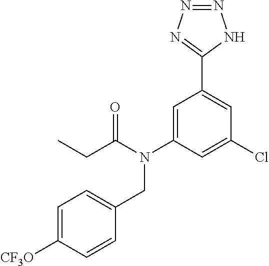 |
| 58 | 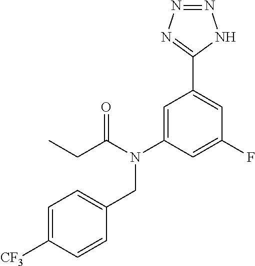 |
| 59 | 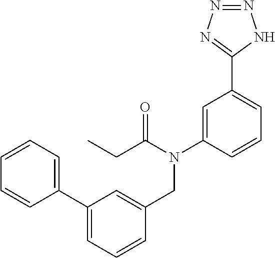 |
| 60 | 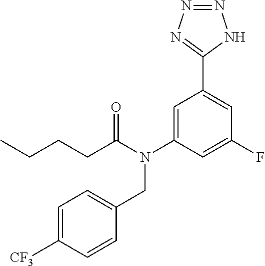 |
| 61 | 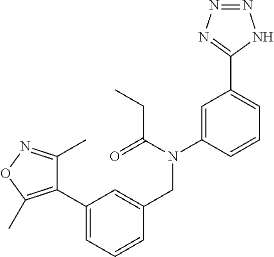 |
| 62 | 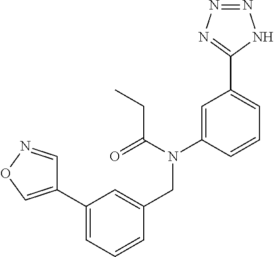 |
| 63 | 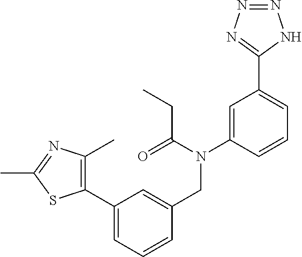 |
| 64 | 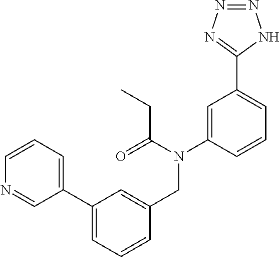 |
| 65 | 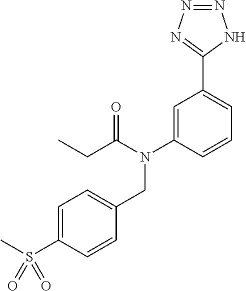 |
| 66 | 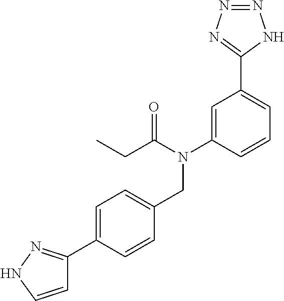 |
| 67 | 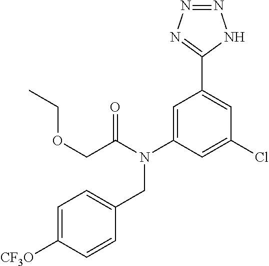 |
| 68 | 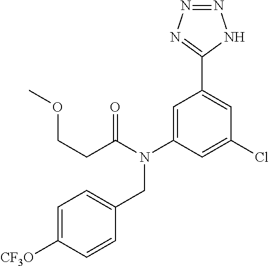 |
| 69 | 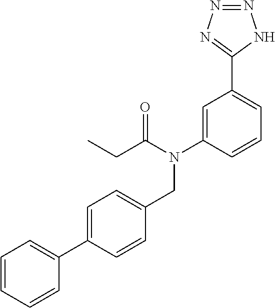 |
| 70 | 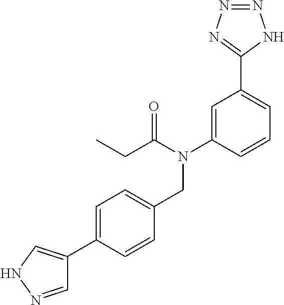 |
| 71 | 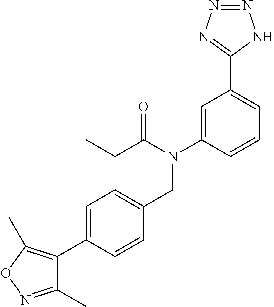 |
| 72 | 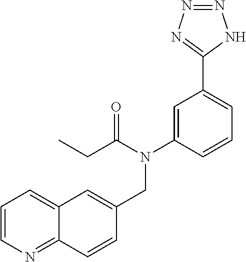 |
| 73 | 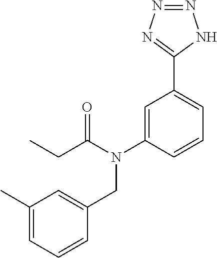 |
| 74 | 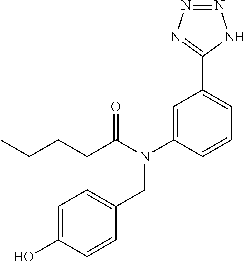 |
| 75 | 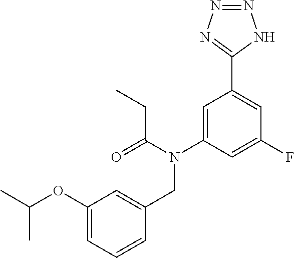 |
| 76 | 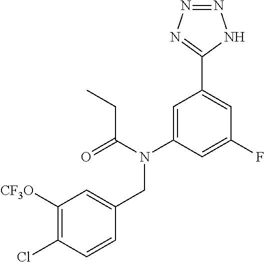 |
| 77 | 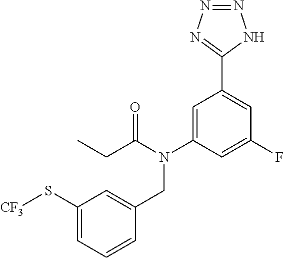 |
| 78 | 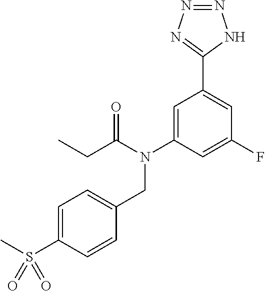 |
| 79 | 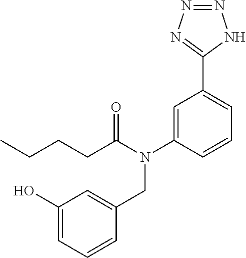 |
| 80 | 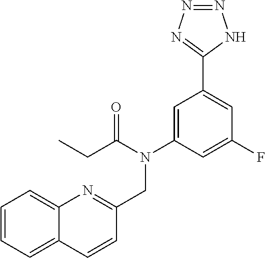 |
| 81 | 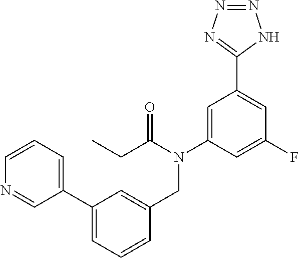 |
| 82 | 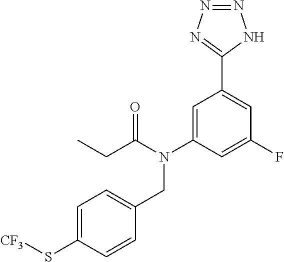 |
| 83 | 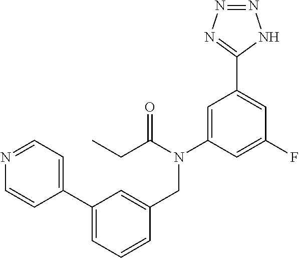 |
| 84 | 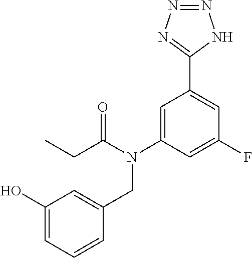 |
| 85 | 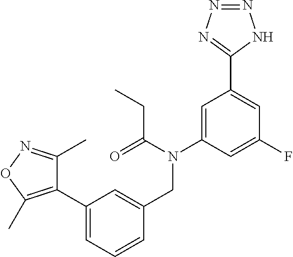 |
| 86 | 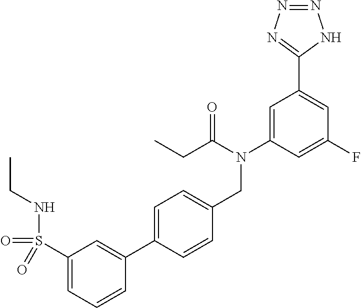 |
| 87 | 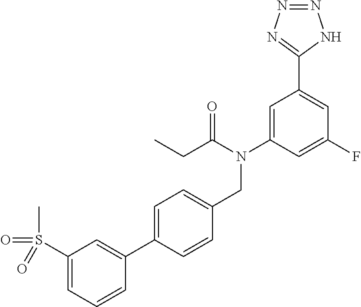 |
| 88 | 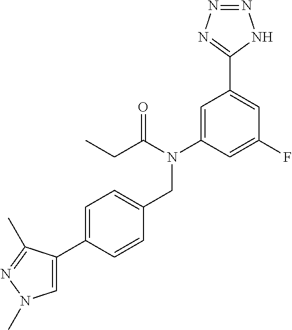 |
| 89 | 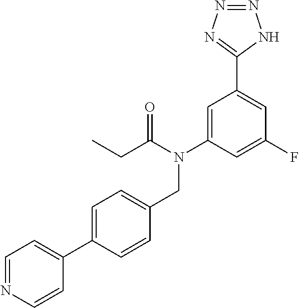 |
| 90 | 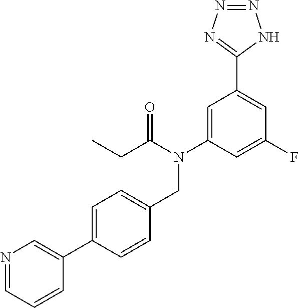 |
| 91 | 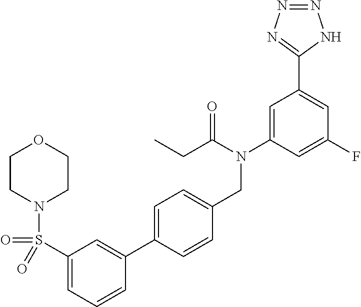 |
| 92 | 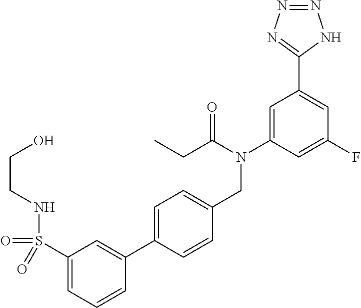 |
| 93 | 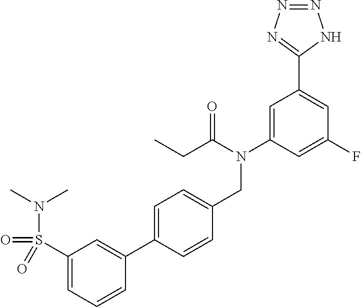 |
| 94 | 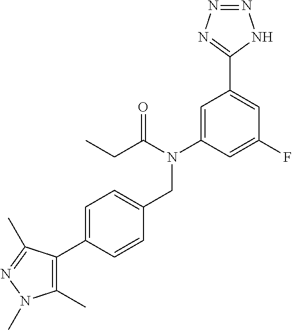 |
| 95 | 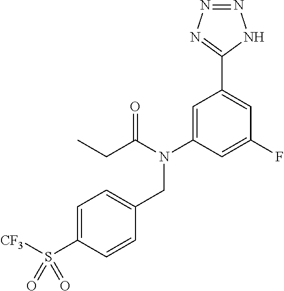 |
| 96 | 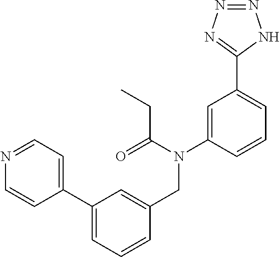 |
| 97 | 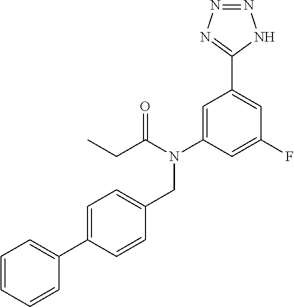 |
| 98 | 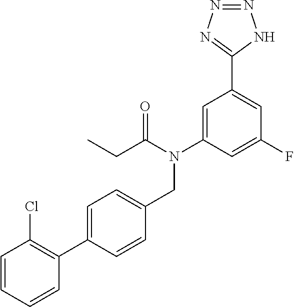 |
| 99 | 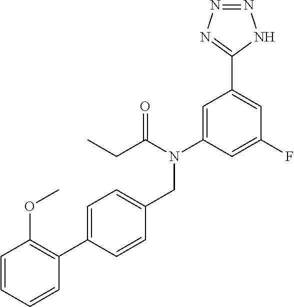 |
| 100 | 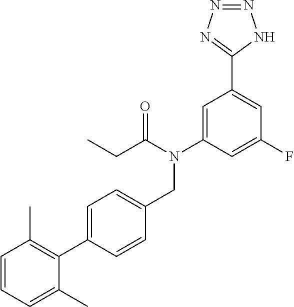 |
| 101 | 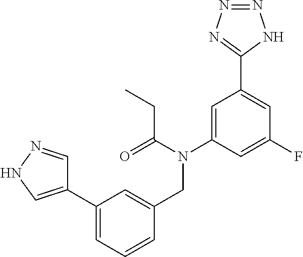 |
| 102 | 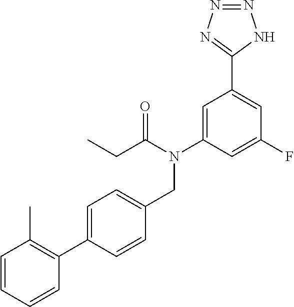 |
| 103 | 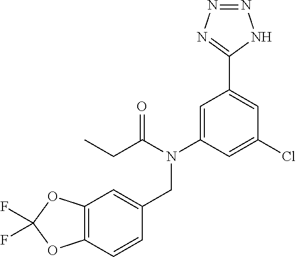 |
| 104 | 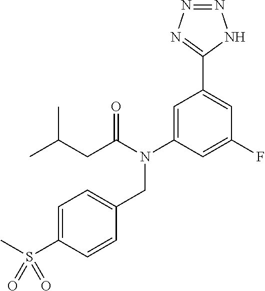 |
| 105 | 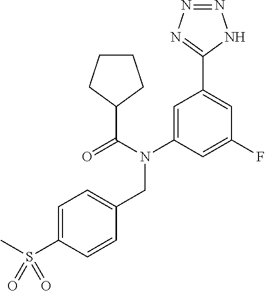 |
| 106 | 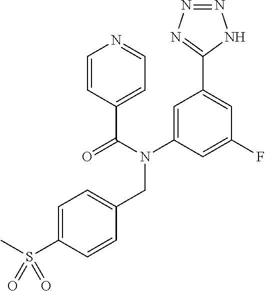 |
| 107 | 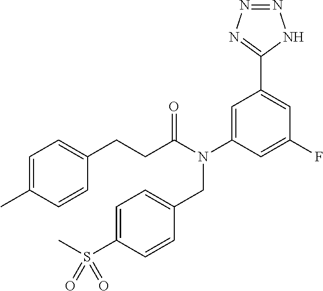 |
| 108 | 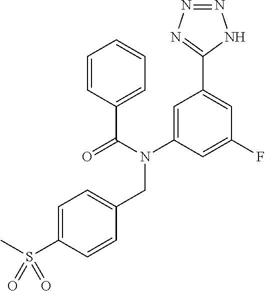 |
| 109 | 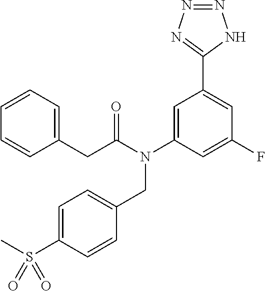 |
| 110 | 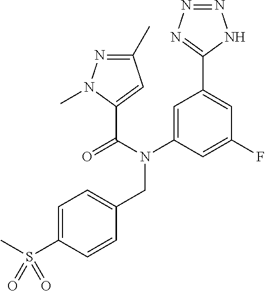 |
| 111 | 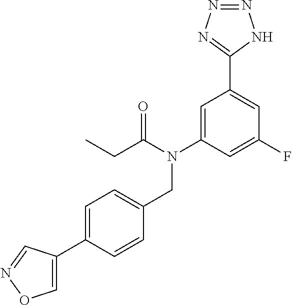 |
| 112 | 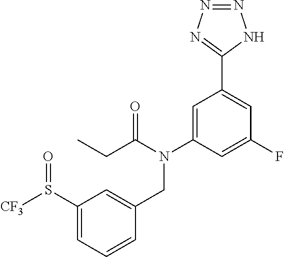 |
| 113 | 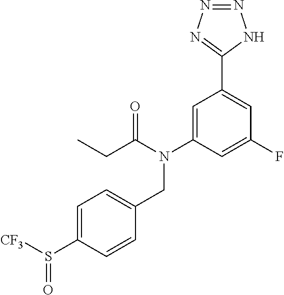 |
| 114 | 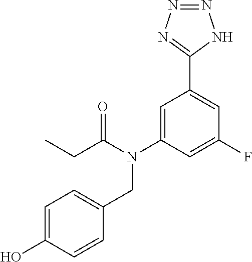 |
| 115 | 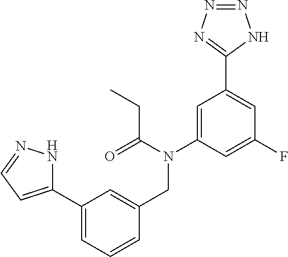 |
| 116 | 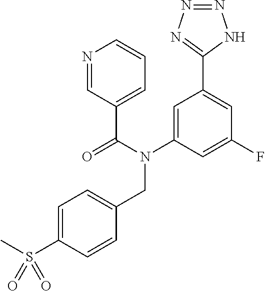 |
| 117 | 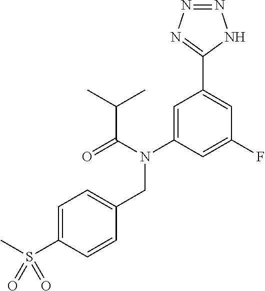 |
| 118 | 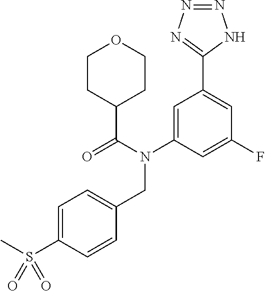 |
| 119 | 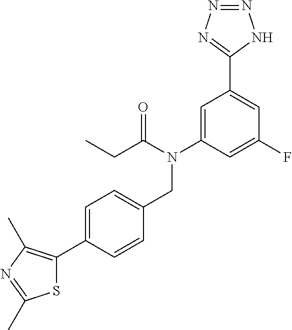 |

“Pharmaceutically acceptable cationic salts or complexes” is intended to define such salts as the alkali metal salts, (e.g. sodium and potassium), alkaline earth metal salts (e.g. calcium or magnesium), aluminium salts, ammonium salts and salts with organic amines such as with methylamine, 2-N-morpholinoethanol, dimethylamine, trimethylamine, ethylamine, triethylamine, morpholine, N-Me-D-glucamine, N,N′-bis(phenylmethyl)-1,2-ethanediamine, ethanolamine, diethanolamine, ethylenediamine, N-methylmorpholine, piperidine, benzathine (N,N′-dibenzylethylenediamine), choline, ethylene-diamine, benethamine (N-benzylphenethylamine), diethylamine, piperazine, thromethamine (2-amino-2-hydroxymethyl-1,3-propanediol), procaine as well as amines of formula -NRR′R″ wherein R, R′, R″ is independently hydrogen, alkyl or benzyl.

“Pharmaceutically acceptable salts or complexes” refers to salts or complexes of the below-identified compounds of Formula I that retain the desired biological activity. Examples of such salts include, but are not restricted to, acid addition salts formed with inorganic acids (e.g. hydrochloric acid, hydrobromic acid, sulfuric acid, phosphoric acid, nitric acid, and the like), and salts formed with organic acids such as acetic acid, oxalic acid, tartaric acid, succinic acid, malic acid, fumaric acid, maleic acid, ascorbic acid, benzoic acid, tannic acid, pamoic acid, alginic acid, polyglutamic acid, naphthalene sulfonic acid, naphthalene disul-fonic acid, and poly-galacturonic acid. Said compounds can also be administered as pharmaceutically acceptable quaternary salts known by a person skilled in the art, which specifically include the quarternary ammonium salt of the Formula -NRR′R″+Z—, wherein R, R′, R″ is independently hydrogen, alkyl, or benzyl, and Z is a counterion, including chloride, bromide, iodide, —O-alkyl, toluenesulfonate, methylsulfonate, sulfonate, phosphate, or carboxylate (such as benzoate, succinate, acetate, glycolate, maleate, malate, fumarate, citrate, tartrate, ascorbate, cinnamoate, mandeloate, and diphenylacetate).

“Pharmaceutically active derivative” or “pharmaceutically usable derivative” refers to any compound that, upon administration to the recipient, is capable of providing directly or indirectly, the activity disclosed herein.

Throughout the specification, the term leaving group preferably denotes Cl, Br, I or a reactively modified OH group, such as, for example, an activated ester, an imidazolide or alkylsulfonyloxy having 1-6 carbon atoms (preferably methylsulfonyloxy or trifluoromethylsulfonyloxy) or arylsulfonyloxy having 6-10 carbon atoms (preferably phenyl- or p-tolylsulfonyloxy).

Radicals of this type for activation of the carboxyl group in typical acylation reactions are described in the literature (for example in the standard works, such as Houben-Weyl, Methoden der organischen Chemie [Methods of Organic Chemistry], Georg-Thieme-Verlag, Stuttgart).

Activated esters are advantageously formed in situ, for example through addition of HOBt or N-hydroxysuccinimide

The term “solvates” is taken to mean adductions of inert solvent molecules onto the compounds which form owing to their mutual attractive force. Solvates are, for example, mono- or dihydrates or alcoholates.

The formula (I) and related formulae also encompass mixtures of the compounds of the formula (I), for example mixtures of two enantiomers or diastereomers, for example in the ratio 1:1, 1:2, 1:3, 1:4, 1:5, 1:10, 1:100 or 1:1000.

In a first aspect, the invention provides spiro derivatives according to Formula (I) and related formulae that are useful in the treatment and/or prevention of diseases selected from allergic diseases such as allergic asthma, allergic rhinitis, allergic conjunctivitis, and inflammatory dermatoses such as atopic dermatitis, contact hypersensitivity, allergic contact dermatitis, chronic urticaria/chronic idiopathic/autoimmune urticaria, drug-induced exanthems (e.g. toxic epidermal necrolysis or Lyell's syndrome/Stevens-Johnson syndrome/drug hypersensitivity syndrome), photodermatosis or polymorphous light eruption (e.g. photo-irritant contact dermatitis, photoallergic contact dermatitis, chronic actinic dermatitis), and myositis neurodegenerative disorders such as neuropatic pain and other diseases with an inflammatory component such as rheumatoid arthritis, multiple sclerosis, osteoarthritis, and inflammatory bowel disease (IBD).

In one embodiment the compounds according to Formula (I) are suitable as modulators of CRTH2. Therefore, the compounds of the present invention are also particularly useful for the treatment and/or prevention of disorders, which are mediated by CRTH2 activity. Said treatment involves the modulation of CRTH2 in mammals and particular in humans. The modulators of CRTH2 are selected from the group consisting of an inverse agonist, an antagonist, a partial agonist and an agonist of CRTH2.

In one embodiment, the modulators of CRTH2 are inverse agonists of CRTH2.

In another embodiment, the modulators of CRTH2 are antagonists of CRTH2.

In another embodiment, the modulators of CRTH2 are partial agonists of CRTH2.

In another embodiment, the modulators of CRTH2 are agonists of CRTH2.

The compounds according to Formula (I) are suitable for use as a medicament.

Compounds of Formula (I) include also their geometrical isomers, their optically active forms as enantiomers, diastereomers, its racemate forms, as well as pharmaceutically acceptable salts thereof,

In a second aspect, the invention provides the use of a tetrazole derivative according to Formula (I) and related formulae, for the preparation of a medicament for the treatment and/or prevention of a disease selected from allergic diseases such as allergic asthma, allergic rhinitis, allergic conjunctivitis, and inflammatory dermatoses such as atopic dermatitis, contact hypersensitivity, allergic contact dermatitis, chronic urticaria/chronic idiopathic/autoimmune urticaria, drug-induced exanthems (e.g. toxic epidermal necrolysis or Lyell's syndrome/Stevens-Johnson syndrome/drug hypersensitivity syndrome), photodermatosis or polymorphous light eruption (e.g. photo-irritant contact dermatitis; photoallergic contact dermatitis; chronic actinic dermatitis), and myositis, neurodegenerative disorders such as neuropatic pain and other diseases with an inflammatory component such as rheumatoid arthritis, multiple sclerosis, osteoarthritis, and inflammatory bowel disease (IBD) and other diseases and disorders associated with CTRH2 activity.

In a third aspect, the invention provides a method for treating and/or preventing a patient suffering from a disease selected from allergic diseases such as allergic asthma, allergic rhinitis, allergic conjunctivitis, and inflammatory dermatoses such as atopic dermatitis, contact hypersensitivity, allergic contact dermatitis, chronic urticaria/chronic idiopathic/autoimmune urticaria, drug-induced exanthems (e.g. toxic epidermal necrolysis or Lyell's syndrome/Stevens-Johnson syndrome/drug hypersensitivity syndrome), photodermatosis or polymorphous light eruption (e.g. photo-irritant contact dermatitis, photoallergic contact dermatitis, chronic actinic dermatitis), and myositis, neurodegenerative disorders such as neuropatic pain and other diseases with an inflammatory component such as rheumatoid arthritis, multiple sclerosis, osteoarthritis, and inflammatory bowel disease (IBD) and other diseases and disorders associated with CTRH2 activity, by administering a compound according to Formula (I) or related formulae.

The term “preventing”, as used herein, should be understood as partially or totally preventing, inhibiting, alleviating, or reversing one or more symptoms or cause(s) of allergic disease or inflammatory dermatitis.

The compounds of the invention, together with a conventionally employed adjuvant, carrier, diluent or excipient may be placed into the form of pharmaceutical compositions and unit dosages thereof, and in such form may be employed as solids, such as tablets or filled capsules, or liquids such as solutions, suspensions, emulsions, elixirs, or capsules filled with the same, all for oral use, or in the form of sterile injectable solutions for parenteral (including subcutaneous use). Such pharmaceutical compositions and unit dosage forms thereof may comprise ingredients in conventional proportions, with or without additional active compounds or principles, and such unit dosage forms may contain any suitable effective amount of the active ingredient commensurate with the intended daily dosage range to be employed.

In a fourth aspect, the invention provides a pharmaceutical composition comprising a tetrazole derivative according to Formulae (I) or related formulae, together with a pharmaceutically acceptable excipient or carrier.

In a fifth aspect, the invention provides a pharmaceutical composition comprising a compound according to Formulae (I) or related formulae, together with a biologically active compound. In particular, the pharmaceutical composition contains a compound of Formula (I) in combination with an anti-allergic drug.

In another embodiment, the pharmaceutical composition contains a compound of Formula (I) in combination with an antihistamine, a decongestant, an anticholinergic, a methylxanthine, a cromolyn, a corticosteroid or a leukotriene modulator.

In another embodiment, the pharmaceutical composition contains a compound of Formula (I) in combination with a drug used in the treatment of disease or disorder associated with CTRH2 activity.

In a sixth aspect, the present invention provides a method of reducing the dose of an anti-allergic drug. In particular, the present invention provides a mean of reducing the dose of antihistamines, decongestants, anticholinergics, methylxanthines, cromolyns, corticosteroids or leukotriene modulators. In another embodiment, the present invention provides a mean to decrease the dose of drug used in the treatment of disease or disorder associated with CTRH2 activity.

The compounds of the invention are typically administered in form of a pharmaceutical composition. Such compositions can be prepared in a manner well known in the pharmaceutical art and comprise at least one active compound. Generally, the compounds of this invention are administered in a pharmaceutically effective amount. The amount of the compound actually administered will typically be determined by a physician, in the light of the relevant circumstances, including the condition to be treated, the chosen route of administration, the actual compound administered, the age, weight, and response of the individual patient, the severity of the patient's symptoms, and the like.

The pharmaceutical compositions of these inventions can be administered by a variety of routes including oral, rectal, transdermal, subcutaneous, intravenous, intramuscular, and intranasal. The compositions for oral administration can take the form of bulk liquid solutions or suspensions, or bulk powders. More commonly, however, the compositions are presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampoules or syringes of the liquid compositions or pills, tablets, capsules or the like in the case of solid compositions. In such compositions, compound according to the invention is usually a minor component (from about 0.1 to about 50% by weight or preferably from about 1 to about 40% by weight) with the remainder being various vehicles or carriers and processing aids helpful for forming the desired dosing form.

Liquid forms suitable for oral administration may include a suitable aqueous or non-aqueous vehicle with buffers, suspending and dispensing agents, colorants, flavors and the like. Solid forms may include, for example, any of the following ingredients, or compounds of a similar nature: a binder such as microcrystalline cellulose, gum tragacanth or gelatine; an excipient such as starch or lactose, a disintegrating agent such as alginic acid, Primogel, or corn starch; a lubricant such as magnesium stearate; a glidant such as colloidal silicon dioxide; a sweetening agent such as sucrose or saccharin; or a flavoring agent such as pepper-mint, methyl salicylate, or orange flavoring.

Injectable compositions are typically based upon injectable sterile saline or phosphate buffered saline or other injectable carriers known in the art. As above mentioned, spiro derivatives of Formula (I) in such compositions is typically a minor component, frequently ranging between 0.05 to 10% by weight with the remainder being the injectable carrier and the like.

The above-described components for orally administered or injectable compositions are merely representative. Further materials as well as processing techniques and the like are set out in Part 5 of Remington's Pharmaceutical Sciences, 20th Edition, 2000, Marck Publishing Company, Easton, Pa., which is incorporated herein by reference.

The compounds of this invention can also be administered in sustained release forms or from sustained release drug delivery systems. A description of representative sustained release materials can also be found in the incorporated materials in Remington's Pharmaceutical Sciences.

Pharmaceutical formulations can be administered in the form of dosage units, which comprise a predetermined amount of active ingredient per dosage unit. Such a unit can comprise, for example, 0.5 mg to 1 g, preferably 1 mg to 700 mg, particularly preferably 5 mg to 100 mg, of a compound according to the invention, depending on the disease condition treated, the method of administration and the age, weight and condition of the patient, or pharmaceutical formulations can be administered in the form of dosage units which comprise a predetermined amount of active ingredient per dosage unit. Preferred dosage unit formulations are those which comprise a daily dose or part-dose, as indicated above, or a corresponding fraction thereof of an active ingredient. Furthermore, pharmaceutical formulations of this type can be prepared using a process, which is generally known in the pharmaceutical art.

In a seventh aspect, the invention provides a method of synthesis of a compound according to Formulae (I) and related formulae.

The tetrazole derivatives exemplified in this invention may be prepared from readily available starting materials using the following general methods and procedures. It will be appreciated that where typical or preferred experimental conditions (i.e. reaction temperatures, time, moles of reagents, solvents etc.) are given, other experimental conditions can also be used unless otherwise stated. Optimum reaction conditions may vary with the particular reactants or solvents used, but such conditions can be determined by the person skilled in the art, using routine optimization procedures.

In a height aspect, the present invention relates to a kit separate packs of
(a) an effective amount of a compound according to formula (I) and/or related formulae and/or pharmaceutically usable derivatives, tautomers, salts, solvates and stereoisomers thereof, including mixtures thereof in all ratios,
   and
(b) an effective amount of a further medicament active ingredient
In one embodiment, the separate packs consist of distinct containers or vessels, each of them containing either the effective amount of formula (I) or an effective amount of a further active ingredient.

In a second embodiment the kit may also comprise a third vessel containing an adjuvant or a diluent.

In a third embodiment, the kit is used to prepare the pharmaceutical composition of the present invention.

In a ninth aspect, the present invention relates to a commercial package consisting of an effective amount of a compound according to formula (I), and/or pharmaceutically usable derivatives, tautomers, salts, solvates and stereoisomers thereof, including mixtures thereof in all ratios, together with instructions for the use thereof in treatment of allergic diseases and inflammatory dermatoses.

The following abbreviations refer to the abbreviations used below:

min (minute), hr (hour), g (gram), MHz (Megahertz), ml (milliliter), mmol (millimole), mM (millimolar), RT (room temperature), AcNH2 (Acetamide), AcOH (Acetic acid), ATP (Adenoside Triphosphate), BSA (Bovine Serum Albumin), Bu4NOH (Tetrabutylammonium hydroxide), CDI (1,1′-Carbonyldiimidazole), DBU (1,8-Dizabicyclo[5.4.0]undec-7-ene), DCM (Dichloromethane), DIPEA (di-isopropyl ethylamine), DMAP (4-Dimethylaminopyridine), DMSO (Dimethyl Sulfoxide), DMF (N,N-Dimethylformamide), CH3NO2 (Nitromethane), CsCO3 (Cesium carbonate), cHex (Cyclohexanes), Et3N (Triethylamine), EtOAc (Ethyl acetate), EtOH (Ethanol), HCl (hydrogen chloride), K2CO3 (Potassium Carbonate), NaI (Sodium Iodine), KCN, (Potassium cyanide), MeOH (Methanol), MgSO4 (Magnesium sulfate), NH3 (ammonia), NaH (Sodium hydride), NaHCO3 (Sodium bicarbonate), NH4Cl (Ammonium chloride), NH4(CO3)2 (ammonium carbonate), TEA (Triethyl amine), TFA (Trifluoroacetic acid), THF (Tetrahydrofuran), tBuOK (Potassium tert-butoxide), PdC12 (Palladium dichloride), PetEther (Petroleum ether), PtO2 (Platinium oxide), TBME (tert-Butyl Methyl Ether), TMSI (Trimethylsilyl iodide), Zn (Zinc powder), rt (room temperature). HPLC (High Performance Liquid Chromatography), FC (Flash Chromatography on silica gel), MS (Mass Spectrometry), NMR (Nuclear Magnetic Resonance), PBS (Phosphate Buffered Saline), SPA (Scintillation Proximity Assay), TLC (Thin Layer Chromatography), UV (Ultraviolet).

If the above set of general synthetic methods is not applicable to obtain compounds according to Formula (I) and/or necessary intermediates for the synthesis of compounds of Formula (I), suitable methods of preparation known by a person skilled in the art should be used. In general, the synthesis pathways for any individual compound of Formula (I) will depend on the specific substituents of each molecule and upon the ready availability of intermediates necessary; again such factors being appreciated by those of ordinary skill in the art. For all the protection and deprotection methods, see Philip J. Kocienski, in “Protecting Groups”, Georg Thieme Verlag Stuttgart, New York, 1994 and, Theodora W. Greene and Peter G. M. Wuts in “Protective Groups in Organic Synthesis”, Wiley Interscience, 3rd Edition 1999. Compounds of this invention can be isolated in association with solvent molecules by crystallization from evaporation of an appropriate solvent. The pharmaceutically acceptable acid addition salts of the compounds of Formula (I), which contain a basic center, may be prepared in a conventional manner. For example, a solution of the free base may be treated with a suitable acid, either neat or in a suitable solution, and the resulting salt isolated either by filtration or by evaporation under vacuum of the reaction solvent. Pharmaceutically acceptable base addition salts may be obtained in an analogous manner by treating a solution of compound of Formula (I) with a suitable base. Both types of salts may be formed or interconverted using ion-exchange resin techniques.

In the following the present invention shall be illustrated by means of some examples, which are not construed to be viewed as limiting the scope of the invention.

### General:

The HPLC data provided in the examples described below were obtained as followed.

Condition A: Column Waters Xbridge™ C₈ 50 mm×4.6 mm at a flow of 2 mL/min; 8 min gradient from 0.1% TFA in H₂O to 0.07% TFA in CH₃CN.

Condition B: C18 BDS (4.6×250)mm, SC\244 at a flow of 0.7 mL/min; 10 min gradient from 0.1% TFA in H₂O to CH₃CN.

UV detection (maxplot) for all conditions.

The MS data provided in the examples described below were obtained as followed: Mass spectrum: LC/MS Waters ZMD (ESI) or a Waters Acquity SQD (ESI)

The NMR data provided in the examples described below were obtained as followed: ¹H-NMR: Bruker DPX-300 MHz or a Bruker DPX 400 MHz.

The microwave chemistry was performed on a single mode microwave reactor Emrys™ Optimiser from Personal Chemistry

Preparative HPLC purifications were performed with a mass directed autopurification Fractionlynx from Waters equipped with a Sunfire Prep C18 OBD column 19×100 mm 5 μm, unless otherwise reported. All HPLC purifications were performed with a gradient of ACN/H₂O or ACN/H₂O/HCOOH (0.1%).

The compounds of invention have been named according to the standards used in the program “ACD/Name Batch” from Advanced Chemistry Development Inc., ACD/Labs (7.00 Release). Product version: 7.10, build: 15 Sep. 2003

### Intermediate 1: 3-Amino-4-fluoro benzonitrile

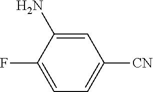

A solution of 4-fluoro-3-nitro benzonitrile (Combi-Blocks; 5.0 g; 30 mmol) and saturated solution of ammonium chloride (16 g; 0.3 mol in 20 ml of water) in iPrOH (100 ml) was treated with iron powder (8.4 g; 0.15 mol) and refluxed for 4 h. The mixture was cooled and diluted with ethyl acetate (200 ml) and the organic phase was washed with water, brine and dried on MgSO₄. The solvents were evaporated under reduced pressure and the residue was purified by flash column chromatography (silica), eluting with cyclohexane containing increasing amounts of EtOAc to give the Title compound (3.5 g, 87%) as a solid.

¹H NMR (300 MHz, DMSO-d₆) δ [ppm] 7.21-7.17 (m, 1H), 7.10-7.07 (m, 1H), 6.98-6.94 (m, 1H), 5.70 (bs, 2H). MS (ESI⁺): 136.9.

### Intermediate 2: [2-Fluoro-5-(1H-tetrazol-5-yl)phenyl]amine hydrochloride

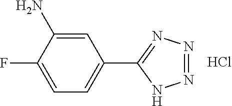

A solution of 3-amino-4-fluoro-benzonitrile (Intermediate 1; 2.0 g; 14.7 mmol) in dry toluene (75 ml) was treated with sodium azide (2.86 g; 44 mmol) and triethylamine hydrochloride (6.0 g) and the mixture was refluxed for 24 h under nitrogen. The reaction mass was cooled and water (25 ml) was added to it. The aqueous phase was separated and acidified with an aqueous (6 N) HCl solution till pH=2. A solid precipitated, which was filtered, washed with cold water and dried under suction to afford the Title compound (2.5 g, 80%) as a solid.

¹H NMR (300 MHz, DMSO-d₆) δ [ppm] 7.75-7.70 (m, 1H), 7.53-7.52 (m, 1H), 7.33-7.28 (m, 1H), 5.56 (bs, 2H). MS (ESI⁺): 179.9. HPLC (Condition B): Rt 4.47 min (HPLC purity 99.8%).

### Intermediate 3: N-(3-ethynylbenzyl)-2-fluoro-5-(1H-tetrazol-5-yl)aniline

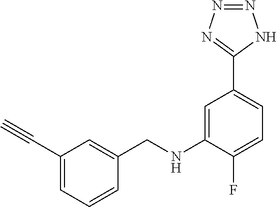

A suspension 2-fluoro-5-(1H-tetrazol-5-yl)-phenylamine hydrochloride (Intermediate 2; 504 mg; 2.34 mmol) in AcOH (4 ml) was treated with a solution of 3-ethynylbenzaldehyde (Zerenex; 360 mg; 2.77 mmol) in DMF (6 ml), followed by treatment with NaBH₃CN (453 mg; 7.21 mmol). The reaction mixture was stirred for 16 h then concentrated under vacuum and the residue azeotropically dried with toluene. The concentrated residue was dissolved in EtOAc then washed with 1N NaOH (aq). The organic phase was concentrated under vacuum, dissolved in DCM, filtered then redissolved in MeOH and purified by SCX strong acidic (sulfonic acid) ion exchange chromatography, eluting first with MeOH, then with a 2 N solution of NH₃ in MeOH. The title compound was obtained as a yellow solid (552 mg, 81%).

¹H NMR (300 MHz, DMSO-d₆) δ [ppm] 7.47 (s, 1H), 7.45-7.39 (m, 1H), 7.37-7.29 (m, 2H), 7.25-7.13 (m, 2H), 7.06 (dd, J=11.8, J=8.3 Hz, 1H), 4.39 (s, 2H), 4.12 (s, 1H). MS (ESI⁻): 292.2. HPLC (Condition A): Rt 3.66 min (HPLC purity 65.1%).

### Intermediate 4: N-(2,3-dihydro-1,4-benzodioxin-6-ylmethyl)-3-(1H-tetrazol-5-yl)aniline

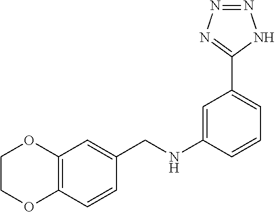

Following the general method as outlined in Intermediate 3, starting from 5-(3-aminophenyl)tetrazole (Avocado) and 1,4-benzodioxan-6-carboxaldehyde (ABCR), the title compound was obtained as a white foam.

¹H NMR (300 MHz, DMSO-d₆) δ [ppm] 7.27-7.23 (m, 1H), 7.19-7.13 (m, 1H), 7.07 (t, J=7.8 Hz, 1H), 6.87-6.74 (m, 3H), 6.56-6.49 (m, 1H), 4.17 (s, 6H). MS (ESI⁺): 310.0. HPLC (Condition A): Rt 2.70 min (HPLC purity 92.9%).

### Intermediate 5: 2-fluoro-N-(2-naphthylmethyl)-5-(1H-tetrazol-5-yl)aniline

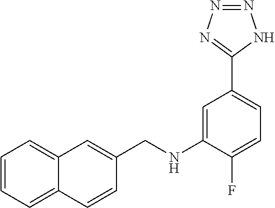

Following the general method as outlined in Intermediate 3, starting from 2-fluoro-5-(1H-tetrazol-5-yl)-phenylamine hydrochloride (Intermediate 2) and 2-naphthaldehyde (Aldrich), the title compound was obtained as brown after preparative HPLC.

¹H NMR (300 MHz, DMSO-d₆) δ [ppm] 7.93-7.81 (m, 4H), 7.57, J=8.4, J=1.5 Hz 1H), 7.51-7.41 (m, 2H), 7.34-7.15 (m, 3H), 6.78-6.69 (m, 1H), 4.60 (d, J=6.2 Hz, 2H). MS (ESI⁺): 320.1. HPLC (Condition A): Rt 3.93 min (HPLC purity 97.6%).

### Intermediate 6: N-(2-naphthylmethyl)-3-(1H-tetrazol-5-yl)aniline

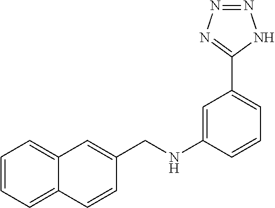

Following the general method as outlined in Intermediate 3, starting from 5-(3-aminophenyl)tetrazole (Avocado) and 2-naphthaldehyde (Aldrich), the title compound was obtained as a white solid.

¹H NMR (300 MHz, DMSO-d₆) δ [ppm] 7.91-7.80 (m, 4H), 7.55 (dd, J=8.5, J=1.5 Hz, 1H), 7.51-7.40 (m, 2H), 7.36-7.31 (m, 1H), 7.21-7.14 (m, 1H), 7.06 (t, J=7.9 Hz, 1H), 6.60-6.52 (m, 1H), 4.47 (s, 2H). MS (ESI⁺): 302.0. HPLC (Condition A): Rt 3.81 min (HPLC purity 92.1%).

### Intermediate 7: 2-fluoro-N-{[1-(phenylsulfonyl)-1H-pyrrol-2-yl]methyl}-5-(1H-tetrazol-5-yl)aniline

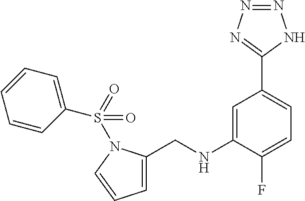

Following the general method as outlined in Intermediate 3, starting from 2-fluoro-5-(1H-tetrazol-5-yl)-phenylamine hydrochloride (Intermediate 2) and 1-(phenylsulfonyl)-2-pyrrolecarboxaldehyde (Aldrich), the title compound was obtained as an off-white solid which was used without further purification. MS (ESI⁻): 397.2. HPLC (Condition A): Rt 3.92 min (HPLC purity 61.6%).

### Intermediate 8: 3-[(2,3-dihydro-1,4-benzodioxin-6-ylmethyl)amino]-4-fluorobenzonitrile

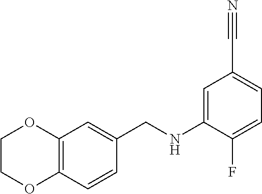

A suspension of 1,4-benzodioxan-6-carboxaldehyde (ABCR; 1.03 g; 6.27 mmol) and 3-amino-4-fluorobenzonitrile (Intermediate 1; 997 mg; 7.32 mmol) in Toluene (100 ml) was treated with p-toluenesulphonic acid monohydrate (10 mg; 0.06 mmol). A Dean-Stark trap was added and the suspension heated at reflux during 20 h, after which the reaction solution was cooled and concentrated to give a yellow solid. This was dissolved in MeOH (100 ml) and DCM (300 ml). The solution was cooled to −10° C. then treated with three portions of NaBH₄ (441 mg; 11.7 mmol each, 20 minutes apart). After stirring at room temperature for 3 h, the solution was concentrated then dissolved in DCM and washed with aqueous HCl (1 N). The layers were separated and the organic layer dried on MgSO₄ and concentrated to give the Title compound (1.88 g, 90%) as an orange oil, which was used without further purification.

¹H NMR (300 MHz, DMSO-d₆) δ [ppm] 7.27-7.16 (m, 1H), 7.02-6.93 (m, 2H), 6.90-6.68 (m, 4H), 4.25 (d, J=6.3 Hz, 2H), 4.20 (s, 4H). HPLC (Condition A): Rt 4.04 min (HPLC purity 89.2%).

### Intermediate 9: 3-[(3-thienylmethyl)amino]benzonitrile

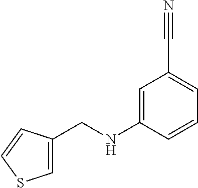

Following the general method as outlined in Intermediate 8, starting from 3-aminobenzonitrile (ABCR) and thiophene-3-carboxaldehyde (Aldrich), the title compound was obtained as a brown solid in 84% yield.

¹H NMR (300 MHz, DMSO-d₆) δ [ppm] 7.49 (dd, J=4.9, 2.9 Hz, 1H), 7.39-7.35 (m, 1H), 7.27-7.19 (m, 1H), 7.09 (dd, J=4.9, J=1.2 Hz, 1H), 6.95-6.88 (m, 3H), 6.66 (t, J=5.8 Hz, 1H), 4.28 (d, J=5.8 Hz, 2H). MS (ESI⁻): 213.0. HPLC (Condition A): Rt 3.85 min (HPLC purity 97.5%).

### Intermediate 10: 3-[(4-methoxybenzyl)amino]benzonitrile

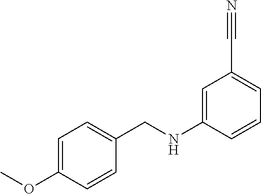

Following the general method as outlined in Intermediate 8, starting from 3-aminobenzonitrile (ABCR) and p-anisaldehyde (Aldrich), the title compound was obtained as an orange solid in quantitative yield.

¹H NMR (300 MHz, DMSO-d₆) δ [ppm] 7.32-7.16 (m, 3H), 6.93-6.84 (m, 5H), 6.70 (br s, 1H), 4.21 (s, 2H), 3.72 (s, 3H). MS (ESI⁻): 237.1. HPLC (Condition A): Rt 3.95 min (HPLC purity 93.8%).

### Intermediate 11: 5-amino-2,4-difluorobenzonitrile

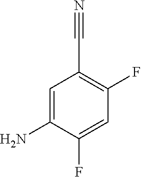

Following the general method as outlined in Intermediate 1, starting from 2,4-difluoro-5-nitrobenzonitrile (Apollo), the title compound was obtained as a brown solid.

¹H NMR (300 MHz, DMSO-d₆) δ [ppm] 7.49-7.38 (m, 1H), 7.14-7.05 (m, 1H), 5.55 (br s, 2H). MS (ESI⁻): 153.0. HPLC (Condition A): Rt 2.13 min (HPLC purity 70.1%).

### Intermediate 12: 5-[(2,3-dihydro-1,4-benzodioxin-6-ylmethyl)amino]-2,4-difluorobenzonitrile

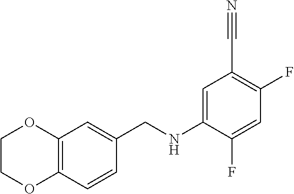

Following the general method as outlined in Intermediate 8, starting from 5-amino-2,4-difluorobenzonitrile (Intermediate 11) and 1,4-benzodioxan-6-carboxaldehyde (ABCR), the title compound was obtained as a brown oil in 86% yield, which was used without further purification.

MS (ESI⁻): 301.1. HPLC (Condition A): Rt 4.20 min (HPLC purity 50.2%).

### Intermediate 13: 3-[(2,3-dihydro-1,4-benzodioxin-6-ylmethyl)amino]-5-fluorobenzonitrile

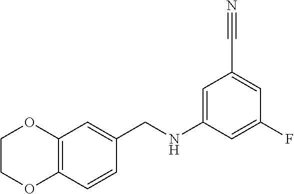

Following the general method as outlined in Intermediate 8, starting from 3-amino-5-fluorobenzonitrile (Intermediate 1) and 1,4-benzodioxan-6-carboxaldehyde (ABCR), the title compound was obtained as an orange oil, which was used without further purification.

MS (ESI⁻): 283.1. HPLC (Condition A): Rt 4.30 min (HPLC purity 72.3%).

### Intermediate 14: 3-[(1-benzofuran-2-ylmethyl)amino]-4-fluorobenzonitrile

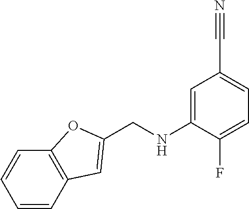

Following the general method as outlined in Intermediate 8, starting from 3-amino-4-fluorobenzonitrile

(Intermediate 1) and 2-benzofurancarboxaldehyde (Aldrich), the title compound was obtained as a brown solid, which was used without further purification.

¹H NMR (300 MHz, DMSO-d₆) δ [ppm] 7.61-7.55 (m, 1H), 7.55-7.49 (m, 1H), 7.32-7.13 (m, 4H), 7.08-7.00 (m, 1H), 6.85-6.74 (m, 2H), 4.58 (J=6.2 Hz, 2H). MS (ESI⁻): 265.0. HPLC (Condition A): Rt 4.13 min (HPLC purity 76.6%).

### Intermediate 15: 3-[(3-methoxybenzyl)amino]benzonitrile

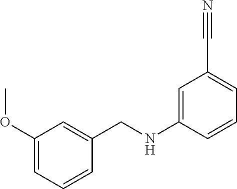

Following the general method as outlined in Intermediate 8, starting from 3-amino-benzonitrile (ABCR) and m-anisaldehyde (Aldrich), the title compound was obtained as a brown oil, which was used without further purification.

¹H NMR (300 MHz, DMSO-d₆) δ [ppm] 7.30-7.17 (m, 2H), 6.98-6.85 (m, 5H), 6.84-6.72 (m, 2H), 4.27 (d, J=5.9 Hz, 2H), 3.73 (s, 3H). MS (ESI⁻): 237.2. HPLC (Condition A): Rt 4.04 min (HPLC purity 99.0%).

### Intermediate 16: 3-[(2-thienylmethyl)amino]benzonitrile

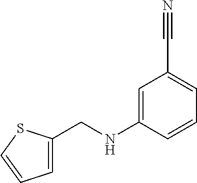

Following the general method as outlined in Intermediate 8, starting from 3-amino-benzonitrile (ABCR) and 2-thiophenecarboxaldehyde (Fluka), the title compound was obtained as a brown oil in 90% yield.

¹H NMR (300 MHz, DMSO-d₆) δ [ppm] 7.39 (dd, J=2.1, J=1.2 Hz, 1H), 7.29-7.20 (m, 1H), 7.10-7.04 (m, 1H), 7.01-6.90 (m, 4H), 6.81 (t, J=5.8 Hz, 1H), 4.49 (d, J=5.8 Hz, 2H). MS (ESI⁻): 213.0. HPLC (Condition A): Rt 3.92 min (HPLC purity 94.9%).

### Intermediate 17: 3-[(2,3-dihydro-1,4-benzodioxin-6-ylmethyl)amino]benzonitrile

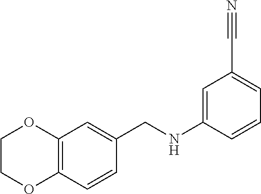

Following the general method as outlined in Intermediate 8, starting from 3-amino-benzonitrile (ABCR) and 1,4-benzodioxan-6-carboxaldehyde (ABCR), the title compound was obtained as an orange oil in 84% yield.

¹H NMR (300 MHz, DMSO-d₆) δ [ppm] 7.21 (t, J=7.9 Hz, 1H), 6.92-6.78 (m, 6H), 6.71 (t, J=6.0 Hz, 1H), 4.24-4.13 (m, 6H). MS (ESI⁻): 265.1. HPLC (Condition A): Rt 4.06 min (HPLC purity 100%).

Intermediate 18: N-(3-cyanophenyl)propanamide

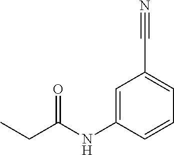

A cooled (−10° C.) solution of 3-aminobenzonitrile (ABCR; 10.13 g; 85.75 mmol) and NMM (30 ml) in DCM (300 ml) was treated over 5 minutes with propionyl chloride (Alfa Aesar; 8.0 ml; 92.5 mmol). The solution was stirred at −10° C. for 2 h then poured into an aqueous solution (1 N) of HCl. The organic layer was separated and washed with aqueous HCl solution (1 N), aqueous NaOH solution (1N) and brine, dried over MgSO₄ then concentrated to give the Title compound as an orange solid (13.12 g, 88%).

¹H NMR (300 MHz, DMSO-d₆) δ [ppm] 10.21 (br s, 1H), 8.10 (s, 1H), 7.79 (dt, J=7.3, J=2.1 Hz, 1H), 7.56-7.45 (m, 2H), 2.35 (q, J=7.5 Hz, 2H), 1.08 (t, J=7.5 Hz, 3H).

MS (ESI⁻): 173.1. HPLC (Condition A): Rt 2.30 min (HPLC purity 99.4%).

### Intermediate 19: N-(3-cyano-5-fluorophenyl)pentanamide

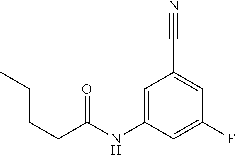

Following the general method as outlined in Intermediate 18, starting from 5-amino-3-fluorobenzonitrile (Oakwood) and n-valeryl chloride (Merck Kgaa), the title compound was obtained as a yellow solid in 91% yield.

¹H NMR (300 MHz, DMSO-d₆) δ [ppm] 10.40 (br s, 1H), 7.84-7.76 (m, 2H), 7.50 (ddd, J=8.3 Hz, J=2.4 Hz, J=1.4 Hz, 1H), 2.34 (t, J=7.4 Hz, 2H), 1.65-1.50 (m, 2H), 1.32 (sextet, J=7.3 Hz, 2H), 0.89 (t, J=7.3 Hz, 3H). MS (ESI⁻): 219.1. HPLC (Condition A): Rt 3.84 min (HPLC purity 100%).

### Intermediate 20: N-(3-cyano-5-fluorophenyl)propanamide

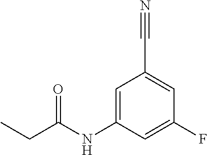

Following the general method as outlined in Intermediate 18, starting from 5-amino-3-fluorobenzonitrile (Oakwood) and propionyl chloride (Alfa Aesar), the title compound was obtained as a white solid in 93% yield.

¹H NMR (300 MHz, DMSO-d₆) δ [ppm] 10.40 (br s, 1H), 7.84-7.77 (m, 2H), 7.53-7.47 (m, 1H), 2.36 (q, J=7.5 Hz, 2H), 1.08 (t, J=7.5 Hz, 3H). MS (ESI⁻): 191.0. HPLC (Condition A): Rt 2.83 min (HPLC purity 99.0%).

### Intermediate 21: N-(3-chloro-5-cyanophenyl)pentanamide

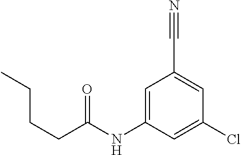

Following the general method as outlined in Intermediate 18, starting from 3-amino-5-chlorobenzonitrile (Combi-Blocks) and n-valeryl chloride (Merck Kgaa), the title compound was obtained as a yellow solid in 86% yield.

¹H NMR (300 MHz, DMSO-d₆) δ [ppm] 10.38 (br s, 1H), 7.99 (t, J=1.9 Hz, 1H), 7.93 (t, J=1.9 Hz, 1H), 7.69 (t, J=1.9 Hz, 1H), 2.34 (t, J=7.4 Hz, 2H), 1.57 (quintet, J=7.4 Hz, 2H), 1.32 (hex, J=7.4 Hz, 2H), 0.89 (t, J=7.4 Hz, 3H). MS (ESI⁻): 235.0. HPLC (Condition A): Rt 3.95 min (HPLC purity 96.2%).

### Intermediate 22: N-(3-cyanophenyl)-N-(3-iodobenzyl)propanamide

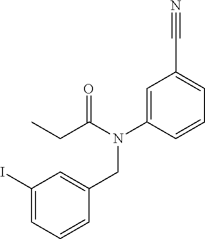

A cooled (−10° C.) solution of N-(3-cyanophenyl)propanamide (Intermediate 18; 1.89 g; 10.85 mmol) in DMF (40 ml) was treated with NaH (60% dispersion in mineral oil, 0.55 g; 13.8 mmol), followed after 10 min by treatment with a solution of 3-iodo-benzyl bromide (VWR; 3.24 g; 10.9 mmol) in DMF (5 ml). The reaction mixture was stirred at −10° C. for 2 h then IPrOH (20 ml) was added to quenche the reaction. The solvents were removed under vacuum, EtOAc was added and the organic phase washed with aqueous (1 N) HCl, dried (MgSO₄) and concentrated and the residue purified by chromatography to give the Title compound (3.43 g, 81%) as a clear oil which crystallised as a white solid on standing.

¹H NMR (300 MHz, DMSO-d₆) δ [ppm]. ¹H NMR (DMSO-d₆) δ 7.87-7.73 (m, 2H), 7.63-7.50 (m, 4H), 7.20 (d, J=7.7 Hz, 1H), 7.09 (t, J=7.7 Hz, 1H), 4.87 (s, 2H), 2.21-2.02 (m, 2H), 0.95 (t, J=7.4 Hz, 3H). MS (ESI⁺): 391.0. HPLC (Condition A): Rt 4.47 min (HPLC purity 97.0%).

### Intermediate 23: N-(3-iodobenzyl)-N-[3-(1H-tetrazol-5-yl)phenyl]propanamide

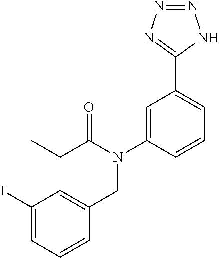

A suspension of N-(3-cyanophenyl)-N-(3-iodobenzyl)propanamide (Intermediate 22; 2.57 g; 6.59 mmol; 1.00 eq.) and dibutyltin oxide (10 mg; 0.04 mmol; 0.09 eq) in Toluene (50.00 ml) was treated with trimethylsilyl azide (1.40 ml; 10.69 mmol; 1.62 eq.). The suspension was heated at 80° C. for 16 h. The solvent was removed under vacuum, the residue was dissolved in EtOAc, washed with aqueous (1 N) HCl, dried on MgSO₄ and concentrated to give the Title compound as a yellow foam.

¹H NMR (300 MHz, DMSO-d₆) δ [ppm] 7.98 (d, J=7.8 Hz, 1H), 7.87 (s, 1H), 7.68-7.54 (m, 3H), 7.46-7.36 (m, 1H), 7.23 (d, J=7.7 Hz, 1H), 7.10 (t, J=8.0 Hz, 1H), 4.89 (s, 2H), 2.24-2.06 (m, 2H), 0.97 (t, J=7.4 Hz, 3H). MS (ESI⁻): 432.1. HPLC (Condition A): Rt 3.68 min (HPLC purity 95.2%).

### Intermediate 24: 3-chloro-5-{[4-(trifluoromethoxy)benzyl]amino}benzonitrile

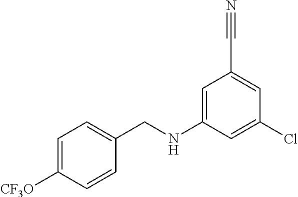

Following the general method as outlined in Intermediate 8, starting from 3-amino-5-chlorobenzonitrile and (trifluoromethoxy)benzaldehyde, the title compound was obtained as a yellow solid after purification by column chromatography (silica), eluting with cyclohexane containing increasing amounts of EtOAc.

¹H NMR (300 MHz, DMSO-d6) δ [ppm] 7.47 (d, J=8.4 Hz, 2H), 7.35 (d, J=8.4 Hz, 2H), 7.11 (t, J=6.0 Hz, 1H), 7.02 (t, J=1.6 Hz, 1H), 6.93-6.91 (m, 2H), 4.36 (d, J=6.0 Hz, 2H). HPLC (Condition A): Rt 5.14 min (HPLC purity 97.4%).

### Intermediate 25: 3-[(quinolin-6-ylmethyl)amino]benzonitrile

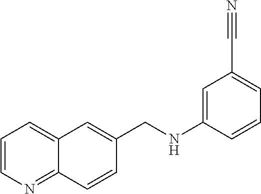

Following the general method as outlined in Intermediate 8, starting from 3-aminobenzonitrile and 6-quinolinecarbaldehyde, the title compound was obtained as a clear oil after purification by column chromatography (silica), eluting with cyclohexane containing increasing amounts of EtOAc.

¹H NMR (300 MHz, DMSO-d6) δ [ppm] 8.86 (d, J=4.0 Hz, 1H), 8.32 (d, J=8.2 Hz, 1H), 8.00 (d, J=8.6 Hz, 1H), 7.91 (s, 1H), 7.75 (d, J=8.2 Hz, 1H), 7.51 (dd, J=8.2, 4.2 Hz, 1H), 7.23 (t, J=8.2 Hz, 1H), 7.02-6.86 (m, 4H), 4.52 (, J=6.0 Hz, 2H). HPLC (Condition A): Rt 2.24 min (HPLC purity 100%).

### Intermediate 26: 3-[(4-methoxybenzyl)amino]benzonitrile

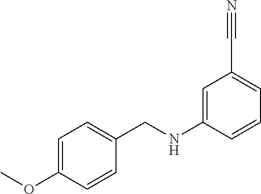

Following the general method as outlined in Intermediate 8, starting from 3-aminobenzonitrile and p-anisaldehyde, the title compound was obtained as an orange solid in quantitative yield.

¹H NMR (300 MHz, DMSO-d6) δ [ppm] 7.32-7.16 (m, 3H), 6.93-6.84 (m, 5H), 6.70 (br s, 1H), 4.21 (s, 2H), 3.72 (s, 3H). HPLC (Condition A): Rt 3.95 min (HPLC purity 93.8%).

### Intermediate 27: 3-chloro-5-{[(2,2-difluoro-1,3-benzodioxol-5-yl)methyl]amino}benzonitrile

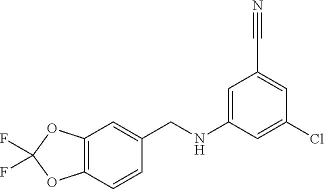

Following the general method as outlined in Intermediate 8, starting from 3-amino-5-chlorobenzonitrile and 2,2-difluoro-5-formylbenzodioxole, the title compound was obtained as an oil after purification by column chromatography (silica), eluting with cyclohexane containing increasing amounts of EtOAc.

¹H NMR (300 MHz, DMSO-d6) δ [ppm] 7.38 (d, J=1.4 Hz, 1H), 7.37 (d, J=7.0 Hz, 1H), 7.19 (dd, J=1.4, 8.2 Hz, 1H), 7.08 (t, J=6.0 Hz, 1H), 7.02 (t, J=1.6 Hz, 1H), 6.90 (m, 2H), 4.33 (d, J=6.0 Hz, 2H).

### Intermediate 28: 3-fluoro-5-{[4-(methylsulfonyl)benzyl]amino}benzonitrile

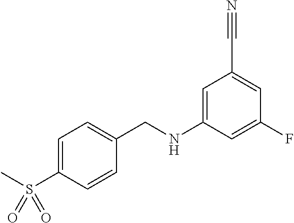

Following the general method as outlined in Intermediate 8, starting from 3-amino-5-fluoro-benzonitrile and 4-(methylsulfonyl)benzaldehyde, the title compound was obtained after purification by column chromatography (silica), eluting with cyclohexane containing increasing amounts of EtOAc.

¹H NMR (300 MHz, DMSO-d6) δ 7.91 (d, J=8.2 Hz, 2H), 7.60 (d, J=8.2 Hz, 2H), 7.25 (t, J=6.1 Hz, 1H), 6.90-6.78 (m, 2H), 6.76-6.61 (m, 1H), 4.47 (d, J=6.2 Hz, 2H), 3.20 (s, 3H). MS (ESI⁻): 303.5

### Intermediate 29: N-(3-cyanophenyl)-N-(4-iodobenzyl)propanamide

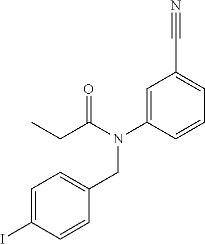

Following the general method as outlined in Intermediate 22, starting from N-(3-cyanophenyl)propanamide (Intermediate 18) and 4-iodobenzyl bromide, the title compound was obtained as a solid in 89% yield after purification by column chromatography (silica), eluting with cyclohexane containing increasing amounts of EtOAc.

¹H NMR (300 MHz, DMSO-d6) δ 7.84 (s, 1H), 7.78 (d, J=6.8 Hz, 1H), 7.71-7.48 (m, 4H), 7.00 (d, J=8.0 Hz, 2H), 4.86 (s, 2H), 2.10 (m, 2H), 0.95 (t, J=7.3 Hz, 3H). MS (ESI⁺): 391.1

### Intermediate 30: N-(3-cyano-5-fluorophenyl)-N-(3-iodobenzyl)propanamide

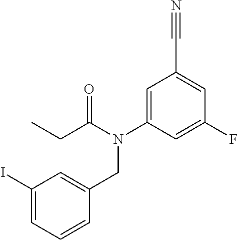

Following the general method as outlined in Intermediate 22, starting from N-(3-cyano-5-fluorophenyl)propanamide (Intermediate 20) and 3-iodobenzyl bromide, the title compound was obtained as a yellow oil in 80% yield after purification by column chromatography (silica), eluting with cyclohexane containing increasing amounts of EtOAc.

¹H NMR (300 MHz, DMSO-d6) δ [ppm] 7.90-7.83 (m, 1H), 7.77 (s, 1H), 7.69 (d t, J=9.9, 2.2 Hz, 1H), 7.66-7.59 (m, 2H), 7.25 (d, J=7.8 Hz, 1H), 7.13 (t, J=7.8 Hz, 1H), 4.92 (s, 2H), 2.23 (q, J=7.2 Hz, 2H), 1.00 (t, J=7.2 Hz, 3H). HPLC (max plot) 97.0%; Rt 4.50 min.

### Intermediate 31: N-(3-cyano-5-fluorophenyl)-N-(4-iodobenzyl)propanamide

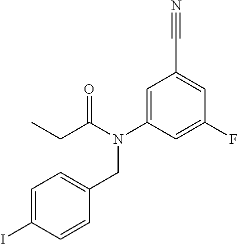

Following the general method as outlined in Intermediate 22, starting from N-(3-cyano-5-fluorophenyl)propanamide (Intermediate 20) and 4-iodobenzyl bromide, the title compound was obtained in 83% yield after purification by column chromatography (silica), eluting with cyclohexane containing increasing amounts of EtOAc.

¹H NMR (300 MHz, DMSO-d6) δ 7.88-7.76 (m, 1H), 7.73 (t, J=1.4 Hz, 1H), 7.70-7.59 (m, 3H), 7.02 (d, J=8.3 Hz, 2H), 4.88 (s, 2H), 2.18 (m, 2H), 0.96 (t, J=7.3 Hz, 3H). MS (ESI⁺): 409.1

### EXAMPLE 1

### N-(3-ethynylbenzyl)-N-[2-fluoro-5-(1H-tetrazol-5-yl)phenyl]pentanamide

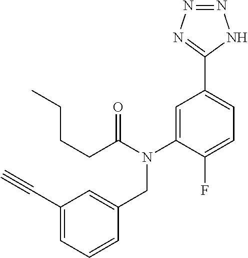

A solution of N-(3-ethynylbenzyl)-2-fluoro-5-(1H-tetrazol-5-yl)aniline (Intermediate 3; 275 mg; 0.56 mmol) and NMM (0.30 ml) in DMF (5.50 ml) was treated over 5 minutes with valeroyl chloride (Merck Kgaa; 0.20 ml; 1.69 mmol). The solution was heated at 50° C. for 24 h then the solvents were removed under vacuum. The residue was redissolved in EtOAc and washed with aqueous HCl solution (1 N), aqueous NaOH solution (1N) and brine, dried over MgSO₄ then concentrated to give a residue, which was purified by preparative HPLC to give the Title compound as a white solid.

¹H NMR (300 MHz, DMSO-d₆) δ [ppm] 8.10-7.91 (m, 2H), 7.57 (t, 1H), 7.39-7.17 (m, 4H), 4.85 (s, 2H), 4.12 (s, 1H), 2.20-1.94 (m, 2H), 1.47 (quintet, J=7.6 Hz, 2H), 1.17 (sextet, J=7.4 Hz, 2H), 0.75 (t, J=7.3 Hz, 3H). MS (ESI⁻): 376.3. HPLC (Condition A): Rt 4.13 min (HPLC purity 95.2%).

### EXAMPLE 2

### N-(3-ethynylbenzyl)-N-[2-fluoro-5-(1H-tetrazol-5-yl)phenyl]acetamide

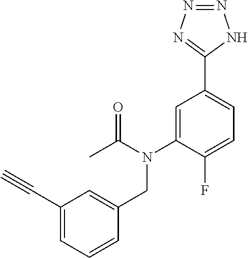

Following the general method as outlined in Example 1, starting from N-(3-ethynylbenzyl)-2-fluoro-5-(1H-tetrazol-5-yl)aniline (Intermediate 3) and acetyl chloride (Aldrich), the title compound was obtained as a yellow solid.

¹H NMR (300 MHz, DMSO-d₆) δ [ppm] 8.12-7.87 (m, 2H), 7.57 (t, J=9.2 Hz, 1H), 7.39-7.20 (m, 4H), 5.07-4.74 (m, 2H), 4.11 (s, 1H), 1.87 (s, 3H). MS (ESI⁻): 334.2. HPLC (Condition A): Rt 3.28 min (HPLC purity 99.6%).

### EXAMPLE 3

### N-(2-naphthylmethyl)-N-[3-(1H-tetrazol-5-yl)phenyl]pentanamide

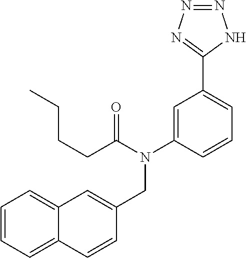

Following the general method as outlined in Example 1, starting from N-(2-naphthylmethyl)-3-(1H-tetrazol-5-yl)aniline (Intermediate 6) and valeroyl chloride (Merck Kgaa), the title compound was obtained as a white solid in 79% yield.

¹H NMR (300 MHz, DMSO-d₆) δ [ppm] 7.98-7.77 (m, 5H), 7.66 (s, 1H), 7.57 (t, J=7.9 Hz, 1H), 7.50-7.36 (m, 4H), 5.10 (s, 2H), 2.17 (br s, 2H), 1.52 (quintet, J=7.6 Hz, 2H), 1.20 (m, 2H), 0.76 (t, J=7.4 Hz, 3H). MS (ESI⁻): 384.2. HPLC (Condition A): Rt 4.18 min (HPLC purity 96.7%).

### EXAMPLE 4

### N-[2-fluoro-5-(1H-tetrazol-5-yl)phenyl]-N-{[1-(phenylsulfonyl)-1H-pyrrol-2-yl]methyl}pentanamide

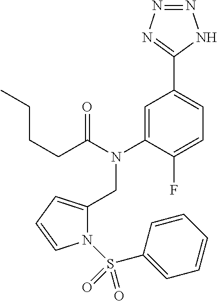

Following the general method as outlined in Example 1, starting from 2-fluoro-N-{[1-(phenylsulfonyl)-1H-pyrrol-2-yl]methyl}-5-(1H-tetrazol-5-yl)aniline (Intermediate 7) and valeroyl chloride (Merck KgAa), the title compound was obtained as a white solid.

¹H NMR (300 MHz, DMSO-d₆) δ [ppm] 8.07-7.97 (m, 1H), 7.94-7.87 (m, 1H), 7.85-7.76 (m, 2H), 7.66 (t, J=7.3 Hz, 1H), 7.60-7.49 (m, 3H), 7.34 (s, 1H), 6.25 (t, J=3.4 Hz, 1H), 6.20 (s, 1H), 5.16 (d, J=16.0 Hz, 1H), 4.81 (d, J=16.0 Hz, 1H), 2.19-1.89 (m, 2H), 1.46 (quintet, J=7.3 Hz, 2H), 1.17 (sextet, J=7.3 Hz, J=2H), 0.75 (t, J=7.3 Hz, 3H). MS (ESI⁻): 481.2. HPLC (Condition A): Rt 4.26 min (HPLC purity 99.7%).

### EXAMPLE 5

### N-(2,3-dihydro-1,4-benzodioxin-6-ylmethyl)-N-[3-(1H-tetrazol-5-yl)phenyl]pentanamide

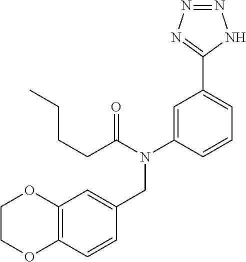

Following the general method as outlined in Example 1, starting from N-(2,3-dihydro-1,4-benzodioxin-6-ylmethyl)-3-(1H-tetrazol-5-yl)aniline (Intermediate 4) and valeroyl chloride (Merck Kgaa), the title compound was obtained as a white solid.

¹H NMR (300 MHz, DMSO-d₆) δ [ppm] 7.96 (d, J=7.7 Hz, 1H), 7.80 (s, 1H), 7.61 (t, J=7.9 Hz, 1H), 7.34 (d, J=8.4 Hz, 1H), 6.73 (d, 1H), 6.69-6.58 (m, 2H), 4.78 (s, 2H), 4.16 (s, 4H), 2.09 (br s, 2H), 1.46 (quintet, J=7.5 Hz, 2H), 1.16 (q, J=7.1 Hz, 2H), 0.74 (t, J=7.3 Hz, 3H). MS (ESI⁻): 392.2. HPLC (Condition A): Rt 3.60 min (HPLC purity 100.0%).

### EXAMPLE 6

### N-[2-fluoro-5-(1H-tetrazol-5-yl)phenyl]-N-(2-naphthylmethyl)pentanamide

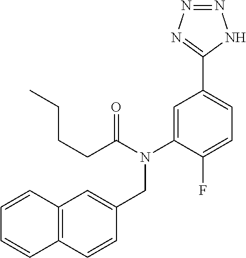

Following the general method as outlined in Example 1, starting from 2-fluoro-N-(2-naphthylmethyl)-5-(1H-tetrazol-5-yl)aniline (Intermediate 5) and valeroyl chloride (Merck Kgaa), the title compound was obtained as a white solid.

¹H NMR (300 MHz, DMSO-d₆) δ [ppm] 8.07-7.97 (m, 2H), 7.92-7.75 (m, 3H), 7.67 (s, 1H), 7.60-7.36 (m, 4H), 5.05 (s, 2H), 2.24-1.99 (m, 2H), 1.58-1.45 (m, 2H), 1.28-1.13 (m, 2H), 0.77 (t, J=7.3 Hz, 3H). MS (ESI⁻): 402.0. HPLC (Condition A): Rt 4.53 min (HPLC purity 99.1%).

### EXAMPLE 7

### N-(2,3-dihydro-1,4-benzodioxin-6-ylmethyl)-N-[2-fluoro-5-(1H-tetrazol-5-yl)phenyl]-3-methylbutanamide

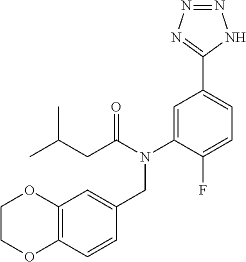

### Step 1: N-(5-cyano-2-fluorophenyl)-N-(2,3-dihydro-1,4-benzodioxin-6-ylmethyl)-3-methylbutanamide

A solution of 3-[(2,3-dihydro-1,4-benzodioxin-6-ylmethyl)amino]-4-fluorobenzonitrile (Intermediate 8; 392 mg; 1.38 mmol) and NMM (0.60 ml) in DMF (5 ml) was treated with isovaleryl chloride (Aldrich; 0.60 ml; 4.9 mmol). The reaction solution was heated at 50° C. for 2 h then cooled and the solvent removed under vacuum. The residue was dissolved in DCM and washed with an aqueous solution (1 N) of HCl then with a saturated aqueous solution of NaHCO₃. The organic layer was dried with MgSO₄ then concentrated to give a residue, which was purified by flash column chromatography (silica), eluting with cyclohexane containing increasing amounts of EtOAc, to give the Title compound as a yellow solid

¹H NMR (300 MHz, DMSO-d₆) δ [ppm] 8.01-7.73 (m, 2H), 7.56 (t, J=9.1 Hz, 1H), 6.83-6.51 (m, 3H), 4.98-4.56 (m, 2H), 4.18 (s, 4H), 2.17-1.75 (m, 2H), 1.03-0.66 (m, 7H). MS (ESI⁺): 369.2. HPLC (Condition A): Rt 4.32 min (HPLC purity 93.1%).

### Step 2: N-(2,3-dihydro-1,4-benzodioxin-6-ylmethyl)-N-[2-fluoro-5-(1H-tetrazol-5-yl)phenyl]-3-methylbutanamide

A solution of N-(5-cyano-2-fluorophenyl)-N-(2,3-dihydro-1,4-benzodioxin-6-ylmethyl)-3-methylbutanamide (338 mg; 0.92 mmol) and trimethylsilyl azide (0.30 ml; 2.29 mmol) in Toluene (20 ml) was treated with dibutyltin oxide (12 mg; 0.05 mmol). The solution was heated at reflux for 8 h then cooled and the solvent removed under vacuum. The residue was dissolved in DCM then washed with an aqueous solution (1 N) of HCl. The organic layer was dried on MgSO₄, concentrated under vacuum and the residue purified by Preparative HPLC to give the Title compound as a white solid.

¹H NMR (300 MHz, DMSO-d₆) δ [ppm] 8.11-7.94 (m, 1H), 7.92-7.84 (m, 1H), 7.59 (t, J=9.2 Hz, 1H), 6.82-6.56 (m, 3H), 4.82 (d, 1H), 4.66 (d, 1H), 4.17 (s, 4H), 2.16-1.83 (m, 3H), 0.87-0.69 (m, 6H). MS (ESI⁻): 410.2. HPLC (Condition A): Rt 3.75 min (HPLC purity 98.9%).

### EXAMPLE 8

### N-(2,3-dihydro-1,4-benzodioxin-6-ylmethyl)-N-[2-fluoro-5-(1H-tetrazol-5-yl)phenyl]butanamide

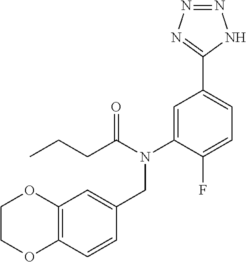

### Step 1: N-(5-cyano-2-fluorophenyl)-N-(2,3-dihydro-1,4-benzodioxin-6-ylmethyl)butanamide

Following the general method as outlined in Example 7 (Step 1), starting from 3-[(2,3-dihydro-1,4-benzodioxin-6-ylmethyl)amino]-4-fluorobenzonitrile (Intermediate 8) and butyryl chloride (Aldrich), the title compound was obtained as a clear oil.

¹H NMR (300 MHz, DMSO-d₆) δ [ppm] 8.03-7.88 (m, 2H), 7.64-7.48 (m, 1H), 6.81-6.53 (m, 3H), 4.94-4.59 (m, 2H), 4.18 (s, 4H), 2.15-1.81 (m, 2H), 1.61-1.39 (m, 2H), 0.78 (t, J=7.3 Hz, 3H). MS (ESI⁺): 355.0. HPLC (Condition A): Rt 4.03 min (HPLC purity 88.5%).

### Step 2: N-(2,3-dihydro-1,4-benzodioxin-6-ylmethyl)-N-[2-fluoro-5-(1H-tetrazol-5-yl)phenyl]butanamide

Following the general method as outlined in Example 7 (Step 2), starting from of N-(5-cyano-2-fluorophenyl)-N-(2,3-dihydro-1,4-benzodioxin-6-ylmethyl)butanamide, the title compound was obtained as a white solid.

¹H NMR (300 MHz, DMSO-d₆) δ [ppm] 8.11-8.00 (m, 1H), 7.96-7.87 (m, 1H), 7.59 (t, J=9.2 Hz, 1H), 6.80-6.57 (m, 3H), 4.81 (d, J=14.6 Hz, 1H), 4.67 (d, J=14.6 Hz, 1H), 4.17 (s, 4H), 2.17-1.90 (m, 2H), 1.59-1.43 (m, 2H), 0.78 (t, J=7.4 Hz, 3H). MS (ESI⁻): 396.1. HPLC (Condition A): Rt 3.50 min (HPLC purity 99.3%).

### EXAMPLE 9

### N-(2,3-dihydro-1,4-benzodioxin-6-ylmethyl)-N-[2-fluoro-5-(1H-tetrazol-5-yl)phenyl]propanamide

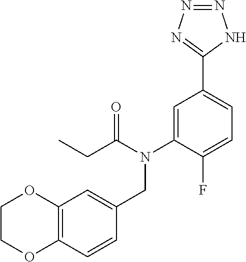

### Step 1: N-(5-cyano-2-fluorophenyl)-N-(2,3-dihydro-1,4-benzodioxin-6-ylmethyl)propanamide

Following the general method as outlined in Example 7 (Step 1), starting from 3-[(2,3-dihydro-1,4-benzodioxin-6-ylmethyl)amino]-4-fluorobenzonitrile (Intermediate 8) and propionyl chloride (Alfa Aesar), the title compound was obtained as a clear oil in 82% yield.

¹H NMR (300 MHz, DMSO-d₆) δ [ppm] 8.00-7.85 (m, 2H), 7.55 (t, J=8.9 Hz, 1H), 6.80-6.52 (m, 3H), 4.70 (s, 2H), 4.18 (s, 4H), 2.19-1.80 (m, 2H), 0.94 (t, 3H). MS (ESI⁺): 341.0. HPLC (Condition A): Rt 3.73 min (HPLC purity 98.1%).

### Step 2: N-(2,3-dihydro-1,4-benzodioxin-6-ylmethyl)-N-[2-fluoro-5-(1H-tetrazol-5-yl)phenyl]propanamide

Following the general method as outlined in Example 7 (Step 2), starting from of N-(5-cyano-2-fluorophenyl)-N-(2,3-dihydro-1,4-benzodioxin-6-ylmethyl)propanamide, the title compound was obtained as a white solid.

¹H NMR (300 MHz, DMSO-d₆) δ [ppm] 8.12-7.86 (m, 2H), 7.59 (t, J=9.3 Hz, 1H), 6.84-6.55 (m, 3H), 4.80 (d, J=14.4 Hz, 1H), 4.68 (d, J=14.4 Hz, 1H), 4.17 (s, 4H), 2.23-1.89 (m, 2H), 0.96 (t, J=7.3 Hz, 3H). MS (ESI⁻): 382.2. HPLC (Condition A): Rt 3.21 min (HPLC purity 100.0%).

### EXAMPLE 10

### N-[2,4-difluoro-5-(1H-tetrazol-5-yl)phenyl]-N-(2,3-dihydro-1,4-benzodioxin-6-ylmethyl)pentanamide

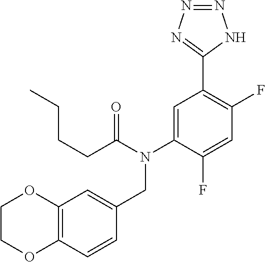

### Step 1: N-(5-cyano-2,4-difluorophenyl)-N-(2,3-dihydro-1,4-benzodioxin-6-ylmethyl)pentanamide

Following the general method as outlined in Example 7 (Step 1), starting from 5-[(2,3-dihydro-1,4-benzodioxin-6-ylmethyl)amino]-2,4-difluorobenzonitrile (Intermediate 12) and valeroyl chloride (Merck Kgaa), the title compound was obtained as a yellow oil.

MS (ESI⁺): 387.2. HPLC (Condition A): Rt 4.51 min (HPLC purity 76.9%).

### Step 2: N-[2,4-difluoro-5-(1H-tetrazol-5-yl)phenyl]-N-(2,3-dihydro-1,4-benzodioxin-6-ylmethyl)pentanamide

Following the general method as outlined in Example 7 (Step 2), starting from of N-(5-cyano-2,4-difluorophenyl)-N-(2,3-dihydro-1,4-benzodioxin-6-ylmethyl)pentanamide, the title compound was obtained as a white solid.

¹H NMR (300 MHz, DMSO-d₆) δ [ppm] 7.92 (t, J=8.0 Hz, 1H), 7.72 (t, J=10.2 Hz, 1H), 6.83-6.57 (m, 3H), 4.76 (d, J=14.6 Hz, 1H), 4.66 (d, J=14.6 Hz, 1H), 4.18 (s, 4H), 2.18-1.89 (m, 2H), 1.47 (quintet, J=7.4 Hz, 2H), 1.18 (sextet, J=7.5 Hz, 2H), 0.77 (t, J=7.3 Hz, 3H). MS (ESI⁻): 428.1. HPLC (Condition A): Rt 3.88 min (HPLC purity 99.6%).

### EXAMPLE 11

### N-(2,3-dihydro-1,4-benzodioxin-6-ylmethyl)-N-[3-fluoro-5-(1H-tetrazol-5-yl)phenyl]pentanamide

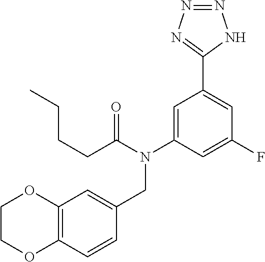

### Step 1: N-(3-cyano-5-fluorophenyl)-N-(2,3-dihydro-1,4-benzodioxin-6-ylmethyl)pentanamide

Following the general method as outlined in Example 7 (Step 1), starting from 3-[(2,3-dihydro-1,4-benzodioxin-6-ylmethyl)amino]-5-fluorobenzonitrile (Intermediate 13) and valeroyl chloride (Merck Kgaa), the title compound was obtained as a clear oil.

¹H NMR (300 MHz, DMSO-d₆) δ [ppm] 7.85-7.78 (m, 1H), 7.69-7.64 (m, 1H), 7.58 (dt, J=9.8, J=2.1 Hz, 1H), 6.74 (d, J=8.2 Hz, 1H), 6.67 (d, J=2.0 Hz, 1H), 6.60 (dd, J=8.2, J=2.0 Hz, 1H), 4.79 (br s, 2H), 4.19 (br s, 4H), 2.27-2.08 (m, 2H), 1.54-1.37 (m, 2H), 1.19 (hex, J=7.59 Hz, 2H), 0.78 (t, J=7.3 Hz, 3H). MS (ESI⁺): 369.0. HPLC (Condition A): Rt 4.43 min (HPLC purity 96.6%).

### Step 2: N-(2,3-dihydro-1,4-benzodioxin-6-ylmethyl)-N-[3-fluoro-5-(1H-tetrazol-5-yl)phenyl]pentanamide

Following the general method as outlined in Example 7 (Step 2), starting from of N-(3-cyano-5-fluorophenyl)-N-(2,3-dihydro-1,4-benzodioxin-6-ylmethyl)pentanamide, the title compound was obtained as a yellow solid.

¹H NMR (300 MHz, DMSO-d₆) δ [ppm] 7.78 (d, J=9.8 Hz, 1H), 7.67 (s, 1H), 7.38 (dt, J=9.8, J=2.0 Hz, 1H), 6.75 (d, J=8.2 Hz, 1H), 6.70 (d, J=2.0 Hz, 1H), 6.63 (dd, J=8.2, 2.0 Hz, 1H), 4.81 (s, 2H), 4.18 (s, 4H), 2.26-2.11 (m, 2H), 1.49 (quintet, J=7.8 Hz, 2H), 1.19 (hex, J=7.4 Hz, 2H), 0.77 (t, J=7.3 Hz, 3H). MS (ESI⁻): 410.2. HPLC (Condition A): Rt 3.87 min (HPLC purity 93.6%).

### EXAMPLE 12

### N-(1-benzofuran-2-ylmethyl)-N-[2-fluoro-5-(1H-tetrazol-5-yl)phenyl]pentanamide

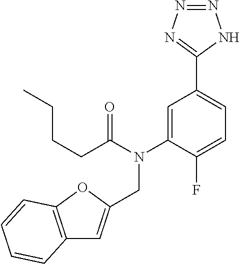

### Step 1: N-(1-benzofuran-2-ylmethyl)-N-(5-cyano-2-fluorophenyl)pentanamide

Following the general method as outlined in Example 7 (Step 1), starting from 3-[(1-benzofuran-2-ylmethyl)amino]-4-fluorobenzonitrile (Intermediate 14) and valeroyl chloride (Merck Kgaa), the title compound was obtained as a yellow oil in 71% yield.

¹H NMR (300 MHz, DMSO-d₆) δ [ppm] 8.19-7.80 (m, 2H), 7.66-7.38 (m, 3H), 7.33-7.11 (m, 2H), 6.69 (s, 1H), 5.10 (d, J=15.7 Hz, 1H), 4.92 (d, J=15.7 Hz, 1H), 2.21-1.86 (m, 2H), 1.67-1.30 (m, 2H), 1.29-1.06 (m, 2H), 0.77 (t, J=7.3 Hz, 3H). MS (ESI⁺): 351.0. HPLC (Condition A): Rt 4.88 min (HPLC purity 95.6%).

### Step 2: N-(1-benzofuran-2-ylmethyl)-N-[2-fluoro-5-(1H-tetrazol-5-yl)phenyl]pentanamide

Following the general method as outlined in Example 7 (Step 2), starting from of N-(1-benzofuran-2-ylmethyl)-N-(5-cyano-2-fluorophenyl)pentanamide, the title compound was obtained as a yellow solid.

¹H NMR (300 MHz, DMSO-d₆) δ [ppm] 8.15-7.92 (m, 2H), 7.66-7.45 (m, 3H), 7.32-7.13 (m, 2H), 6.70 (s, 1H), 5.20-4.95 (m, 2H), 2.22-1.94 (m, 2H), 1.67-1.42 (m, 2H), 1.41-1.09 (m, 2H), 0.98-0.68 (m, 3H). MS (ESI⁻): 392.2. HPLC (Condition A): Rt 4.15 min (HPLC purity 98.5%).

### EXAMPLE 13

### N-(3-methoxybenzyl)-N-[3-(1H-tetrazol-5-yl)phenyl]pentanamide

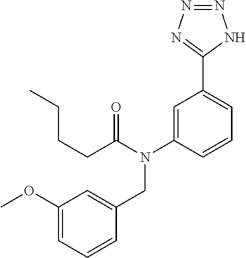

### Step 1: N-(3-cyanophenyl)-N-(3-methoxybenzyl)pentanamide

Following the general method as outlined in Example 7 (Step 1), starting from 3-[(3-methoxybenzyl)amino]benzonitrile (Intermediate 15) and valeroyl chloride (Merck Kgaa), the title compound was obtained as a clear oil.

¹H NMR (300 MHz, DMSO-d₆) δ [ppm] 7.83-7.73 (m, 2H), 7.61-7.48 (m, 2H), 7.19 (t, J=7.8 Hz, 1H), 6.83-6.68 (m, 3H), 4.87 (s, 2H), 3.69 (s, 3H), 2.22-2.02 (m, 2H), 1.54 (m, 2H), 1.27-1.11 (m, 2H), 0.77 (t, 3H). MS (ESI⁺): 323.0. HPLC (Condition A): Rt 4.33 min (HPLC purity 98.5%).

### Step 2: N-(3-methoxybenzyl)-N-[3-(1H-tetrazol-5-yl)phenyl]pentanamide

Following the general method as outlined in Example 7 (Step 2), starting from of N-(3-cyanophenyl)-N-(3-methoxybenzyl)pentanamide, the title compound was obtained as a yellow solid.

¹H NMR (300 MHz, DMSO-d₆) δ [ppm] 7.96 (d, J=7.8 Hz, 1H), 7.86 (s, 1H), 7.61 (t, J=7.9 Hz, 1H), 7.44-7.33 (m, 1H), 7.20 (t, J=7.8 Hz, 1H), 6.84-6.70 (m, 3H), 4.90 (s, 2H), 3.69 (s, 3H), 2.24-2.04 (br s, 2H), 1.49 (quintet, J=7.5 Hz, 2H), 1.19 (hex, J=7.5 Hz, 2H), 0.76 (t, J=7.3 Hz, 3H). MS (ESI⁻): 364.1. HPLC (Condition A): Rt 3.34 min (HPLC purity 96.6%).

### EXAMPLE 14

### N-(4-methoxybenzyl)-N-[3-(1H-tetrazol-5-yl)phenyl]pentanamide

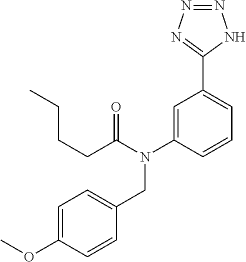

### Step 1: N-(3-cyanophenyl)-N-(4-methoxybenzyl)pentanamide

Following the general method as outlined in Example 7 (Step 1), starting from 3-[(2,3-dihydro-1,4-benzodioxin-6-ylmethyl)amino]-4-fluorobenzonitrile (Intermediate 10) and valeroyl chloride (Merck Kgaa), the title compound was obtained as a clear oil in 75% yield.

¹H NMR (300 MHz, DMSO-d₆) δ [ppm] 7.82-7.71 (m, 2H), 7.55 (t, J=8.1 Hz, 1H), 7.45 (d, J=8.1 Hz, 1H), 7.06 (d, J=8.6 Hz, 2H), 6.82 (d, J=8.6 Hz, 2H), 4.82 (s, 2H), 3.70 (s, 3H), 2.16-1.97 (m, 2H), 1.54-1.35 (m, 2H), 1.26-1.09 (m, 2H), 0.77 (t, J=7.3 Hz, 3H). MS (ESI⁺): 323.2. HPLC (Condition A): Rt 4.32 min (HPLC purity 94.5%).

### Step 2: N-(4-methoxybenzyl)-N-[3-(1H-tetrazol-5-yl)phenyl]pentanamide

Following the general method as outlined in Example 7 (Step 2), starting from of N-(3-cyanophenyl)-N-(3-methoxybenzyl)pentanamide, the title compound was obtained as a white solid.

¹H NMR (300 MHz, DMSO-d₆) δ [ppm] 7.96 (d, J=7.8 Hz, 1H), 7.82 (s, 1H), 7.59 (t, J=7.9 Hz, 1H), 7.31 (d, J=8.0 Hz, 1H), 7.10 (d, J=8.6 Hz, 2H), 6.83 (d, J=8.6 Hz, 2H), 4.85 (s, 2H), 3.10 (3, 3H), 2.22-2.01 (m, 2H), 1.48 (quintet, J=7.5 Hz, 2H), 1.28-1.08 (m, 2H), 0.76 (t, 3H). MS (ESI⁻): 364.1. HPLC (Condition A): Rt 3.72 min (HPLC purity 99.2%).

### EXAMPLE 15

### N-(2,3-dihydro-1,4-benzodioxin-6-ylmethyl)-N-[2-fluoro-5-(1H-tetrazol-5-yl)phenyl]pentanamide

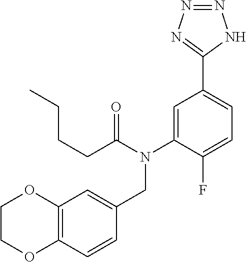

### Step 1: N-(5-cyano-2-fluorophenyl)-N-(2,3-dihydro-1,4-benzodioxin-6-ylmethyl)pentanamide

Following the general method as outlined in Example 7 (Step 1), starting from 3-[(2,3-dihydro-1,4-benzodioxin-6-ylmethyl)amino]-4-fluorobenzonitrile (Intermediate 8) and valeroyl chloride (Merck Kgaa), the title compound was obtained as a yellow oil.

¹H NMR (300 MHz, DMSO-d₆) δ [ppm] 7.99-7.88 (m, 2H), 7.56 (t, J=9.0 Hz, 1H), 6.78-6.53 (m, 3H), 4.79-4.62 (m, 2H), 4.18 (s, 4H), 2.15-1.84 (m, 2H), 1.45 (quintet, J=7.4 Hz, 2H), 1.23-1.10 (m, 2H), 0.76 (t, J=7.2 Hz, 3H). MS (ESI⁻): 369.2. HPLC (Condition A): Rt 4.35 min (HPLC purity 83.2%).

### Step 2: N-(2,3-dihydro-1,4-benzodioxin-6-ylmethyl)-N-[2-fluoro-5-(1H-tetrazol-5-yl)phenyl]pentanamide

Following the general method as outlined in Example 7 (Step 2), starting from of N-(5-cyano-2-fluorophenyl)-N-(2,3-dihydro-1,4-benzodioxin-6-ylmethyl)pentanamide, the title compound was obtained as a white solid.

¹H NMR (300 MHz, DMSO-d₆) δ [ppm] 8.10-8.00 (m, 1H), 7.98-7.86 (m, 1H), 7.59 (t, J=9.2 Hz, 1H), 6.82-6.56 (m, 3H), 4.81 (d, J=14.6 Hz, 1H), 4.67 (d, J=14.6 Hz, 1H), 4.17 (s, 4H), 2.17-1.92 (m, 2H), 1.55-1.40 (m, 2H), 1.26-1.08 (m, 2H), 0.75 (t, J=7.3 Hz, 3H). MS (ESI⁻): 410.2. HPLC (Condition A): Rt 3.79 min (HPLC purity 99.9%).

### EXAMPLE 16

### N-[3-(1H-tetrazol-5-yl)phenyl]-N-(2-thienylmethyl)pentanamide

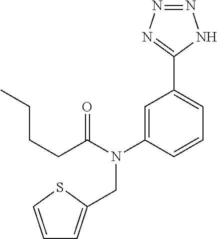

### Step 1: N-(3-cyanophenyl)-N-(2-thienylmethyl)pentanamide

A solution of 3-[(2-thienylmethyl)amino]benzonitrile (Intermediate 16; 527.00 mg; 2.46 mmol) and NMM (0.50 ml) in DMF (2 ml) was treated with valeryl chloride (Merck Kgaa; 0.50 ml; 4.2 mmol). The reaction solution was heated at 50° C. for 16 h then cooled and the solvent removed under vacuum. The residue was dissolved in DCM and washed with an aqueous solution (1 N) of HCl then with a saturated aqueous solution of NaHCO₃. The organic layer was dried with MgSO₄ then concentrated to give a residue, which was purified by flash column chromatography (silica), eluting with cyclohexane containing increasing amounts of EtOAc, to give the Title compound (520 mg, 71%) as a yellow oil.

¹H NMR (300 MHz, DMSO-d₆) δ [ppm] 7.80 (d, J=7.7 Hz, 1H), 7.74 (t, J=1.8 Hz, 1H), 7.59 (t, J=7.9 Hz, 1H), 7.50-7.44 (m, 1H), 7.42 (dd, J=5.1, 1.3 Hz, 1H), 6.89 (dd, J=5.1, J=3.4 Hz, 1H), 6.84-6.79 (m, 1H), 5.02 (s, 2H), 2.14-1.95 (m, 2H), 1.45 (quintet, J=7.4 Hz, 2H), 1.18 (sextet, J=7.4 Hz, 2H), 0.76 (t, 3H). MS (ESI⁺): 299.0. HPLC (Condition A): Rt 3.93 min (HPLC purity 99.4%).

### Step 2: N-[3-(1H-tetrazol-5-yl)phenyl]-N-(2-thienylmethyl)pentanamide

A suspension of N-(3-cyanophenyl)-N-(2-thienylmethyl)pentanamide (520 mg; 1.74 mmol), Copper (I) oxide (7 mg; 0.05 mmol) in DMF (3 ml) and MeOH (0.3 ml) was placed in a sealed vial and treated with trimethylsily azide (0.50 ml; 3.82 mmol). After stirring for 10 minutes at RT, the mixture was heated at 80° C. for 16. The solution was concentrated under vacuum, then diluted with EtOAc and washed with an aqueous solution (1 N) of HCl. After extraction with an aqueous solution (0.1 N) of NaOH (3 times), the combined aqueous phases were acidified with aqueous HCl (1 N) until pH 2 and extracted with EtOAc. The organic phase was dried on MgSO₄ and concentrated to give the Title compound as a white solid.

¹H NMR (300 MHz, DMSO-d₆) δ [ppm] 8.00 (d, J=7.8 Hz, 1H), 7.83 (s, 1H), 7.64 (t, J=7.8 Hz, 1H), 7.42 (dd, J=5.1, J=1.3 Hz, 1H), 7.39-7.32 (m, 1H), 6.89 (dd, J=5.1, J=1.3 Hz, 1H), 6.85-6.80 (m, 1H), 5.04 (s, 2H), 2.20-1.94 (m, 2H), 1.47 (q, J=7.4 Hz, 2H), 1.17 (sextet, J=7.3 Hz, 2H), 0.75 (t, J=7.3 Hz, 3H). MS (ESI⁻): 340.1. HPLC (Condition A): Rt 3.61 min (HPLC purity 99.8%).

### EXAMPLE 17

### N-[3-(1H-tetrazol-5-yl)phenyl]-N-(2-thienylmethyl)propanamide

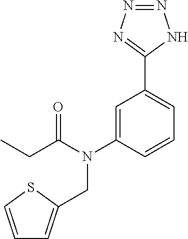

### Step 1: N-(3-cyanophenyl)-N-(2-thienylmethyl)propanamide

Following the general method as outlined in Example 16 (Step 1), starting from 3-[(2-thienylmethyl)amino]benzonitrile (Intermediate 16) and propionyl chloride (Alfa-Aesar), the title compound was obtained as a yellow oil.

¹H NMR (300 MHz, DMSO-d₆) δ [ppm] 7.81 (d, J=7.7 Hz, 1H), 7.75 (t, J=1.6 Hz, 1H), 7.59 (t, J=7.8 Hz, 1H), 7.53-7.46 (m, 1H), 7.41 (dd, J=5.1, J=1.3 Hz, 1H), 6.89 (dd, J=5.1, J=1.6 Hz, 1H), 6.82 (d, J=2.8 Hz, 1H), 5.03 (s, 2H), 2.15-2.00 (m, 2H), 0.94 (t, J=7.4 Hz, 3H). MS (ESI⁺): 270.9. HPLC (Condition A): Rt 3.59 min (HPLC purity 98.4%).

### Step 2: N-[3-(1H-tetrazol-5-yl)phenyl]-N-(2-thienylmethyl)propanamide

Following the general method as outlined in Example 16 (Step 2), starting from N-(3-cyanophenyl)-N-(2-thienylmethyl)propanamide, the title compound was obtained as a white solid.

¹H NMR (300 MHz, DMSO-d₆) δ [ppm] 8.00 (d, J=7.8 Hz, 1H), 7.84 (s, 1H), 7.64 (t, J=7.9 Hz, 1H), 7.44-7.34 (m, 2H), 6.89 (dd, J=5.1, 3.4 Hz, 1H), 6.85-6.80 (m, 1H), 5.05 (s, 2H), 2.21-1.93 (m, 2H), 0.96 (t, J=7.4 Hz, 3H). MS (ESI⁻): 312.1. HPLC (Condition A): Rt 3.00 min (HPLC purity 100.0%).

### EXAMPLE 18

### N-[3-(1H-tetrazol-5-yl)phenyl]-N-(3-thienylmethyl)propanamide

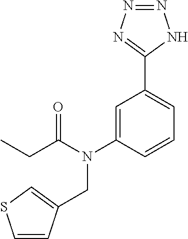

### Step 1: N-(3-cyanophenyl)-N-(3-thienylmethyl)propanamide

Following the general method as outlined in Example 16 (Step 1), starting from 3-[(3-thienylmethyl)amino]benzonitrile (Intermediate 9) and propionyl chloride (Alfa Aesar), the title compound was obtained as a yellow solid in 83% yield.

¹H NMR (300 MHz, DMSO-d₆) δ [ppm] 7.81-7.74 (m, 2H), 7.61-7.50 (m, 2H), 7.46 (dd, J=5.0, J=3.0 Hz, 1H), 7.23 (s, 1H), 6.95 (dd, J=5.0, J=1.2 Hz, 1H), 4.87 (s, 2H), 2.25-1.93 (m, 2H), 0.95 (t, J=7.4 Hz, 3H). MS (ESI⁻): 270.9. HPLC (Condition A): Rt 3.58 min (HPLC purity 99.3%).

### Step 2: N-[3-(1H-tetrazol-5-yl)phenyl]-N-(3-thienylmethyl)propanamide

Following the general method as outlined in Example 16 (Step 2), starting from of N-(3-cyanophenyl)-N-(3-thienylmethyl)propanamide, the title compound was obtained as a white solid in 71% yield.

¹H NMR (300 MHz, DMSO-d₆) δ [ppm] 7.97 (d, J=7.8 Hz, 1H), 7.85 (s, 1H), 7.62 (t, J=7.9 Hz, 1H), 7.47 (dd, J=4.9, J=2.9 Hz, 1H), 7.43-7.36 (m, 1H), 7.24 (s, 1H), 6.98 (dd, J=4.9, J=1.2 Hz, 1H), 4.89 (s, 2H), 2.21-2.00 (m, 2H), 0.96 (t, J=7.4 Hz 3H). MS (ESI⁻): 312.1. HPLC (Condition A): Rt 2.94 min (HPLC purity 99.5%).

### EXAMPLE 19

### N-(2,3-dihydro-1,4-benzodioxin-6-ylmethyl)-N-[3-(1H-tetrazol-5-yl)phenyl]butanamide

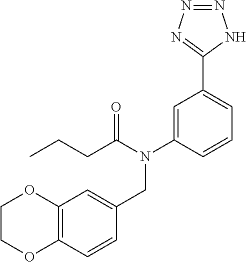

### Step 1: N-(3-cyanophenyl)-N-(2,3-dihydro-1,4-benzodioxin-6-ylmethyl)butanamide

Following the general method as outlined in Example 16 (Step 1), starting from 3-[(2,3-dihydro-1,4-benzodioxin-6-ylmethyl)amino]benzonitrile (Intermediate 9) and butyryl chloride (Aldrich), the title compound was obtained as a yellow oil in quantitative yield.

¹H NMR (300 MHz, DMSO-d₆) δ [ppm] 7.83-7.73 (m, 2H), 7.57 (t, J=8.1 Hz, 1H), 7.48 (d, J=8.4 Hz, 1H), 6.74 (d, J=8.2 Hz, 1H), 6.65 (d, J=2.0 Hz, 1H), 6.59 (dd, J=8.2, J=1.9 Hz, 1H), 4.77 (s, 2H), 4.18 (s, 4H), 2.13-1.97 (m, 2H), 1.56-1.42 (m, 2H), 0.78 (t, J=7.4 Hz, 3H). MS (ESI⁺): 337.1. HPLC (Condition A): Rt 3.93 min (HPLC purity 94.1%).

### Step 2: N-(2,3-dihydro-1,4-benzodioxin-6-ylmethyl)-N-[3-(1H-tetrazol-5-yl)phenyl]butanamide

Following the general method as outlined in Example 16 (Step 2), starting from of N-(3-cyanophenyl)-N-(2,3-dihydro-1,4-benzodioxin-6-ylmethyl)butanamide, the title compound was obtained as a yellow solid.

¹H NMR (300 MHz, DMSO-d₆) δ [ppm] 7.97 (d, J=7.8 Hz, 1H), 7.83 (s, 1H), 7.61 (t, J=7.8 Hz, 1H), 7.39-7.31 (m, 1H), 6.74 (d, J=8.2 Hz, 1H), 6.68 (d, J=1.9 Hz, 1H), 6.62 (dd, J=8.2, J=1.9 Hz, 1H), 4.80 (s, 2H), 4.18 (s, 4H), 2.17-2.00 (m, 2H), 1.51 (sextet, J=7.3 Hz, 2H), 0.78 (t, J=7.3 Hz, 3H). MS (ESI⁻): 378.2. HPLC (Condition A): Rt 3.29 min (HPLC purity 99.5%).

### EXAMPLE 20

### N-(2,3-dihydro-1,4-benzodioxin-6-ylmethyl)-N-[3-(1H-tetrazol-5-yl)phenyl]propanamide

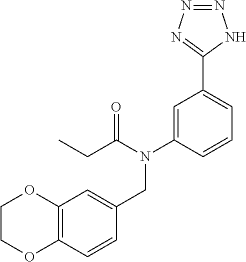

### Step 1: N-(3-cyanophenyl)-N-(2,3-dihydro-1,4-benzodioxin-6-ylmethyl)propanamide

Following the general method as outlined in Example 16 (Step 1), starting from 3-[(2,3-dihydro-1,4-benzodioxin-6-ylmethyl)amino]benzonitrile (Intermediate 4) and propionyl chloride (Alfa Aesar), the title compound was obtained as a clear oil.

¹H NMR (300 MHz, DMSO-d₆) δ [ppm] 7.81-7.72 (m, 2H), 7.61-7.46 (m, 2H), 6.74 (d, J=8.2 Hz, 1H), 6.66 (d, J=2.0 Hz, 1H), 6.59 (dd, J=8.2, 2.0 Hz, 1H), 4.77 (s, 2H), 4.18 (s, 4H), 2.21-2.00 (m, 2H), 0.94 (t, J=7.4 Hz, 3H). MS (ESI⁺): 323.1. HPLC (Condition A): Rt 3.70 min (HPLC purity 100.0%).

### Step 2: N-(2,3-dihydro-1,4-benzodioxin-6-ylmethyl)-N-[3-(1H-tetrazol-5-yl)phenyl]propanamide

Following the general method as outlined in Example 16 (Step 2), starting from of N-(3-cyanophenyl)-N-(2,3-dihydro-1,4-benzodioxin-6-ylmethyl)propanamide, the title compound was obtained as a white solid.

¹H NMR (300 MHz, DMSO-d₆) δ [ppm] 7.96 (d, J=7.6 Hz, 1H), 7.84 (s, 1H), 7.61 (t, J=7.9 Hz, 1H), 7.41-7.34 (m, 1H), 6.74 (d, J=8.2 Hz, 1H), 6.69 (d, J=1.9 Hz, 1H), 6.62 (dd, J=8.2, J=1.9 Hz, 1H), 4.80 (s, 2H), 4.18 (s, 4H), 2.20-2.03 (m, 2H), 0.96 (t, J=7.4 Hz, 3H). MS (ESI⁻): 364.2. HPLC (Condition A): Rt 2.55 min (HPLC purity 96.9%).

### EXAMPLE 21

### N-[3-(1H-tetrazol-5-yl)phenyl]-N-(3-thienylmethyl)pentanamide

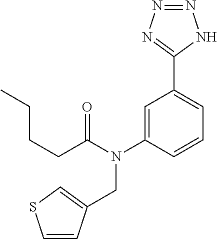

### Step 1: N-(3-cyanophenyl)-N-(3-thienylmethyl)pentanamide

Following the general method as outlined in Example 16 (Step 1), starting from 3-[(3-thienylmethyl)amino]benzonitrile (Intermediate 9) and valeroyl chloride (Merck Kgaa), the title compound was obtained as a yellow oil in 78% yield.

¹H NMR (300 MHz, DMSO-d₆) δ [ppm] 7.83-7.73 (m, 2H), 7.63-7.43 (m, 3H), 7.22 (s, 1H), 6.94 (dd, J=5.0, J=1.2 Hz, 1H), 4.86 (s, 2H), 2.24-1.85 (m, 2H), 1.45 (quintet, J=7.4 Hz, 2H), 1.28-1.08 (m, 2H), 0.76 (t, J=7.3 Hz, 3H). MS (ESI⁺): 299.0. HPLC (Condition A): Rt 4.23 min (HPLC purity 95.1%).

### Step 2: N-[3-(1H-tetrazol-5-yl)phenyl]-N-(3-thienylmethyl)pentanamide

Following the general method as outlined in Example 16 (Step 2), starting from of N-(3-cyanophenyl)-N-(3-thienylmethyl)pentanamide, the title compound was obtained as a white solid in 78% yield.

¹H NMR (300 MHz, DMSO-d₆) δ [ppm] 7.98 (d, J=7.7 Hz, 1H), 7.84 (s, 1H), 7.62 (t, J=7.9 Hz, 1H), 7.47 (dd, J=4.9, 3.0 Hz, 1H), 7.41-7.34 (m, 1H), 7.23 (s, 1H), 6.97 (dd, J=4.9, J=1.2 Hz, 1H), 4.89 (s, 2H), 2.21-1.98 (m, 2H), 1.48 (quintet, J=7.4 Hz, 2H), 1.17 (hex, J=7.3 Hz, 2H), 0.75 (t, J=7.3 Hz, 3H). MS (ESI⁻): 340.2. HPLC (Condition A): Rt 3.67 min (HPLC purity 97.7%).

### EXAMPLE 22

### N-[3-(1H-tetrazol-5-yl)phenyl]-N-[4-(trifluoromethoxy)benzyl]propanamide

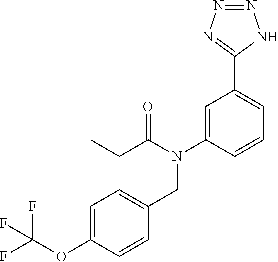

### Step 1: N-(3-cyanophenyl)-N-[4-(trifluoromethoxy)benzyl]propanamide

A cooled (0° C.) solution of N-(3-cyanophenyl)propanamide (Intermediate 18; 200 mg; 1.15 mmol) in DMF (5 ml) was treated with NaH (60% dispersion in mineral oil; 60 mg; 1.15 mmol), followed after 10 min by treatment of 4-(trifluoromethoxy)benzyl bromide (Aldrich; 230 μl; 1.44 mmol). The reaction was stirred at 0° C. for 2 h, then iPrOH was added to quench the reaction. The solution was diluted with EtOAc and washed four times with brine. The organic phase was dried on MgSO₄ and concentrated under vacuum, to give a residue which was purified by flash column chromatography (silica), eluting with cyclohexane containing increasing amounts of EtOAc, to give the Title compound (348 mg, 87%) as a colourless oil.

¹H NMR (300 MHz, DMSO-d₆) δ [ppm] 7.61 (m, 1H), 7.55 (m, 1H), 7.36-7.33 (m, 2H), 7.09 (d, J=8.8, 2H), 7.05 (d, J=8.8, 2H), 4.70 (s, 2H), 1.88 (m, 2H), 0.73 (t, J=7.3 Hz, 3H). MS (ESI⁻): 348.9. HPLC (Condition A): Rt 4.01 min (HPLC purity 99.7%).

### Step 2: N-[3-(1H-tetrazol-5-yl)phenyl]-N-[4-(trifluoromethoxy)benzyl]propanamide

A suspension of N-(3-cyanophenyl)-N-[4-(trifluoromethoxy)benzyl]propanamide (348.00 mg; 1.00 mmol), Copper (I) oxide (7 mg; 0.05 mmol) in DMF (2.7 ml) and MeOH (0.3 ml) was placed in a sealed vial and treated with trimethylsily azide (170 □1; 1.30 mmol). After stirring for 10 minutes at RT, the mixture was heated at 80° C. for 16. The solution was concentrated under vacuum, then diluted with EtOAc and washed with an aqueous solution (1 N) of HCl. After extraction with an aqueous solution (0.1 N) of NaOH (3 times), the combined aqueous phases were acidified with aqueous HCl (1 N) until pH 2 and extracted with EtOAc. The organic phase was dried on MgSO₄ and concentrated to give the Title compound as a white solid.

¹H NMR (300 MHz, DMSO-d₆) δ [ppm] 7.91 (d, J=7.7 Hz, 1H), 7.82 (t, J=1.8 Hz, 1H), 7.56 (t, J=7.9 Hz, 1H), 7.47 (ddd, J=7.9, J=1.8, J=1.0 Hz, 1H), 7.28 (d, J=8.8, 2H), 7.23 (d, J=8.8, 2H), 4.89 (s, 2H), 2.09 (m, 2H), 0.92 (t, J=7.3 Hz, 3H). MS (ESI⁻): 390.1. HPLC (Condition A): Rt 3.21 min (HPLC purity 99.8%).

### EXAMPLE 23

### N-[(5-methyl-3-phenylisoxazol-4-yl)methyl]-N-[3-(1H-tetrazol-5-yl)phenyl]propanamide

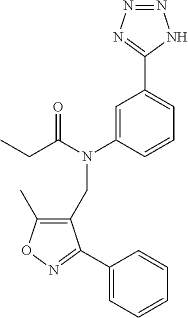

### Step 1: N-(3-cyanophenyl)-N-[(5-methyl-3-phenylisoxazol-4-yl)methyl]propanamide

Following the general method as outlined in Example 22 (Step 1), starting from N-(3-cyanophenyl)propanamide (Intermediate 18) and 4-(bromomethyl)-5-methyl-3-phenylisoxazole (ABCR), the title compound was obtained as a yellow gum in 86% yield.

¹H NMR (300 MHz, DMSO-d₆) δ [ppm] 7.64 (dt, J=8.0 Hz, J=1.3 Hz, 1H), 7.51-7.28 (m, 7H), 7.23 (ddd, J=8.0 Hz, J=2.0 Hz, J=1.0 Hz, 1H), 4.89 (s, 2H), 2.31 (s, 3H), 1.86 (m, 2H), 0.85 (t, J=7.4 Hz, 3H). MS (ESI⁺): 346.0. HPLC (Condition A): Rt 3.75 min (HPLC purity 95.4%).

### Step 2: N-[(5-methyl-3-phenylisoxazol-4-yl)methyl]-N-[3-(1H-tetrazol-5-yl)phenyl]propanamide

Following the general method as outlined in Example 22 (Step 2), starting from of N-(3-cyanophenyl)-N-[(5-methyl-3-phenylisoxazol-4-yl)methyl]propanamide, the title compound was obtained as a white solid.

¹H NMR (300 MHz, DMSO-d₆) δ [ppm]. 1H NMR (DMSO-d6) δ 7.86 (dt, J=7.9 Hz, J=1.2 Hz, 1H), 7.53 (t, J=1.8 Hz, 1H), 7.46 (t, J=7.9 Hz, 1H), 7.31-7.23 (m, 5H), 7.13 (d, J=7.9 Hz, 1H), 4.93 (s, 2H), 2.28 (s, 3H), 1.91 (m, 2H), 0.87 (t, J=7.3 Hz, 3H). MS (ESI⁻): 387.2. HPLC (Condition A): Rt 3.11 min (HPLC purity 99.3%).

### EXAMPLE 24

### N-[(3-methyl-5-phenylisoxazol-4-yl)methyl]-N-[3-(1H-tetrazol-5-yl)phenyl]propanamide

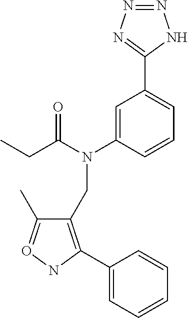

### Step 1: N-(3-cyanophenyl)-N-[(3-methyl-5-phenylisoxazol-4-yl)methyl]propanamide

Following the general method as outlined in Example 22 (Step 1), starting from N-(3-cyanophenyl)propanamide (Intermediate 18) and 4-(bromomethyl)-3-methyl-5-phenylisoxazole (Acros), the title compound was obtained as a yellow gum in 84% yield.

¹H NMR (300 MHz, DMSO-d₆) δ [ppm] 7.65 (dt, J=7.70 Hz, J=1.3 Hz, 1H), 7.58 (m, 1H), 7.47-7.29 (m, 7H), 4.99 (s, 2H), 2.15 (s, 3H), 1.94 (m, 2H), 0.91 (t, J=7.4 Hz, 3H). MS (ESI⁺): 346.1. HPLC (Condition A): Rt 3.97 min (HPLC purity 94.8%).

### Step 2: N-[(3-methyl-5-phenylisoxazol-4-yl)methyl]-N-[3-(1H-tetrazol-5-yl)phenyl]propanamide

Following the general method as outlined in Example 22 (Step 2), starting from of N-(3-cyanophenyl)-N-[(3-methyl-5-phenylisoxazol-4-yl)methyl]propanamide, the title compound was obtained as a white solid.

¹H NMR (300 MHz, DMSO-d₆) δ [ppm] 7.86 (d, J=7.9 Hz, 1H), 7.64 (t, J=1.7 Hz, 1H), 7.47 (t, J=7.9 Hz, 1H), 7.35-7.25 (m, 5H), 7.21 (d, J=7.9 Hz, 1H), 5.02 (s, 2H), 2.13 (s, 3H), 2.00 (m, 2H), 0.93 (t, J=7.4 Hz, 3H). MS (ESI⁻): 387.2. HPLC (Condition A): Rt 3.21 min (HPLC purity 99.1%).

### EXAMPLE 25

### N-(3-methoxybenzyl)-N-[3-(1H-tetrazol-5-yl)phenyl]propanamide

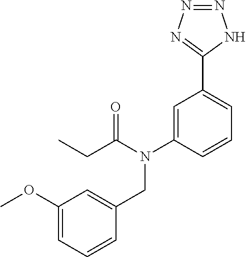

### Step 1: N-(3-cyanophenyl)-N-(3-methoxybenzyl)propanamide

Following the general method as outlined in Example 22 (Step 1), starting from N-(3-cyanophenyl)propanamide (Intermediate 18) and 3-methoxybenzyl bromide (Aldrich), the title compound was obtained as a yellow gum in 72% yield.

¹H NMR (300 MHz, DMSO-d₆) δ [ppm] 7.81 (m, 1H), 7.76 (m, 1H), 7.59-7.52 (m, 2H), 7.19 (t, J=7.9 Hz, 1H), 6.80-6.71 (m, 3H), 4.88 (s, 2H), 3.69 (s, 3H), 2.11 (m, 2H), 0.96 (t, J=7.4 Hz, 3H). MS (ESI⁺): 295.0. HPLC (Condition A): Rt 3.90 min (HPLC purity 97.6%).

### Step 2: N-(3-methoxybenzyl)-N-[3-(1H-tetrazol-5-yl)phenyl]propanamide

Following the general method as outlined in Example 22 (Step 2), starting from of N-(3-cyanophenyl)-N-(3-methoxybenzyl)propanamide, the title compound was obtained as a white solid.

¹H NMR (300 MHz, DMSO-d₆) δ [ppm] 7.95 (d, J=7.9 Hz, 1H), 7.88 (s, 1H), 7.60 (t, J=7.9 Hz, 1H), 7.41 (t, J=7.9 Hz, 1H), 7.20 (t, J=7.9 Hz, 1H), 6.80-6.75 (m, 3H), 4.91 (s, 2H), 3.68 (s, 3H), 2.16 (m, 2H), 0.98 (t, J=7.3 Hz, 3H). MS (ESI⁻): 336.2. HPLC (Condition A): Rt 3.18 min (HPLC purity 98.1%).

### EXAMPLE 26

### N-(4-methoxybenzyl)-N-[3-(1H-tetrazol-5-yl)phenyl]propanamide

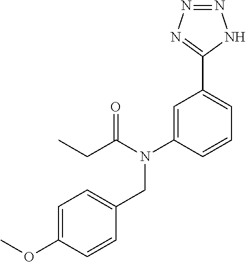

### Step 1: N-(3-cyanophenyl)-N-(4-methoxybenzyl)propanamide

Following the general method as outlined in Example 22 (Step 1), starting from N-(3-cyanophenyl)propanamide (Intermediate 18) and 4-methoxybenzyl bromide (Aldrich), the title compound was obtained as a yellow gum in 87% yield.

¹H NMR (300 MHz, DMSO-d₆) δ [ppm] 7.76 (m, 2H), 7.55 (t, J=8.0 Hz, 1H), 7.46 (dt, J=8.0 Hz, J=1.5 Hz, 1H), 7.07 (d, J=8.7 Hz, 2H), 6.82 (d, J=8.7 Hz, 2H), 4.83 (s, 2H), 3.70 (s, 3H), 2.07 (m, 2H), 0.95 (t, J=7.4 Hz, 3H). MS (ESI⁺): 295.0. HPLC (Condition A): Rt 3.81 min (HPLC purity 94.7%).

### Step 2: N-(4-methoxybenzyl)-N-[3-(1H-tetrazol-5-yl)phenyl]propanamide

Following the general method as outlined in Example 22 (Step 2), starting from of N-(3-cyanophenyl)-N-(4-methoxybenzyl)propanamide, the title compound was obtained as a white solid.

¹H NMR (300 MHz, DMSO-d₆) δ [ppm] 7.95 (d, J=7.9 Hz, 1H), 7.83 (s, 1H), 7.59 (t, J=7.9 Hz, 1H), 7.33 (d, J=7.9 Hz, 1H), 7.10 (d, J=8.6 Hz, 2H), 6.83 (d, J=8.6 Hz, 2H), 4.85 (s, 2H), 3.69 (s, 3H), 2.10 (m, 2H), 0.96 (t, J=7.4 Hz, 3H). MS (ESI⁻): 336.1. HPLC (Condition A): Rt 3.11 min (HPLC purity 95.9%).

### EXAMPLE 27

### N-[3-(1H-tetrazol-5-yl)phenyl]-N-[4-(trifluoromethyl)benzyl]propanamide

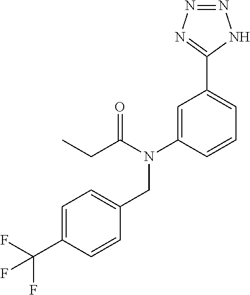

### Step 1: N-(3-cyanophenyl)-N-[4-(trifluoromethyl)benzyl]propanamide

Following the general method as outlined in Example 22 (Step 1), starting from N-(3-cyanophenyl)propanamide (Intermediate 18) and 4-(trifluoromethyl)benzyl bromide (Aldrich), the title compound was obtained as a yellow gum.

¹H NMR (300 MHz, DMSO-d₆) δ [ppm] 7.88 (m, 1H), 7.79-7.76 (m, 1H), 7.65 (d, J=8.0 Hz, 2H), 7.60-7.57 (m, 2H), 7.43 (d, J=8.0 Hz, 2H), 4.99 (s, 2H), 2.13 (m, 2H), 0.96 (t, J=7.3 Hz, 3H). MS (ESI⁺): 333.0. HPLC (Condition A): Rt 4.40 min (HPLC purity 95.4%).

### Step 2: N-[3-(1H-tetrazol-5-yl)phenyl]-N-[4-(trifluoromethyl)benzyl]propanamide

Following the general method as outlined in Example 22 (Step 2), starting from of N-(3-cyanophenyl)-N-[4-(trifluoromethyl)benzyl]propanamide, the title compound was obtained as a white solid.

¹H NMR (300 MHz, DMSO-d₆) δ [ppm] δ 7.97 (d, J=7.8 Hz, 1H), 7.91 (t, J=1.6 Hz, 1H), 7.68-7.59 (m, 3H), 7.47-7.43 (m, 3H), 5.02 (s, 2H), 2.17 (m, 2H), 0.98 (t, J=7.4 Hz, 3H). MS (ESI⁻): 374.3. HPLC (Condition A): Rt 3.76 min (HPLC purity 99.6%).

### EXAMPLE 28

### N-[3-(1H-tetrazol-5-yl)phenyl]-N-[3-(trifluoromethyl)benzyl]propanamide

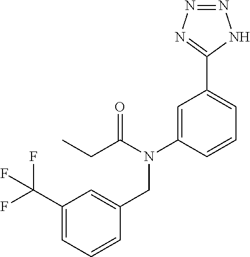

### Step 1: N-(3-cyanophenyl)-N-[3-(trifluoromethyl)benzyl]propanamide

Following the general method as outlined in Example 22 (Step 1), starting from N-(3-cyanophenyl)propanamide (Intermediate 18) and 3-(trifluoromethyl)benzyl bromide (Aldrich), the title compound was obtained as a yellow gum.

¹H NMR (300 MHz, DMSO-d₆) δ [ppm] 7.84 (m, 1H), 7.78 (m, 1H), 7.60-7.50 (m, 6H), 5.00 (s, 2H), 2.13 (m, 2H), 0.96 (t, J=7.3 Hz, 3H). MS (ESI⁺): 333.1. HPLC (Condition A): Rt 4.51 min (HPLC purity 100.0%).

### Step 2: N-[3-(1H-tetrazol-5-yl)phenyl]-N-[3-(trifluoromethyl)benzyl]propanamide

Following the general method as outlined in Example 22 (Step 2), starting from of N-(3-cyanophenyl)-N-[3-(trifluoromethyl)benzyl]propanamide, the title compound was obtained as a white solid in 72% yield.

¹H NMR (300 MHz, DMSO-d₆) δ [ppm] 7.97 (d, J=7.8 Hz, 1H), 7.87 (m, 1H), 7.64-7.53 (m, 5H), 7.42 (ddd, J=7.8 Hz, J=2.0 Hz, J=1.0 Hz, 1H), 5.02 (s, 2H), 2.16 (m, 2H), 0.98 (t, J=7.4 Hz, 3H). MS (ESI⁻): 374.1. HPLC (Condition A): Rt 4.31 min (HPLC purity 99.7%).

### EXAMPLE 29

### N-[3-fluoro-5-(1H-tetrazol-5-yl)phenyl]-N-[3-(trifluoromethoxy)benzyl]pentanamide

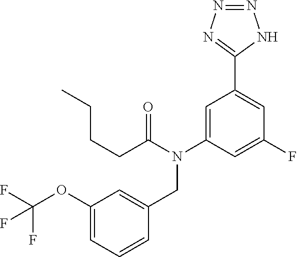

### Step 1: N-(3-cyano-5-fluorophenyl)-N-[3-(trifluoromethoxy)benzyl]pentanamide

Following the general method as outlined in Example 22 (Step 1), starting from N-(3-cyano-5-fluorophenyl)pentanamide (Intermediate 19) and 3-(trifluoromethoxy)benzyl bromide (ABCR), the title compound was obtained as a yellow gum.

¹H NMR (300 MHz, DMSO-d₆) δ [ppm] 7.82 (d, J=8.3 Hz, 1H), 7.70 (t, J=1.5 Hz, 1H), 7.64 (dt, J=9.7 Hz, J=2.2 Hz, 1H), 7.43 (t, J=8.0 Hz, 1H), 7.26-7.21 (m, 2H), 7.16 (m, 1H), 4.97 (s, 2H), 2.19 (m, 2H), 1.47 (quintet, J=7.5 Hz, 2H), 1.20 (sextet, J=7.5 Hz, 2H), 0.78 (t, J=7.5 Hz, 3H). MS (ESI⁺): 395.2. HPLC (Condition A): Rt 5.38 min (HPLC purity 98.8%).

### Step 2: N-[3-fluoro-5-(1H-tetrazol-5-yl)phenyl]-N-[3-(trifluoromethoxy)benzyl]pentanamide

Following the general method as outlined in Example 22 (Step 2), starting from of N-(3-cyano-5-fluorophenyl)-N-[3-(trifluoromethoxy)benzyl]pentanamide, the title compound was obtained as a beige gum.

¹H NMR (300 MHz, DMSO-d₆) δ [ppm] 7.78 (d, J=9.2 Hz, 1H), 7.70 (s, 1H), 7.47-7.41 (m, 2H), 7.29-7.18 (m, 3H), 4.99 (s, 2H), 2.22 (m, 2H), 1.50 (quintet, J=7.5 Hz, 2H), 1.20 (sextet, J=7.5 Hz, 2H), 0.77 (t, J=7.5 Hz, 3H). MS (ESI⁻): 436.2. HPLC (Condition A): Rt 4.57 min (HPLC purity 97.5%).

### EXAMPLE 30

### N-benzyl-N-[3-(1H-tetrazol-5-yl)phenyl]propanamide

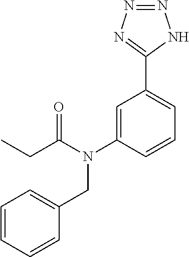

### Step 1: N-benzyl-N-(3-cyanophenyl)propanamide

Following the general method as outlined in Example 22 (Step 1), starting from N-(3-cyanophenyl)propanamide (Intermediate 18) and benzyl bromide (Aldrich), the title compound was obtained as a clear oil which solidified upon standing in 94% yield.

¹H NMR (300 MHz, DMSO-d₆) δ [ppm] 7.83-7.69 (m, 2H), 7.61-7.49 (m, 2H), 7.34-7.13 (m, 5H), 4.91 (s, 2H), 2.24-2.01 (m, 2H), 0.96 (t, J=7.4 Hz, 3H). MS (ESI⁺): 265.1. HPLC (Condition A): Rt 3.73 min (HPLC purity 100.0%).

### Step 2: N-benzyl-N-[3-(1H-tetrazol-5-yl)phenyl]propanamide

Following the general method as outlined in Example 22 (Step 2), starting from of N-benzyl-N-(3-cyanophenyl)propanamide, the title compound was obtained as a white solid.

¹H NMR (300 MHz, DMSO-d₆) δ [ppm]. 1H NMR (DMSO-d6) δ 7.95 (d, J=7.7 Hz, 1H), 7.86 (s, 1H), 7.60 (t, J=7.9 Hz, 1H), 7.43-7.36 (m, 1H), 7.33-7.16 (m, 5H), 4.93 (s, 2H), 2.25-2.06 (m, 2H), 0.98 (t, J=7.4 Hz, 3H). MS (ESI⁻): 306.2. HPLC (Condition A): Rt 3.10 min (HPLC purity 99.9%).

### EXAMPLE 31

### N-(4-fluorobenzyl)-N-[3-(1H-tetrazol-5-yl)phenyl]propanamide

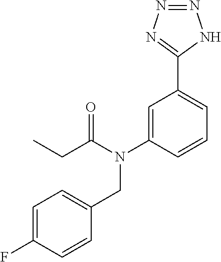

### Step 1: N-(3-cyanophenyl)-N-(4-fluorobenzyl)propanamide

Following the general method as outlined in Example 22 (Step 1), starting from N-(3-cyanophenyl)propanamide (Intermediate 18) and 4-fluorobenzyl bromide (Aldrich), the title compound was obtained as an opaque oil in 72% yield.

¹H NMR (300 MHz, DMSO-d₆) δ [ppm] 7.84-7.72 (m, 2H), 7.62-7.46 (m, 2H), 7.26-7.16 (m, 2H), 7.15-7.03 (m, 2H), 4.88 (s, 2H), 2.21-2.01 (m, 2H), 0.95 (t, 3H). MS (ESI⁺): 283.0. HPLC (Condition A): Rt 3.84 min (HPLC purity 99.3%).

### Step 2: N-(4-fluorobenzyl)-N-[3-(1H-tetrazol-5-yl)phenyl]propanamide

Following the general method as outlined in Example 22 (Step 2), starting from of N-(3-cyanophenyl)-N-(4-fluorobenzyl)propanamide, the title compound was obtained as a white solid.

¹H NMR (300 MHz, DMSO-d₆) δ [ppm] 7.96 (d, J=7.9 Hz, 1H), 7.84 (s, 1H), 7.60 (t, J=7.9 Hz, 1H), 7.42-7.33 (m, 1H), 7.29-7.19 (m, 2H), 7.16-7.05 (m, 2H), 4.91 (s, 2H), 2.22-2.05 (m, 2H), 0.97 (t, J=7.4 Hz, 3H). MS (ESI⁻): 324.2. HPLC (Condition A): Rt 3.22 min (HPLC purity 97.5%).

### EXAMPLE 32

### N-(3-fluorobenzyl)-N-[3-(1H-tetrazol-5-yl)phenyl]propanamide

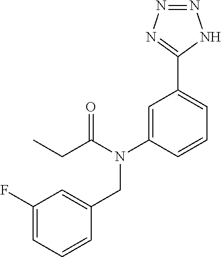

### Step 1: N-(3-cyanophenyl)-N-(3-fluorobenzyl)propanamide

Following the general method as outlined in Example 22 (Step 1), starting from N-(3-cyanophenyl)propanamide (Intermediate 18) and 3-fluorobenzyl bromide (VWR), the title compound was obtained as an opaque oil in 91% yield.

¹H NMR (300 MHz, DMSO-d₆) δ [ppm] 7.88-7.83 (m, 1H), 7.81-7.73 (m, 1H), 7.62-7.53 (m, 2H), 7.38-7.27 (m, 1H), 7.10-6.98 (m, 3H), 4.92 (s, 2H), 2.22-2.03 (m, 2H), 0.96 (t, 3H). MS (ESI⁺): 283.0. HPLC (Condition A): Rt 3.81 min (HPLC purity 100.0%).

### Step 2: N-(3-fluorobenzyl)-N-[3-(1H-tetrazol-5-yl)phenyl]propanamide

Following the general method as outlined in Example 22 (Step 2), starting from of N-(3-cyanophenyl)-N-(3-fluorobenzyl)propanamide, the title compound was obtained as a white solid.

¹H NMR (300 MHz, DMSO-d₆) δ [ppm] 7.97 (d, J=7.9 Hz, 1H), 7.91-7.86 (m, 1H), 7.62 (t, J=7.9 Hz, 1H), 7.48-7.40 (m, 1H), 7.39-7.28 (m, 1H), 7.12-6.99 (m, 3H), 4.94 (s, 2H), 2.27-2.04 (m, 2H), 0.98 (t, J=7.4 Hz, 3H). MS (ESI⁻): 324.2. HPLC (Condition A): Rt 3.21 min (HPLC purity 99.0%).

### EXAMPLE 33

### N-(2-fluorobenzyl)-N-[3-(1H-tetrazol-5-yl)phenyl]propanamide

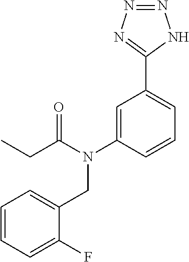

### Step 1: N-(3-cyanophenyl)-N-(2-fluorobenzyl)propanamide

Following the general method as outlined in Example 22 (Step 1), starting from N-(3-cyanophenyl)propanamide (Intermediate 18) and 2-fluorobenzyl bromide (Aldrich), the title compound was obtained as an opaque oil in 98% yield.

¹H NMR (300 MHz, DMSO-d₆) δ [ppm] 7.84 (s, 1H), 7.80-7.73 (m, 1H), 7.62-7.53 (m, 2H), 7.38-7.24 (m, 2H), 7.18-7.04 (m, 2H), 4.95 (s, 2H), 2.21-2.01 (m, 2H), 0.95 (t, J=7.4 Hz, 3H). MS (ESI⁺): 283.0. HPLC (Condition A): Rt 3.74 min (HPLC purity 99.7%).

### Step 2: N-(2-fluorobenzyl)-N-[3-(1H-tetrazol-5-yl)phenyl]propanamide

Following the general method as outlined in Example 22 (Step 2), starting from of N-(3-cyanophenyl)-N-(2-fluorobenzyl)propanamide, the title compound was obtained as a white solid.

¹H NMR (300 MHz, DMSO-d₆) δ [ppm] 7.98 (d, J=7.9 Hz, 1H), 7.90-7.85 (m, 1H), 7.60 (t, J=7.9 Hz, 1H), 7.48-7.41 (m, 1H), 7.40-7.22 (m, 2H), 7.19-7.04 (m, 2H), 4.98 (s, 2H), 2.26-2.02 (m, 2H), 0.97 (t, J=7.4 Hz, 3H). MS (ESI⁻): 324.2. HPLC (Condition A): Rt 3.12 min (HPLC purity 99.7%).

### EXAMPLE 34

### N-(1,3-benzodioxol-5-ylmethyl)-N-[3-(1H-tetrazol-5-yl)phenyl]propanamide

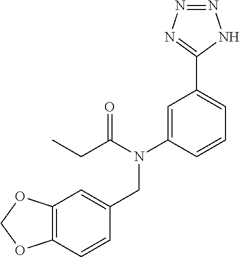

### Step 1: N-(1,3-benzodioxol-5-ylmethyl)-N-(3-cyanophenyl)propanamide

Following the general method as outlined in Example 22 (Step 1), starting from N-(3-cyanophenyl)propanamide (Intermediate 18) and 3,4-methylenedioxybenzyl chloride (ABCR), the title compound was obtained as a clear oil in 97% yield.

¹H NMR (300 MHz, DMSO-d₆) δ [ppm] 7.83-7.73 (m, 2H), 7.61-7.47 (m, 2H), 6.81-6.73 (m, 2H), 6.58 (dd, J=8.0, J=1.4 Hz, 1H), 5.97 (s, 2H), 4.80 (s, 2H), 2.19-1.97 (m, 2H), 0.95 (t, J=7.4 Hz, 3H). MS (ESI⁺): 308.9. HPLC (Condition A): Rt 3.74 min (HPLC purity 99.4%).

### Step 2: N-(1,3-benzodioxol-5-ylmethyl)-N-[3-(1H-tetrazol-5-yl)phenyl]propanamide

Following the general method as outlined in Example 22 (Step 2), starting from of N-(1,3-benzodioxol-5-ylmethyl)-N-(3-cyanophenyl)propanamide, the title compound was obtained as a white solid.

¹H NMR (300 MHz, DMSO-d₆) δ [ppm] 7.97 (d, J=7.7 Hz, 1H), 7.84 (s, 1H), 7.61 (t, J=7.9 Hz, 1H), 7.42-7.34 (m, 1H), 6.82-6.75 (m, 2H), 6.62 (dd, J=8.0, J=1.4 Hz, 1H), 5.97 (s, 2H), 4.83 (s, 2H), 2.21-2.04 (m, 2H), 0.97 (t, 3H). MS (ESI⁻): 350.2. HPLC (Condition A): Rt 3.09 min (HPLC purity 100.0%).

### EXAMPLE 35

### N-[3-fluoro-5-(1H-tetrazol-5-yl)phenyl]-N-[4-(trifluoromethoxy)benzyl]pentanamide

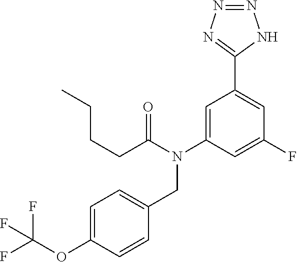

### Step 1: N-(3-cyano-5-fluorophenyl)-N-[4-(trifluoromethoxy)benzyl]pentanamide

Following the general method as outlined in Example 22 (Step 1), starting from N-(3-cyano-5-fluorophenyl)pentanamide (Intermediate 19) and 4-(trifluoromethoxy)benzyl bromide (Aldrich), the title compound was obtained as a clear oil.

¹H NMR (300 MHz, DMSO-d₆) δ [ppm] 7.83 (d, J=8.3 Hz, 1H), 7.72 (s, 1H), 7.66 (dt, J=9.9, J=2.2 Hz, 1H), 7.36-7.24 (m, 4H), 4.94 (s, 2H), 2.23-2.12 (m, 2H), 1.47 (quintet, J=7.4 Hz, 2H), 1.20 (sextet, J=7.5 Hz, 2H), 0.78 (t, J=7.3 Hz, 3H). MS (ESI⁺): 395.2. HPLC (Condition A): Rt 5.12 min (HPLC purity 99.4%).

### Step 2: N-[3-fluoro-5-(1H-tetrazol-5-yl)phenyl]-N-[4-(trifluoromethoxy)benzyl]pentanamide

Following the general method as outlined in Example 22 (Step 2), starting from of N-(3-cyano-5-fluorophenyl)-N-[4-(trifluoromethoxy)benzyl]pentanamide, the title compound was obtained as a white solid.

¹H NMR (300 MHz, DMSO-d₆) δ [ppm] 7.79 (d, J=8.9 Hz, 1H), 7.71 (s, 1H), 7.47 (dt, J=9.7, J=2.1 Hz, 1H), 7.35 (d, J=8.5 Hz, 2H), 7.29 (d, J=8.5 Hz, 2H), 4.97 (s, 2H), 2.27-2.15 (m, 2H), 1.50 (quin, J=7.5 Hz, 2H), 1.20 (septet, J=7.5 Hz, 2H), 0.77 (t, J=7.3 Hz, 3H). MS (ESI⁻): 436.2. HPLC (Condition A): Rt 4.61 min (HPLC purity 99.0%).

### EXAMPLE 36

### N-[3-fluoro-5-(1H-tetrazol-5-yl)phenyl]-N-[4-(trifluoromethoxy)benzyl]propanamide

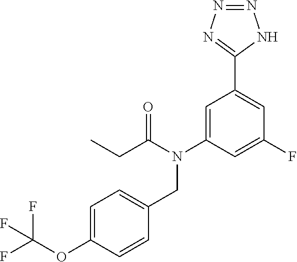

### Step 1: N-(3-cyano-5-fluorophenyl)-N-[4-(trifluoromethoxy)benzyl]propanamide

Following the general method as outlined in Example 22 (Step 1), starting from N-(3-cyano-5-fluorophenyl)propanamide (Intermediate 20) and 4-(trifluoromethoxy)benzyl bromide (Aldrich), the title compound was obtained as a clear oil.

¹H NMR (300 MHz, DMSO-d₆) δ [ppm] 7.87-7.78 (m, 1H), 7.73 (s, 1H), 7.66 (dt, J=9.9, J=2.2 Hz, 1H), 7.39-7.23 (m, 4H), 4.94 (s, 2H), 2.27-2.11 (m, 2H), 0.96 (t, J=7.3 Hz, 3H). MS (ESI⁺): 367.2. HPLC (Condition A): Rt 4.64 min (HPLC purity 95.9%).

### Step 2: N-[3-fluoro-5-(1H-tetrazol-5-yl)phenyl]-N-[4-(trifluoromethoxy)benzyl]propanamide

Following the general method as outlined in Example 22 (Step 2), starting from of N-(3-cyano-5-fluorophenyl)-N-[4-(trifluoromethoxy)benzyl]propanamide, the title compound was obtained as a white solid.

¹H NMR (300 MHz, DMSO-d₆) δ [ppm] 7.83-7.75 (m, 1H), 7.73 (s, 1H), 7.49 (dt, J=9.8, J=2.1 Hz, 1H), 7.36 (d, J=8.5 Hz, 2H), 7.29 (d, J=8.5 Hz, 2H), 4.97 (s, 2H), 2.29-2.15 (m, 2H), 0.98 (t, J=7.3 Hz, 3H). MS (ESI⁻): 408.1. HPLC (Condition A): Rt 4.12 min (HPLC purity 98.0%).

### EXAMPLE 37

### N-[3-fluoro-5-(1H-tetrazol-5-yl)phenyl]-N-(3-methoxybenzyl)pentanamide

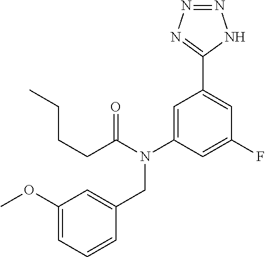

### Step 1: N-(3-cyano-5-fluorophenyl)-N-(3-methoxybenzyl)pentanamide

Following the general method as outlined in Example 22 (Step 1), starting from N-(3-cyano-5-fluorophenyl)pentanamide (Intermediate 19) and 3-(methoxy)benzyl bromide (Aldrich), the title compound was obtained as a clear oil.

¹H NMR (300 MHz, DMSO-d₆) δ [ppm] 7.85-7.77 (m, 1H), 7.70 (s, 1H), 7.62 (d t, J=9.9, J=2.2 Hz, 1H), 7.20 (t, J=7.8 Hz, 1H), 6.83-6.69 (m, 3H), 4.90 (s, 2H), 3.70 (s, 3H), 2.28-2.12 (m, 2H), 1.48 (quintet, J=7.4 Hz, 2H), 1.20 (hex, J=7.4 Hz, 2H), 0.79 (t, J=7.3 Hz, 3H). MS (ESI⁺): 341.2. HPLC (Condition A): Rt 4.54 min (HPLC purity 99.4%).

### Step 2: N-[3-fluoro-5-(1H-tetrazol-5-yl)phenyl]-N-(3-methoxybenzyl)pentanamide

Following the general method as outlined in Example 22 (Step 2), starting from of N-(3-cyano-5-fluorophenyl)-N-(3-methoxybenzyl)pentanamide, the title compound was obtained as a white solid in 74% yield.

¹H NMR (300 MHz, DMSO-d₆) δ [ppm] 7.83-7.70 (m, 2H), 7.44 (dt, J=9.8, J=2.1 Hz, 1H), 7.21 (t, J=7.8 Hz, 1H), 6.83-6.73 (m, 3H), 4.92 (s, 2H), 3.69 (s, 3H), 2.31-2.15 (m, 2H), 1.50 (quintet, J=7.4 Hz, 2H), 1.21 (sextet, J=7.4 Hz, 2H), 0.78 (t, J=7.3 Hz, 3H). MS (ESI⁻): 382.3. HPLC (Condition A): Rt 3.98 min (HPLC purity 98.7%). m.p.=118-120° C.

### EXAMPLE 38

### N-[3-fluoro-5-(1H-tetrazol-5-yl)phenyl]-N-(4-methoxybenzyl)pentanamide

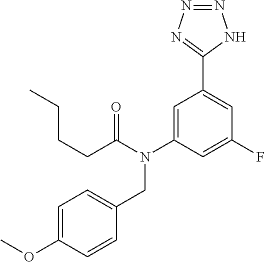

### Step 1: N-(3-cyano-5-fluorophenyl)-N-(4-methoxybenzyl)pentanamide

Following the general method as outlined in Example 22 (Step 1), starting from N-(3-cyano-5-fluorophenyl)pentanamide (Intermediate 19) and 4-(methoxy)benzyl bromide (Aldrich), the title compound was obtained as a clear oil.

¹H NMR (300 MHz, DMSO-d₆) δ [ppm] 7.85-7.77 (m, 1H), 7.64 (s, 1H), 7.56 (d t, J=9.9, J=2.2 Hz, 1H), 7.08 (d, J=8.6 Hz, 2H), 6.83 (d, J=8.6 Hz, 2H), 4.85 (s, 2H), 3.70 (s, 3H), 2.24-2.07 (m, 2H), 1.47 (quintet, J=7.4 Hz, 2H), 1.19 (sextet, J=7.4 Hz, 2H), 0.78 (t, J=7.3 Hz, 3H). MS (ESI⁺): 341.3. HPLC (Condition A): Rt 4.70 min (HPLC purity 91.2%).

### Step 2: N-[3-fluoro-5-(1H-tetrazol-5-yl)phenyl]-N-(4-methoxybenzyl)pentanamide

Following the general method as outlined in Example 22 (Step 2), starting from of N-(3-cyano-5-fluorophenyl)-N-(4-methoxybenzyl)pentanamide, the title compound was obtained as a yellow solid.

¹H NMR (300 MHz, DMSO-d₆) δ [ppm] 7.77 (d, J=8.9 Hz, 1H), 7.67 (s, 1H), 7.42-7.34 (m, 1H), 7.11 (d, J=8.6 Hz, 2H), 6.83 (d, J=8.6 Hz, 2H), 4.87 (s, 2H), 3.69 (s, 3H), 2.27-2.11 (m, 2H), 1.49 (quintet, J=7.5 Hz, 2H), 1.20 (sextet, J=7.2 Hz, 2H), 0.77 (t, J=7.3 Hz, 3H). MS (ESI⁻): 382.4. HPLC (Condition A): Rt 3.97 min (HPLC purity 94.6%). m.p.=122-125° C.

### EXAMPLE 39

### N-[3-fluoro-5-(1H-tetrazol-5-yl)phenyl]-N-(3-methoxybenzyl)propanamide

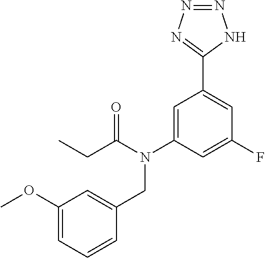

### Step 1: N-(3-cyano-5-fluorophenyl)-N-(3-methoxybenzyl)propanamide

Following the general method as outlined in Example 22 (Step 1), starting from 3-[(2,3-dihydro-1,4-benzodioxin-6-ylmethyl)amino]-4-fluorobenzonitril N-(3-cyano-5-fluorophenyl)propanamide (Intermediate 20) and 3-(methoxy)benzyl bromide (Aldrich), the title compound was obtained as a clear oil.

¹H NMR (300 MHz, DMSO-d₆) δ [ppm] 7.84-7.77 (m, 1H), 7.71 (s, 1H), 7.63 (dt, J=10.0, J=2.2 Hz, 1H), 7.20 (t, J=7.8 Hz, 1H), 6.83-6.70 (m, 3H), 4.90 (s, 2H), 3.70 (s, 3H), 2.29-2.13 (m, 2H), 0.97 (t, J=7.3 Hz, 3H). MS (ESI⁺): 313.0. HPLC (Condition A): Rt 3.95 min (HPLC purity 99.5%).

### Step 2: N-[3-fluoro-5-(1H-tetrazol-5-yl)phenyl]-N-(3-methoxybenzyl)propanamide

Following the general method as outlined in Example 22 (Step 2), starting from of N-(3-cyano-5-fluorophenyl)-N-(3-methoxybenzyl)propanamide, the title compound was obtained as a white solid in 94% yield.

¹H NMR (300 MHz, DMSO-d₆) δ [ppm] 7.82-7.71 (m, 2H), 7.45 (dt, J=9.8, J=2.1 Hz, 1H), 7.21 (t, J=7.8 Hz, 1H), 6.85-6.72 (m, 3H), 4.92 (s, 2H), 3.69 (s, 3H), 2.33-2.14 (m, 2H), 0.99 (t, J=7.4 Hz, 3H). MS (ESI⁻): 354.3. HPLC (Condition A): Rt 3.42 min (HPLC purity 97.9%).

### EXAMPLE 40

### N-[3-chloro-5-(1H-tetrazol-5-yl)phenyl]-N-[4-(trifluoromethoxy)benzyl]pentanamide

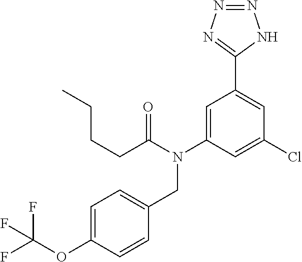

### Step 1: N-(3-chloro-5-cyanophenyl)-N-[4-(trifluoromethoxy)benzyl]pentanamide

Following the general method as outlined in Example 22 (Step 1), starting from N-(3-chloro-5-cyanophenyl)pentanamide (Intermediate 21) and 4-(trifluoromethoxy)benzyl bromide (Aldrich), the title compound was obtained as a yellow solid.

¹H NMR (300 MHz, DMSO-d₆) δ [ppm] 8.00 (s, 1H), 7.86-7.77 (m, 2H), 7.38-7.24 (m, 4H), 4.95 (s, 2H), 2.25-2.08 (m, 2H), 1.50 (quintet, J=7.3 Hz, 2H), 1.21 (sextet, J=7.3 Hz, 2H), 0.78 (t, J=7.3 Hz, 3H). MS (ESI⁺): 411.2. HPLC (Condition A): Rt 5.33 min (HPLC purity 99.2%).

### Step 2: N-[3-chloro-5-(1H-tetrazol-5-yl)phenyl]-N-[4-(trifluoromethoxy)benzyl]pentanamide

Following the general method as outlined in Example 22 (Step 2), starting from of N-(3-chloro-5-cyanophenyl)-N-[4-(trifluoromethoxy)benzyl]pentanamide, the title compound was obtained as a yellow solid.

¹H NMR (300 MHz, DMSO-d₆) δ [ppm] 8.00 (s, 1H), 7.81 (s, 1H), 7.63 (t, J=1.9 Hz, 1H), 7.39-7.26 (m, 4H), 4.96 (s, 2H), 2.29-2.11 (s, 2H), 1.49 (quintet, J=7.3 Hz, 2H), 1.20 (sextet, J=7.3 Hz, 2H), 0.77 (t, J=7.3 Hz, 3H). MS (ESI⁻): 452.1. HPLC (Condition A): Rt 4.79 min (HPLC purity 96.7%). m.p.=177-180° C.

### EXAMPLE 41

### N-[3-fluoro-5-(1H-tetrazol-5-yl)phenyl]-N-(4-methoxybenzyl)propanamide

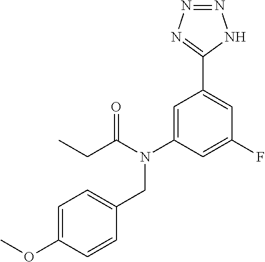

### Step 1: N-(3-cyano-5-fluorophenyl)-N-(4-methoxybenzyl)propanamide

Following the general method as outlined in Example 22 (Step 1), starting from N-(3-cyano-5-fluorophenyl)propanamide (Intermediate 20) and 4-(methoxy)benzyl bromide (Aldrich), the title compound was obtained as a clear oil.

¹H NMR (300 MHz, DMSO-d₆) δ [ppm] 7.83-7.77 (m, 1H), 7.65 (s, 1H), 7.56 (d t, J=9.9, J=2.2 Hz, 1H), 7.09 (d, J=8.6 Hz, 2H), 6.83 (d, J=8.6 Hz, 2H), 4.85 (s, 2H), 3.70 (s, 3H), 2.25-2.08 (m, 2H), 0.96 (t, J=7.3 Hz, 3H). MS (ESI⁻): 313.0. HPLC (Condition A): Rt 3.90 min (HPLC purity 96.2%).

### Step 2: N-[3-fluoro-5-(1H-tetrazol-5-yl)phenyl]-N-(4-methoxybenzyl)propanamide

Following the general method as outlined in Example 22 (Step 2), starting from of N-(3-cyano-5-fluorophenyl)-N-(4-methoxybenzyl)propanamide, the title compound was obtained as a white solid.

¹H NMR (300 MHz, DMSO-d₆) δ [ppm] 7.77 (d, J=8.9 Hz, 1H), 7.69 (s, 1H), 7.39 (d t, J=9.7, J=2.0 Hz, 1H), 7.12 (d, J=8.6 Hz, 2H), 6.83 (d, J=8.6 Hz, 2H), 4.88 (s, 2H), 3.69 (s, 3H), 2.30-2.10 (m, 2H), 0.98 (t, J=7.4 Hz, 3H). MS (ESI⁻): 354.3. HPLC (Condition A): Rt 3.39 min (HPLC purity 97.9%). m.p.=158-160° C.

### EXAMPLE 42

### N-[3-(1H-tetrazol-5-yl)phenyl]-N-[3-(trifluoromethoxy)benzyl]propanamide

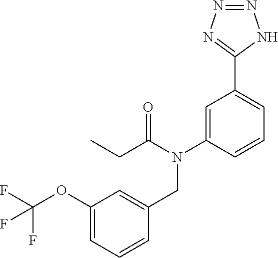

### Step 1: N-(3-cyanophenyl)-N-[3-(trifluoromethoxy)benzyl]propanamide

Following the general method as outlined in Example 22 (Step 1), starting from N-(3-cyanophenyl)propanamide (Intermediate 18) and 3-(trifluoromethoxy)benzyl bromide (ABCR), the title compound was obtained as a clear oil which solidified upon standing in 80% yield.

¹H NMR (300 MHz, DMSO-d₆) δ [ppm] 7.59 (m, 1H), 7.55 (s, 1H), 7.35-7.33 (m, 2H), 7.20 (t, J=7.9, 1H), 7.04-6.98 (m, 2H), 6.93 (s, 1H), 4.73 (s, 2H), 1.89 (m, 2H), 0.73 (t, J=7.3 Hz, 3H). MS (ESI⁺): 349.0. HPLC (Condition A): Rt 4.33 min (HPLC purity 97.6%).

### Step 2: N-[3-(1H-tetrazol-5-yl)phenyl]-N-[3-(trifluoromethoxy)benzyl]propanamide

Following the general method as outlined in Example 22 (Step 2), starting from of N-(3-cyanophenyl)-N-[3-(trifluoromethoxy)benzyl]propanamide, the title compound was obtained as a white solid.

¹H NMR (300 MHz, DMSO-d₆) δ [ppm] 7.97 (d, J=7.8, 1H), 7.86 (s, 1H), 7.62 (t, J=7.8, 1H), 7.47-7.41 (m, 2H), 7.29-7.21 (m, 2H), 7.17 (s, 1H), 4.98 (s, 2H), 2.16 (m, 2H), 0.98 (t, J=7.3 Hz, 3H). MS (ESI⁻): 390.2. HPLC (Condition A): Rt 3.95 min (HPLC purity 99.8%).

### EXAMPLE 43

### N-(1,3-benzothiazol-2-ylmethyl)-N-[3-(1H-tetrazol-5-yl)phenyl]propanamide

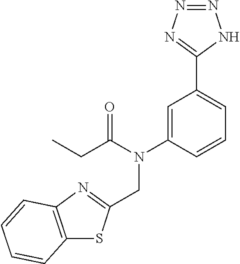

### Step 1: N-(1,3-benzothiazol-2-ylmethyl)-N-(3-cyanophenyl)propanamide

Following the general method as outlined in Example 22 (Step 1), starting from N-(3-cyanophenyl)propanamide (Intermediate 18) and 2-(bromomethyl)-1,3-benzothiazole (Acros), the title compound was obtained as a clear oil, which was used without further purification.

MS (ESI⁺): 322.0.

### Step 2: N-(1,3-benzothiazol-2-ylmethyl)-N-[3-(1H-tetrazol-5-yl)phenyl]propanamide

Following the general method as outlined in Example 22 (Step 2), starting from of N-(1,3-benzothiazol-2-ylmethyl)-N-(3-cyanophenyl)propanamide, the title compound was obtained as a white powder.

¹H NMR (300 MHz, DMSO-d₆) δ [ppm] 8.11-8.01 (m, 3H), 7.92 (d, J=7.8, 1H), 7.67 (t, J=7.8, 1H), 7.58 (d, J=8.1, 1H), 7.52-7.41 (m, 2H), 5.31 (s, 2H), 2.21 (m, 2H), 1.00 (t, J=7.3 Hz, 3H). MS (ESI⁻): 363.1. HPLC (Condition A): Rt 3.19 min (HPLC purity 97.7%).

### EXAMPLE 44

### N-[(4-methyl-1,2,5-oxadiazol-3-yl)methyl]-N-[3-(1H-tetrazol-5-yl)phenyl]propanamide

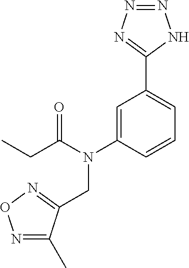

### Step 1: N-(3-cyanophenyl)-N-[(4-methyl-1,2,5-oxadiazol-3-yl)methyl]propanamide

Following the general method as outlined in Example 22 (Step 1), starting from N-(3-cyanophenyl)propanamide (Intermediate 18) and 3-(chloromethyl)-4-methyl-1,2,5-oxadiazole (Art-Chem), the title compound was obtained as a clear oil.

¹H NMR (300 MHz, DMSO-d₆) δ [ppm] 7.94 (s, 1H), 7.86 (d, J=7.3, 1H), 7.72-7.62 (m, 2H), 5.06 (s, 2H), 2.08 (m, 2H), 0.93 (t, J=7.3 Hz, 3H). MS (ESI⁺): 271.1. HPLC (Condition A): Rt 3.35 min (HPLC purity 99.8%).

### Step 2: N-[(4-methyl-1,2,5-oxadiazol-3-yl)methyl]-N-[3-(1H-tetrazol-5-yl)phenyl]propanamide

Following the general method as outlined in Example 22 (Step 2), starting from of N-(3-cyanophenyl)-N-[(4-methyl-1,2,5-oxadiazol-3-yl)methyl]propanamide, the title compound was obtained as a white solid.

¹H NMR (300 MHz, DMSO-d₆) δ [ppm] 8.02 (d, J=7.7, 1H), 7.98 (t, J=1.8, 1H), 7.68 (t, J=7.7, 1H), 7.54 (m, 1H), 5.08 (s, 2H), 2.10 (m, 2H), 0.93 (t, J=7.3 Hz, 3H). MS (ESI⁻): 312.2. HPLC (Condition A): Rt 2.60 min (HPLC purity 100.0%).

### EXAMPLE 45

### N-[3-chloro-4-(trifluoromethoxy)benzyl]-N-[3-(1H-tetrazol-5-yl)phenyl]propanamide

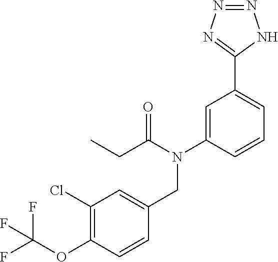

### Step 1: N-[3-chloro-4-(trifluoromethoxy)benzyl]-N-(3-cyanophenyl)propanamide

Following the general method as outlined in Example 22 (Step 1), starting from N-(3-cyanophenyl)propanamide (Intermediate 18) and 3-chloro-4-(trifluoromethoxy)benzyl bromide (ABCR), the title compound was obtained as a clear oil in 87% yield.

¹H NMR (300 MHz, DMSO-d₆) δ [ppm] 7.90 (m, 1H), 7.79 (m, 1H), 7.63-7.59 (m, 4H), 7.30 (dd, J=8.5, J=2.1, 1H), 4.92 (s, 2H), 2.12 (m, 2H), 0.95 (t, J=7.3 Hz, 3H).

MS (ESI⁻): 381.2. HPLC (Condition A): Rt 4.74 min (HPLC purity 100%).

### Step 2: N-[3-chloro-4-(trifluoromethoxy)benzyl]-N-[3-(1H-tetrazol-5-yl)phenyl]propanamide

Following the general method as outlined in Example 22 (Step 2), starting from of N-[3-chloro-4-(trifluoromethoxy)benzyl]-N-(3-cyanophenyl)propanamide, the title compound was obtained as a white powder.

¹H NMR (300 MHz, DMSO-d₆) δ [ppm] 7.98 (d, J=7.7, 1H), 7.90 (s, 1H), 7.64 (t, J=7.7, 1H), 7.54-7.44 (m, 3H), 7.33 (dd, J=8.4, J=1.7, 1H), 4.95 (s, 2H), 2.16 (m, 2H), 0.98 (t, J=7.3 Hz, 3H). MS (ESI⁻): 424.2. HPLC (Condition A): Rt 4.16 min (HPLC purity 99.4%).

### EXAMPLE 46

### N-[3-chloro-5-(1H-tetrazol-5-yl)phenyl]-N-(4-methoxybenzyl)pentanamide

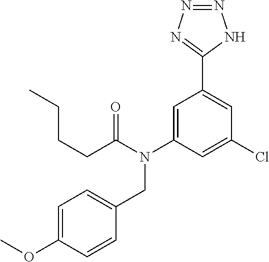

### Step 1: N-(3-chloro-5-cyanophenyl)-N-(4-methoxybenzyl)pentanamide

Following the general method as outlined in Example 22 (Step 1), starting from N-(3-chloro-5-cyanophenyl)pentanamide (Intermediate 21) and 4-methoxybenzyl bromide (Aldrich), the title compound was obtained as an oil which solidified upon standing.

¹H NMR (300 MHz, DMSO-d₆) δ [ppm] 7.98 (m, 1H), 7.75 (t, J=1.7; 1H), 7.70 (t, J=1.9; 1H), 7.08 (d, J=8.6; 2H), 6.84 (d, J=8.6; 2H), 4.85 (s, 2H), 2.15 (m, 2H), 1.47 (m, 2H), 1.20 (m, 2H), 0.79 (t, J=7.3 Hz, 3H).

### Step 2: N-[3-chloro-5-(1H-tetrazol-5-yl)phenyl]-N-(4-methoxybenzyl)pentanamide

Following the general method as outlined in Example 22 (Step 2), starting from of N-(3-chloro-5-cyanophenyl)-N-(4-methoxybenzyl)pentanamide, the title compound was obtained as a white solid in 81% yield.

¹H NMR (300 MHz, DMSO-d₆) δ [ppm] 7.98 (m, 1H), 7.76 (m; 1H), 7.54 (t, J=1.9; 1H), 7.10 (d, J=8.6; 2H), 6.83 (d, J=8.6; 2H), 4.86 (s, 2H), 2.16 (m, 2H), 1.48 (quintet, J=7.3 Hz, 2H), 1.18 (sextet, J=7.3 Hz, 2H), 0.76 (t, J=7.3 Hz, 3H). MS (ESI⁻): 398.2. HPLC (Condition A): Rt 4.31 min (HPLC purity 93.6%).

### EXAMPLE 47

### N-[3-(3-methoxyprop-1-yn-1-yl)benzyl]-N-[3-(1H-tetrazol-5-yl)phenyl]propanamide

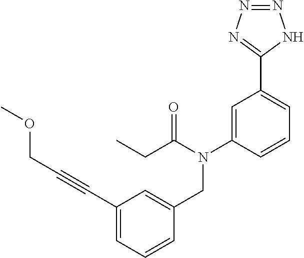

A suspension of N-(3-iodobenzyl)-N-[3-(1H-tetrazol-5-yl)phenyl]propanamide (Intermediate 23; 96 mg; 0.22 mmol) and dichlorobis(triphenylphosphine)palladium(II) (8 mg; 0.01 mmol) in triethylamine (0.20 ml; 1.44 mmol) was treated with methyl propargyl ether (Fluka; 0.20 ml; 3.97 mmol). The reaction suspension was heated at 60° C. for 4 h, then cooled, diluted with EtOAc and filtered through Celite. The organic phase was washed with a saturated NH₄Cl solution and brine, dried then concentrated to give a residue which was purified by preparative HPLC to give a the Title compound as a white solid.

¹H NMR (300 MHz, DMSO-d₆) δ [ppm] 7.96 (d, J=7.8 Hz, 1H), 7.86 (m, 1H), 7.60 (t, J=7.8 Hz, 1H), 7.41-7.35 (m, 1H), 7.34-7.27 (m, 3H), 7.27-7.20 (m, 1H), 4.92 (s, 2H), 4.30 (s, 2H), 3.30 (s, 3H), 2.26-2.05 (m, 2H), 0.98 (t, J=7.4 Hz, 3H). MS (ESI⁻): 374.3. HPLC (Condition A): Rt 3.43 min (HPLC purity 98.2%).

### EXAMPLE 48

### N-[3-(3-hydroxyprop-1-yn-1-yl)benzyl]-N-[3-(1H-tetrazol-5-yl)phenyl]propanamide

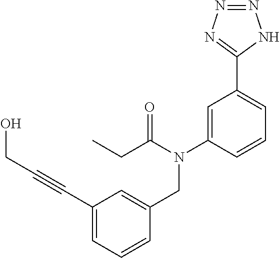

Following the general method as outlined in Example 47, starting from N-(3-iodobenzyl)-N-[3-(1H-tetrazol-5-yl)phenyl]propanamide (Intermediate 23) and propargyl alcohol (Fluka), the title compound was obtained as a yellow solid.

¹H NMR (300 MHz, DMSO-d₆) δ [ppm] 7.95 (d, J=7.8 Hz, 1H), 7.81 (s, 1H), 7.55 (t, J=7.8 Hz, 1H), 7.34-7.24 (m, 4H), 7.23-7.16 (m, 1H), 5.32 (br s, 1H), 4.90 (s, 2H), 4.27 (s, 2H), 2.24-2.04 (m, 2H), 0.97 (t, J=7.4 Hz, 3H). MS (ESI⁻): 360.2. HPLC (Condition A): Rt 2.00 min (HPLC purity 100.0%).

### EXAMPLE 49

### N-(3-pent-1-yn-1-ylbenzyl)-N-[3-(1H-tetrazol-5-yl)phenyl]propanamide

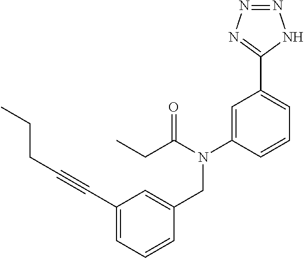

Following the general method as outlined in Example 47, starting from N-(3-iodobenzyl)-N-[3-(1H-tetrazol-5-yl)phenyl]propanamide (Intermediate 23) and 1-pentyne (Aldrich), the title compound was obtained as a yellow solid.

¹H NMR (300 MHz, DMSO-d₆) δ [ppm] 7.96 (d, J=7.4 Hz, 1H), 7.85 (s, 1H), 7.60 (t, J=7.8 Hz, 1H), 7.36 (d, J=8.0 Hz, 1H), 7.31-7.12 (m, 4H), 4.90 (s, 2H), 2.36 (t, J=7.0 Hz, 2H), 2.23-2.04 (m, 2H), 1.52 (hex, J=7.2 Hz, 2H), 1.03-0.89 (m, 6H). MS (ESI⁻): 372.2. HPLC (Condition A): Rt 4.23 min (HPLC purity 95.6%).

### EXAMPLE 50

### N-[3-(3,3-dimethylbut-1-yn-1-yl)benzyl]-N-[3-(1H-tetrazol-5-yl)phenyl]propanamide

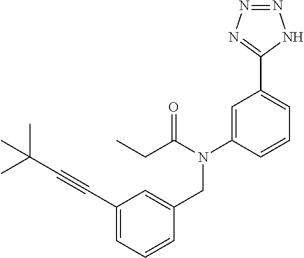

Following the general method as outlined in Example 47, starting from N-(3-iodobenzyl)-N-[3-(1H-tetrazol-5-yl)phenyl]propanamide (Intermediate 23) and 3,3-dimethyl-1-butyne (Aldrich), the title compound was obtained as a yellow solid.

¹H NMR (300 MHz, DMSO-d₆) δ [ppm] 7.97 (d, J=7.8 Hz, 1H), 7.85 (s, 1H), 7.61 (t, J=7.8 Hz, 1H), 7.38 (d, J=7.9 Hz, 1H), 7.30-7.10 (m, 4H), 4.90 (s, 2H), 2.25-2.03 (m, 2H), 1.25 (s, 9H), 0.97 (t, 3H). MS (ESI⁻): 386.2. HPLC (Condition A): Rt 4.44 min (HPLC purity 99.5%).

### EXAMPLE 51

### N-(pyridin-3-ylmethyl)-N-[3-(1H-tetrazol-5-yl)phenyl]propanamide

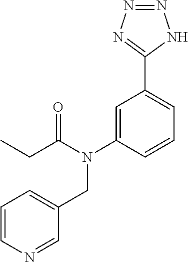

### Step 1: N-(3-cyanophenyl)-N-(pyridin-3-ylmethyl)propanamide

Following the general method as outlined in Example 22 (Step 1), starting from (3-cyanophenyl)propanamide (Intermediate 18) and 3-(bromomethyl)pyridine hydrobromide, the title compound was obtained after purification by preparative HPLC.

MS (ESI⁺): 266.0.

### Step 2: N-(pyridin-3-ylmethyl)-N-[3-(1H-tetrazol-5-yl)phenyl]propanamide

Following the general method as outlined in Example 7 (Step 2), starting from N-(3-cyanophenyl)-N-(pyridin-3-ylmethyl)propanamide, the title compound was obtained as a beige solid after purification by preparative HPLC.

¹H NMR (300 MHz, DMSO-d6) δ [ppm] 8.44 (1H, d, J=4.0 Hz), 8.40 (1H, s), 7.97 (1H, d, J=7.8 Hz), 7.87 (1H, t, J=1.8 Hz), 7.64 (1H, dt, J=7.8 Hz, J=1.8 Hz), 7.61 (1H, t, J=7.8 Hz), 7.42 (1H, ddd, J=7.8 Hz, J=2.0 Hz, J=1.0 Hz), 7.33 (1H, dd, J=7.8 Hz, J=4.8 Hz), 4.95 (2H, s), 2.14 (2H, m), 0.97 (3H, t, J=7.4 Hz). MS (ESI⁺): 309.1. HPLC (Condition A): Rt 4.45 min (HPLC purity 99.2%).

### EXAMPLE 52

### N-(pyridin-4-ylmethyl)-N-[3-(1H-tetrazol-5-yl)phenyl]propanamide

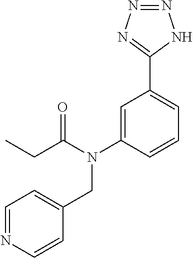

### Step 1: N-(3-cyanophenyl)-N-(pyridin-4-ylmethyl)propanamide

Following the general method as outlined in Example 22 (Step 1), starting from (3-cyanophenyl)propanamide (Intermediate 18) and 4-(bromomethyl)pyridine hydrobromide, the title compound was obtained as a white solid after purification by preparative HPLC.

MS (ESI⁺): 266.0. HPLC (Condition A): Rt 1.66 min (HPLC purity 95.6%).

### Step 2: N-(pyridin-4-ylmethyl)-N-[3-(1H-tetrazol-5-yl)phenyl]propanamide

Following the general method as outlined in Example 7 (Step 2), starting from N-(3-cyanophenyl)-N-(pyridin-4-ylmethyl)propanamide, the title compound was obtained as a white solid after purification by preparative HPLC.

¹H NMR (300 MHz, DMSO-d6) δ [ppm] 8.49 (2H, m), 7.98-7.93 (2H, m), 7.62 (1H, t, J=8.0 Hz), 7.50 (1H, dd, J=8.0 Hz, J=2.0 Hz, J=1.0 Hz), 7.27 (2H, d, J=5.8 Hz), 4.95 (2H, s), 2.20 (2H, m), 0.98 (3H, t, J=7.4 Hz). MS (ESI⁺): 309.1. HPLC (Condition A): Rt 4.46 min (HPLC purity 99.2%).

### EXAMPLE 53

### N-[3-fluoro-5-(1H-tetrazol-5-yl)phenyl]-N-{[6-(trifluoromethyl)pyridin-3-yl]methyl}propanamide

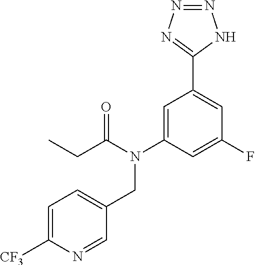

### Step 1: N-(3-cyano-5-fluorophenyl)-N-{[6-(trifluoromethyl)pyridin-3-yl]methyl}propanamide

Following the general method as outlined in Example 22 (Step 1), starting from N-(3-cyano-5-fluorophenyl)propanamide (Intermediate 20) and 3-(chloromethyl)-6-(trifluoromethyl)pyridine, the title compound was obtained as a yellow oil after purification by column chromatography (silica), eluting with cyclohexane containing increasing amounts of EtOAc.

¹H NMR (300 MHz, DMSO-d6) δ [ppm] δ 8.63 (d, J=1.4 Hz, 1H), 7.93 (dd, J=8.1, 1.7 Hz, 1H), 7.89-7.82 (m, 3H), 7.78 (dt, J=9.9, 2.1 Hz, 1H), 5.03 (s, 2H), 2.26-2.11 (m, 2H), 0.96 (t, J=7.3 Hz, 3H). HPLC (Condition A): Rt 3.90 min (HPLC purity 83.4%).

### Step 2: N-[3-fluoro-5-(1H-tetrazol-5-yl)phenyl]-N-{[6-(trifluoromethyl)pyridin-3-yl]methyl}propanamide

Following the general method as outlined in Example 16 (Step 2), starting from N-(3-cyano-5-fluorophenyl)-N-{[6-(trifluoromethyl)pyridin-3-yl]methyl}propanamide, the title compound was obtained as a grey solid after purification by preparative HPLC.

¹H NMR (300 MHz, DMSO-d6) δ [ppm] 8.65 (m, 1H), 8.02-7.77 (m, 4H), 7.59 (d, J=8.9 Hz, 1H), 5.07 (s, 2H), 2.33-2.13 (m, 2H), 1.04-0.90 (m, 3H). MS (ESI⁺): 395.1. HPLC (Condition A): Rt 3.40 min (HPLC purity 99.5%).

### EXAMPLE 54

### N-[3-chloro-4-(trifluoromethoxy)benzyl]-N-[3-fluoro-5-(1H-tetrazol-5-yl)phenyl]pentanamide

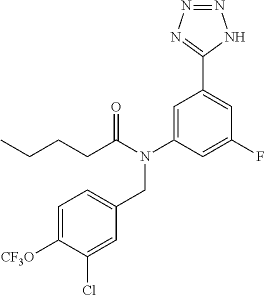

### Step 1: N-[3-chloro-4-(trifluoromethoxy)benzyl]-N-(3-cyano-5-fluorophenyl)pentanamide

Following the general method as outlined in Example 22 (Step 1), starting from N-(3-cyano-5-fluorophenyl)pentanamide (Intermediate 19) and 3-chloro-4-(trifluoromethoxy)benzyl bromide, the title compound was obtained as a clear oil after purification by column chromatography (silica), eluting with cyclohexane containing increasing amounts of EtOAc.

¹H NMR (300 MHz, DMSO-d6) δ [ppm] 7.88-7.82 (m, 1H), 7.78 (s, 1H), 7.72 (d t, J=9.9, 2.1 Hz, 1H), 7.53 (d, 1H), 7.49 (d d, J=8.4, 1.4 Hz, 1H), 7.30 (d d, J=8.4, 2.1 Hz, 1H), 4.93 (s, 2H), 2.18 (t, J=7.1 Hz, 2H), 1.47 (quin, J=7.4 Hz, 2H), 1.21 (sex, J=7.4 Hz, 2H), 0.78 (t, J=7.3 Hz, 3H). HPLC (Condition A): Rt 5.35 min (HPLC purity 97.4%).

### Step 2: N-[3-chloro-4-(trifluoromethoxy)benzyl]-N-[3-fluoro-5-(1H-tetrazol-5-yl)phenyl]pentanamide

Following the general method as outlined in Example 16 (Step 2), starting from N-[3-chloro-4-(trifluoromethoxy)benzyl]-N-(3-cyano-5-fluorophenyl)pentanamide, the title compound was obtained as a white solid after precipitation from Et₂O-pentane.

¹H NMR (300 MHz, DMSO-d6) δ [ppm] 7.81 (m, J=8.8 Hz, 1H), 7.74 (s, 1H), 7.58-7.46 (m, 3H), 7.33 (dd, J=8.5, 2.0 Hz, 1H), 4.96 (s, 2H), 2.22 (t, J=7.2 Hz, 2H), 1.49 (quin, J=7.4 Hz, 2H), 1.19 (sex, J=7.2 Hz, 2H), 0.77 (t, J=7.3 Hz, 3H). MS (ESI⁻): 470.2. HPLC (Condition A): Rt 4.83 min (HPLC purity 95.3%). m.p.=137° C.

### EXAMPLE 55

### N-[3-chloro-5-(1H-tetrazol-5-yl)phenyl]-N-[4-(trifluoromethoxy)benzyl]butanamide

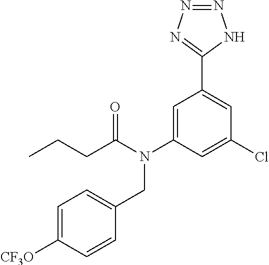

### Step 1: N-(3-chloro-5-cyanophenyl)-N-[4-(trifluoromethoxy)benzyl]butanamide

Following the general method as outlined in Example 7 (Step 1), starting from 3-chloro-5-{[4-(trifluoromethoxy)benzyl]amino}benzonitrile (Intermediate 24) and butyryl chloride, the title compound was obtained as a white solid in 78% yield after column chromatography (silica), eluting with cyclohexane containing increasing amounts of EtOAc.

¹H NMR (300 MHz, DMSO-d6) δ [ppm] 8.00 (s, 1H), 7.83 (t, J=1.6 Hz, 1H), 7.79 (t, J=1.9 Hz, 1H), 7.38-7.22 (m, 4H), 4.94 (s, 2H), 2.23-2.06 (m, 2H), 1.50 (sex, J=7.3 Hz, 2H), 0.80 (t, J=7.3 Hz, 3H). HPLC (Condition A): Rt 5.10 min (HPLC purity 97.0%).

### Step 2: N-[3-chloro-5-(1H-tetrazol-5-yl)phenyl]-N-[4-(trifluoromethoxy)benzyl]butanamide

Following the general method as outlined in Example 16 (Step 2), starting from N-(3-chloro-5-cyanophenyl)-N-[4-(trifluoromethoxy)benzyl]butanamide, the title compound was obtained as a yellow solid.

¹H NMR (300 MHz, DMSO-d6) δ [ppm] 8.01 (s, 1H), 7.81 (s, 1H), 7.63 (t, J=1.9 Hz, 1H), 7.35 (d, J=8.5 Hz, 2H), 7.29 (d, J=8.5 Hz, 2H), 4.97 (s, 2H), 2.28-2.07 (m, 2H), 1.53 (sex, J=7.3 Hz, 2H), 0.81 (t, J=7.4 Hz, 3H). MS (ESI⁺): 440.1. HPLC (Condition A): Rt 4.57 min (HPLC purity 96.5%). m.p.=179° C.

### EXAMPLE 56

### N-[3-chloro-4-(trifluoromethoxy)benzyl]-N-[3-fluoro-5-(1H-tetrazol-5-yl)phenyl]propanamide

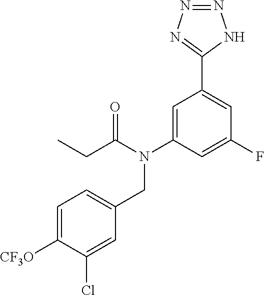

### Step 1: N-[3-chloro-4-(trifluoromethoxy)benzyl]-N-(3-cyano-5-fluorophenyl)propanamide

Following the general method as outlined in Example 22 (Step 1), starting from N-(3-cyano-5-fluorophenyl)propanamide (Intermediate 20) and 3-chloro-4-(trifluoromethoxy)benzyl bromide, the title compound was obtained as a clear oil after purification by column chromatography (silica), eluting with cyclohexane containing increasing amounts of EtOAc.

¹H NMR (300 MHz, DMSO-d6) δ [ppm] 7.41-7.35 (m, 1H), 7.34 (d, J=2.1 Hz, 1H), 7.27 (dd, J=8.4, 1.4 Hz, 1H), 7.20 (s, 1H), 7.10 (dd, J=8.4, 2.1 Hz, 1H), 7.04 (dt, J=8.7, 2.1 Hz, 1H), 4.85 (s, 2H), 2.13 (q, J=7.3 Hz, 2H), 1.12 (t, J=7.3 Hz, 3H). HPLC (Condition A): Rt 4.90 min (HPLC purity 100%).

### Step 2: N-[3-chloro-4-(trifluoromethoxy)benzyl]-N-[3-fluoro-5-(1H-tetrazol-5-yl)phenyl]propanamide

Following the general method as outlined in Example 16 (Step 2), starting from N-[3-chloro-4-(trifluoromethoxy)benzyl]-N-(3-cyano-5-fluorophenyl)propanamide, the title compound was obtained as a white solid.

¹H NMR (300 MHz, DMSO-d6) δ [ppm] 7.89-7.78 (m, 2H), 7.63-7.51 (m, 3H), 7.38 (dd, J=8.5, 2.1 Hz, 1H), 5.00 (s, 2H), 2.27 (q, J=7.2 Hz, 2H), 1.02 (t, J=7.2 Hz, 3H). MS (ESI⁻): 442.3. HPLC (Condition A): Rt 4.38 min (HPLC purity 95.9%).

### EXAMPLE 57

### N-[3-chloro-5-(1H-tetrazol-5-yl)phenyl]-N-[4-(trifluoromethoxy)benzyl]propanamide

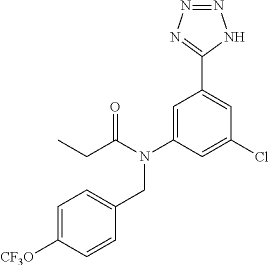

### Step 1: N-(3-chloro-5-cyanophenyl)-N-[4-(trifluoromethoxy)benzyl]propanamide

Following the general method as outlined in Example 7 (Step 1), starting from 3-chloro-5-{[4-(trifluoromethoxy)benzyl]amino}benzonitrile (Intermediate 24) and propionyl chloride, the title compound was obtained as a white solid in 72% yield after purification by column chromatography (silica), eluting with cyclohexane containing increasing amounts of EtOAc.

¹H NMR (300 MHz, DMSO-d6) δ [ppm] 7.99 (s, 1H), 7.84 (t, J=1.6 Hz, 1H), 7.80 (t, J=2.0 Hz, 1H), 7.37-7.26 (m, 4H), 4.94 (s, 2H), 2.27-2.10 (m, 2H), 0.96 (t, J=7.3 Hz, 3H). HPLC (Condition A): Rt 4.87 min (HPLC purity 95.4%).

### Step 2: N-[3-chloro-5-(1H-tetrazol-5-yl)phenyl]-N-[4-(trifluoromethoxy)benzyl]propanamide

Following the general method as outlined in Example 16 (Step 2), starting from N-(3-chloro-5-cyanophenyl)-N-[4-(trifluoromethoxy)benzyl]propanamide, the title compound was obtained as a white solid after precipitation from DCM/pentane.

¹H NMR (300 MHz, DMSO-d6) δ [ppm] 8.00 (t, J=1.6 Hz, 1H), 7.83 (t, J=1.6 Hz, 1H), 7.65 (t, J=1.9 Hz, 1H), 7.36 (d, J=8.5 Hz, 2H), 7.29 (d, J=8.5 Hz, 2H), 4.97 (s, 2H), 2.30-2.11 (m, 2H), 0.98 (t, J=7.3 Hz, 3H). MS (ESI⁺): 426.1. HPLC (Condition A): Rt 4.35 min (HPLC purity 98.7%). m.p.=144° C.

### EXAMPLE 58

### N-[3-fluoro-5-(1H-tetrazol-5-yl)phenyl]-N-[4-(trifluoromethyl)benzyl]propanamide

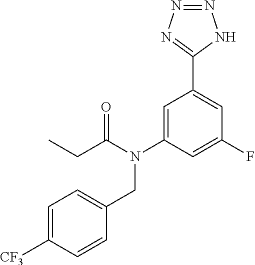

### Step 1: N-(3-cyano-5-fluorophenyl)-N-[4-(trifluoromethyl)benzyl]propanamide

Following the general method as outlined in Example 22 (Step 1), starting from N-(3-cyano-5-fluorophenyl)propanamide (Intermediate 20) and 4-(trifluoromethyl)benzyl bromide, the title compound was obtained as a yellow solid in 85% yield after purification by column chromatography (silica), eluting with cyclohexane containing increasing amounts of EtOAc.

MS (ESI⁻): 348.9. HPLC (Condition A): Rt 5.22 min (HPLC purity 98.1%).

### Step 2: N-[3-fluoro-5-(1H-tetrazol-5-yl)phenyl]-N-[4-(trifluoromethyl)benzyl]propanamide

Following the general method as outlined in Example 16 (Step 2), starting from N-(3-cyano-5-fluorophenyl)-N-[4-(trifluoromethyl)benzyl]propanamide, the title compound was obtained as a white foam in 95% yield.

¹H NMR (300 MHz, DMSO-d6) δ 7.86-7.72 (m, 2H), 7.67 (d, J=8.1, 2H), 7.59-7.50 (m, 1H), 7.47 (d, J=8.0, 2H), 5.04 (s, 2H), 3.45 (brs, J=9.9, OH), 2.36-2.13 (m, 2H), 0.99 (t, J=7.4, 3H). MS (ESI⁻): 392.0. HPLC (Condition A): Rt 4.21 min (HPLC purity 98.5%).

### EXAMPLE 59

### N-(biphenyl-3-ylmethyl)-N-[3-(1H-tetrazol-5-yl)phenyl]propanamide

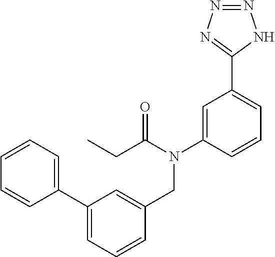

### Step 1: N-(biphenyl-3-ylmethyl)-N-(3-cyanophenyl)propanamide

A solution of phenyl boronic acid (70 mg; 0.57 mmol), N-(3-cyanophenyl)-N-(3-iodobenzyl)propanamide (114 mg; 0.29 mmol) in a mixture of dioxane (3 mL) and water (1.5 mL) was treated with dichlorobis(triphenylphosphine)palladium(II) (16 mg; 0.02 mmol) and cesium fluoride (132 mg; 0.87 mmol). The reaction vessel was sealed and heated in a microwave apparatus at 120° C. for 30 min. On cooling the reaction solution was diluted with EtOAc then washed with 1 N aqueous HCl and brine then dried on MgSO₄, filtered through celite and the solvents were removed under reduced pressure to give a yellow oil which was purified by flash column chromatography (silica), eluting with cyclohexane containing increasing amounts of EtOAc to give the title compound as a clear oil

¹H NMR (300 MHz, DMSO-d6) δ [ppm] 7.89-7.84 (m, 1H), 7.81-7.72 (m, 1H), 7.62-7.56 (m, 4H), 7.55-7.32 (m, 6H), 7.18 (d, J=7.4 Hz, 1H), 4.99 (s, 2H), 2.22-2.05 (m, 2H), 0.97 (t, J=7.4 Hz, 3H). HPLC (Condition A): Rt 4.59 min (HPLC purity 98.5%).

### Step 2: N-(biphenyl-3-ylmethyl)-N-[3-(1H-tetrazol-5-yl)phenyl]propanamide

Following the general method as outlined in Example 16 (Step 2), starting from N-(biphenyl-3-ylmethyl)-N-(3-cyanophenyl)propanamide, the title compound was obtained as a white solid after precipitation from DCM/pentane.

¹H NMR (300 MHz, DMSO-d6) δ [ppm] 8.00-7.91 (m, 2H), 7.66-7.31 (m, 10H), 7.22 (d, J=8.0 Hz, 1H), 5.01 (s, 2H), 2.26-2.09 (m, 2H), 0.99 (t, J=7.4 Hz, 3H). MS (ESI⁻): 382.2. HPLC (Condition A): Rt 4.00 min (HPLC purity 98.6%). m.p.=91° C.

### EXAMPLE 60

### N-[3-fluoro-5-(1H-tetrazol-5-yl)phenyl]-N-[4-(trifluoromethyl)benzyl]pentanamide

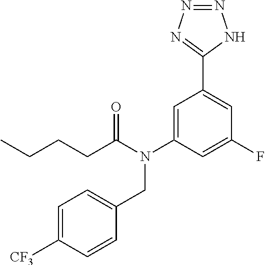

### Step 1: N-(3-cyano-5-fluorophenyl)-N-[4-(trifluoromethyl)benzyl]pentanamide

Following the general method as outlined in Example 22 (Step 1), starting from N-(3-cyano-5-fluorophenyl)pentanamide (Intermediate 19) and 4-(trifluoromethyl)benzyl bromide, the title compound was obtained as a clear oil after purification by column chromatography (silica), eluting with cyclohexane containing increasing amounts of EtOAc.

¹H NMR (300 MHz, DMSO-d6) δ [ppm] 7.88-7.80 (m, 1H), 7.78 (s, 1H), 7.74-7.62 (m, 3H), 7.44 (d, J=8.0 Hz, 2H), 5.01 (s, 2H), 2.26-2.12 (m, 2H), 1.48 (quin, J=7.4 Hz, 2H), 1.20 (sex, J=7.4 Hz, 2H), 0.79 (t, 3H). HPLC (Condition A): Rt 5.26 min (HPLC purity 99.8%).

### Step 2: N-[3-fluoro-5-(1H-tetrazol-5-yl)phenyl]-N-[4-(trifluoromethyl)benzyl]pentanamide

Following the general method as outlined in Example 16 (Step 2), starting from N-(3-cyano-5-fluorophenyl)-N-[4-(trifluoromethyl)benzyl]pentanamide, the title compound was obtained as a white solid in 83% yield after precipitation from DCM-pentane.

¹H NMR (300 MHz, DMSO-d6) δ [ppm] 7.82-7.74 (m, 2H), 7.67 (d, J=8.0 Hz, 2H), 7.54-7.43 (m, 3H), 5.04 (s, 2H), 2.30-2.17 (m, 2H), 1.50 (quin, J=7.4 Hz, 2H), 1.20 (sex, J=7.4 Hz, 2H), 0.78 (t, J=7.3 Hz, 3H). MS (ESI⁻): 420.4. HPLC (Condition A): Rt 4.53 min (HPLC purity 96.2%).

### EXAMPLE 61

### N-[3-(3,5-dimethylisoxazol-4-yl)benzyl]-N-[3-(1H-tetrazol-5-yl)phenyl]propanamide

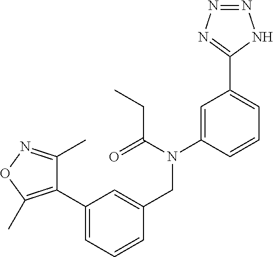

### Step 1: N-(3-cyanophenyl)-N-[3-(3,5-dimethylisoxazol-4-yl)benzyl]propanamide

Following the general method as outlined in Example 59 (Step 1), starting from N-(3-cyanophenyl)-N-(3-iodobenzyl)propanamide (Intermediate 22) and 3,5-dimethylisoxazole-4-boronic acid, the title compound was obtained as a clear oil after purification by column chromatography (silica), eluting with cyclohexane containing increasing amounts of EtOAc.

¹H NMR (300 MHz, DMSO-d6) δ [ppm] 7.83 (s, 1H), 7.80-7.74 (m, 1H), 7.62-7.55 (m, 2H), 7.40 (t, J=7.5 Hz, 1H), 7.27-7.20 (m, 2H), 7.13 (s, 1H), 4.96 (s, 2H), 2.31 (s, 3H), 2.19-2.08 (m, 5H), 0.96 (t, J=7.3 Hz, 3H). HPLC (Condition A): Rt 4.00 min (HPLC purity 99.8%).

### Step 2: N-[3-(3,5-dimethylisoxazol-4-yl)benzyl]-N-[3-(1H-tetrazol-5-yl)phenyl]propanamide

Following the general method as outlined in Example 16 (Step 2), starting from N-(3-cyanophenyl)-N-[3-(3,5-dimethylisoxazol-4-yl)benzyl]propanamide, the title compound was obtained as a white solid after precipitation from DCM/pentane.

¹H NMR (300 MHz, DMSO-d6) δ [ppm] 7.95 (d, J=6.4 Hz, 1H), 7.85 (s, 1H), 7.62 (t, J=7.9 Hz, 1H), 7.48-7.36 (m, 2H), 7.30-7.21 (m, 2H), 7.14 (s, 1H), 4.98 (s, 2H), 2.28 (s, 3H), 2.24-2.07 (m, 5H), 0.98 (t, J=7.3 Hz, 3H). MS (ESI⁺): 403.1. HPLC (Condition A): Rt 3.41 min (HPLC purity 97.7%).

### EXAMPLE 62

### N-(3-isoxazol-4-ylbenzyl)-N-[3-(1H-tetrazol-5-yl)phenyl]propanamide

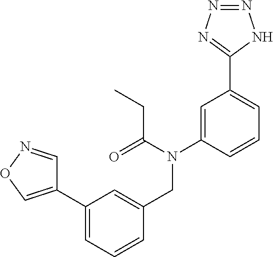

### Step 1: N-(3-cyanophenyl)-N-(3-isoxazol-4-ylbenzyl)propanamide

Following the general method as outlined in Example 59 (Step 1), starting from N-(3-cyanophenyl)-N-(3-iodobenzyl)propanamide (Intermediate 22) and 4-isoxazoleboronic acid pinacol ester, the title compound was obtained as a clear oil after purification by column chromatography (silica), eluting with cyclohexane containing increasing amounts of EtOAc.

¹H NMR (300 MHz, DMSO-d6) δ [ppm] 9.42 (s, 1H), 9.12 (s, 1H), 7.90 (s, 1H), 7.75 (d, J=6.6 Hz, 1H), 7.66-7.51 (m, 3H), 7.46 (s, 1H), 7.35 (t, J=7.5 Hz, 1H), 7.15 (d, J=7.7 Hz, 1H), 4.95 (s, 2H), 2.27-2.02 (m, 2H), 0.97 (t, J=7.2 Hz, 3H). HPLC (Condition A): Rt 3.82 min (HPLC purity 94.8%).

### Step 2: N-(3-isoxazol-4-ylbenzyl)-N-[3-(1H-tetrazol-5-yl)phenyl]propanamide

Following the general method as outlined in Example 16 (Step 2), starting from N-(3-cyanophenyl)-N-(3-isoxazol-4-ylbenzyl)propanamide, the title compound was obtained as a white solid after purification by preparative HPLC.

¹H NMR (300 MHz, DMSO-d6) δ [ppm] 9.40 (s, 1H), 9.10 (s, 1H), 7.83-7.91 (m, 2H), 7.64-7.42 (m, 4H), 7.37 (t, J=7.7 Hz, 1H), 7.19 (d, J=7.7 Hz, 1H), 4.98 (s, 2H), 2.26-2.09 (m, 2H), 0.99 (t, J=7.4 Hz, 3H). MS (ESI⁺): 375.3. HPLC (Condition A): Rt 3.19 min (HPLC purity 95.2%).

### EXAMPLE 63

### N-[3-(2,4-dimethyl-1,3-thiazol-5-yl)benzyl]-N-[3-(1H-tetrazol-5-yl)phenyl]propanamide

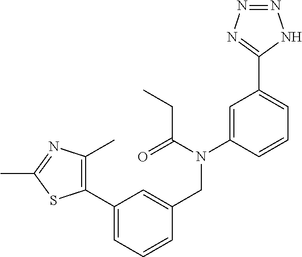

### Step 1: N-(3-cyanophenyl)-N-[3-(2,4-dimethyl-1,3-thiazol-5-yl)benzyl]propanamide

Following the general method as outlined in Example 59 (Step 1), starting from N-(3-cyanophenyl)-N-(3-iodobenzyl)propanamide (Intermediate 22) and 2,4-dimethyl-5-(4,4,5,5,-tetramethyl-1,3,2-dioxaborolan-2-yl)-1,3-thiazole, the title compound was obtained as a yellow oil after purification by column chromatography (silica), eluting with cyclohexane containing increasing amounts of EtOAc.

¹H NMR (300 MHz, DMSO-d6) δ [ppm] 7.86-7.81 (m, 1H), 7.80-7.72 (m, 1H), 7.62-7.53 (m, 2H), 7.42-7.33 (m, 1H), 7.32-7.25 (m, 1H), 7.24-7.16 (m, 2H), 4.95 (s, 2H), 2.60 (s, 3H), 2.28 (s, 3H), 2.21-2.04 (m, 2H), 0.96 (t, J=7.4 Hz, 3H). HPLC (Condition A): Rt 3.15 min (HPLC purity 99.5%).

### Step 2: N-[3-(2,4-dimethyl-1,3-thiazol-5-yl)benzyl]-N-[3-(1H-tetrazol-5-yl)phenyl]propanamide

Following the general method as outlined in Example 16 (Step 2), starting from N-(3-cyanophenyl)-N-[3-(2,4-dimethyl-1,3-thiazol-5-yl)benzyl]propanamide, the title compound was obtained as a white solid after purification by preparative HPLC.

¹H NMR (300 MHz, DMSO-d6) δ [ppm] 7.96 (d, J=7.8 Hz, 1H), 7.85 (s, 1H), 7.60 (t, J=7.9 Hz, 1H), 7.43-7.35 (m, 2H), 7.29 (d, J=7.7 Hz, 1H), 7.25-7.19 (m, 2H), 4.97 (s, 2H), 2.59 (s, 3H), 2.25 (s, 3H), 2.23-2.08 (m, 2H), 0.98 (t, 3H). MS (ESI⁺): 419.3. HPLC (Condition A): Rt 2.69 min (HPLC purity 96.0%).

### EXAMPLE 64

### N-(3-pyridin-3-ylbenzyl)-N-[3-(1H-tetrazol-5-yl)phenyl]propanamide

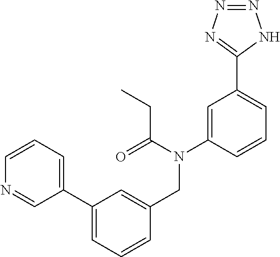

### Step 1: N-(3-cyanophenyl)-N-(3-pyridin-3-ylbenzyl)propanamide

Following the general method as outlined in Example 59 (Step 1), starting from N-(3-cyanophenyl)-N-(3-iodobenzyl)propanamide (Intermediate 22) and pyridine-3-boronic acid, the title compound was obtained as a clear oil after purification by column chromatography (silica), eluting with cyclohexane containing increasing amounts of EtOAc.

¹H NMR (300 MHz, DMSO-d6) δ [ppm] 8.82 (s, 1H), 8.57 (d, J=3.8 Hz, 1H), 8.00 (d, J=8.0 Hz, 1H), 7.89 (s, 1H), 7.76 (d, J=7.3 Hz, 1H), 7.68-7.37 (m, 6H), 7.25 (d, J=7.4 Hz, 1H), 5.00 (s, 2H), 2.25-2.05 (m, 2H), 0.97 (t, J=7.3 Hz, 3H). HPLC (Condition A): Rt 2.48 min (HPLC purity 93.7%).

### Step 2: N-(3-pyridin-3-ylbenzyl)-N-[3-(1H-tetrazol-5-yl)phenyl]propanamide

Following the general method as outlined in Example 16 (Step 2), starting from N-(3-cyanophenyl)-N-(3-pyridin-3-ylbenzyl)propanamide, the title compound was obtained as a white solid after purification by preparative HPLC.

¹H NMR (300 MHz, DMSO-d6) δ [ppm] 8.80 (s, 1H), 8.61-8.53 (m, 1H), 8.04-7.88 (m, 3H), 7.65-7.36 (m, 6H), 7.29 (d, J=8.1 Hz, 1H), 5.02 (s, 2H), 2.28-2.07 (m, 2H), 0.98 (t, J=7.3 Hz, 3H). MS (ESI⁺): 385.3. HPLC (Condition A): Rt 2.17 min (HPLC purity 98.9%).

### EXAMPLE 65

### N-[4-(methylsulfonyl)benzyl]-N-[3-(1H-tetrazol-5-yl)phenyl]propanamide

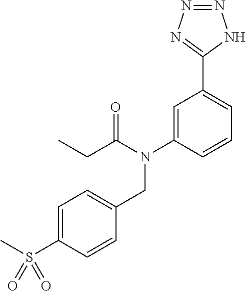

### Step 1: N-(3-cyanophenyl)-N-[4-(methylsulfonyl)benzyl]propanamide

Following the general method as outlined in Example 22 (Step 1), starting from (3-cyanophenyl)propanamide (Intermediate 18) and methanesulphonylbenzyl bromide, the title compound was obtained as a clear oil in 76% yield after purification by column chromatography (silica), eluting with cyclohexane containing increasing amounts of EtOAc.

MS (ESI⁺): 343.3. HPLC (Condition A): Rt 3.07 min (HPLC purity 95.7%).

### Step 2: N-[4-(methylsulfonyl)benzyl]-N-[3-(1H-tetrazol-5-yl)phenyl]propanamide

Following the general method as outlined in Example 16 (Step 2), starting from N-(3-cyanophenyl)-N-[4-(methylsulfonyl)benzyl]propanamide, the title compound was obtained as a white solid in 74% yield.

¹H NMR (300 MHz, DMSO-d6) δ [ppm] 7.96 (d, J=7.8 Hz, 1H), 7.92 (s, 1H), 7.84 (d, J=8.1 Hz, 2H), 7.62 (t, J=7.8 Hz, 1H), 7.51-7.46 (m, 3H), 5.03 (s, 2H), 3.17 (s, 3H), 2.17 (m, 2H), 0.97 (t, J=7.3 Hz, 3H). MS (ESI⁺): 386.3. HPLC (Condition A): Rt 2.53 min (HPLC purity 99.6%).

### EXAMPLE 66

### N-[4-(1H-pyrazol-3-yl)benzyl]-N-[3-(1H-tetrazol-5-yl)phenyl]propanamide

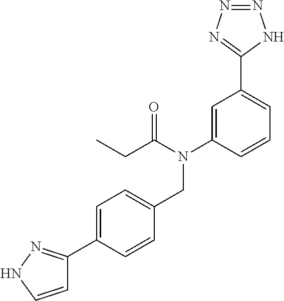

### Step 1: N-(3-cyanophenyl)-N-[4-(1H-pyrazol-3-yl)benzyl]propanamide

Following the general method as outlined in Example 59 (Step 1), starting from N-(3-cyanophenyl)-N-(4-iodobenzyl)propanamide (Intermediate 29) and 1H-pyrazole-3-boronic acid, the title compound was obtained after purification by column chromatography (silica), eluting with cyclohexane containing increasing amounts of EtOAc.

MS (ESI⁺): 331.3

### Step 2: N-[4-(1H-pyrazol-3-yl)benzyl]-N-[3-(1H-tetrazol-5-yl)phenyl]propanamide

Following the general method as outlined in Example 16 (Step 2), starting from N-(3-cyanophenyl)-N-[4-(1H-pyrazol-3-yl)benzyl]propanamide, the title compound was obtained as a white solid after purification by preparative HPLC.

¹H NMR (300 MHz, DMSO-d6) δ 12.86 (s, 1H), 8.03-7.86 (m, 3H), 7.77-7.66 (m, 3H), 7.59 (t, J=7.9 Hz, 1H), 7.38 (d, J=8.4 Hz, 1H), 7.32-7.17 (m, 2H), 6.66 (d, J=2.0 Hz, 1H), 4.95 (s, 2H), 2.28-2.03 (m, 2H), 0.99 (t, J=7.4 Hz, 3H). MS (ESI⁺): 374.3. HPLC (Condition A): Rt 2.65 min (HPLC purity 92.9%).

### EXAMPLE 67

### N-[3-chloro-5-(1H-tetrazol-5-yl)phenyl]-2-ethoxy-N-[4-(trifluoromethoxy)benzyl]acetamide

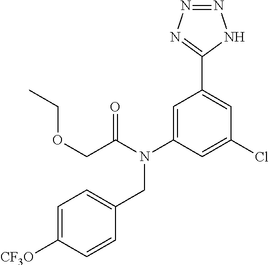

### Step 1: N-(3-chloro-5-cyanophenyl)-2-ethoxy-N-[4-(trifluoromethoxy)benzyl]acetamide

A solution of ethoxyacetic acid (24 mg; 0.23 mmol) in DCM (1 mL) was treated with oxalyl chloride (16 μl; 0.18 mmol) and DMF (2 μl). The reaction mixture was stirred until the end of the gas evolution, then was treated with 3-chloro-5-{[4-(trifluoromethoxy)benzyl]amino}benzonitrile (Intermediate 24; 50.00 mg; 0.15 mmol) and TEA (40 μl; 0.31 mmol). The reaction mixture was stirred at 100° C. for 3 h. On cooling, the reaction solution was diluted with DCM, washed with a saturated NH₄Cl aqueous solution then with a saturated NaHCO₃ aqueous solution, dried on MgSO₄, filtered and the solvents were removed under reduced pressure to give the title compound (54.6 mg, 86%).

MS (ESI⁺): 413.2

### Step 2: N-[3-chloro-5-(1H-tetrazol-5-yl)phenyl]-2-ethoxy-N-[4-(trifluoromethoxy)benzyl]acetamide

Following the general method as outlined in Example 16 (Step 2), starting from N-(3-chloro-5-cyanophenyl)-2-ethoxy-N-[4-(trifluoromethoxy)benzyl]acetamide, the title compound was obtained as an oil.

¹H NMR (300 MHz, DMSO-d6) δ 8.00 (s, 1H), 7.87 (s, 1H), 7.67 (s, 1H), 7.44-7.22 (m, 4H), 4.97 (s, 2H), 4.02 (s, 2H), 3.53-3.13 (m, 2H), 0.99 (t, J=7.0 Hz, 3H). MS (ESI⁻): 454.3. HPLC (Condition A): Rt 4.35 min (HPLC purity 96.2%).

### EXAMPLE 68

### N-[3-chloro-5-(1H-tetrazol-5-yl)phenyl]-3-methoxy-N-[4-(trifluoromethoxy)benzyl]propanamide

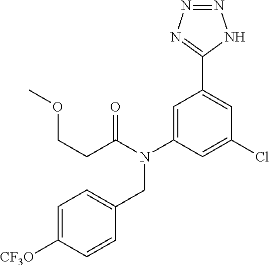

### Step 1: N-(3-chloro-5-cyanophenyl)-3-methoxy-N-[4-(trifluoromethoxy)benzyl]propanamide

Following the general method as outlined in Example 67 (Step 1), starting from 3-chloro-5-{[4-(trifluoromethoxy)benzyl]amino}benzonitrile (Intermediate 24) and ethoxyacetic acid, the title compound was obtained in 99% yield after purification by column chromatography (silica), eluting with cyclohexane containing increasing amounts of EtOAc.

MS (ESI⁻): 411.3

### Step 2: N-[3-chloro-5-(1H-tetrazol-5-yl)phenyl]-3-methoxy-N-[4-(trifluoromethoxy)benzyl]propanamide

Following the general method as outlined in Example 16 (Step 2), starting from N-(3-chloro-5-cyanophenyl)-3-methoxy-N-[4-(trifluoromethoxy)benzyl]propanamide, the title compound was obtained as an oil after precipitation from acetonitrile/diethyl ether.

¹H NMR (300 MHz, DMSO-d6) δ 8.02 (s, 1H), 7.86 (s, 1H), 7.63 (s, 1H), 7.33 (q, J=9.03 Hz, 4H), 4.99 (s, 2H), 3.56 (t, J=6.1 Hz, 2H), 3.18 (s, 3H), 2.50 (m, 2H). MS (ESI⁻): 454.3. HPLC (Condition A): Rt 4.31 min (HPLC purity 93.3%). m.p.=146-151° C.

### EXAMPLE 69

### N-(biphenyl-4-ylmethyl)-N-[3-(1H-tetrazol-5-yl)phenyl]propanamide

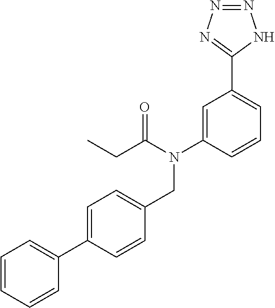

### Step 1: N-(biphenyl-4-ylmethyl)-N-(3-cyanophenyl)propanamide

Following the general method as outlined in Example 59 (Step 1), starting from N-(3-cyanophenyl)-N-(4-iodobenzyl)propanamide (Intermediate 29) and phenylboronic acid, the title compound was obtained in 56% yield after purification by column chromatography (silica), eluting with cyclohexane containing increasing amounts of EtOAc.

MS (ESI⁺): 341.3

### Step 2: N-(biphenyl-4-ylmethyl)-N-[3-(1H-tetrazol-5-yl)phenyl]propanamide

Following the general method as outlined in Example 16 (Step 2), starting from N-(biphenyl-4-ylmethyl)-N-(3-cyanophenyl)propanamide, the title compound was obtained as a white solid after purification by preparative HPLC.

¹H NMR (300 MHz, DMSO-d6) δ 8.10-7.85 (m, 2H), 7.73-7.52 (m, 5H), 7.44 (t, J=6.7 Hz, 3H), 7.39-7.23 (m, 3H), 4.98 (s, 2H), 2.18 (m, 2H), 0.99 (t, J=7.3 Hz, 3H). MS (ESI⁻): 382.4. HPLC (Condition A): Rt 4.00 min (HPLC purity 97%)

### EXAMPLE 70

### N-[4-(1H-pyrazol-4-yl)benzyl]-N-[3-(1H-tetrazol-5-yl)phenyl]propanamide

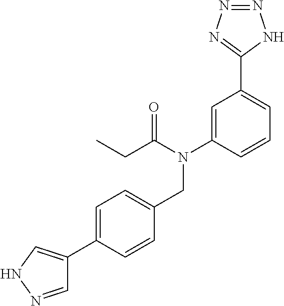

### Step 1: N-(3-cyanophenyl)-N-[4-(1H-pyrazol-4-yl)benzyl]propanamide

Following the general method as outlined in Example 59 (Step 1), starting from N-(3-cyanophenyl)-N-(4-iodobenzyl)propanamide (Intermediate 29) and pyrazole-4-boronic acid, the title compound was obtained after purification by column chromatography (silica), eluting with cyclohexane containing increasing amounts of EtOAc.

MS (ESI⁺): 331.1

### Step 2: N-[4-(1H-pyrazol-4-yl)benzyl]-N-[3-(1H-tetrazol-5-yl)phenyl]propanamide

Following the general method as outlined in Example 16 (Step 2), starting from N-(3-cyanophenyl)-N-[4-(1H-pyrazol-4-yl)benzyl]propanamide, the title compound was obtained as a white solid after purification by preparative HPLC.

¹H NMR (300 MHz, DMSO-d6) δ 8.11-7.83 (m, 4H), 7.61 (t, J=7.9 Hz, 1H), 7.51 (d, J=8.1 Hz, 2H), 7.40 (d, J=8.0 Hz, 1H), 7.17 (d, J=8.2 Hz, 2H), 4.92 (s, 2H), 3.35 (m, 2H), 2.15 (m, 3H), 0.99 (t, J=7.4 Hz, 3H). MS (ESI⁻): 372.3. HPLC (Condition A): Rt 2.67 min (HPLC purity 95.9%).

### EXAMPLE 71

### N-[4-(3,5-dimethylisoxazol-4-yl)benzyl]-N-[3-(1H-tetrazol-5-yl)phenyl]propanamide

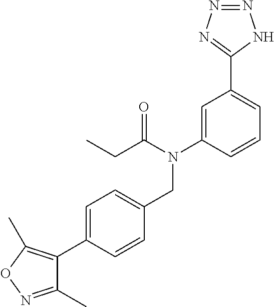

### Step 1: N-(3-cyanophenyl)-N-[4-(3,5-dimethylisoxazol-4-yl)benzyl]propanamide

Following the general method as outlined in Example 59 (Step 1), starting from N-(3-cyanophenyl)-N-(4-iodobenzyl)propanamide (Intermediate 29) and 3,5-dimethylisoxazole-4-boronic acid, the title compound was obtained after purification by column chromatography (silica), eluting with cyclohexane containing increasing amounts of EtOAc.

MS (ESI⁺): 360.3

### Step 2: N-[4-(3,5-dimethylisoxazol-4-yl)benzyl]-N-[3-(1H-tetrazol-5-yl)phenyl]propanamide

Following the general method as outlined in Example 16 (Step 2), starting from N-(3-cyanophenyl)-N-[4-(3,5-dimethylisoxazol-4-yl)benzyl]propanamide, the title compound was obtained as a white solid after purification by preparative HPLC.

¹H NMR (300 MHz, DMSO-d6) δ 7.98 (d, J=7.8 Hz, 1H), 7.86 (s, 1H), 7.64 (t, J=7.9 Hz, 1H), 7.54-7.43 (m, 1H), 7.31 (s, 4H), 4.97 (s, 2H), 2.35 (s, 3H), 2.27-2.10 (m, 5H), 0.99 (t, J=7.4 Hz, 3H). MS (ESI⁻): 401.4. HPLC (Condition A): Rt 3.40 min (HPLC purity 99.0%).

### EXAMPLE 72

### N-(quinolin-6-ylmethyl)-N-[3-(1H-tetrazol-5-yl)phenyl]propanamide

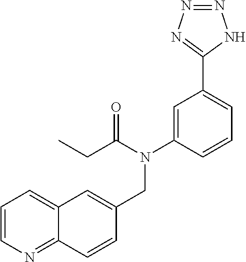

### Step 1: N-(3-cyanophenyl)-N-(quinolin-6-ylmethyl)propanamide

Following the general method as outlined in Example 7 (Step 1), starting from 3-[(quinolin-6-ylmethyl)amino]benzonitrile (Intermediate 25) and propionyl chloride, the title compound was obtained as an oil after purification by column chromatography (silica), eluting with cyclohexane containing increasing amounts of EtOAc.

¹H NMR (300 MHz, DMSO-d6) δ [ppm] 8.90 (dd, J=4.2, 1.7 Hz, 1H), 8.12-8.02 (m, 2H), 7.64-7.54 (m, 3H), 7.48-7.35 (m, 3H), 7.21 (d, J=7.5 Hz, 1H), 5.08 (s, 2H), 2.19-2.01 (m, 2H), 1.12 (t, J=7.4 Hz, 3H). HPLC (Condition A): Rt 2.09 min (HPLC purity 100%).

### Step 2: N-(quinolin-6-ylmethyl)-N-[3-(1H-tetrazol-5-yl)phenyl]propanamide

Following the general method as outlined in Example 16 (Step 2), starting from N-(3-cyanophenyl)-N-(quinolin-6-ylmethyl)propanamide, the title compound was obtained as a white solid after purification by column chromatography (silica), eluting with cyclohexane containing increasing amounts of EtOAc.

¹H NMR (300 MHz, DMSO-d6) δ [ppm] 8.85 (d, J=2.6 Hz, 1H), 8.30 (d, J=7.4 Hz, 1H), 8.00-7.82 (m, 3H), 7.76 (s, 1H), 7.66 (dd, J=8.7, 1.9 Hz, 1H), 7.52-7.41 (m, 2H), 7.23 (d, J=8.0 Hz, 1H), 5.11 (s, 2H), 2.25-2.10 (m, 2H), 1.01 (t, J=7.4 Hz, 3H). MS (ESI⁺): 359.3. HPLC (Condition A): Rt 1.83 min (HPLC purity 97.9%).

### EXAMPLE 73

### N-(3-methylbenzyl)-N-[3-(1H-tetrazol-5-yl)phenyl]propanamide

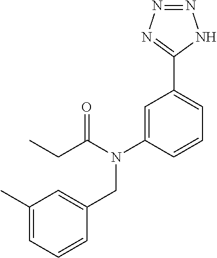

### Step 1: N-(3-cyanophenyl)-N-(3-methylbenzyl)propanamide

Following the general method as outlined in Example 59 (Step 1), starting from N-(3-cyanophenyl)-N-(3-iodobenzyl)propanamide (Intermediate 22) and methylboronic acid, the title compound was obtained as an oil after purification by column chromatography (silica), eluting with cyclohexane containing increasing amounts of EtOAc.

¹H NMR (300 MHz, DMSO-d6) δ [ppm] 7.84-7.72 (m, 2H), 7.62-7.50 (m, 2H), 7.16 (t, J=7.4 Hz, 1H), 7.06-6.90 (m, 3H), 4.87 (s, 2H), 2.24 (s, 3H), 2.20-2.02 (m, 2H), 0.96 (t, J=7.4 Hz, 3H). HPLC (Condition A): Rt 4.66 min (HPLC purity 77.7%).

### Step 2: N-(3-methylbenzyl)-N-[3-(1H-tetrazol-5-yl)phenyl]propanamide

Following the general method as outlined in Example 16 (Step 2), starting from N-(3-cyanophenyl)-N-(3-methylbenzyl)propanamide, the title compound was obtained as a yellow solid after purification by preparative HPLC.

¹H NMR (300 MHz, DMSO-d6) δ [ppm] 7.95 (d, J=7.6 Hz, 1H), 7.87 (s, 1H), 7.59 (t, J=7.9 Hz, 1H), 7.38 (d, J=7.9 Hz, 1H), 7.16 (t, J=7.8 Hz, 1H), 7.06-6.93 (m, 3H), 4.89 (s, 2H), 2.24 (s, 3H), 2.21-2.06 (m, 2H), 0.98 (t, J=7.4 Hz, 3H). MS (ESI⁺): 322.1. HPLC (Condition A): Rt 3.37 min (HPLC purity 98.2%).

### EXAMPLE 74

### N-(4-hydroxybenzyl)-N-[3-(1H-tetrazol-5-yl)phenyl]pentanamide

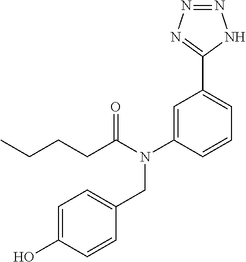

A cooled (−78° C.) solution of N-(4-methoxybenzyl)-N-[3-(1H-tetrazol-5-yl)phenyl]pentanamide

(Example 14; 80 mg; 0.22 mmol) in DCM (20 ml) was treated with a solution of BBr₃ (0.5 mL) in DCM (10 mL). The suspension was warmed slowly to RT and stirred for 16 h, then poured in aqueous (1 N) HCl and extracted with EtOAc. The collected organics were dried and concentrated under reduced pressure to give a residue which was purified by preparative HPLC to give the title compound as a white solid.

¹H NMR (300 MHz, DMSO-d6) δ [ppm] 9.31 (s, 1H), 7.95 (d, J=7.8 Hz, 1H), 7.79 (s, 1H), 7.59 (t, J=7.8 Hz, 1H), 7.30 (d, J=8.7 Hz, 1H), 6.96 (d, J=8.4 Hz, 2H), 6.64 (d, J=8.4 Hz, 2H), 4.80 (s, 2H), 2.17-1.97 (m, 2H), 1.47 (quip, J=7.3 Hz, 2H), 1.17 (sex, J=7.3 Hz, 2H), 0.75 (t, J=7.3 Hz, 3H). MS (ESI⁺): 352.1. HPLC (Condition A): Rt 3.08 min (HPLC purity 95.4%).

### EXAMPLE 75

### N-[3-fluoro-5-(1H-tetrazol-5-yl)phenyl]-N-(3-isopropoxybenzyl)propanamide

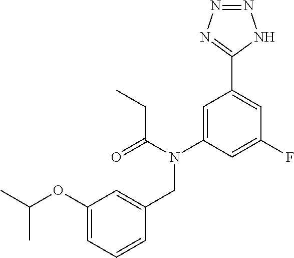

### Step 1: N-(3-cyano-5-fluorophenyl)-N-(3-isopropoxybenzyl)propanamide

Following the general method as outlined in Example 22 (Step 1), starting from N-(3-cyano-5-fluorophenyl)propanamide (Intermediate 20) and 1-(bromomethyl)-3-(propan-2-yloxy)benzene, the title compound was obtained as a yellow oil in 79% yield after purification by column chromatography (silica), eluting with cyclohexane containing increasing amounts of EtOAc.

¹H NMR (300 MHz, DMSO-d6) δ [ppm] 7.84-7.76 (m, 1H), 7.69 (s, 1H), 7.61 (d t, J=10.0, 2.2 Hz, 1H), 7.17 (t, J=7.8 Hz, 1H), 6.80-6.67 (m, 3H), 4.88 (s, 2H), 4.54 (sep, J=6.0 Hz, 1H), 2.29-2.11 (m, 2H), 1.20 (d, J=6.0 Hz, 6H), 0.96 (t, J=7.3 Hz, 3H). HPLC (Condition A): Rt 4.46 min (HPLC purity 92.3%).

### Step 2: N-[3-fluoro-5-(1H-tetrazol-5-yl)phenyl]-N-(3-isopropoxybenzyl)propanamide

Following the general method as outlined in Example 16 (Step 2), starting from N-(3-cyano-5-fluorophenyl)-N-(3-isopropoxybenzyl)propanamide, the title compound was obtained as a yellow foam.

¹H NMR (300 MHz, DMSO-d6) δ [ppm] 7.82-7.69 (m, 2H), 7.44 (d t, J=9.8, 2.1 Hz, 1H), 7.18 (t, J=7.8 Hz, 1H), 6.81-6.69 (m, 3H), 4.91 (s, 2H), 4.51 (sep, J=6.0 Hz, 1H), 2.32-2.14 (m, 2H), 1.17 (d, J=6.0 Hz, 6H), 0.99 (t, J=7.4 Hz, 3H). MS (ESI⁺): 384.2. HPLC (Condition A): Rt 3.92 min (HPLC purity 95.5%).

### EXAMPLE 76

### N-[4-chloro-3-(trifluoromethoxy)benzyl]-N-[3-fluoro-5-(1H-tetrazol-5-yl)phenyl]propanamide

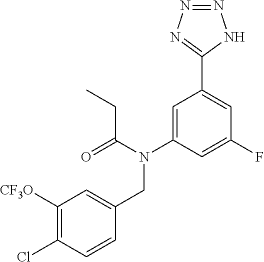

### Step 1: N-[4-chloro-3-(trifluoromethoxy)benzyl]-N-(3-cyano-5-fluorophenyl)propanamide

Following the general method as outlined in Example 22 (Step 1), starting from N-(3-cyano-5-fluorophenyl)propanamide (Intermediate 20) and 4-chloro-3-(trifluoromethoxy)benzyl bromide, the title compound was obtained as a clear oil after purification by column chromatography (silica), eluting with cyclohexane containing increasing amounts of EtOAc.

¹H NMR (300 MHz, DMSO-d6) δ [ppm] 7.43 (d, J=8.2 Hz, 1H), 7.40-7.35 (m, 1H), 7.19-7.08 (m, 3H), 7.00 (dt, J=8.7, 2.1 Hz, 1H), 4.87 (s, 2H), 2.12 (q, J=7.3 Hz, 2H), 1.12 (t, J=7.3 Hz, 3H). (Condition A): Rt 5.11 min (HPLC purity 100%).

### Step 2: N-[4-chloro-3-(trifluoromethoxy)benzyl]-N-[3-fluoro-5-(1H-tetrazol-5-yl)phenyl]propanamide

Following the general method as outlined in Example 16 (Step 2), starting from N-[4-chloro-3-(trifluoromethoxy)benzyl]-N-(3-cyano-5-fluorophenyl)propanamide, the title compound was obtained as a white solid.

¹H NMR (300 MHz, DMSO-d6) δ [ppm] 7.79 (d, J=8.2 Hz, 1H), 7.71 (s, 1H), 7.63 (d, J=8.2 Hz, 1H), 7.48 (d t, J=9.8, 2.1 Hz, 1H), 7.40-7.31 (m, 2H), 4.98 (s, 2H), 2.28-2.17 (m, 2H), 0.98 (t, J=7.4 Hz, 3H). MS (ESI⁻): 442.3. HPLC (Condition A): Rt 4.37 min (HPLC purity 96.2%).

### EXAMPLE 77

### N-[3-fluoro-5-(1H-tetrazol-5-yl)phenyl]-N-{3-[(trifluoromethyl)thio]benzyl}propanamide

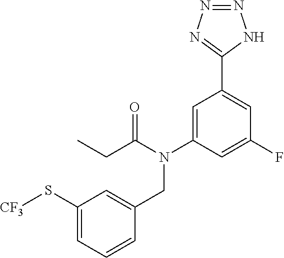

### Step 1: N-(3-cyano-5-fluorophenyl)-N-{3-[(trifluoromethyl)thio]benzyl}propanamide

Following the general method as outlined in Example 22 (Step 1), starting from N-(3-cyano-5-fluorophenyl)propanamide (Intermediate 20) and 3-(trifluoromethylthio)benzyl chloride, the title compound was obtained as a yellow oil after purification by column chromatography (silica), eluting with cyclohexane containing increasing amounts of EtOAc.

¹H NMR (300 MHz, DMSO-d6) δ [ppm] 7.85-7.78 (m, 1H), 7.67 (s, 1H), 7.66-7.44 (m, 5H), 4.99 (s, 2H), 2.26-2.14 (m, 2H), 0.96 (t, 3H). HPLC (Condition A): Rt 4.78 min (HPLC purity 59.3%).

### Step 2: N-[3-fluoro-5-(1H-tetrazol-5-yl)phenyl]-N-{3-[(trifluoromethyl)thio]benzyl}propanamide

Following the general method as outlined in Example 16 (Step 2), starting from N-(3-cyano-5-fluorophenyl)-N-{3-[(trifluoromethyl)thio]benzyl}propanamide, the title compound was obtained as a white solid after purification by preparative HPLC.

¹H NMR (300 MHz, DMSO-d6) δ [ppm] 7.84-7.77 (m, 1H), 7.73 (s, 1H), 7.66-7.50 (m, 4H), 7.47-7.40 (m, 1H), 5.04 (s, 2H), 2.35-2.20 (m, 2H), 1.02 (t, J=7.2 Hz, 3H). MS (ESI⁻): 424.2. HPLC (Condition A): Rt 4.26 min (HPLC purity 96.1%).

### EXAMPLE 78

### N-[3-fluoro-5-(1H-tetrazol-5-yl)phenyl]-N-[4-(methylsulfonyl)benzyl]propanamide

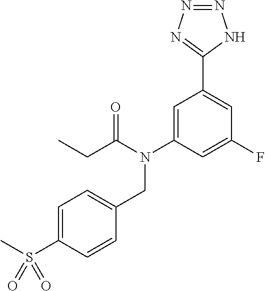

### Step 1: N-(3-cyano-5-fluorophenyl)-N-[4-(methylsulfonyl)benzyl]propanamide

Following the general method as outlined in Example 22 (Step 1), starting from N-(3-cyano-5-fluorophenyl)propanamide (Intermediate 20) and 4-methylsulphonylbenzyl bromide, the title compound was obtained as a white solid after purification by column chromatography (silica), eluting with cyclohexane containing increasing amounts of EtOAc.

¹H NMR (300 MHz, DMSO-d6) δ [ppm] 7.92 (d, J=8.4 Hz, 2H), 7.45-7.35 (m, 3H), 7.21 (s, 1H), 7.05 (d, J=8.6 Hz, 1H), 4.97 (s, 2H), 3.07 (s, 3H), 2.16 (q, J=7.4 Hz, 2H), 1.14 (t, J=7.4 Hz, 3H). HPLC (Condition A): Rt 3.20 min (HPLC purity 99.8%).

### Step 2: N-[3-fluoro-5-(1H-tetrazol-5-yl)phenyl]-N-[4-(methylsulfonyl)benzyl]propanamide

Following the general method as outlined in Example 16 (Step 2), starting from N-(3-cyano-5-fluorophenyl)-N-[4-(methylsulfonyl)benzyl]propanamide, the title compound was obtained as a white solid in 63% yield.

¹H NMR (300 MHz, DMSO-d6) δ [ppm] 7.90 (d, J=8.4 Hz, 2H), 7.86-7.80 (m, 2H), 7.64-7.53 (m, 3H), 5.10 (s, 2H), 3.22 (s, 3H), 2.37-2.23 (m, 2H), 1.03 (t, J=7.4 Hz, 3H). MS (ESI⁺): 404.3. HPLC (Condition A): Rt 2.77 min (HPLC purity 99.0%).

### EXAMPLE 79

### N-(3-hydroxybenzyl)-N-[3-(1H-tetrazol-5-yl)phenyl]pentanamide

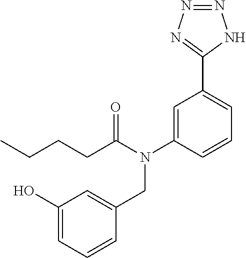

Following the general method as outlined in Example 74, starting from N-(3-methoxybenzyl)-N-[3-(1H-tetrazol-5-yl)phenyl]pentanamide (Example 13), the title compound was obtained as a yellow solid after purification by preparative HPLC.

¹H NMR (300 MHz, DMSO-d6) δ [ppm] 9.38 (s, 1H), 8.00 (d, J=7.6 Hz, 1H), 7.89 (s, 1H), 7.65 (t, J=7.9 Hz, 1H), 7.40 (d, J=7.9 Hz, 1H), 7.10 (t, J=7.6 Hz, 1H), 6.71-6.60 (m, 3H), 4.88 (s, 2H), 2.28-2.06 (m, 2H), 1.53 (quip, J=7.5 Hz, 2H), 1.30-1.17 (m, 2H), 0.80 (t, J=7.3 Hz, 3H). MS (ESI⁺): 352.3. HPLC (Condition A): Rt 3.16 min (HPLC purity 98.9%).

### EXAMPLE 80

### N-[3-fluoro-5-(1H-tetrazol-5-yl)phenyl]-N-(quinolin-2-ylmethyl)propanamide

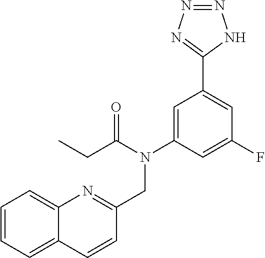

### Step 1: N-(3-cyano-5-fluorophenyl)-N-(quinolin-2-ylmethyl)propanamide

Following the general method as outlined in Example 22 (Step 1), starting from N-(3-cyano-5-fluorophenyl)propanamide (Intermediate 20) and 2-(chloromethyl)quinoline hydrochloride, the title compound was obtained as a clear oil after purification by column chromatography (silica), eluting with cyclohexane containing increasing amounts of EtOAc.

¹H NMR (300 MHz, DMSO-d6) δ [ppm] 8.15 (d, J=8.4 Hz, 1H), 8.00 (d, J=8.4 Hz, 1H), 7.80 (dd, J=8.1, 1.1 Hz, 1H), 7.74-7.66 (m, 1H), 7.57-7.49 (m, 2H), 7.48-7.41 (m, 2H), 7.34-7.26 (m, 1H), 5.16 (s, 2H), 2.34-2.17 (m, 2H), 1.13 (t, J=7.4 Hz, 3H). HPLC (Condition A): Rt 2.73 min (HPLC purity 98.5%).

### Step 2: N-[3-fluoro-5-(1H-tetrazol-5-yl)phenyl]-N-(quinolin-2-ylmethyl)propanamide

Following the general method as outlined in Example 16 (Step 2), starting from N-(3-cyano-5-fluorophenyl)-N-(quinolin-2-ylmethyl)propanamide, the title compound was obtained as a yellow solid after purification by preparative HPLC.

¹H NMR (300 MHz, DMSO-d6) δ [ppm] 8.39 (d, J=8.4 Hz, 1H), 8.06 (s, 1H), 8.02-7.96 (m, 2H), 7.83-7.73 (m, 2H), 7.69 (d, J=9.6 Hz, 1H), 7.65-7.57 (m, 2H), 5.26 (s, 2H), 2.42-2.24 (m, 2H), 1.05 (t, J=7.4 Hz, 3H). MS (ESI⁺): 377.4. HPLC (Condition A): Rt 2.38 min (HPLC purity 99.8%).

### EXAMPLE 81

### N-[3-fluoro-5-(1H-tetrazol-5-yl)phenyl]-N-(3-pyridin-3-ylbenzyl)propanamide

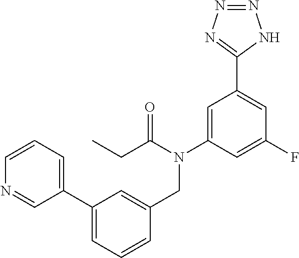

### Step 1: N-(3-cyano-5-fluorophenyl)-N-(3-pyridin-3-ylbenzyl)propanamide

Following the general method as outlined in Example 59 (Step 1), starting from N-(3-cyano-5-fluorophenyl)-N-(3-iodobenzyl)propanamide (Intermediate 30) and pyridine-4-boronic acid, the title compound was obtained as a clear oil in 84% yield.

¹H NMR (300 MHz, DMSO-d6) δ [ppm] 8.67 (d, J=1.6 Hz, 1H), 8.61 (dd, J=4.8, 1.6 Hz, 1H), 8.09-8.03 (m, 1H), 7.87-7.81 (m, 2H), 7.75 (dt, J=9.9, 2.1 Hz, 1H), 7.62 (d, J=7.8 Hz, 1H), 7.56 (s, 1H), 7.55-7.49 (m, 1H), 7.46 (t, J=7.7 Hz, 1H), 7.29 (d, J=7.8 Hz, 1H), 5.06 (s, 2H), 2.34-2.20 (m, 2H), 1.02 (t, J=7.3 Hz, 3H). HPLC (Condition A): Rt 2.62 min (HPLC purity 96.6%).

### Step 2: N-[3-fluoro-5-(1H-tetrazol-5-yl)phenyl]-N-(3-pyridin-3-ylbenzyl)propanamide

Following the general method as outlined in Example 16 (Step 2), starting from N-(3-cyano-5-fluorophenyl)-N-(3-pyridin-3-ylbenzyl)propanamide, the title compound was obtained as a white solid after purification using an SCX strong acidic (sulfonic acid) ion-exchange SPE column.

¹H NMR (300 MHz, DMSO-d6) δ [ppm] 8.88-8.84 (m, 1H), 8.60 (dd, J=4.8, 1.6 Hz, 1H), 8.08-8.02 (m, 1H), 7.86-7.77 (m, 2H), 7.66-7.43 (m, 5H), 7.33 (d, J=7.8 Hz, 1H), 5.08 (s, 2H), 2.39-2.21 (m, 2H), 1.04 (t, J=7.4 Hz, 3H). MS (ESI⁺): 403.2. HPLC (Condition A): Rt 2.36 min (HPLC purity 92.8%).

### EXAMPLE 82

### N-[3-fluoro-5-(1H-tetrazol-5-yl)phenyl]-N-{4-[(trifluoromethyl)thio]benzyl}propanamide

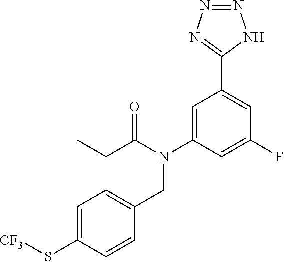

### Step 1: N-(3-cyano-5-fluorophenyl)-N-{4-[(trifluoromethyl)thio]benzyl}propanamide

Following the general method as outlined in Example 22 (Step 1), starting from N-(3-cyano-5-fluorophenyl)propanamide (Intermediate 20) and 4-(trifluoromethylthio)benzyl chloride, the title compound was obtained as a yellow oil after purification by column chromatography (silica), eluting with cyclohexane containing increasing amounts of EtOAc.

¹H NMR (300 MHz, DMSO-d6) δ [ppm] 7.61 (d, J=8.2 Hz, 2H), 7.37-7.32 (m, 1H), 7.24 (d, J=8.2 Hz, 2H), 7.18 (s, 1H), 7.02 (d, J=8.8 Hz, 1H), 4.92 (s, 2H), 2.14 (q, J=7.3 Hz, 2H), 1.13 (t, J=7.3 Hz, 3H). HPLC (Condition A): Rt 4.84 min (HPLC purity 99.6%).

### Step 2: N-[3-fluoro-5-(1H-tetrazol-5-yl)phenyl]-N-{4-[(trifluoromethyl)thio]benzyl}propanamide

Following the general method as outlined in Example 16 (Step 2), starting from N-(3-cyano-5-fluorophenyl)-N-{4-[(trifluoromethyl)thio]benzyl}propanamide, the title compound was obtained as a yellow foam.

¹H NMR (300 MHz, DMSO-d6) δ [ppm] 7.82-7.75 (m, 2H), 7.69 (d, J=8.1 Hz, 2H), 7.50-7.41 (m, 3H), 5.04 (s, 2H), 2.28 (q, J=7.4 Hz, 2H), 1.03 (t, J=7.4 Hz, 3H). MS (ESI⁻): 424.2. HPLC (Condition A): Rt 4.32 min (HPLC purity 97.9%).

### EXAMPLE 83

### N-[3-fluoro-5-(1H-tetrazol-5-yl)phenyl]-N-(3-pyridin-4-ylbenzyl)propanamide

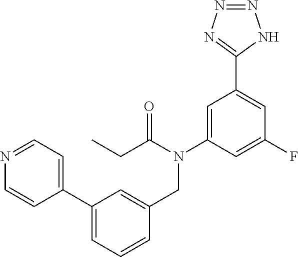

### Step 1: N-(3-cyano-5-fluorophenyl)-N-(3-pyridin-4-ylbenzyl)propanamide

Following the general method as outlined in Example 59 (Step 1), starting from N-(3-cyano-5-fluorophenyl)-N-(3-iodobenzyl)propanamide (Intermediate 30) and pyridine-4-boronic acid, the title compound was obtained as a clear oil in 96% yield.

¹H NMR (300 MHz, DMSO-d6) δ [ppm] 8.69-8.61 (m, 2H), 7.86-7.78 (m, 2H), 7.75-7.72 (m, 1H), 7.71-7.64 (m, 3H), 7.61 (s, 1H), 7.47 (t, J=7.7 Hz, 1H), 7.32 (d, J=7.7 Hz, 1H), 5.04 (s, 2H), 2.34-2.16 (m, 2H), 1.00 (t, J=7.3 Hz, 3H). HPLC (Condition A): Rt 2.60 min (HPLC purity 87.4%).

### Step 2: N-[3-fluoro-5-(1H-tetrazol-5-yl)phenyl]-N-(3-pyridin-4-ylbenzyl)propanamide

Following the general method as outlined in Example 16 (Step 2), starting from N-(3-cyano-5-fluorophenyl)-N-(3-pyridin-4-ylbenzyl)propanamide, the title compound was obtained as a white solid after purification by preparative HPLC.

¹H NMR (300 MHz, DMSO-d6) δ [ppm] 8.69-8.63 (m, 2H), 7.86-7.77 (m, 2H), 7.74-7.64 (m, 4H), 7.59-7.46 (m, 2H), 7.39 (d, J=7.7 Hz, 1H), 5.09 (s, 2H), 2.39-2.20 (m, 2H), 1.04 (t, J=7.4 Hz, 3H). MS (ESI⁺): 403.2. HPLC (Condition A): Rt 1.73 min (HPLC purity 98.8%).

### EXAMPLE 84

### N-[3-fluoro-5-(1H-tetrazol-5-yl)phenyl]-N-(3-hydroxybenzyl)propanamide

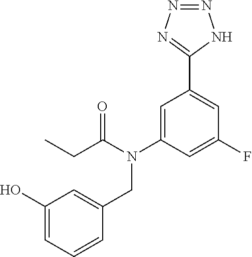

### Step 1: N-(3-cyano-5-fluorophenyl)-N-[3-(4,4,5,5-tetramethyl-1,3,2-dioxaborolan-2-yl)benzyl]propanamide

A solution of N-(3-cyano-5-fluorophenyl)-N-(3-iodobenzyl)propanamide (Intermediate 30; 304 mg; 0.74 mmol), bis(pinacolato)diboron (415 mg; 1.63 mmol) in DMSO (4 mL) was treated with [1,1′-bis(diphenylphosphino)ferrocene]dichloropalladium (II) (50 mg; 0.07 mmol) and potassium acetate (273 mg; 2.78 mmol) and heated in a microwave reactor at 120° C. for 30 min. The resulting mixture was cooled to RT, diluted with EtOAc and washed with brine. The collected organics were dried and concentrated under reduced pressure to give a residue which was by column chromatography (silica), eluting with cyclohexane containing increasing amounts of EtOAc, to give the title compound as a clear oil.

¹H NMR (300 MHz, DMSO-d6) δ [ppm] 7.88-7.82 (m, 1H), 7.70 (t, J=1.4 Hz, 1H), 7.64 (dt, J=9.9, 2.2 Hz, 1H), 7.59-7.54 (m, 1H), 7.50 (s, 1H), 7.37-7.33 (m, 2H), 4.98 (s, 2H), 2.30-2.16 (m, 2H), 1.31 (s, 12H), 1.00 (t, J=7.3 Hz, 3H). HPLC (Condition A): Rt 4.80 min (HPLC purity 75.2%).

### Step 2: N-(3-cyano-5-fluorophenyl)-N-(3-hydroxybenzyl)propanamide

A solution of N-(3-cyano-5-fluorophenyl)-N-[3-(4,4,5,5-tetramethyl-1,3,2-dioxaborolan-2-yl)benzyl]propanamide (143 mg; 0.35 mmol) and MeOH (20 ml) was treated with a 33% aqueous solution of H₂O₂ (10.00 ml). The mixture was stirred for 1 h then carefully poured into a Na₂S₂O₃ (sat) solution. The solution was extracted with EtOAc (3 times), dried and concentrated under reduced pressure to give a clear oil, which was used without further purification.

¹H NMR (300 MHz, DMSO-d6) δ [ppm] 7.33 (d, J=7.2 Hz, 1H), 7.22-7.11 (m, 2H), 7.04 (d, J=8.7 Hz, 1H), 6.84-6.76 (m, 2H), 6.60 (d, J=7.4 Hz, 1H), 4.83 (s, 2H), 2.22-2.08 (m, 2H), 1.13 (t, J=7.4 Hz, 3H). HPLC (Condition A): Rt 3.26 min (HPLC purity 90.6%).

### Step 3: N-[3-fluoro-5-(1H-tetrazol-5-yl)phenyl]-N-(3-hydroxybenzyl)propanamide

Following the general method as outlined in Example 16 (Step 2), starting from N-(3-cyano-5-fluorophenyl)-N-(3-hydroxybenzyl)propanamide, the title compound was obtained as a white solid after purification by preparative HPLC.

¹H NMR (300 MHz, DMSO-d6) δ [ppm] 9.38 (s, 1H), 7.83-7.64 (m, 2H), 7.33 (d, J=9.7 Hz, 1H), 7.09 (t, J=7.7 Hz, 1H), 6.68-6.58 (m, 3H), 4.87 (m, 2H), 2.33-2.14 (m, 2H), 1.01 (t, J=7.3 Hz, 3H). MS (ESI⁺): 342.1. HPLC (Condition A): Rt 2.83 min (HPLC purity 95.2%).

### EXAMPLE 85

### N-[3-(3,5-dimethylisoxazol-4-yl)benzyl]-N-[3-fluoro-5-(1H-tetrazol-5-yl)phenyl]propanamide

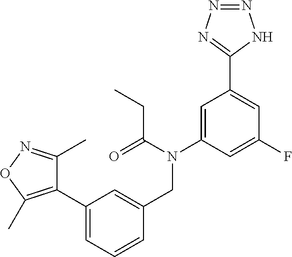

### Step 1: N-(3-cyano-5-fluorophenyl)-N-[3-(3,5-dimethylisoxazol-4-yl)benzyl]propanamide

Following the general method as outlined in Example 59 (Step 1), starting from N-(3-cyano-5-fluorophenyl)-N-(3-iodobenzyl)propanamide (Intermediate 30) and 3,5-dimethylisoxazole-4-boronic acid, the title compound was obtained as a clear oil after purification by column chromatography (silica), eluting with cyclohexane containing increasing amounts of EtOAc.

¹H NMR (300 MHz, DMSO-d6) δ [ppm] 7.89-7.81 (m, 1H), 7.78 (s, 1H), 7.72 (d t, J=9.9, 2.2 Hz, 1H), 7.44 (t, J=7.6 Hz, 1H), 7.29 (d, J=1.6 Hz, 1H), 7.26 (d, J=1.6 Hz, 1H), 7.19 (s, 1H), 5.02 (s, 2H), 2.36 (s, 3H), 2.33-2.20 (m, 2H), 2.18 (s, 3H), 1.08 (t, 3H). HPLC (Condition A): Rt 4.29 min (HPLC purity 90.8%).

### Step 2: N-[3-(3,5-dimethylisoxazol-4-yl)benzyl]-N-[3-fluoro-5-(1H-tetrazol-5-yl)phenyl]propanamide

Following the general method as outlined in Example 16 (Step 2), starting from N-(3-cyano-5-fluorophenyl)-N-[3-(3,5-dimethylisoxazol-4-yl)benzyl]propanamide, the title compound was obtained as a white solid in 79% yield.

¹H NMR (300 MHz, DMSO-d6) δ [ppm] 7.83-7.76 (m, 1H), 7.73 (s, 1H), 7.51 (dt, J=9.7, 2.1 Hz, 1H), 7.43 (t, J=7.7 Hz, 1H), 7.32-7.23 (m, 2H), 7.18 (s, 1H), 5.02 (s, 2H), 2.34-2.20 (m, 5H), 2.12 (s, 3H), 1.01 (t, J=7.4 Hz, 3H). MS (ESI⁺): 421.2. HPLC (Condition A): Rt 3.73 min (HPLC purity 98.4%).

### EXAMPLE 86

### N-({3′-[(ethylamino)sulfonyl]biphenyl-4-yl}methyl)-N-[3-fluoro-5-(1H-tetrazol-5-yl)phenyl]propanamide

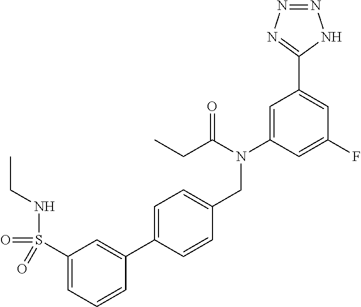

### Step 1: N-(3-cyano-5-fluorophenyl)-N-({3′-[(ethylamino)sulfonyl]fluorophenyl)-4-yl}methyl)propanamide

Following the general method as outlined in Example 59 (Step 1), starting from N-(3-cyano-5-fluorophenyl)-N-(4-iodobenzyl)propanamide (Intermediate 31) and 3-(N-ethylsulfamoyl)phenyl boronic acid, the title compound was obtained after purification by column chromatography (silica), eluting with cyclohexane containing increasing amounts of EtOAc.

MS (ESI⁻): 464.0

### Step 2: N-({3′-[ethylamino)sulfonyl]biphenyl-4-yl}methyl)-N-[3-fluoro-5-(1H-tetrazol-5-yl)phenyl]propanamide

Following the general method as outlined in Example 16 (Step 2), starting from N-(3-cyano-5-fluorophenyl)-N-({3′-[(ethylamino)sulfonyl]biphenyl-4-yl}methyl)propanamide, the title compound was obtained as a beige solid after purification by preparative HPLC.

¹H NMR (300 MHz, DMSO-d6) δ 8.22-7.24 (m, 12H), 5.00 (s, 2H), 2.88-2.67 (m, 2H), 2.36-2.11 (m, 2H), 1.07-0.86 (m, 6H). MS (ESI⁻): 507.0. HPLC (Condition A): Rt 3.86 min (HPLC purity 83.1%).

### EXAMPLE 87

### N-[3-fluoro-5-(1H-tetrazol-5-yl)phenyl]-N-{[3′-(methylsulfonyl)biphenyl-4-yl]methyl}propanamide

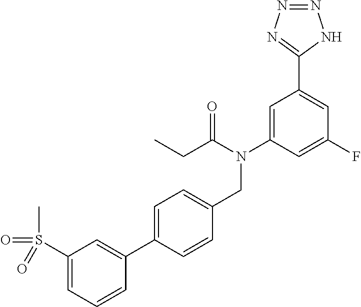

### Step 1: N-(3-cyano-5-fluorophenyl)-N-{[3′-(methylsulfonyl)biphenyl-4-yl]methyl}propanamide

Following the general method as outlined in Example 59 (Step 1), starting from N-(3-cyano-5-fluorophenyl)-N-(4-iodobenzyl)propanamide (Intermediate 31) and 3-(methylsulfonyl)phenyl boronic acid, the title compound was obtained after purification by column chromatography (silica), eluting with cyclohexane containing increasing amounts of EtOAc.

MS (ESI⁻): 434.9

### Step 2: N-[3-fluoro-5-(1H-tetrazol-5-yl)phenyl]-N-{[3′-(methylsulfonyl)biphenyl-4-yl]methyl}propanamide

Following the general method as outlined in Example 16 (Step 2), starting from N-(3-cyano-5-fluorophenyl)-N-{[3′-(methylsulfonyl)biphenyl-4-yl]methyl}propanamide, the title compound was obtained as a white solid.

¹H NMR (300 MHz, DMSO-d6) δ 8.12 (brs, 1H), 8.00 (d, J=7.8 Hz, 1H), 7.90 (d, J=7.8 Hz, 1H), 7.85-7.64 (m, 5H), 7.52 (brd, J=9.6 Hz, 1H), 7.38 (d, J=8.1 Hz, 2H), 5.03 (s, 2H), 3.28 (s, 3H), 2.36-2.17 (m, 2H), 1.01 (t, J=7.3 Hz, 3H). MS (ESI⁻): 477.9. HPLC (Condition A): Rt 3.62 min (HPLC purity 88.5%).

### EXAMPLE 88

### N-[4-(1,3-dimethyl-1H-pyrazol-4-yl)benzyl]-N-[3-fluoro-5-(1H-tetrazol-5-yl)phenyl]propanamide

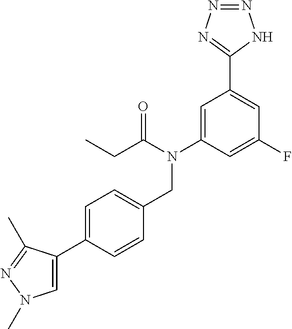

### Step 1: N-(3-cyano-5-fluorophenyl)-N-[4-(1,3-dimethyl-1H-pyrazol-4-yl)benzyl]propanamide

Following the general method as outlined in Example 59 (Step 1), starting from N-(3-cyano-5-fluorophenyl)-N-(4-iodobenzyl)propanamide (Intermediate 31) and 1,3-dimethyl-1H-pyrazole-4-boronic acid, pinacol ester, the title compound was obtained in 78% yield after purification by column chromatography (silica), eluting with cyclohexane containing increasing amounts of EtOAc.

MS (ESI⁺): 376.9

### Step 2: N-[4-(1,3-dimethyl-1H-pyrazol-4-yl)benzyl]-N-[3-fluoro-5-(1H-tetrazol-5-yl)phenyl]propanamide

Following the general method as outlined in Example 16 (Step 2), starting from N-(3-cyano-5-fluorophenyl)-N-[4-(1,3-dimethyl-1H-pyrazol-4-yl)benzyl]propanamide, the title compound was obtained as a white solid after purification by preparative HPLC.

¹H NMR (300 MHz, DMSO-d6) δ 7.85 (s, 1H), 7.82-7.71 (m, 2H), 7.50-7.43 (m, 1H), 7.34 (d, J=8.2 Hz, 2H), 7.22 (d, J=8.2 Hz, 2H), 4.95 (s, 2H), 3.76 (s, 3H), 2.26 (m, 5H), 1.00 (t, J=7.3 Hz, 3H). MS (ESI⁻): 418.0. HPLC (Condition A): Rt 3.21 min (HPLC purity 90.7%).

### EXAMPLE 89

### N-[3-fluoro-5-(1H-tetrazol-5-yl)phenyl]-N-(4-pyridin-4-ylbenzyl)propanamide

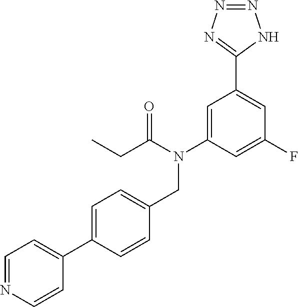

### Step 1: N-(3-cyano-5-fluorophenyl)-N-(4-pyridin-4-ylbenzyl)propanamide

Following the general method as outlined in Example 59 (Step 1), starting from N-(3-cyano-5-fluorophenyl)-N-(4-iodobenzyl)propanamide (Intermediate 31) and pyridine-4-boronic acid, the title compound was obtained in quantitative yield after purification by column chromatography (silica), eluting with cyclohexane containing increasing amounts of EtOAc.

MS (ESI⁻): 358.0

### Step 2: N-[3-fluoro-5-(1H-tetrazol-5-yl)phenyl]-N-(4-pyridin-4-ylbenzyl)propanamide

Following the general method as outlined in Example 16 (Step 2), starting from N-(3-cyano-5-fluorophenyl)-N-(4-pyridin-4-ylbenzyl)propanamide, the title compound was obtained as a white solid after purification by preparative HPLC.

MS (ESI⁻): 401.0. HPLC (Condition A): Rt 2.27 min (HPLC purity 98.4%).

### EXAMPLE 90

### N-[3-fluoro-5-(1H-tetrazol-5-yl)phenyl]-N-(4-pyridin-3-ylbenzyl)propanamide

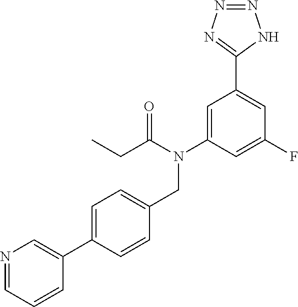

### Step 1: N-(3-cyano-5-fluorophenyl)-N-(4-pyridin-3-ylbenzyl)propanamide

Following the general method as outlined in Example 59 (Step 1), starting from N-(3-cyano-5-fluorophenyl)-N-(4-iodobenzyl)propanamide (Intermediate 31) and pyridine-3-boronic acid, the title compound was obtained in quantitative yield after purification by column chromatography (silica), eluting with cyclohexane containing increasing amounts of EtOAc.

MS (ESI⁻): 358.0

### Step 2: N-[3-fluoro-5-(1H-tetrazol-5-yl)phenyl]-N-(4-pyridin-3-ylbenzyl)propanamide

Following the general method as outlined in Example 16 (Step 2), starting from N-(3-cyano-5-fluorophenyl)-N-(4-pyridin-3-ylbenzyl)propanamide, the title compound was obtained as a white solid after purification by preparative HPLC.

MS (ESI⁻): 401.0. HPLC (Condition A): Rt 2.29 min (HPLC purity 98.2%).

### EXAMPLE 91

### N-[3-fluoro-5-(1H-tetrazol-5-yl)phenyl]-N-{[3′-(morpholin-4-ylsulfonyl)biphenyl-4-yl]methyl}propanamide

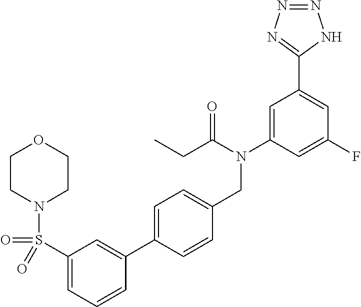

### Step 1: N-(3-cyano-5-fluorophenyl)-N-{[3′-(morpholin-4-ylsulfonyl)biphenyl-4-yl]methyl}propanamide

Following the general method as outlined in Example 59 (Step 1), starting from N-(3-cyano-5-fluorophenyl)-N-(4-iodobenzyl)propanamide (Intermediate 31) and 3-(N-morpholinylsulfonamidophenyl)boronic acid, the title compound was obtained after purification by column chromatography (silica), eluting with cyclohexane containing increasing amounts of EtOAc.

MS (ESI⁻): 505.9

### Step 2: N-[3-fluoro-5-(1H-tetrazol-5-yl)phenyl]-N-{[3′-(morpholin-4-ylsulfonyl)biphenyl-4-yl]methyl}propanamide

Following the general method as outlined in Example 16 (Step 2), starting from N-(3-cyanophenyl)-N-[4-(1H-pyrazol-3-yl)benzyl]propanamide, the title compound was obtained as a white solid after purification by preparative HPLC.

¹H NMR (300 MHz, DMSO-d6) δ 8.06-7.99 (m, 1H), 7.90-7.84 (m, 1H), 7.83-7.64 (m, 6H), 7.49 (d, J=9.6 Hz, 1H), 7.38 (d, J=8.1 Hz, 2H), 5.03 (s, 2H), 3.68-3.57 (m, 4H), 2.90 (s, 4H), 2.27 (m, 2H), 1.01 (t, J=7.3 Hz, 3H).

MS (ESI⁻): 549.0. HPLC (Condition A): Rt 3.94 min (HPLC purity 91.5%).

### EXAMPLE 92

### N-[3-fluoro-5-(1H-tetrazol-5-yl)phenyl]-N-[(3′-{[(2-hydroxyethyl)amino]sulfonyl}biphenyl-4-yl)methyl]propanamide

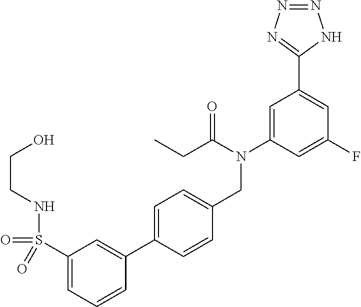

### Step 1: N-(3-cyano-5-fluorophenyl)-N-[(3′-{[(2-hydroxyethyl)amino]sulfonyl}biphenyl-4-yl)methyl]propanamide

Following the general method as outlined in Example 59 (Step 1), starting from N-(3-cyano-5-fluorophenyl)-N-(4-iodobenzyl)propanamide (Intermediate 31) and 3-[N-(2-hydroxyethylsulfamoyl)]phenylboronic acid, the title compound was obtained after purification by column chromatography (silica), eluting with cyclohexane containing increasing amounts of EtOAc.

MS (ESI⁻): 479.9

### Step 2: N-[3-fluoro-5-(1H-tetrazol-5-yl)phenyl]-N-[(3′-{[(2-hydroxyethyl)amino]sulfonyl}biphenyl-4-yl)methyl]propanamide

Following the general method as outlined in Example 16 (Step 2), starting from N-(3-cyano-5-fluorophenyl)-N-[(3′-{[(2-hydroxyethyl)amino]sulfonyl}biphenyl-4-yl)methyl]propanamide, the title compound was obtained as a white solid after purification by preparative HPLC.

¹H NMR (300 MHz, DMSO-d6) δ 8.09-7.99 (m, 1H), 7.96-7.86 (m, 1H), 7.83-7.62 (m, 7H), 7.58-7.42 (m, 2H), 7.38 (d, J=8.2 Hz, 2H), 5.03 (s, 2H), 4.70 (brs, 1H), 3.40-3.24 (m, 2H), 2.86-2.75 (m, 2H), 2.34-2.19 (m, 2H), 1.01 (t, J=7.3 Hz, 3H). MS (ESI⁻): 523.0. HPLC (Condition A): Rt 3.29 min (HPLC purity 86.4%).

### EXAMPLE 93

### N-({3′-[(dimethylamino)sulfonyl]biphenyl-4-yl}methyl)-N-[3-fluoro-5-(1H-tetrazol-5-yl)phenyl]propanamide

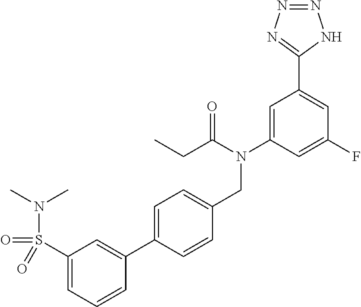

### Step 1: N-(3-cyano-5-fluorophenyl)-N-({3′-[(dimethylamino)sulfonyl]biphenyl-4-yl}methyl)propanamide

Following the general method as outlined in Example 59 (Step 1), starting from N-(3-cyano-5-fluorophenyl)-N-(4-iodobenzyl)propanamide (Intermediate 31) and 3-(N,N-dimethylsulfonamidophenyl)boronic acid, the title compound was obtained after purification by column chromatography (silica), eluting with cyclohexane containing increasing amounts of EtOAc.

MS (ESI⁻): 463.9

### Step 2: N-({3′-[(dimethylamino)sulfonyl]biphenyl-4-yl}methyl)-N-[3-fluoro-5-(1H-tetrazol-5-yl)phenyl]propanamide

Following the general method as outlined in Example 16 (Step 2), starting from N-(3-cyano-5-fluorophenyl)-N-({3′-[(dimethylamino)sulfonyl]biphenyl-4-yl}methyl)propanamide, the title compound was obtained after purification by solid-phase extraction (eluting first on a thiol SPE cartridge, then on a reverse-phase SPE cartridge).

¹H NMR (300 MHz, DMSO-d6) δ 8.03-7.95 (m, 1H), 7.91-7.84 (m, 1H), 7.84-7.75 (m, 2H), 7.75-7.64 (m, 4H), 7.54-7.46 (m, 1H), 7.38 (d, J=8.2 Hz, 2H), 5.03 (s, 2H), 2.64 (s, 6H), 2.36-2.14 (m, 2H), 1.01 (t, J=7.4 Hz, 3H). MS (ESI⁻): 507.0. HPLC (Condition A): Rt 4.01 min (HPLC purity 97.2%).

### EXAMPLE 94

### N-[3-fluoro-5-(1H-tetrazol-5-yl)phenyl]-N-[4-(1,3,5-trimethyl-1H-pyrazol-4-yl)benzyl]propanamide

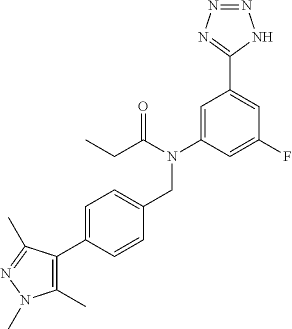

### Step 1: N-(3-cyano-5-fluorophenyl)-N-[4-(1,3,5-trimethyl-1H-pyrazol-4-yl)benzyl]propanamide

Following the general method as outlined in Example 59 (Step 1), starting from N-(3-cyano-5-fluorophenyl)-N-(4-iodobenzyl)propanamide (Intermediate 31) and 1,3,5-trimethyl-4-(4,4,5,5-tetramethyl-[1,3,2]dioxoborolan-2yl, the title compound was obtained after purification by column chromatography (silica), eluting with cyclohexane containing increasing amounts of EtOAc.

MS (ESI⁺): 391.0

### Step 2: N-[3-fluoro-5-(1H-tetrazol-5-yl)phenyl]-N-[4-(1,3,5-trimethyl-1H-pyrazol-4-yl)benzyl]propanamide

Following the general method as outlined in Example 16 (Step 2), starting from N-(3-cyano-5-fluorophenyl)-N-[4-(1,3,5-trimethyl-1H-pyrazol-4-yl)benzyl]propanamide, the title compound was obtained as a white solid after purification by preparative HPLC.

¹H NMR (300 MHz, DMSO-d6) δ 7.83-7.75 (m, 1H), 7.71 (brs, 1H), 7.55-7.44 (m, 1H), 7.30-7.09 (m, 4H), 4.96 (s, 2H), 3.67 (s, 3H), 2.32-2.17 (m, 2H), 2.15 (m, 4H), 2.06 (s, 3H), 1.00 (t, J=7.4 Hz, 3H). MS (ESI⁻): 432.0. HPLC (Condition A): Rt 2.93 min (HPLC purity 94.4%).

### EXAMPLE 95

### N-[3-fluoro-5-(1H-tetrazol-5-yl)phenyl]-N-{4-[(trifluoromethyl)sulfonyl]benzyl}propanamide

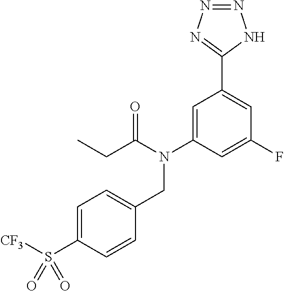

### Step 1: N-(3-cyano-5-fluorophenyl)-N-{4-[(trifluoromethyl)sulfonyl]benzyl}propanamide

A cooled solution (−15° C.) of N-(3-cyano-5-fluorophenyl)-N-{4-[(trifluoromethyl)thio]benzyl}propanamide (Example 82, Step 1, 100 mg; 0.26 mmol) in DCM (10 ml) was treated with 3-chloroperbenzoic acid (130 mg; 0.53 mmol). The reaction suspension was allowed to warm to RT and stirred for 16 h, then treated with a further portion of 3-chloroperbenzoic acid (230 mg; 1.33 mmol) and with DCM (10 ml). After 72 h the reaction mixture was poured into an aqueous (1 N) solution of NaOH and the phases separated. The organic layer was dried and concentrated under reduced pressure to give the title compound as a clear oil in 71% yield.

¹H NMR (300 MHz, DMSO-d6) δ [ppm] 8.08 (d, J=8.3 Hz, 2H), 7.91-7.85 (m, 1H), 7.83 (s, 1H), 7.78 (dt, J=9.8, 2.2 Hz, 1H), 7.72 (d, J=8.3 Hz, 2H), 5.11 (s, 2H), 2.33 (q, J=7.3 Hz, 2H), 0.99 (t, J=7.3 Hz, 3H). HPLC (Condition A): Rt 4.70 min (HPLC purity 94.4%).

### Step 2: N-[3-fluoro-5-(1H-tetrazol-5-yl)phenyl]-N-{4-[(trifluoromethyl)sulfonyl]benzyl}propanamide

Following the general method as outlined in Example 16 (Step 2), starting from N-(3-cyano-5-fluorophenyl)-N-{4-[(trifluoromethyl)sulfonyl]benzyl}propanamide, the title compound was obtained as a white solid.

¹H NMR (300 MHz, DMSO-d6) δ [ppm] 8.09 (d, J=8.4 Hz, 2H), 7.86-7.78 (m, 2H), 7.75 (d, J=8.4 Hz, 2H), 7.61 (dt, J=9.8, 2.0 Hz, 1H), 5.14 (s, 2H), 2.28 (q, J=7.4 Hz, 2H), 1.01 (t, J=7.4 Hz, 3H). MS (ESI⁻): 456.2. HPLC (Condition A): Rt 4.21 min (HPLC purity 94.8%).

### EXAMPLE 96

### N-(3-pyridin-4-ylbenzyl)-N-[3-(1H-tetrazol-5-yl)phenyl]propanamide

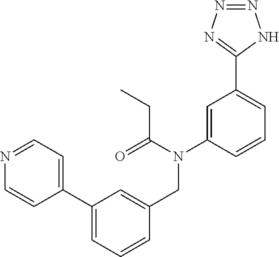

### Step 1: N-(3-cyanophenyl)-N-(3-pyridin-4-ylbenzyl)propanamide

Following the general method as outlined in Example 59 (Step 1), starting from N-(3-cyanophenyl)-N-(3-iodobenzyl)propanamide (Intermediate 22) and pyridine-4-boronic acid, the title compound was obtained as an oil after purification by column chromatography (silica), eluting with cyclohexane containing increasing amounts of EtOAc.

¹H NMR (300 MHz, DMSO-d6) δ [ppm] 8.62 (d, J=4.9 Hz, 2H), 7.88 (s, 1H), 7.76 (d, J=6.5 Hz, 1H), 7.71-7.52 (m, 6H), 7.44 (t, J=7.6 Hz, 1H), 7.29 (d, J=7.7 Hz, 1H), 5.00 (s, 2H), 2.24-2.05 (m, 2H), 0.97 (t, J=7.3 Hz, 3H). HPLC (Condition A): Rt 2.44 min (HPLC purity 98.1%).

### Step 2: N-(3-pyridin-4-ylbenzyl)-N-[3-(1H-tetrazol-5-yl)phenyl]propanamide

Following the general method as outlined in Example 16 (Step 2), starting from N-(3-cyanophenyl)-N-(3-pyridin-4-ylbenzyl)propanamide, the title compound was obtained as a white solid after purification by preparative HPLC.

¹H NMR (300 MHz, DMSO-d6) δ [ppm] 8.63 (d, J=4.7 Hz, 2H), 8.02-7.92 (m, 2H), 7.72-7.58 (m, 5H), 7.52-7.42 (m, 2H), 7.35 (d, J=7.7 Hz, 1H), 5.05 (s, 2H), 2.28-2.11 (m, 2H), 1.01 (t, 3H). MS (ESI⁺): 385.0. HPLC (Condition A): Rt 2.16 min (HPLC purity 95.9%).

### EXAMPLE 97

### N-(biphenyl-4-ylmethyl)-N-[3-fluoro-5-(1H-tetrazol-5-yl)phenyl]propanamide

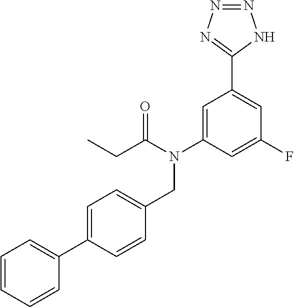

### Step 1: N-(biphenyl-4-ylmethyl)-N-(3-cyano-5-fluorophenyl)propanamide

Following the general method as outlined in Example 22 (Step 1), starting from N-(3-cyano-5-fluorophenyl)propanamide (Intermediate 20) and 4-bromomethylbiphenyl, the title compound was obtained as an oil in 89% yield after purification by column chromatography (silica), eluting with cyclohexane containing increasing amounts of EtOAc.

HPLC (Condition A): Rt 4.97 min (HPLC purity 98.6%).

### Step 2: N-(biphenyl-4-ylmethyl)-N-[3-fluoro-5-(1H-tetrazol-5-yl)phenyl]propanamide

Following the general method as outlined in Example 16 (Step 2), starting from N-(biphenyl-4-ylmethyl)-N-(3-cyano-5-fluorophenyl)propanamide, the title compound was obtained as a white foam in 81% yield.

¹H NMR (300 MHz, DMSO-d6) δ [ppm] 7.81-7.74 (m, 2H), 7.65-7.56 (m, 4H), 7.53-7.40 (m, 3H), 7.37-7.28 (m, 3H), 5.00 (s, 2H), 2.31-2.2 (m, 2H), 1.00 (t, J=7 Hz, 3H). MS (ESI⁺): 402.2. HPLC (Condition A): Rt 4.43 min (HPLC purity 98.7%).

### EXAMPLE 98

### N-[(2′-chlorobiphenyl-4-yl)methyl]-N-[3-fluoro-5-(1H-tetrazol-5-yl)phenyl]propanamide

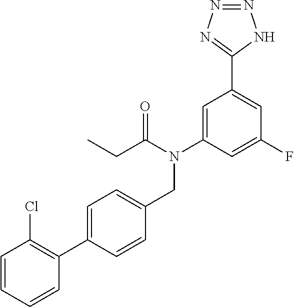

### Step 1: N-[(2′-chlorobiphenyl-4-yl)methyl]-N-(3-cyano-5-fluorophenyl)propanamide

Following the general method as outlined in Example 59 (Step 1), starting from N-(3-cyano-5-fluorophenyl)-N-(4-iodobenzyl)propanamide (Intermediate 31 and 2-chlorophenylboronic acid, the title compound was obtained as a white solid in 86% yield after purification by column chromatography (silica), eluting with cyclohexane containing increasing amounts of EtOAc.

¹H NMR (300 MHz, DMSO-d6) δ [ppm] 7.89-7.81 (m, 1H), 7.77 (t, J=1.5 Hz, 1H), 7.73 (dt, J=9.9, 2.2 Hz, 1H), 7.61-7.54 (m, 1H), 7.50-7.34 (m, 5H), 7.31 (d, J=8.3 Hz, 2H), 5.01 (s, 2H), 2.33-2.16 (m, 2H), 1.00 (t, J=7.3 Hz, 3H). HPLC (Condition A): Rt 4.93 min (HPLC purity 97.6%).

### Step 2: N-[(2′-chlorobiphenyl-4-yl)methyl]-N-[3-fluoro-5-(1H-tetrazol-5-yl)phenyl]propanamide

Following the general method as outlined in Example 16 (Step 2), starting from N-[(2′-chlorobiphenyl-4-yl)methyl]-N-(3-cyano-5-fluorophenyl)propanamide, the title compound was obtained as a white solid.

¹H NMR (300 MHz, DMSO-d6) δ [ppm] 7.86-7.77 (m, 2H), 7.60-7.50 (m, 2H), 7.46-7.29 (m, 7H), 5.03 (s, 2H), 2.35-2.19 (m, 2H), 1.02 (t, J=7.4 Hz, 3H). MS (ESI⁺): 436.2. HPLC (Condition A): Rt 4.43 min (HPLC purity 96.5%).

### EXAMPLE 99

### N-[3-fluoro-5-(1H-tetrazol-5-yl)phenyl]-N-[(2′-methoxybiphenyl-4-yl)methyl]propanamide

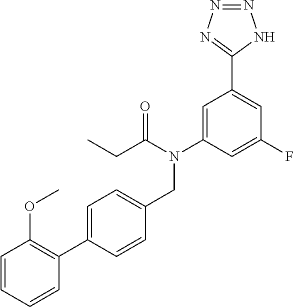

### Step 1: N-(3-cyano-5-fluorophenyl)-N-[(2′-methoxybiphenyl-4-yl)methyl]propanamide

Following the general method as outlined in Example 59 (Step 1), starting from N-(3-cyano-5-fluorophenyl)-N-(4-iodobenzyl)propanamide (Intermediate 31) and 2-methoxyphenylboronic acid, the title compound was obtained as a white solid after purification by column chromatography (silica), eluting with cyclohexane containing increasing amounts of EtOAc.

MS (ESI⁺): 389.2. HPLC (Condition A): Rt 4.75 min (HPLC purity 80.9%).

### Step 2: N-[3-fluoro-5-(1H-tetrazol-5-yl)phenyl]-N-[(2′-methoxybiphenyl-4-yl)methyl]propanamide

Following the general method as outlined in Example 16 (Step 2), starting from N-(3-cyano-5-fluorophenyl)-N-[(2′-methoxybiphenyl-4-yl)methyl]propanamide, the title compound was obtained as a white solid.

¹H NMR (300 MHz, DMSO-d6) δ [ppm] 7.86-7.78 (m, 2H), 7.55 (dt, J=9.8, 2.1 Hz, 1H), 7.42 (d, J=8.2 Hz, 2H), 7.38-7.23 (m, 4H), 7.10 (d, 1H), 7.05-6.98 (m, 1H), 5.01 (s, 2H), 3.74 (s, 3H), 2.36-2.21 (m, 2H), 1.02 (t, J=7.4 Hz, 3H). MS (ESI⁺): 432.2. HPLC (Condition A): Rt 4.23 min (HPLC purity 93.7%).

### EXAMPLE 100

### N-[2′,6′-dimethylbiphenyl-4-yl)methyl]-N-[3-fluoro-5-(1H-tetrazol-5-yl)phenyl]propanamide

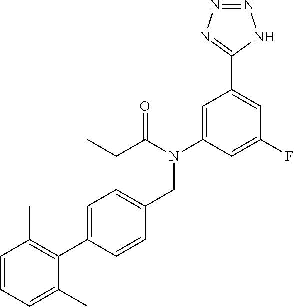

### Step 1: N-(3-cyano-5-fluorophenyl)-N-[(2′,6′-dimethylbiphenyl-4-yl)methyl]propanamide

Following the general method as outlined in Example 59 (Step 1), starting from N-(3-cyano-5-fluorophenyl)-N-(4-iodobenzyl)propanamide (Intermediate 31) and 2,6-dimethylphenylboronic acid, the title compound was obtained as a white solid after purification by column chromatography (silica), eluting with cyclohexane containing increasing amounts of EtOAc.

¹H NMR (300 MHz, DMSO-d6) δ [ppm] 7.88-7.80 (m, 1H), 7.66-7.58 (m, 2H), 7.25 (d, J=8.0 Hz, 2H), 7.19-7.03 (m, 5H), 4.98 (s, 2H), 2.26-2.15 (m, 2H), 1.93 (s, 6H), 1.00 (t, J=7.3 Hz, 3H). HPLC (Condition A): Rt 5.19 min (HPLC purity 95.9%).

### Step 2: N-[(2′,6′-dimethylbiphenyl-4-yl)methyl]-N-[3-fluoro-5-(1H-tetrazol-5-yl)phenyl]propanamide

Following the general method as outlined in Example 16 (Step 2), starting from N-(3-cyano-5-fluorophenyl)-N-[(2′,6′-dimethylbiphenyl-4-yl)methyl]propanamide, the title compound was obtained as a white solid in 85% yield.

¹H NMR (300 MHz, DMSO-d6) δ [ppm] 7.84-7.76 (m, 1H), 7.63 (s, 1H), 7.46 (d t, J=9.6, 2.1 Hz, 1H), 7.27 (d, J=8.0 Hz, 2H), 7.17-7.00 (m, 5H), 5.01 (s, 2H), 2.31-2.18 (m, 2H), 1.86 (s, 6H), 1.03 (t, J=7.4 Hz, 3H). MS (ESI⁺): 430.3. HPLC (Condition A): Rt 4.78 min (HPLC purity 95.5%).

### EXAMPLE 101

### N-[3-fluoro-5-(1H-tetrazol-5-yl)phenyl]-N-[3-(1H-pyrazol-4-yl)benzyl]propanamide

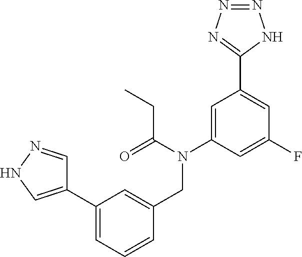

### Step 1: N-(3-cyano-5-fluorophenyl)-N-[3-(1H-pyrazol-4-yl)benzyl]propanamide

Following the general method as outlined in Example 59 (Step 1), starting from N-(3-cyano-5-fluorophenyl)-N-(4-iodobenzyl)propanamide (Intermediate 30) and 1-Boc-4-4(4,4,5,5,-tetramethyl-1,3,2-dioxaborolan-2-yl)pyrazole, the title compound was obtained as a yellow solid in 83% yield after purification by column chromatography (silica), eluting with cyclohexane containing increasing amounts of EtOAc.

¹H NMR (300 MHz, DMSO-d6) δ [ppm] 12.96 (br s, 1H), 8.17 (s, 1H), 7.95-7.63 (m, 4H), 7.47 (d, J=7.5 Hz, 1H), 7.38 (s, 1H), 7.28 (t, J=7.5 Hz, 1H), 7.03 (d, J=7.2 Hz, 1H), 4.98 (s, 2H), 2.35-2.16 (m, 2H), 1.00 (t, J=7.2 Hz, 3H). HPLC (Condition A): Rt 3.39 min (HPLC purity 99.5%).

### Step 2: N-[3-fluoro-5-(1H-tetrazol-5-yl)phenyl]-N-[3-(1H-pyrazol-4-yl)benzyl]propanamide

Following the general method as outlined in Example 16 (Step 2), starting from N-(3-cyano-5-fluorophenyl)-N-[3-(1H-pyrazol-4-yl)benzyl]propanamide, the title compound was obtained as a white foam after purification by preparative HPLC.

¹H NMR (300 MHz, DMSO-d6) δ [ppm] 12.94 (br s, 1H), 8.18-8.09 (m, 1H), 7.91-7.81 (m, 1H), 7.70-7.60 (m, 2H), 7.50-7.40 (m, 2H), 7.30 (t, J=7.6 Hz, 1H), 7.14 (d t, J=9.8, 2.2 Hz, 1H), 7.07 (d, J=7.6 Hz, 1H), 4.96 (s, 2H), 2.33-2.17 (m, 2H), 1.02 (t, J=7.4 Hz, 3H). MS (ESI⁺): 392.0. HPLC (Condition A): Rt 2.93 min (HPLC purity 97.5%).

### EXAMPLE 102

### N-[3-fluoro-5-(1H-tetrazol-5-yl)phenyl]-N-[(2′-methylbiphenyl-4-yl)methyl]propanamide

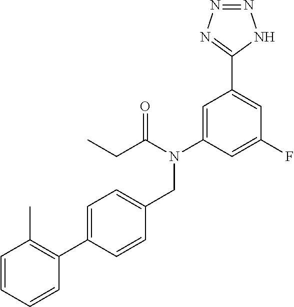

### Step 1: N-(3-cyano-5-fluorophenyl)-N-[(2′-methylbiphenyl-4-yl)methyl]propanamide

Following the general method as outlined in Example 59 (Step 1), starting from N-(3-cyano-5-fluorophenyl)-N-(4-iodobenzyl)propanamide (Intermediate 30) and o-tolylboronic acid, the title compound was obtained as a clear oil in 85% yield after purification by column chromatography (silica), eluting with cyclohexane containing increasing amounts of EtOAc.

¹H NMR (300 MHz, DMSO-d6) δ [ppm] 7.88-7.81 (m, 1H), 7.73 (t, J=1.5 Hz, 1H), 7.69 (d t, J=9.9, 2.2 Hz, 1H), 7.33-7.22 (m, 7H), 7.20-7.14 (m, 1H), 4.99 (s, 2H), 2.29-2.17 (m, 5H), 1.00 (t, J=7.3 Hz, 3H). HPLC (Condition A): Rt 4.98 min (HPLC purity 96.1%).

### Step 2: N-[3-fluoro-5-(1H-tetrazol-5-yl)phenyl]-N-[(2′-methylbiphenyl-4-yl)methyl]propanamide

Following the general method as outlined in Example 16 (Step 2), starting from N-(3-cyano-5-fluorophenyl)-N-[(2′-methylbiphenyl-4-yl)methyl]propanamide, the title compound was obtained as a white solid.

¹H NMR (300 MHz, DMSO-d6) δ [ppm] 7.85-7.78 (m, 1H), 7.74 (s, 1H), 7.53 (d t, J=9.8, 2.1 Hz, 1H), 7.33-7.20 (m, 7H), 7.19-7.13 (m, 1H), 5.02 (s, 2H), 2.33-2.19 (m, 3H), 2.16 (s, 3H), 1.02 (t, 3H). MS (ESI⁺): 416.2. HPLC (Condition A): Rt 4.58 min (HPLC purity 95.2%).

### EXAMPLE 103

### N-[3-chloro-5-(1H-tetrazol-5-yl)phenyl]-N-[(2,2-difluoro-1,3-benzodioxol-5-yl)methyl]propanamide

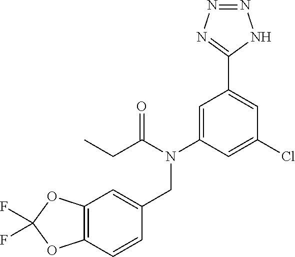

### Step 1: N-(3-chloro-5-cyanophenyl)-N-[(2,2-difluoro-1,3-benzodioxol-5-yl)methyl]propanamide

Following the general method as outlined in Example 7 (Step 1), starting from 3-chloro-5-{[(2,2-difluoro-1,3-benzodioxol-5-yl)methyl]amino}benzonitrile (Intermediate 27) and propionyl chloride, the title compound was obtained in 93% yield.

MS (ESI⁺): 378.9. HPLC (Condition A): Rt 5.45 min (HPLC purity 87.7%).

### Step 2: N-[3-chloro-5-(1H-tetrazol-5-yl)phenyl]-N-[(2,2-difluoro-1,3-benzodioxol-5-yl)methyl]propanamide

Following the general method as outlined in Example 16 (Step 2), starting from N-(3-chloro-5-cyanophenyl)-N-[(2,2-difluoro-1,3-benzodioxol-5-yl)methyl]propanamide, the title compound was obtained as a yellow foam.

¹H NMR (300 MHz, DMSO-d6) δ [ppm] 8.00 (t, J=1.6 Hz, 1H), 7.84 (t, J=1.6 Hz, 1H), 7.69 (t, J=1.9 Hz, 1H), 7.32 (d, J=3.1 Hz, 1H), 7.30 (d, J=3.6 Hz, 1H), 7.05 (m, 1H), 4.94 (s, 2H), 3.33 (brs, H), 2.21-2.17 (m, 2H), 0.97 (t, J=7.1 Hz, 3H). MS (ESI⁻): 420.2. HPLC (Condition A): Rt 4.42 min (HPLC purity 96.8%).

### EXAMPLE 104

### N-[3-fluoro-5-(1H-tetrazol-5-yl)phenyl]-3-methyl-N-[4-(methylsulfonyl)benzyl]butanamide

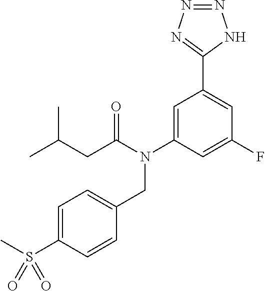

### Step 1: N-(3-cyano-5-fluorophenyl)-3-methyl-N-[4-(methylsulfonyl)benzyl]butanamide

Following the general method as outlined in Example 7 (Step 1), starting from 3-fluoro-5-{[4-(methylsulfonyl)benzyl]amino}benzonitrile (Intermediate 28) and isovaleryl chloride, the title compound was obtained after purification by column chromatography (silica), eluting with cyclohexane containing increasing amounts of EtOAc.

MS (ESI⁻): 387.0

### Step 2: N-[3-fluoro-5-(1H-tetrazol-5-yl)phenyl]-3-methyl-N-[4-(methylsulfonyl)benzyl]butanamide

Following the general method as outlined in Example 16 (Step 2), starting from N-(3-cyano-5-fluorophenyl)-3-methyl-N-[4-(methylsulfonyl)benzyl]butanamide, the title compound was obtained as a white solid after purification by preparative HPLC.

¹H NMR (300 MHz, DMSO-d6) δ 8.14-7.65 (m, 4H), 7.62-7.37 (m, 3H), 5.06 (brs, 2H), 3.18 (s, 3H), 2.22-1.92 (m, 3H), 0.93-0.72 (m, 6H). MS (ESI⁻): 430.0. HPLC (Condition A): Rt 3.37 min (HPLC purity 96.7%).

### EXAMPLE 105

### N-[3-fluoro-5-(1H-tetrazol-5-yl)phenyl]-N-[4-(methylsulfonyl)benzyl]cyclopentanecarboxamide

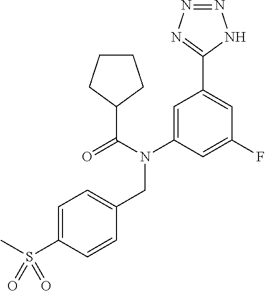

### Step 1: N-(3-cyano-5-fluorophenyl)-N-[4-(methylsulfonyl)benzyl]cyclopentanecarboxamide

Following the general method as outlined in Example 67 (Step 1), starting from 3-fluoro-5-{[4-(methylsulfonyl)benzyl]amino}benzonitrile (Intermediate 28) and cyclopentanecarboxylic acid, the title compound was obtained in 76% yield after purification by column chromatography (silica), eluting with cyclohexane containing increasing amounts of EtOAc.

MS (ESI⁻): 399.0

### Step 2: N-[3-fluoro-5-(1H-tetrazol-5-yl)phenyl]-N-[4-(methylsulfonyl)benzyl]cyclopentanecarboxamide

Following the general method as outlined in Example 16 (Step 2), starting from N-(3-cyano-5-fluorophenyl)-N-[4-(methylsulfonyl)benzyl]cyclopentanecarboxamide, the title compound was obtained as a white solid after purification by preparative HPLC.

¹H NMR (300 MHz, DMSO-d6) δ 7.95-7.65 (m, 4H), 7.64-7.42 (m, 3H), 5.04 (brs, 2H), 3.20 (s, 3H), 2.78 (brs, 1H), 1.87-1.24 (m, 8H). MS (ESI⁻): 442.0. HPLC (Condition A): Rt 3.44 min (HPLC purity 86.9%).

### EXAMPLE 106

### N-[3-fluoro-5-(1H-tetrazol-5-yl)phenyl]-N-[4-(methylsulfonyl)benzyl]isonicotinamide

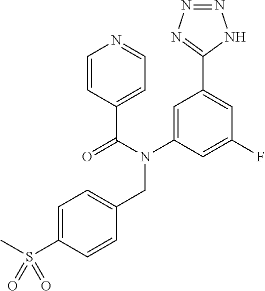

### Step 1: N-(3-cyano-5-fluorophenyl)-N-[4-(methylsulfonyl)benzyl]isonicotinamide

Following the general method as outlined in Example 67 (Step 1), starting from 3-fluoro-5-{[4-(methylsulfonyl)benzyl]amino}benzonitrile (Intermediate 28) and isonicotinic acid, the title compound was obtained after purification by column chromatography (silica), eluting with cyclohexane containing increasing amounts of EtOAc.

MS (ESI⁻): 408.0

### Step 2: N-[3-fluoro-5-(1H-tetrazol-5-yl)phenyl]-N-[4-(methylsulfonyl)benzyl]isonicotinamide

Following the general method as outlined in Example 16 (Step 2), starting from N-(3-cyano-5-fluorophenyl)-N-[4-(methylsulfonyl)benzyl]isonicotinamide, the title compound was obtained as a white solid after purification by preparative HPLC.

¹H NMR (300 MHz, DMSO-d6) δ 8.54 (brs, 2H), 7.88 (d, J=8.3 Hz, 2H), 7.72 (brs, 1H), 7.69-7.56 (m, 3H), 7.50-7.33 (m, 3H), 5.29 (s, 2H), 3.19 (s, 3H). MS (ESI⁻): 450.9. HPLC (Condition A): Rt 1.95 min (HPLC purity 97.3%).

### EXAMPLE 107

### N-[3-fluoro-5-(1H-tetrazol-5-yl)phenyl]-3-(4-methylphenyl)-N-[4-(methylsulfonyl)benzyl]propanamide

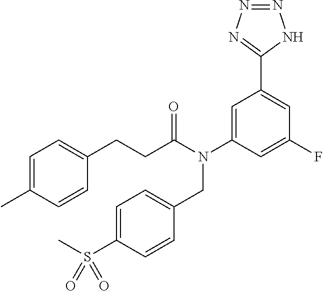

### Step 1: N-(3-cyano-5-fluorophenyl)-3-(4-methylphenyl)-N-[4-(methylsulfonyl)benzyl]propanamide

Following the general method as outlined in Example 67 (Step 1), starting from 3-fluoro-5-{[4-(methylsulfonyl)benzyl]amino}benzonitrile (Intermediate 28) and 3-(4-methylphenyl)propionic acid, the title compound was obtained after purification by column chromatography (silica), eluting with cyclohexane containing increasing amounts of EtOAc.

MS (ESI⁻): 449.0

### Step 2: N-[3-fluoro-5-(1H-tetrazol-5-yl)phenyl]-3-(4-methylphenyl)-N-[4-(methylsulfonyl)benzyl]propanamide

Following the general method as outlined in Example 16 (Step 2), starting from N-(3-cyano-5-fluorophenyl)-3-(4-methylphenyl)-N-[4-(methylsulfonyl)benzyl]propanamide, the title compound was obtained as a white solid after purification by preparative HPLC.

¹H NMR (300 MHz, DMSO-d6) δ 7.83 (d, J=7.0 Hz, 3H), 7.44 (d, J=7.7 Hz, 2H), 7.31-7.16 (m, 1H), 7.07-6.88 (m, 5H), 5.02 (brs, 2H), 3.33 (brs, 2H), 3.19 (s, 3H), 2.87-2.73 (m, 2H), 2.23 (s, 3H). MS (ESI⁻): 492.0. HPLC (Condition A): Rt 3.83 min (HPLC purity 93.0%).

### EXAMPLE 108

### N-[3-fluoro-5-(1H-tetrazol-5-yl)phenyl]-N-[4-(methylsulfonyl)benzyl]benzamide

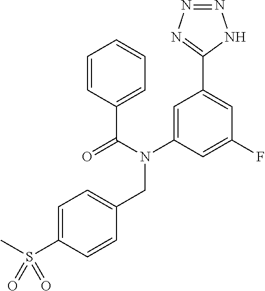

### Step 1: N-(3-cyano-5-fluorophenyl)-N-[4-(methylsulfonyl)benzyl]benzamide

Following the general method as outlined in Example 7 (Step 1), starting from 3-fluoro-5-{[4-(methylsulfonyl)benzyl]amino}benzonitrile (Intermediate 28) and benzoyl chloride, the title compound was obtained in 92% yield after purification by column chromatography (silica), eluting with cyclohexane containing increasing amounts of EtOAc.

MS (ESI⁻): 406.9

### Step 2: N-[3-fluoro-5-(1H-tetrazol-5-yl)phenyl]-N-[4-(methylsulfonyl)benzyl]benzamide

Following the general method as outlined in Example 16 (Step 2), starting from N-(3-cyano-5-fluorophenyl)-N-[4-(methylsulfonyl)benzyl]benzamide, the title compound was obtained as a white solid after purification by preparative HPLC.

¹H NMR (300 MHz, DMSO-d6) δ 7.79 (d, J=8.3 Hz, 2H), 7.66-7.42 (m, 4H), 7.40-7.31 (m, 2H), 7.31-7.11 (m, 4H), 5.20 (s, 2H), 3.09 (s, 3H). MS (ESI⁻): 450.0. HPLC (Condition A): Rt 3.22 min (HPLC purity 89.1%).

### EXAMPLE 109

### N-[3-fluoro-5-(1H-tetrazol-5-yl)phenyl]-N-[4-(methylsulfonyl)benzyl]-2-phenylacetamide

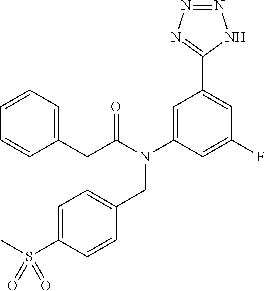

### Step 1: N-(3-cyano-5-fluorophenyl)-N-[4-(methylsulfonyl)benzyl]-2-phenylacetamide

Following the general method as outlined in Example 7 (Step 1), starting from 3-fluoro-5-{[4-(methylsulfonyl)benzyl]amino}benzonitrile (Intermediate 28) and phenylacetyl chloride, the title compound was obtained in quantitative yield after purification by column chromatography (silica), eluting with cyclohexane containing increasing amounts of EtOAc.

MS (ESI⁻): 421.0

### Step 2: N-[3-fluoro-5-(1H-tetrazol-5-yl)phenyl]-N-[4-(methylsulfonyl)benzyl]-2-phenylacetamide

Following the general method as outlined in Example 16 (Step 2), starting from N-(3-cyano-5-fluorophenyl)-N-[4-(methylsulfonyl)benzyl]-2-phenylacetamide, the title compound was obtained as a yellow solid after purification by preparative HPLC.

MS (ESI⁻): 464.0. HPLC (Condition A): Rt 3.43 min (HPLC purity 95.3%).

### EXAMPLE 110

### N-[3-fluoro-5-(1H-tetrazol-5-yl)phenyl]-1,3-dimethyl-N-[4-(methylsulfonyl)benzyl]-1H-pyrazole-5-carboxamide

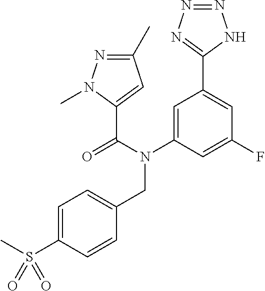

### Step 1: N-(3-cyano-5-fluorophenyl)-1,3-dimethyl-N-[4-(methylsulfonyl)benzyl]-1H-pyrazole-5-carboxamide

Following the general method as outlined in Example 67 (Step 1), starting from 3-fluoro-5-{[4-(methylsulfonyl)benzyl]amino}benzonitrile (Intermediate 28) and 1,3-dimethyl-1H-pyrazole-5-carboxylic acid, the title compound was obtained after purification by column chromatography (silica), eluting with cyclohexane containing increasing amounts of EtOAc.

MS (ESI⁻): 424.9

### Step 2: N-[3-fluoro-5-(1H-tetrazol-5-yl)phenyl]-1,3-dimethyl-N-[4-(methylsulfonyl)benzyl]-1H-pyrazole-5-carboxamide

Following the general method as outlined in Example 16 (Step 2), starting from N-(3-cyano-5-fluorophenyl)-1,3-dimethyl-N-[4-(methylsulfonyl)benzyl]-1H-pyrazole-5-carboxamide, the title compound was obtained as a white solid after purification by preparative HPLC.

¹H NMR (300 MHz, DMSO-d6) δ 7.88 (d, J=8.2 Hz, 2H), 7.84-7.67 (m, 2H), 7.61 (d, J=8.0 Hz, 2H), 7.47 (d, J=9.7 Hz, 1H), 5.63 (s, 1H), 5.25 (s, 2H), 3.94 (s, 3H), 3.19 (s, 3H), 1.93 (d, J=8.9 Hz, 3H). MS (ESI⁻): 468.24. HPLC (Condition A): Rt 2.87 min (HPLC purity 84.2%).

### EXAMPLE 111

### N-[3-fluoro-5-(1H-tetrazol-5-yl)phenyl]-N-(4-isoxazol-4-ylbenzyl)propanamide

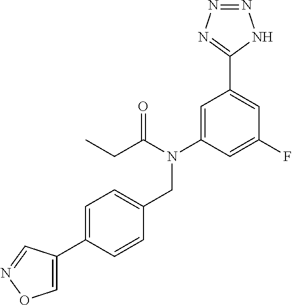

### Step 1: N-(3-cyano-5-fluorophenyl)-N-(4-isoxazol-4-ylbenzyl)propanamide

Following the general method as outlined in Example 59 (Step 1), starting from N-(3-cyano-5-fluorophenyl)-N-(4-iodobenzyl)propanamide (Intermediate 31) and 4-isoxazoleboronic acid pinacol ester, the title compound was obtained as an oil after purification by column chromatography (silica), eluting with cyclohexane containing increasing amounts of EtOAc.

¹H NMR (300 MHz, DMSO-d6) δ [ppm] 9.44 (s, 1H), 9.15 (s, 1H), 7.86-7.79 (m, 1H), 7.78-7.74 (m, 1H), 7.71-7.61 (m, 3H), 7.32-7.22 (m, 2H), 4.97 (s, 2H), 2.31-2.16 (m, 2H), 1.00 (t, J=7.3 Hz, 3H). HPLC (Condition A): Rt 3.89 min (HPLC purity 82.2%).

### Step 2: N-[3-fluoro-5-(1H-tetrazol-5-yl)phenyl]-N-(4-isoxazol-4-ylbenzyl)propanamide

Following the general method as outlined in Example 16 (Step 2), starting from N-(3-cyano-5-fluorophenyl)-N-(4-isoxazol-4-ylbenzyl)propanamide, the title compound was obtained as a white solid after purification by preparative HPLC.

¹H NMR (300 MHz, DMSO-d6) δ [ppm] 9.43 (s, 1H), 9.15 (s, 1H), 7.81-7.74 (m, 2H), 7.64 (d, J=8.2

Hz, 2H), 7.46 (dt, J=9.7, 2.1 Hz, 1H), 7.31 (d, J=8.2 Hz, 2H), 4.99 (s, 2H), 2.33-2.18 (m, 2H), 1.02 (t, J=7.4 Hz, 3H). MS (ESI⁺): 393.0. HPLC (Condition A): Rt 3.42 min (HPLC purity 98.4%).

### EXAMPLE 112

### N-[3-fluoro-5-(1H-tetrazol-5-yl)phenyl]-N-{3-[(trifluoromethyl)sulfinyl]benzyl}propanamide

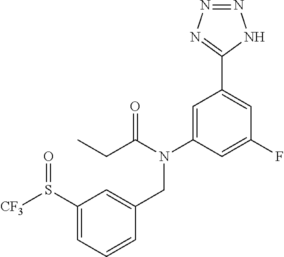

### Step 1: N-(3-cyano-5-fluorophenyl)-N-{3-[(trifluoromethyl)sulfinyl]benzyl}propanamide

A cooled (−15° C.) solution of N-(3-cyano-5-fluorophenyl)-N-{3-[(trifluoromethyl)thio]benzyl}propanamide (Example 82, Step 1; 49 mg; 0.08 mmol) in DCM (10 ml) was treated with 3-chloroperbenzoic acid (72 mg; 0.29 mmol). The reaction mixture was stirred for 16 hours at RT, cooled to −40° C. treated with a further portion of 3-chloroperbenzoic acid (99 mg; 0.57 mmol). The reaction solution was warmed slowly to RT and stirred for 16 h. The reaction mixture was diluted with DCM and washed with an aqueous solution (1 N) of NaOH (twice), then with a saturated solution of Na₂S₂O₃, dried and concentrated under reduced pressure to give the title compound as a yellow oil, used without further purification. MS (ESI⁺): 399.1.

### Step 2: N-[3-fluoro-5-(1H-tetrazol-5-yl)phenyl]-N-{3-[(trifluoromethyl)sulfinyl]benzyl}propanamide

Following the general method as outlined in Example 16 (Step 2), starting from N-(3-cyano-5-fluorophenyl)-N-{3-[(trifluoromethyl)sulfinyl]benzyl}propanamide, the title compound was obtained as a white solid after purification by preparative HPLC.

¹H NMR (300 MHz, DMSO-d6) δ [ppm] 7.81-7.73 (m, 2H), 7.73-7.69 (m, 2H), 7.69-7.64 (m, 2H), 7.40 (dt, J=9.7, 2.1 Hz, 1H), 5.10 (s, 2H), 2.32-2.18 (m, 2H), 1.01 (t, J=7.4 Hz, 3H). MS (ESI⁺): 441.9.

HPLC (Condition A): Rt 3.55 min (HPLC purity 95.0%).

### EXAMPLE 113

### N-[3-fluoro-5-(1H-tetrazol-5-yl)phenyl]-N-{4-[(trifluoromethyl)sulfinyl]benzyl}propanamide

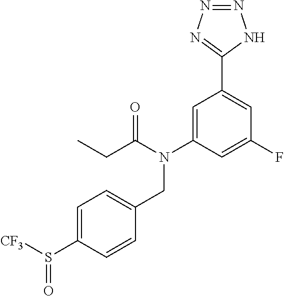

### Step 1: N-(3-cyano-5-fluorophenyl)-N-{4-[(trifluoromethyl)sulfinyl]benzyl}propanamide

Following the general method as outlined in Example 112 (Step 1), starting from N-(3-cyano-5-fluorophenyl)-N-{4-[(trifluoromethyl)thio]benzyl}propanamide (Example 77, Step 1), the title compound was obtained as a clear oil which was used without further purification for the next step.

¹H NMR (300 MHz, DMSO-d6) δ [ppm] 7.76 (d, J=8.3 Hz, 2H), 7.45 (d, J=8.3 Hz, 2H), 7.40-7.34 (m, 1H), 7.19 (s, 1H), 7.06-6.98 (m, 1H), 4.98 (s, 2H), 2.20-2.10 (m, 2H), 1.14 (t, J=7.4 Hz, 3H).

### Step 2: N-[3-fluoro-5-(1H-tetrazol-5-yl)phenyl]-N-{4-[(trifluoromethyl)sulfinyl]benzyl}propanamide

Following the general method as outlined in Example 16 (Step 2), starting from N-(3-cyano-5-fluorophenyl)-N-{4-[(trifluoromethyl)sulfinyl]benzyl}propanamide, the title compound was obtained as a white solid after purification by preparative HPLC.

¹H NMR (300 MHz, DMSO-d6) δ [ppm] 7.84 (d, J=8.2 Hz, 2H), 7.81-7.74 (m, 2H), 7.60 (d, J=8.2 Hz, 2H), 7.54 (m, 1H), 5.08 (s, 2H), 2.32-2.20 (m, 2H), 1.01 (t, 3H). MS (ESI⁺): 442.1. HPLC (Condition A): Rt 3.54 min (HPLC purity 98.3%).

### EXAMPLE 114

### N-[3-fluoro-5-(1H-tetrazol-5-yl)phenyl]-N-(4-hydroxybenzyl)propanamide

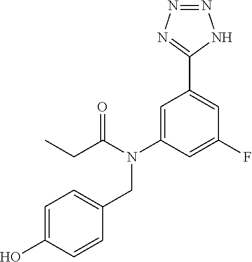

### Step 1: N-(3-cyano-5-fluorophenyl)-N-[4-(4,4,5,5-tetramethyl-1,3,2-dioxaborolan-2-yl)benzyl]propanamide

Following the general method as outlined in Example 84 (Step 1), starting from N-(3-cyano-5-fluorophenyl)-N-(4-iodobenzyl)propanamide (Intermediate 31), the title compound was obtained as a clear oil in 87% yield after purification by column chromatography (silica), eluting with cyclohexane containing increasing amounts of EtOAc.

¹H NMR (300 MHz, DMSO-d6) δ [ppm] 7.87-7.78 (m, 1H), 7.73 (m, 1H), 7.69-7.57 (m, 3H), 7.23 (d, J=8.0 Hz, 2H), 4.98 (s, 2H), 2.30-2.14 (m, 2H), 1.29 (s, 12H), 0.99 (t, J=7.3 Hz, 3H). HPLC (Condition A): Rt 4.76 min (HPLC purity 72.4%).

### Step 2: N-(3-cyano-5-fluorophenyl)-N-(4-hydroxybenzyl)propanamide

Following the general method as outlined in Example 84 (Step 2), starting from N-(3-cyano-5-fluorophenyl)-N-[3-(4,4,5,5-tetramethyl-1,3,2-dioxaborolan-2-yl)benzyl]propanamide, the title compound was obtained as a clear oil in 96% yield (uncorrected for purity) and was used without further purification.

¹H NMR (300 MHz, DMSO-d6) δ [ppm] 9.37 (s, 1H), 7.88-7.79 (m, 1H), 7.65 (s, 1H), 7.57 (dt, J=9.9, 2.1 Hz, 1H), 6.99 (d, J=8.5 Hz, 2H), 6.68 (d, J=8.5 Hz, 2H), 4.83 (s, 2H), 2.30-2.06 (m, 2H), 1.00 (t, J=7.3 Hz, 3H). HPLC (Condition A): Rt 3.15 min (HPLC purity 73.7%).

### Step 3: N-[3-fluoro-5-(1H-tetrazol-5-yl)phenyl]-N-(4-hydroxybenzyl)propanamide

Following the general method as outlined in Example 16 (Step 2), starting from N-(3-cyano-5-fluorophenyl)-N-(4-hydroxybenzyl)propanamide, the title compound was obtained as a white solid after purification by preparative HPLC.

¹H NMR (300 MHz, DMSO-d6) δ [ppm] 9.34 (s, 1H), 7.82-7.74 (m, 1H), 7.68 (s, 1H), 7.38 (dt, J=9.8, 2.1 Hz, 1H), 7.00 (d, J=8.5 Hz, 2H), 6.66 (d, J=8.5 Hz, 2H), 4.84 (s, 2H), 2.29-2.12 (m, 2H), 0.99 (t, J=7.4 Hz, 3H). MS (ESI⁺): 342.1. HPLC (Condition A): Rt 2.69 min (HPLC purity 97.8%).

### EXAMPLE 115

### N-[3-fluoro-5-(1H-tetrazol-5-yl)phenyl]-N-[3-(1H-pyrazol-5-yl)benzyl]propanamide

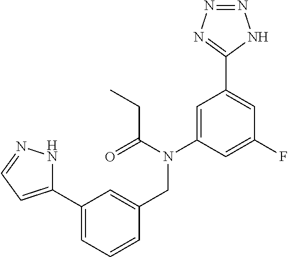

### Step 1: N-(3-cyano-5-fluorophenyl)-N-[3-(1H-pyrazol-5-yl)benzyl]propanamide

Following the general method as outlined in Example 59 (Step 1), starting from N-(3-cyano-5-fluorophenyl)-N-(3-iodobenzyl)propanamide (Intermediate 30) and 1H-pyrazole-3-boronic acid, the title compound was obtained as a yellow solid after purification by column chromatography (silica), eluting with cyclohexane containing increasing amounts of EtOAc.

¹H NMR (300 MHz, DMSO-d6) δ [ppm] 12.90 (br s, 1H), 7.88-7.51 (m, 6H), 7.33 (t, J=7.7 Hz, 1H), 7.13 (d, J=7.7 Hz, 1H), 6.68 (d, J=2.0 Hz, 1H), 5.00 (s, 2H), 2.33-2.17 (m, 2H), 1.00 (t, J=7.3 Hz, 3H). HPLC (Condition A): Rt 3.48 min (HPLC purity 98.7%).

### Step 2: N-[3-fluoro-5-(1H-tetrazol-5-yl)phenyl]-N-[3-(1H-pyrazol-5-yl)benzyl]propanamide

Following the general method as outlined in Example 16 (Step 2), starting from N-(3-cyano-5-fluorophenyl)-N-[3-(1H-pyrazol-5-yl)benzyl]propanamide, the title compound was obtained as a white solid after purification by preparative HPLC.

¹H NMR (300 MHz, DMSO-d6) δ [ppm] 7.79-7.61 (m, 5H), 7.43-7.30 (m, 2H), 7.16 (d, J=7.6 Hz, 1H), 6.65 (d, J=2.2 Hz, 1H), 5.00 (s, 2H), 2.36-2.16 (m, 2H), 1.01 (t, J=7.4 Hz, 3H). MS (ESI⁺): 391.9. HPLC (Condition A): Rt 3.04 min (HPLC purity 93.5%).

### EXAMPLE 116

### N-[3-fluoro-5-(1H-tetrazol-5-yl)phenyl]-N-[4-(methylsulfonyl)benzyl]nicotinamide

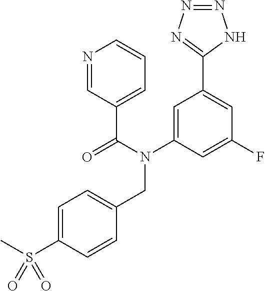

### Step 1: N-(3-cyano-5-fluorophenyl)-N-[4-(methylsulfonyl)benzyl]nicotinamide

Following the general method as outlined in Example 67 (Step 1), starting from 3-fluoro-5-{[4-(methylsulfonyl)benzyl]amino}benzonitrile (Intermediate 28) and nicotinic acid, the title compound was obtained after purification by column chromatography (silica), eluting with cyclohexane containing increasing amounts of EtOAc.

MS (ESI⁻): 407.9

### Step 2: N-[3-fluoro-5-(1H-tetrazol-5-yl)phenyl]-N-[4-(methylsulfonyl)benzyl]nicotinamide

Following the general method as outlined in Example 16 (Step 2), starting from N-(3-cyano-5-fluorophenyl)-N-[4-(methylsulfonyl)benzyl]nicotinamide, the title compound was obtained as a white solid after purification by preparative HPLC.

¹H NMR (300 MHz, DMSO-d6) δ 8.56 (brd, J=33.7 Hz, 2H), 7.96-7.79 (m, 3H), 7.78-7.54 (m, 4H), 7.43 (d, J=10.0 Hz, 1H), 7.37-7.27 (m, 1H), 5.31 (s, 2H), 3.19 (s, 3H). MS (ESI⁻): 450.9, HPLC (Condition A): Rt 2.08 min (HPLC purity 96.6%).

### EXAMPLE 117

### N-[3-fluoro-5-(1H-tetrazol-5-yl)phenyl]-2-methyl-N-[4-(methylsulfonyl)benzyl]propanamide

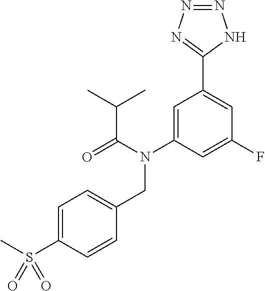

### Step 1: N-(3-cyano-5-fluorophenyl)-2-methyl-N-[4-(methylsulfonyl)benzyl]propanamide

Following the general method as outlined in Example 67 (Step 1), starting from 3-fluoro-5-{[4-(methylsulfonyl)benzyl]amino}benzonitrile (Intermediate 28) and isobutyric acid, the title compound was obtained as an oil in 74% yield after purification by column chromatography (silica), eluting with cyclohexane containing increasing amounts of EtOAc.

MS (ESI⁻): 373.0

### Step 2: N-[3-fluoro-5-(1H-tetrazol-5-yl)phenyl]-2-methyl-N-[4-(methylsulfonyl)benzyl]propanamide

Following the general method as outlined in Example 16 (Step 2), starting from N-(3-cyano-5-fluorophenyl)-2-methyl-N-[4-(methylsulfonyl)benzyl]propanamide, the title compound was obtained as a white solid after purification by preparative HPLC.

¹H NMR (300 MHz, DMSO-d6) δ 7.87 (d, J=8.4 Hz, 2H), 7.81 (d, J=8.3 Hz, 1H), 7.73 (s, 1H), 7.56-7.45 (m, 3H), 5.02 (s, 2H), 3.18 (s, 3H), 2.71-2.53 (m, 1H), 1.03 (d, J=6.7 Hz, 6H). MS (ESI⁻): 416.0. HPLC (Condition A): Rt 2.69 min (HPLC purity 91.5%).

### EXAMPLE 118

### N-[3-fluoro-5-(1H-tetrazol-5-yl)phenyl]-N-[4-(methylsulfonyl)benzyl]tetrahydro-2H-pyran-4-carboxamide

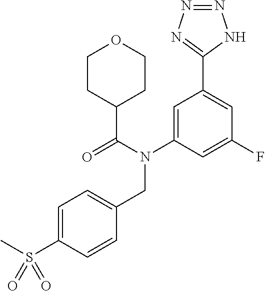

### Step 1: N-(3-cyano-5-fluorophenyl)-N-[4-(methylsulfonyl)benzyl]tetrahydro-2H-pyran-4-carboxamide

Following the general method as outlined in Example 67 (Step 1), starting from 3-fluoro-5-{[4-(methylsulfonyl)benzyl]amino}benzonitrile (Intermediate 28) and tetrahydro-2H-pyran-4-carboxylic acid, the title compound was obtained in 94% yield after purification by column chromatography (silica), eluting with cyclohexane containing increasing amounts of EtOAc.

MS (ESI⁻): 415.0

### Step 2: N-[3-fluoro-5-(1H-tetrazol-5-yl)phenyl]-N-[4-(methylsulfonyl)benzyl]tetrahydro-2H-pyran-4-carboxamide

Following the general method as outlined in Example 16 (Step 2), starting from N-(3-cyano-5-fluorophenyl)-N-[4-(methylsulfonyl)benzyl]tetrahydro-2H-pyran-4-carboxamide, the title compound was obtained as a white foam after purification by preparative HPLC.

¹H NMR (300 MHz, DMSO-d6) δ 7.98-7.79 (m, 3H), 7.76 (brs, 1H), 7.57 (d, J=10.1 Hz, 1H), 7.50 (d, J=8.2 Hz, 2H), 5.03 (s, 2H), 3.78 (d, J=11.0 Hz, 2H), 3.41 (s, 1H), 3.18 (s, 3H), 3.16-3.00 (m, 2H), 1.81-1.41 (m, 4H). MS (ESI⁻): 457.9. HPLC (Condition A): Rt 2.65 min (HPLC purity 99.3%).

### EXAMPLE 119

### N-[4-(2,4-dimethyl-1,3-thiazol-5-yl)benzyl]-N-[3-fluoro-5-(1H-tetrazol-5-yl)phenyl]propanamide

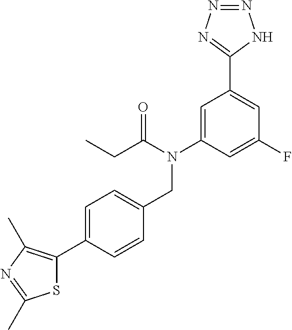

### Step 1: N-(3-cyano-5-fluorophenyl)-N-[4-(2,4-dimethyl-1,3-thiazol-5-yl)benzyl]propanamide

Following the general method as outlined in Example 59 (Step 1), starting from N-(3-cyano-5-fluorophenyl)-N-(4-iodobenzyl)propanamide (Intermediate 31) and 2,4-dimethyl-5-(4,4,5,5,-tetramethyl-1,3,2-dioxaborolan-2-yl)1,3-thiazole, the title compound was obtained as a clear oil in 71% yield after purification by column chromatography (silica), eluting with cyclohexane containing increasing amounts of EtOAc.

¹H NMR (300 MHz, DMSO-d6) δ [ppm] 7.89-7.81 (m, 1H), 7.77 (s, 1H), 7.71 (d t, J=9.9, 2.2 Hz, 1H), 7.38 (d, J=8.2 Hz, 2H), 7.29 (d, J=8.2 Hz, 2H), 4.97 (s, 2H), 2.62 (s, 3H), 2.36 (s, 3H), 2.30-2.16 (m, 2H), 0.99 (t, J=7.3 Hz, 3H). HPLC (Condition A): Rt 3.33 min (HPLC purity 88.9%).

### Step 2: N-[4-(2,4-dimethyl-1,3-thiazol-5-yl)benzyl]-N-[3-fluoro-5-(1H-tetrazol-5-yl)phenyl]propanamide

Following the general method as outlined in Example 16 (Step 2), starting from N-(3-cyano-5-fluorophenyl)-N-[4-(2,4-dimethyl-1,3-thiazol-5-yl)benzyl]propanamide, the title compound was obtained as a white solid after purification by preparative HPLC.

¹H NMR (300 MHz, DMSO-d6) δ [ppm] 7.85-7.74 (m, 2H), 7.52 (dt, J=9.8, 2.1 Hz, 1H), 7.39 (d, J=8.3 Hz, 2H), 7.32 (d, J=8.3 Hz, 2H), 5.00 (s, 2H), 2.62 (s, 3H), 2.34 (s, 3H), 2.32-2.20 (m, 2H), 1.01 (t, J=7.4 Hz, 3H). MS (ESI⁺): 437.1. HPLC (Condition A): Rt 2.84 min (HPLC purity 97.3%).

### EXAMPLE 120

### Preparation of hCRTH₂—CHO Expressing Cell Membranes

Adherent CHO cells expressing hCRTH2 (Euroscreen, Belgium) were cultured in 225 cm² cell culture flasks (Corning, USA) in 30 ml of medium. After two rinses of phosphate buffered saline (PBS), cells were harvested in 10 ml of PBS containing 1 mM EDTA, centrifuged at 500×g for 5 min at 4° C. and frozen at −80° C. The pellet was re-suspended in 50 mM Tris-HCl, pH 7.4, 2 mM EDTA, 250 mM Sucrose, containing protease inhibitor cocktail tablets, (Complete EDTA-free, Roche, Germany) and incubated 30 min at 4° C. Cells were disrupted by nitrogen cavitation (Parr Instruments, USA) at 4° C. (800 p.s.i. for 30 min), and centrifuged at 500×g for 10 min at 4° C. Pellet containing nuclei and cellular debris was discarded and supernatant was centrifuged 60 min at 4° C. at 45000×g. Membrane pellet was re-suspended in storage buffer (10 mM HEPES/KOH pH 7.4, 1 mM EDTA, 250 mM sucrose, protease inhibitor cocktail tablets) using Dounce homogenization and frozen in liquid nitrogen, and stored at 80° C.

### EXAMPLE 121

### Radioligand Binding Assay

The compounds of the present invention inhibit the binding of PGD2 to its receptor CRTH2. The inhibitory activity can be investigated by a radioligand binding Scintillation Proximity Assay (SPA) (Sawyer et al., Br. J. Pharmocol 2002, 137, 1163-72). The SPA radioligand binding assay was performed at room temperature in binding buffer (10 mM HEPES/KOH pH 7.4, 10 mM MnCl2, with protease inhibitor cocktail tablets), containing 1.5 nM [3H]PGD2 (Perkin Elmer), 10-50 μg/ml of hCRTH₂—CHO cell membrane protein and 2 mg/ml of Wheat-germ agglutinin Scintillation Proximity Assay beads (RPNQ0001, GE-Healthcare) in a final volume of 100 μl in 96 well plates (Corning, USA). Non-specific binding was determined in the presence of 10 μM PGD2 (Cayman, USA). Competing Compounds of Formula (I) were diluted in dimethylsulphoxide so that the total volume of dimethylsulfoxide was kept constant at 1% dimethylsulphoxide (Me₂SO). Serial dilutions of 100 μM to 100 μM were prepared and 10 μl each of the compounds of Formula (I) stock solutions were added to the binding assay reagents and incubated for 90 min with agitation at room temperature. Binding activity was determined by using a 1450 Micro-beta scintillation counter (Wallac, UK).

In one embodiment, the compounds of Formula (I) of the present invention inhibit CRTH2 at a concentration of <5 mM. Preferably, the compounds of Formula (I) of the present invention inhibit CRTH2 at a concentration of <1 μM. most preferably, the compounds of Formula (I) of the present invention inhibit CRTH2 at a concentration of <0.1 μM.

### Results:

| | | DP2 Binding assay (IC50, |
|---|---|---|
| EX. | Formula | nM) |
| 1 | 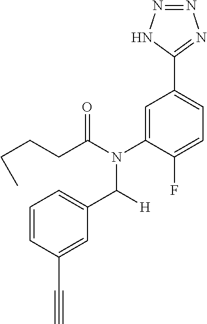 | 12 |
| 2 | 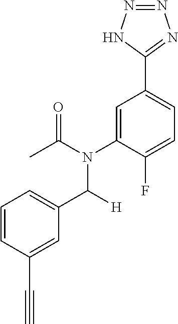 | 175 |
| 3 | 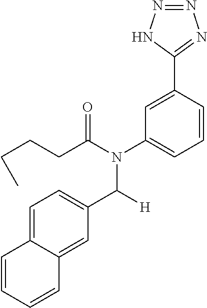 | 49 |
| 4 | 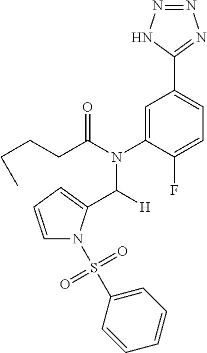 | 51 |
| 5 | 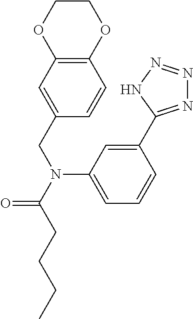 | 8.8 |
| 6 | 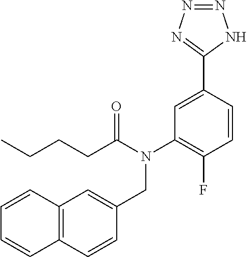 | 23 |
| 7 | 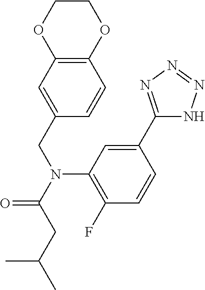 | 7.1 |
| 8 | 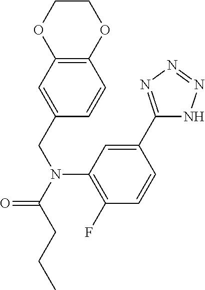 | 6.3 |
| 9 | 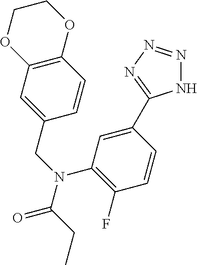 | 15 |
| 10 | 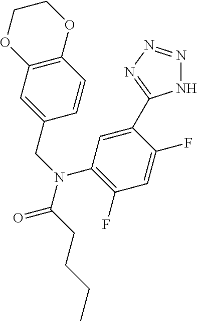 | 54 |
| 11 | 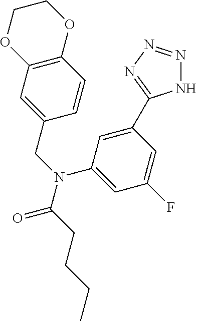 | 2.3 |
| 12 | 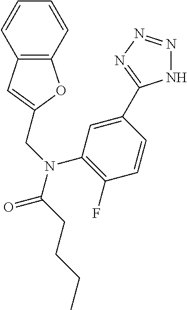 | 8.6 |
| 13 | 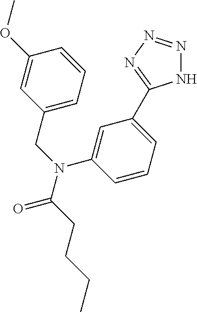 | 53 |
| 14 | 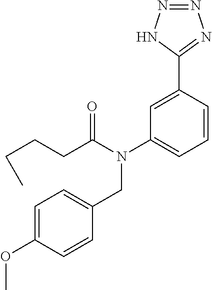 | 61 |
| 15 | 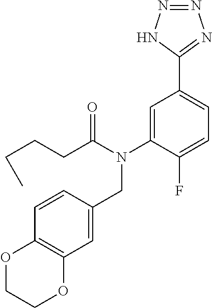 | 4.2 |
| 16 | 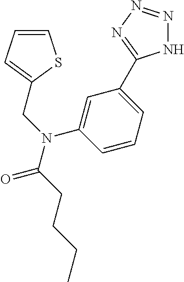 | 588 |
| 17 | 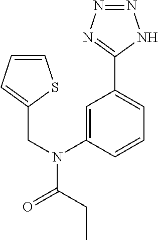 | 1595 |
| 18 | 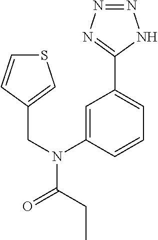 | 2105 |
| 19 | 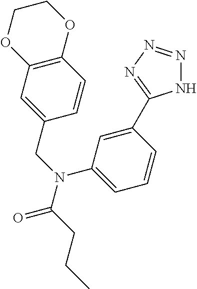 | 15 |
| 20 | 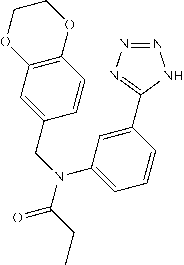 | 51 |
| 21 | 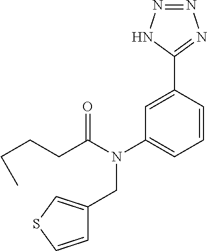 | 759 |
| 22 | 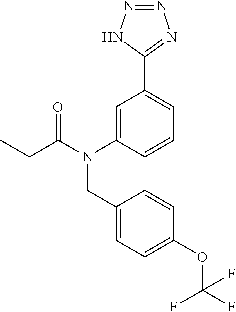 | 132 |
| 23 | 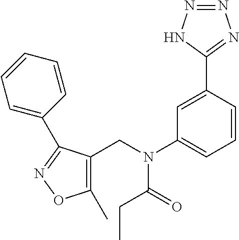 | 2955 |
| 24 | 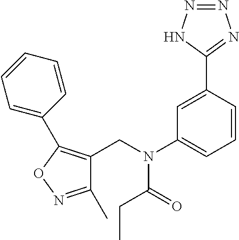 | 2180 |
| 25 | 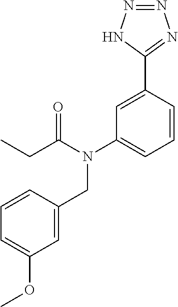 | 444 |
| 26 | 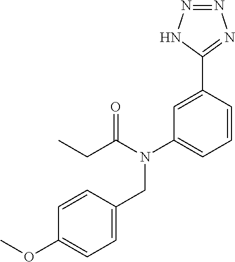 | 268 |
| 27 | 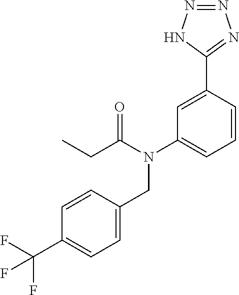 | 129 |
| 28 | 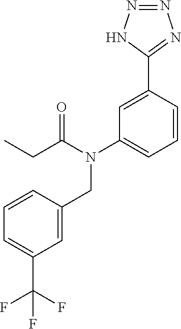 | 268 |
| 29 | 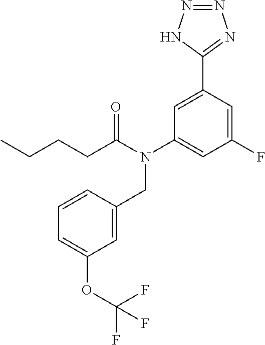 | 5.2 |
| 30 | 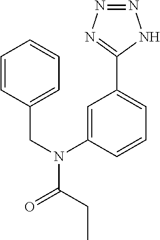 | 2250 |
| 31 | 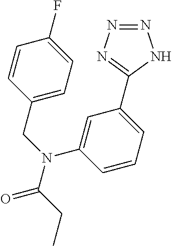 | 1535 |
| 32 | 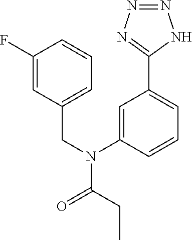 | 1695 |
| 33 | 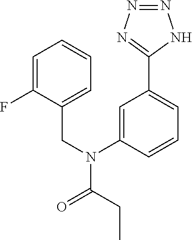 | 1160 |
| 34 | 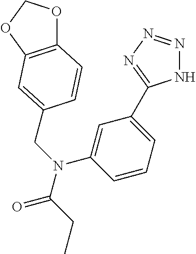 | 147 |
| 35 | 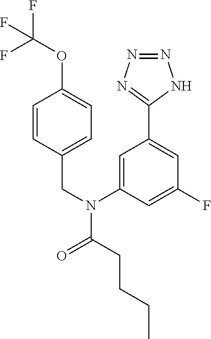 | 8.9 |
| 36 | 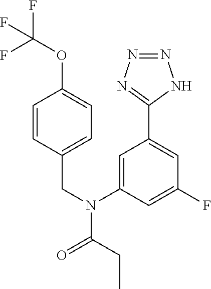 | 9.3 |
| 37 | 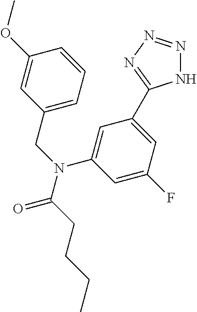 | 3.6 |
| 38 | 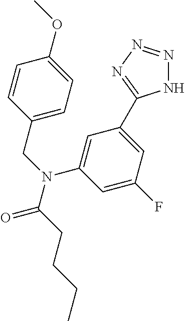 | 3.6 |
| 39 | 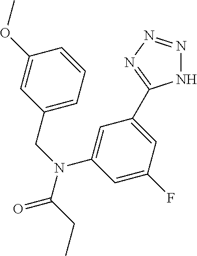 | 9.6 |
| 40 | 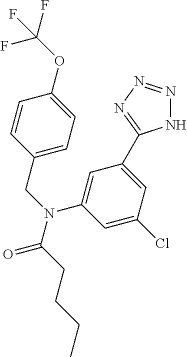 | 25 |
| 41 | 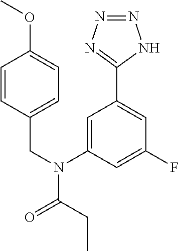 | 9.0 |
| 42 | 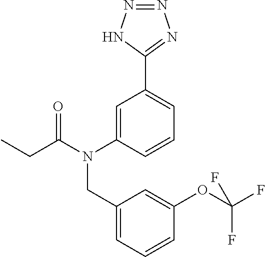 | 245 |
| 43 | 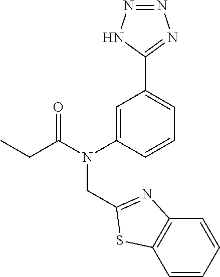 | 663 |
| 44 | 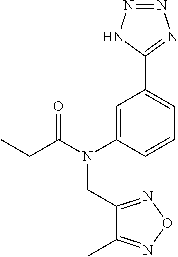 | 2095 |
| 45 | 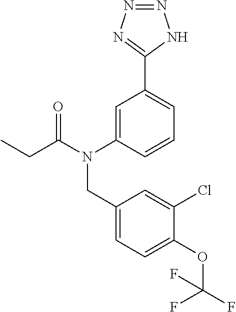 | 83 |
| 46 | 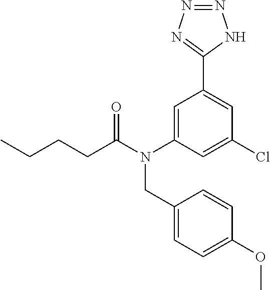 | 4.2 |
| 47 | 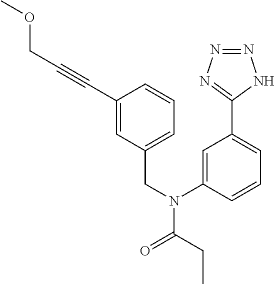 | 1140 |
| 48 | 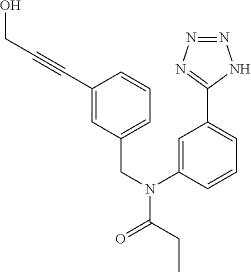 | 1270 |
| 49 | 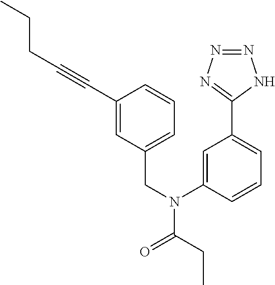 | 618 |
| 50 | 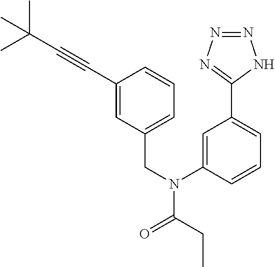 | 1300 |
| 51 | 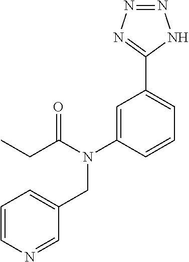 | 6750 |
| 52 | 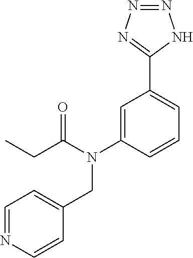 | 6680 |
| 53 | 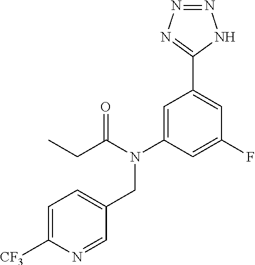 | — |
| 54 | 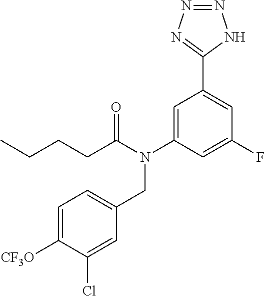 | 15 |
| 55 | 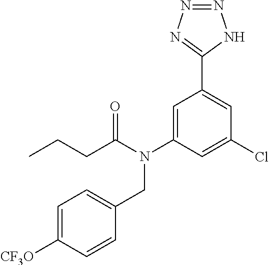 | 16 |
| 56 | 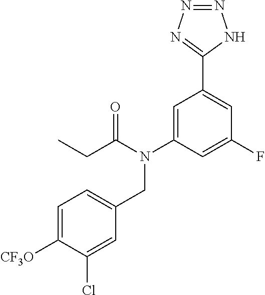 | 24 |
| 57 | 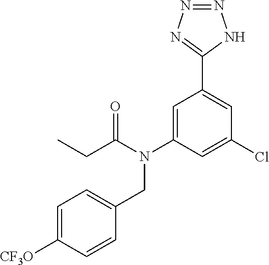 | 15 |
| 58 | 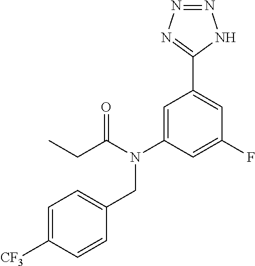 | 7.1 |
| 59 | 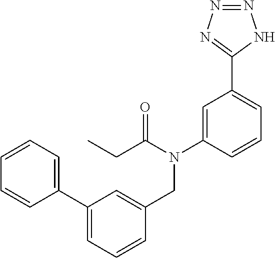 | 230 |
| 60 | 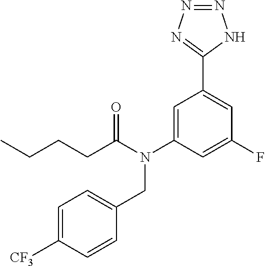 | 2.6 |
| 61 | 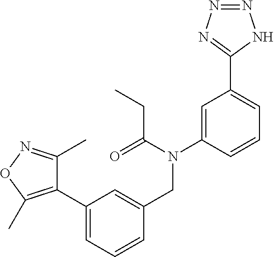 | 305 |
| 62 | 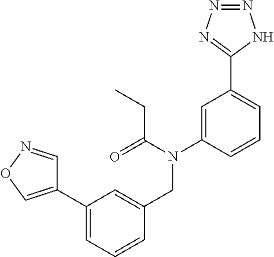 | 769 |
| 63 | 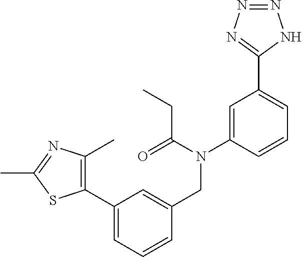 | 235 |
| 64 | 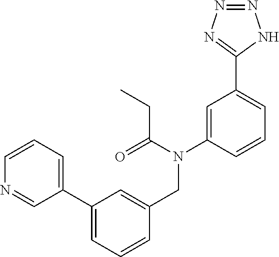 | 403 |
| 65 | 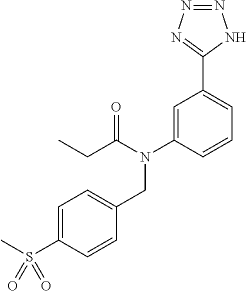 | 75 |
| 66 | 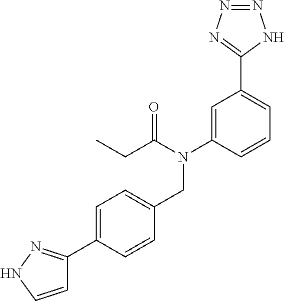 | 973 |
| 67 | 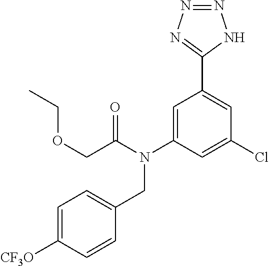 | 16 |
| 68 | 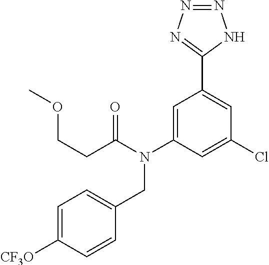 | 7.4 |
| 69 | 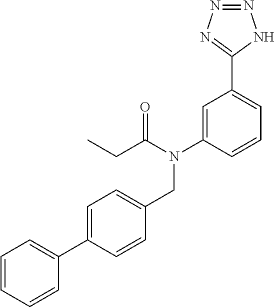 | 506 |
| 70 | 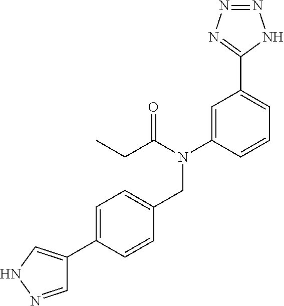 | 455 |
| 71 | 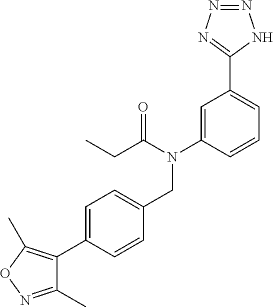 | 182 |
| 72 | 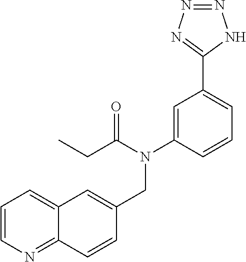 | 1110 |
| 73 | 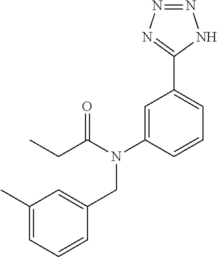 | 662 |
| 74 | 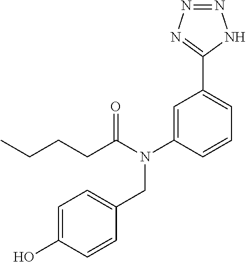 | 169 |
| 75 | 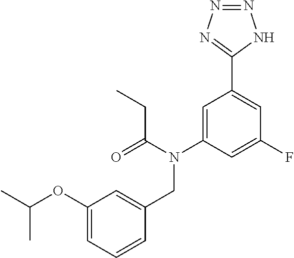 | 7.1 |
| 76 | 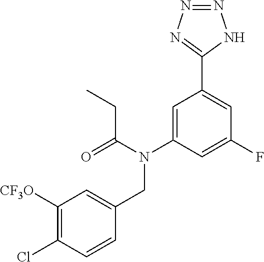 | 18 |
| 77 | 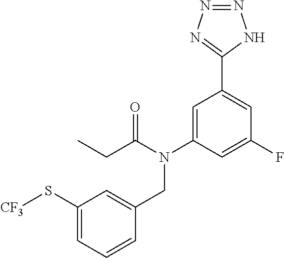 | 16 |
| 78 | 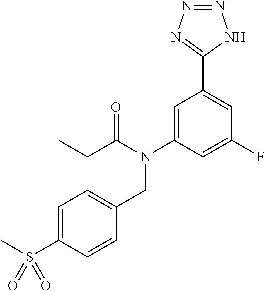 | 3.5 |
| 79 | 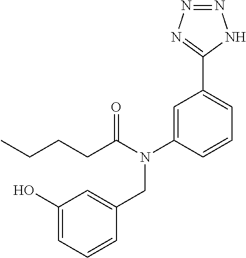 | 140 |
| 80 | 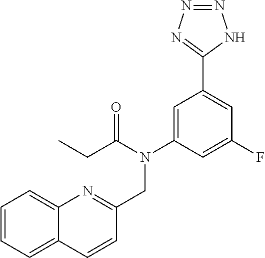 | 65 |
| 81 | 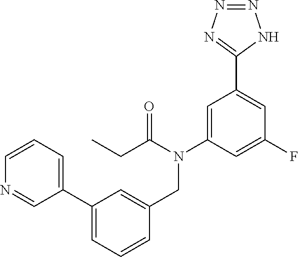 | 34 |
| 82 | 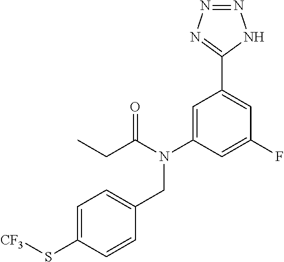 | 2.6 |
| 83 | 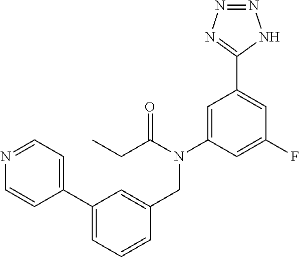 | 25 |
| 84 | 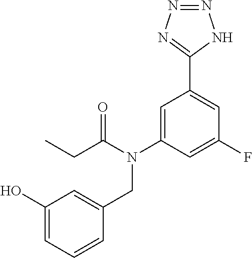 | 28 |
| 85 | 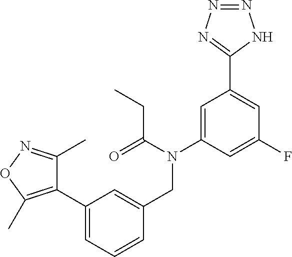 | 19 |
| 86 | 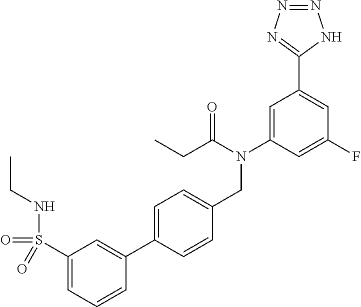 | 170 |
| 87 | 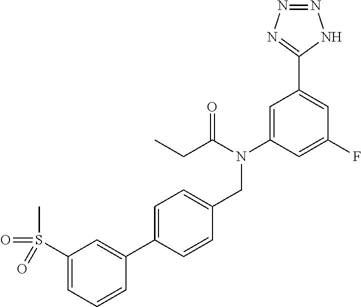 | 185 |
| 88 | 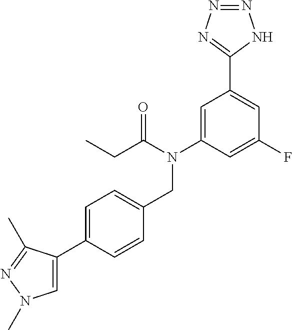 | 21 |
| 89 | 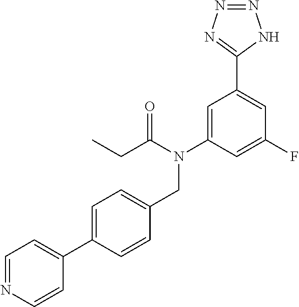 | 57 |
| 90 | 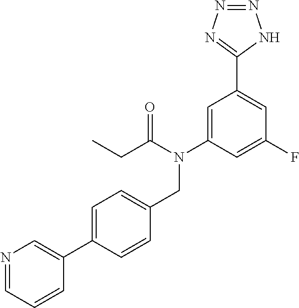 | 140 |
| 91 | 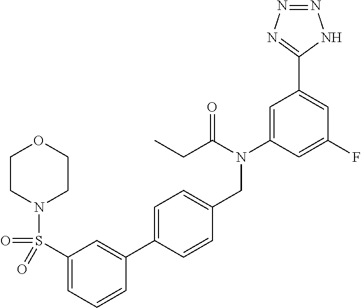 | 145 |
| 92 | 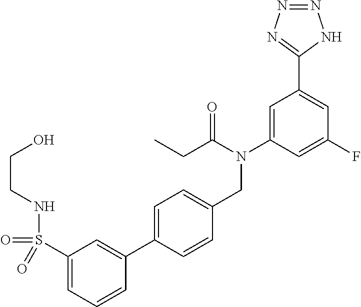 | 160 |
| 93 | 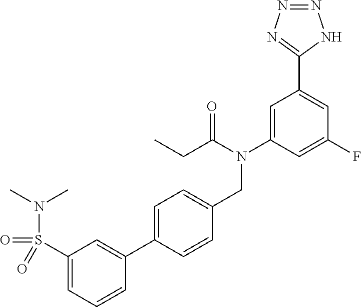 | 125 |
| 94 | 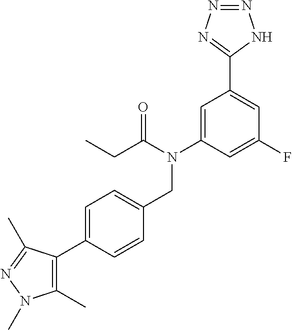 | 21 |
| 95 | 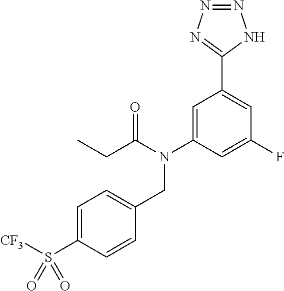 | 5.6 |
| 96 | 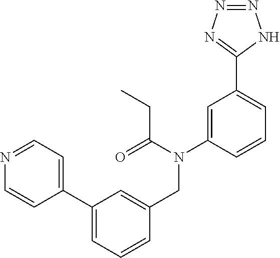 | 330 |
| 97 | 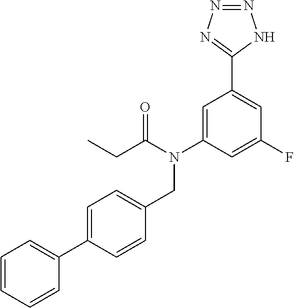 | 70 |
| 98 | 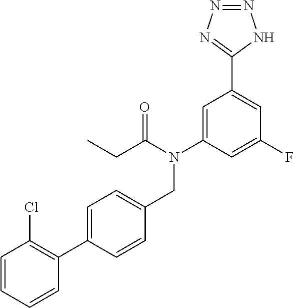 | 31 |
| 99 | 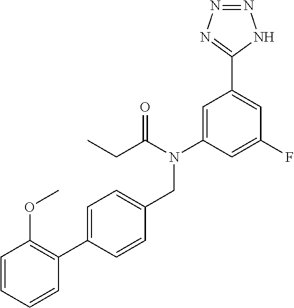 | 13 |
| 100 | 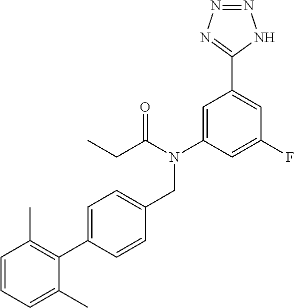 | 8.3 |
| 101 | 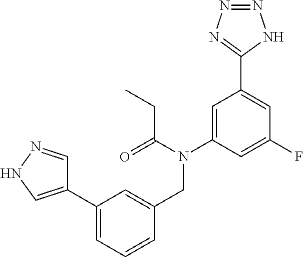 | 19 |
| 102 | 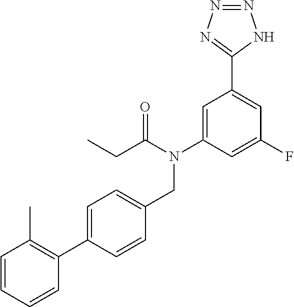 | 8.9 |
| 103 | 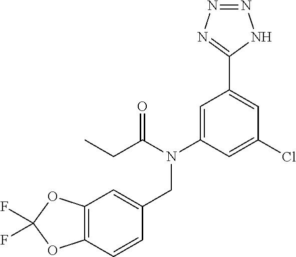 | 13 |
| 104 | 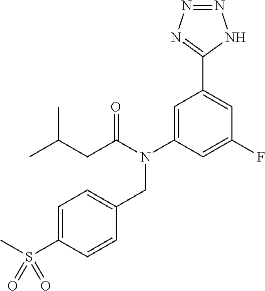 | 3.7 |
| 105 | 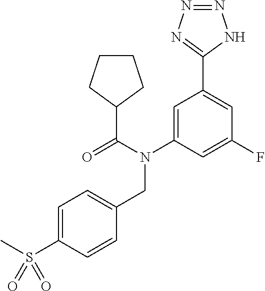 | 6.2 |
| 106 | 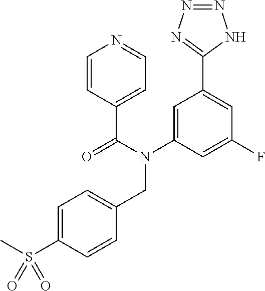 | 20 |
| 107 | 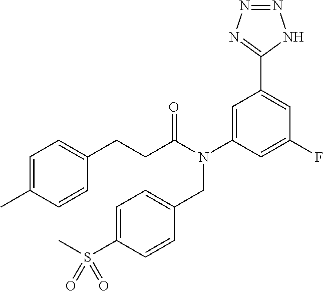 | 2.9 |
| 108 | 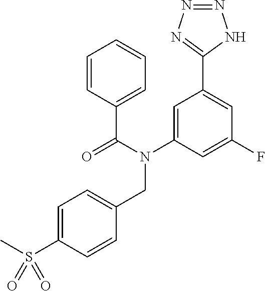 | 2.6 |
| 109 | 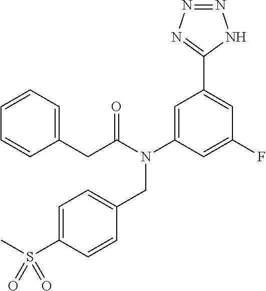 | 1.0 |
| 110 | 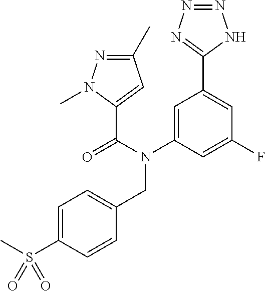 | 56 |
| 111 | 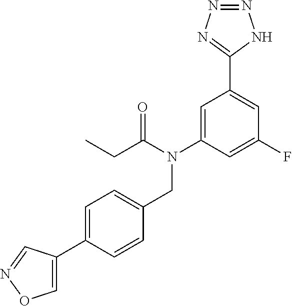 | 113 |
| 112 | 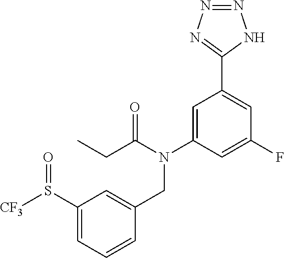 | 19 |
| 113 | 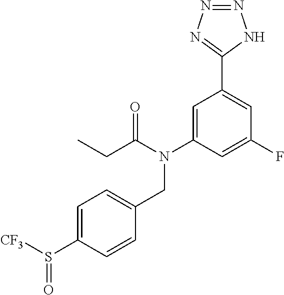 | 3.5 |
| 114 | 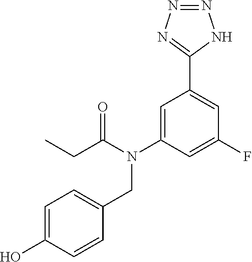 | 25 |
| 115 | 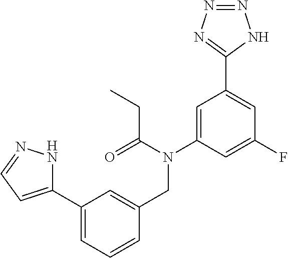 | 30 |
| 116 | 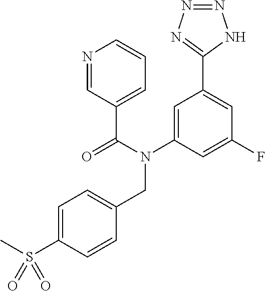 | 4.6 |
| 117 | 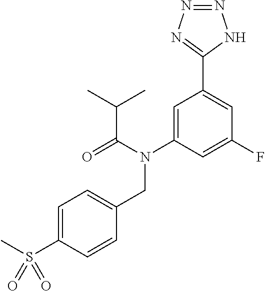 | 19 |
| 118 | 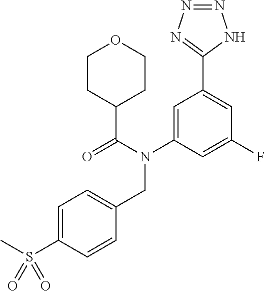 | 32 |
| 119 | 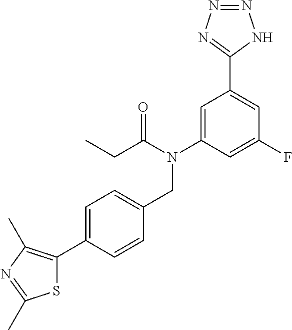 | 5.2 |

### EXAMPLE 122 [³⁵S]GTPγS Binding Assay

The [³⁵S]GTPγS assay measures the increase in guanine nucleotide exchange at G-proteins in cell membranes, resulting from agonist (PGD2) binding to CRTH2. This process can be monitored in vitro by incubating cell membranes containing G-proteins and CRTH2 with GDP and [³⁵S]GTPγS, a radiolabeled, hydrolysis-resistant analogue of GTP (see, Harrison et al., Life Sciences 74, 489-508, 2003). The addition of a Compounds of Formula (I) results in binding to CRTH2 and thus in an inhibition of agonist binding, which can be monitored as inhibition of the stimulation of GTP/GDP exchange. Briefly, Compounds of Formula (I) are incubated in 96-well scintillating white polystyrene plates (Perkin Elmer, USA) in a final volume of 200 μl containing 20 mM HEPES/KOH pH 7.4, 3 mM MgCl₂, 10 μg/ml Saponin, 5 μM GDP, 75 mM NaCl and 2% of dimethylsulphoxide (DMSO). Reaction is triggered by the addition of 5-10 μg of CHO—CRTH2 cell membranes and 0.15 nM [³⁵S]GTPγS. After 60 min incubation at 30° C., reaction is stopped by centrifugation at 700×g, at 4° C. for 10 minutes and supernatant is removed. The radioactivity coming from the [³⁵S]GTPγS bound on centrifuged cell membranes is recorded using a 1450 Micro-beta scintillation counter. For IC50 determination, increasing concentrations of compounds are incubated in presence of a fixed concentration of PGD2 (EC₈₀). For EC₅₀ measurements, compounds are incubated without addition of PGD2. Basal [³⁵S]GTPγS activity is determined without addition of any ligands or compounds. 100% [³⁵S]GTPγS activity is measured by the addition of 1 μM of PGD2.

In one embodiment, the compounds of Formula (I) of the present invention are antagonists of CRTH2. For example the compound of Example 108 had an IC₅₀ of 0.0224 μM.

In another embodiment, the compounds of Formula (I) of the present invention are partial agonists of CRTH2. For example the compound of Example 78 had an Emax of 19% (100% of Emax being the activity measured by the addition of 1 μM of PGD2).

In another embodiment, the compounds of Formula (I) of the present invention are inverse agonists of CRTH2. The results of representative examples are reported in the Table below (100% of Emax being the activity measured by the addition of 1 μM of PGD2).

Results:

| Example | EMax |
|---|---|
| 11 | −14.9% |
| 39 | −11.2% |
| 40 | −9.9% |
| 46 | −9.5% |
| 54 | −12.6% |
| 55 | −13.5% |
| 56 | −13.0% |
| 58 | −8.1% |
| 84 | −17.1% |
| 100 | −16.7% |
| 108 | −10.1% |

### EXAMPLE 123

### Cellular Dielectric Spectroscopy

Cellular Dielectric Spectroscopy (CDS) is a label-free technology based on the measurement of complex impedance changes (delta Z or dZ). Impedance (Z) is related to the ratio of voltage to current as described by Ohm's law (Z=V/I). In order to measure the changes in impedance that occur in response to receptor stimulation, mammalian cells are seeded onto a custom 96-well microtiter plate that contains electrodes at the bottom of each well Key contributors to the impedance measurements are changes in cell-substrate adherence, changes in cell shape and volume, and changes in cell-cell interactions. These factors individually or collectively affect the flow of current, influencing the magnitude and characteristics of the signal measured. G-protein coupled receptors ligand-induced activity can be measured using this technology and specific G protein coupling can be identified. Activities of reference agonist and antagonist molecules of CRTH2 have been measured using this assay and similar results were obtained compared to different functional assays.

CHO—CRTH2 cells are cultured in HAM's F12 (Lonza, Switzerland) supplemented with 10% foetal calf serum (PAA, Australia) and 400 μg/ml Geneticin. 100000 cells/well are seeded in standard 96 W Microplates (MDS Analytical Technologies) and incubated at 37° C. in 5% CO₂ for 24 hours. Cells are washed twice with 135 μl of cell key buffer (Hank's Balanced Salt Solution 1× (HBSS) (Invitrogen) supplemented with 10 mM HEPES pH 7.4 in presence of 1% DMSO). For EC₅₀ determination, 15 μl of increasing concentration of Compounds of Formula (I) diluted in cell key buffer are added to the cells and agonist activity is then recorded for 25 minutes. For IC₅₀ determination, 16.6 μl of a fixed concentration of PGD2 (EC80) diluted in cell key buffer is added to the cells-compounds mixture, and antagonist activity is measured during 25 minutes. Results are expressed as the amplitude between the highest and the lowest signal produced (max-min). Basal and maximum activities are measured, respectively in absence or presence of PGD2 (EC₈₀).

In one embodiment, the compounds of Formula (I) of the present invention are antagonists of CRTH2. The results of representative examples are reported in the Table below.

Results:

| Example | IC₅₀(μM) |
|---|---|
| 1 | 0.096 |
| 4 | 0.220 |
| 5 | 0.076 |
| 78 | 0.014 |
| 108 | 0.049 |

In another embodiment, the compounds of Formula (I) of the present invention are partial agonists of CRTH2. The results of representative examples are reported in the Table below.

Results:

| Example | EC₅₀(μM) | EMax |
|---|---|---|
| 78 | 0.0011 | 42% |
| 104 | 0.0011 | 27% |
| 105 | 0.0024 | 22% |
| 109 | 0.0012 | 32% |

### EXAMPLE 124

### PGD2-Induced Eosinophil Cell Shape Assay in Human Whole Blood

The Compounds of Formula (I) were diluted in dimethylsulphoxide so that the total volume of dimethylsulfoxide was kept constant at 2% dimethylsulphoxide (Me₂SO). Serial dilutions of 200 μM to 0.09 μM were prepared. Samples of 90 μl of human blood from healthy volunteers (Centre de Transfusion Sanguine de Genéve) were pre-incubated in polypropylene Falcon tubes (BD 352063) for 20 minutes in a water bath at 37° C. with 10 μl of diluted compounds. For CRTH2 activation, 100 μl PGD2 (Cayman 12010) at 20 nM was added (10 nM final) to each tube and cells were maintained at 37° C. For negative control cells were treated with PBS. After 10 minutes, cell activation was stopped with 120 μl Formaldehyde 10% (4% final, Fluka 41650) and cells were rested for 10 minutes at room temperature. Fixed cells were transferred into polypropylene tubes and then treated for 1 hour in a water bath at 37° C. with 2 ml of Triton-Surfact-Amps X-100 (Pierce 28314) at 0.166% (0.13% Triton final). After several washes with PBS (red cells lysed progressively during washes, two washes are necessary), cells were analyzed by flow cytometry on a FACSCalibur. In one embodiment, the compounds of Formula (I) of the present invention are capable of blocking the cell shape change of eosinophils induced by PGD2 in Whole Blood. The results of representative examples are reported in the Table below.

Results:

| Example | IC₅₀(μM) |
|---|---|
| 5 | 0.536 |
| 9 | 1.170 |
| 11 | 0.039 |
| 13 | 0.914 |
| 46 | 0.245 |
| 55 | 0.174 |
| 55 | 0.555 |
| 56 | 0.082 |
| 57 | 0.233 |
| 58 | 0.102 |

### EXAMPLE 125

### In Vivo Pharmacokinetic Evaluation in Rat and Mouse

In order to study the pharmacokinetic (PK) profile of test compounds in vivo, Sprague Dawley male rats or C57BL/6 female mice were dosed intravenously or after oral gavage. For both species, test compounds were dosed in solution at 1 mg/kg for i.v. route (10% ethanol, 10% N,N-dimethylacetamide, 30% propylene glycol, 50% water, v/v) and in suspension at 5 mg/kg (0.5% carboxymethylcellulose suspension, containing 0.25% Tween 20 in water) for oral gavage. PK profile in rat was obtained from 3 animals per dosing route and mouse PK profile was determined from 3 animals for each time points. The volume of administration was 2 mL/kg for i.v. dosing in both species and either 5 mL/kg (rat) or 10 mL/kg (mouse) for oral gavage. Blood samples (100 μL/time point) were collected at 0.083 (5 min), 0.25, 0.5, 1, 4, 7 and 24 hours post-dose for i.v. dosing, and at 0.5, 1, 4, 7 and 24 h for oral dosing, into heparin-Li+ containing tubes. For rats, all blood samples were collected trough a catheter in the carotid artery (placed in the artery the day before the experiment), under light isoflurane anesthesia, and stored on ice until centrifugation and plasma isolation. For mouse, blood samples were collected from intracardiac puncture at sacrifice at each time point and processed as described above for the rat. Plasma samples were stored frozen until analysis (−20° C. to −70° C.). For bioanalysis, samples were processed by protein precipitation (acetonitrile, formic acid 0.1%, addition of 3 volumes) after addition of one internal standard and analysed using a sensitive and selective LC/MS/MS method. An aliquot of the resulting supernatant was subject to LC/MS/MS analysis using a reverse phase column (Waters Xterra, C8, (3.5 μm particle size, 2.1×50 mm) and a short gradient (1 min) from (Solvent A) 85% water, 15% acetonitrile and 0.1% formic acid to (Solvent B) 90% acetonitrile, 10% water and 0.1% formic acid followed by isocratic conditions of Solvent B for 3.5 min at 0.4 mL/min. Column effluent was monitored using a Sciex API 4000 triple quadrupole mass spectrometer with a Turbo V electrospray ion source. Unknown concentrations of test compounds were determined using a calibration curve ranging from 1 to 3000 ng/mL.

Pharmacokinetic profile in mice of representative compounds

| | Clearance iv | AUC po | |
|---|---|---|---|
| | (1 mg/Kg) | (5 mg/Kg) | Oral |
| Compound | (L/Kg/h) | (h*ng/ml) | bioavailability |
| Example 1 | 0.1 | 12159 | 33% |
| Example 56 | 0.9 | 1908 | 36% |
| Example 58 | 0.3 | 11212 | 76% |

### EXAMPLE 126

### Preparation of a Pharmaceutical Formulation

### Formulation 1—Tablets

A compound of formula (I) is admixed as a dry powder with a dry gelatin binder in an approximate 1:2 weight ratio. A minor amount of magnesium stearate is added as a lubricant. The mixture is formed into 240-270 mg tablets (80-90 mg of active compound according to the invention per tablet) in a tablet press.

### Formulation 2—Capsules

A compound of formula (I) is admixed as a dry powder with a starch diluent in an approximate 1:1 weight ratio. The mixture is filled into 250 mg capsules (125 mg of active compound according to the invention per capsule).

### Formulation 3—Liquid

A compound of formula (I) (1250 mg), sucrose (1.75 g) and xanthan gum (4 mg) are blended, passed through a No. 10 mesh U.S. sieve, and then mixed with a previously prepared solution of microcrystalline cellulose and sodium carboxymethyl cellulose (11:89, 50 mg) in water. Sodium benzoate (10 mg), flavor, and color are diluted with water and added with stirring. Sufficient water is then added to produce a total volume of 5 mL.

### Formulation 4—Tablets

A compound of formula (I) is admixed as a dry powder with a dry gelatin binder in an approximate 1:2 weight ratio. A minor amount of magnesium stearate is added as a lubricant. The mixture is formed into 450-900 mg tablets (150-300 mg of active compound according to the invention) in a tablet press.

### Formulation 5—Injection

A compound of formula (I) is dissolved in a buffered sterile saline injectable aqueous medium to a concentration of approximately 5 mg/mL.

1-15. (canceled)

16. A compound of formula (I) 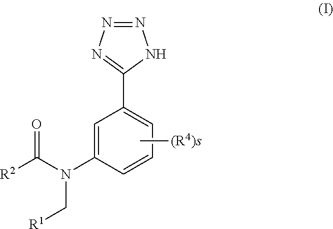
wherein:
R¹ is —(CH₂)ₙ—Ar, —(CH₂)ₙHet, —(CH₂)ₚ—(CHR⁸)ₘ—(CH₂)_{q}—Ar, or —(CH₂)ₚ—(CHR⁸)ₘ—(CH₂)_{q}—Het,
R² is A, Het, Ar, or a cycloalkyl having 1 to 8 carbon atoms,
R⁴ is H, Hal, A, CN, OA, CF₃, OCF₃,
n is 0, 1, 2, 3, or 4,
p, q are 0, 1, 2 or 3,
m is 0, 1 or 2,
s 1, 2 or 3,
R⁸ denotes a group selected from an alkyl having 1 to 8 carbon atoms, —CH₂F, —CF₃, OR³, N(R³)₂, —CH₂OCH₃, —CH₂OCF₃, —CH₂CONH₂, or CN,
A is branched or linear alkyl having 1 to 12 C-atoms, wherein one or more H-atoms may be replaced by Hal, OR³, CN, N(R³)₂, CON(R³)₂, Ar or Het and wherein one or more non-adjacent CH₂— groups may be replaced by O, NR³ or S and/or by —CH═CH— or —C≡C— groups, or denotes cycloalkyl or cycloalkylalkylene having 3 to 7 ring C atoms,
R³ denotes II or A,
Hal is F, Cl, Br or I,
Ar denotes a monocyclic or fused bicyclic, unsaturated or aromatic carbocyclic ring having 6 to 14 carbon atoms which may be unsubstituted or mono substituted, disubstituted or trisubstituted by Hal, A, CH₇OA, —CH₂OR³, OR³, CF₃, OCF₃, N(R³)₂, NO₂, CN, NR³COA, NR³SO₂A, COR³, SO₂N(R³)₂, SOA, SO₂A, SOAr, SO₂Ar, SOHet, SO₂Het, Ar′, Het, or by —CH═CH—R³ or —C≡C—R³,
Het denotes a monocyclic or bicyclic, saturated, unsaturated or aromatic heterocyclic ring, having 1 to 4 N, O and/or S atoms, which may be unsubstituted or monosubstituted, disubstituted or trisubstituted by Hal, A, OR³, —(CH₂)OR³, CF₃, OCF₃, N(R³)₂, NO₂, CN, NR³COA, NR³SO₂A, COR³, SO₂N(R³)₂, SOA, SO₂A, SOAr, SO₂Ar, SOHet, SO₂Het, Ar, Het′, or by —CH═CH—R³ or —C≡C—R³.
Ar′ denotes a monocyclic or bicyclic, unsaturated or aromatic carbocyclic ring having 6 to 14 carbon atoms which may be unsubstituted or monosubstituted, disubstituted or trisubstituted by Hal, A, —(CH₂)OR³, —OR³, —CF₃ or —OCF₃,
Het′ denotes a monocyclic or bicyclic, saturated, unsaturated or aromatic heterocyclic ring, having 1 to 4 N, O and/or S atoms, which may be unsubstituted or monosubstituted, disubstituted or trisubstituted by Hal, A, —(CH₂)OR³, —OR³, —CF₃, or —OCF₃,
derivatives, enantiomers, diastereoisomers, tautomers, salts, solvates and mixtures thereof in all ratios.

17. The compound according to claim 16 wherein Het denotes one of the following groups: 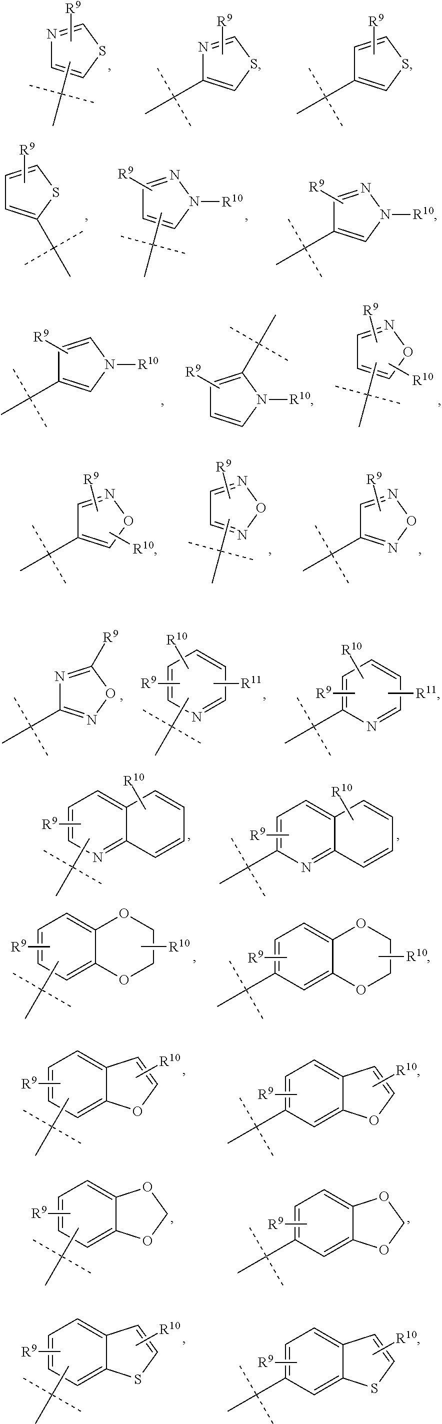 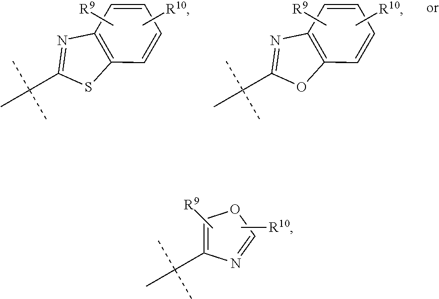
wherein R⁹, R¹⁰ and R¹¹ are independently selected from H, A, Hal, linear or branched alkyl having 1 to 6 carbon atoms, Ar, —OR³, —CN, —CF₃, and —OCF₃ and Ar and R³ are as defined in claim 16.

18. The compound according to claim 17, wherein Ar is selected from the following groups: 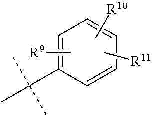
wherein R⁹, R¹⁰ and R¹¹, are independently selected from H, Hal, —CH₂OR³, —OR³, —OCF₃, —CN, —(CH₂)_{T}—C≡C—R⁶, —(CH₂)_{T}—CH═CH—R⁶, Ar′, Het or SO₂(C₁-C₆)alkyl,
R⁶ is H, a linear or branched C₁-C₆ alkyl, or a group selected from —CH₂F, —CF₃, —CH₂OCH₃, —CH₂OCF₃—CH₂CONH₂, CN, Ar or Het,
T is 0, 1, 2 or 3,
and R³ is as defined in claim 16.

19. The compound according to claim 16, wherein R¹ is selected from the following groups:

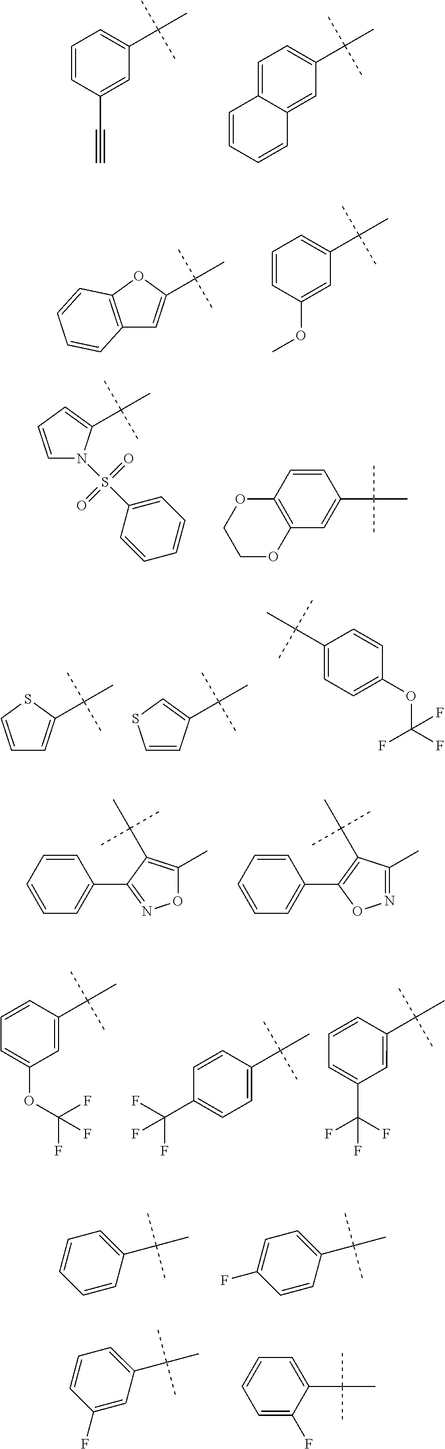

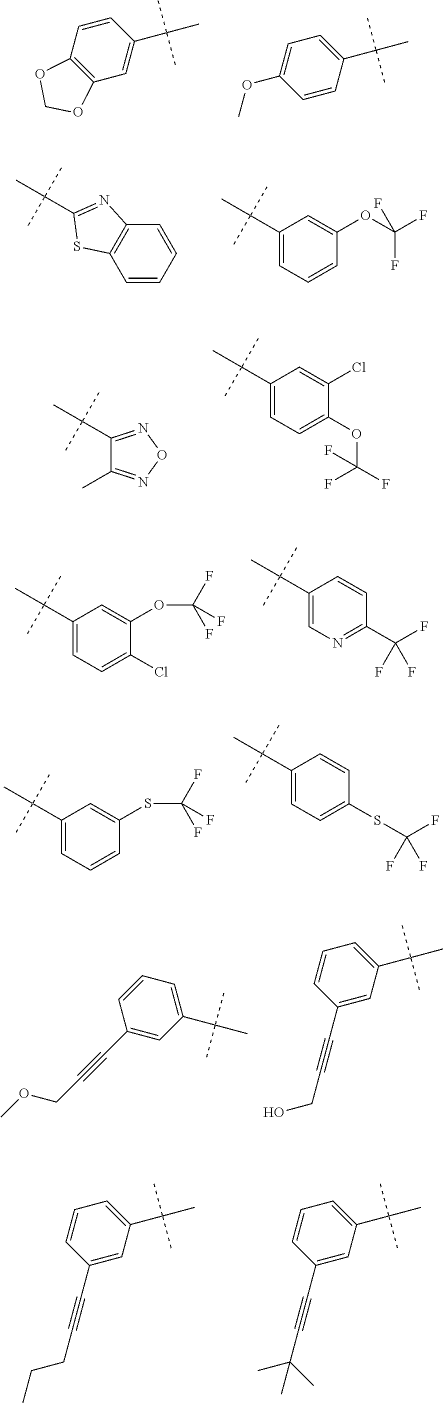

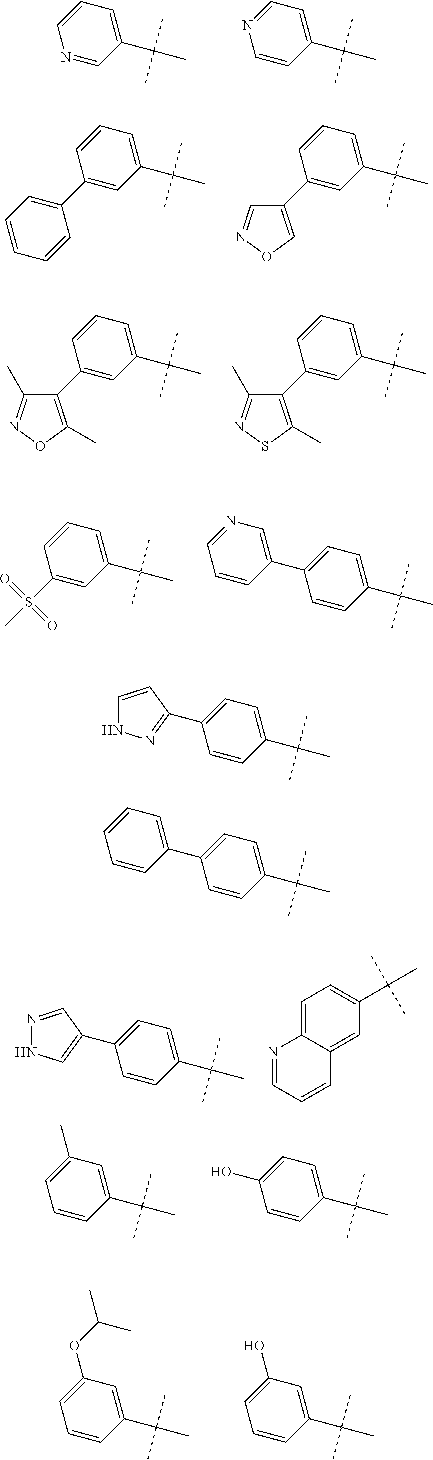

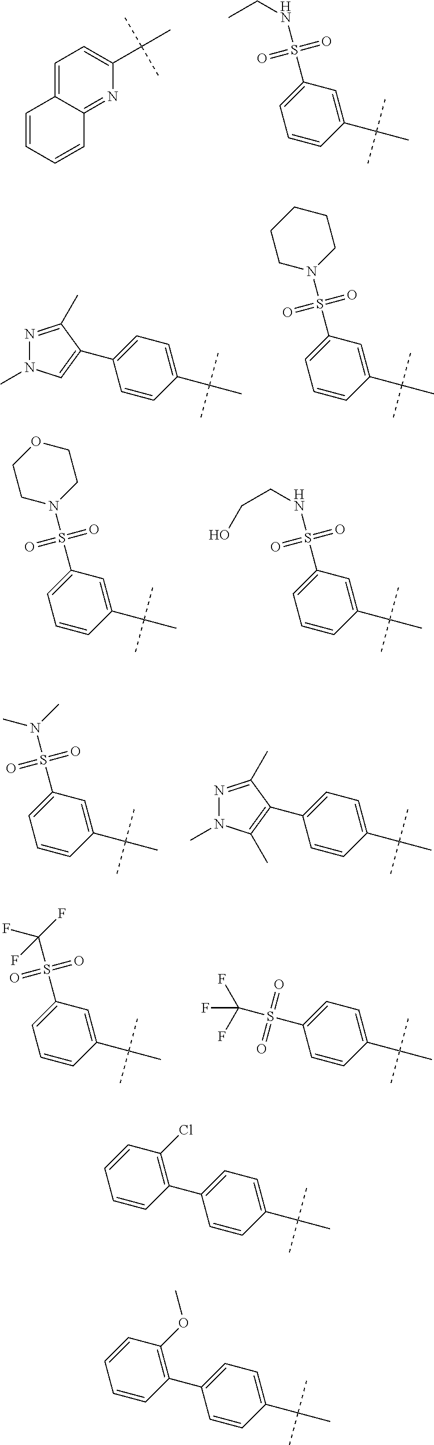

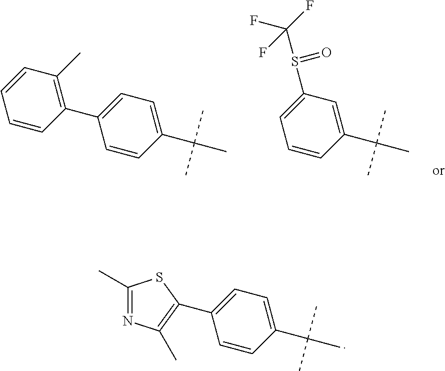

20. The compound according to claim 16, wherein the compound is of formulae (Ia), (Ib) or (Ic) 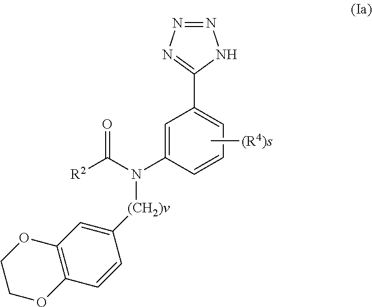
wherein R², R⁴, and s are as defined in claim 16 and v is 1, 2, 3 or 4, 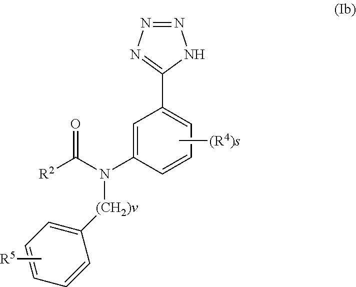
wherein R², R⁴, and s are as defined in claim 16, v is 1, 2, 3 or 4, and
wherein R⁵ denotes H or a group selected from Hal, —OCF₃, —OCH₃, —CF₃, —(CH₂)_{T}—CH═CH—R⁶, or SO₂(C₁-C₆alkyl),
wherein R⁶ is H, a linear or branched C₁-C₆ alkyl, or a group selected from —CH₂F, —CF₃, —CH₂OCH₃, —CH₂OCF₃, —CH₂CONH₂, CN, Ar or Het, and
wherein T is 0, 1, 2 or 3. 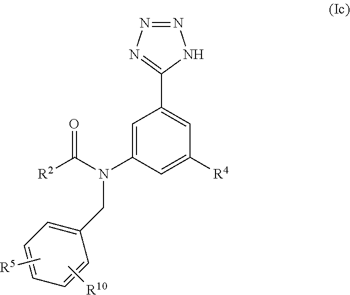
wherein:
R² and R⁴ are as defined in claim 16,
R⁹ denotes H, Hal, CF₃, OCF₃, SO₂(C₁-C₆)alkyl,
R¹⁰ denotes H, or Hal,
or derivatives, enantiomers, diastereoisomers, tautomers, salts, solvates and mixtures thereof.

21. The compound according to claim 16 selected from the following group:

| EX. | Formula |
|---|---|
| 1 | 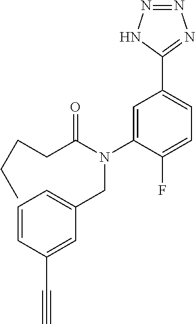 |
| 2 | 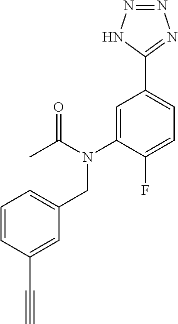 |
| 3 | 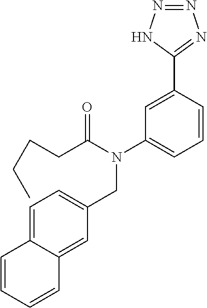 |
| 4 | 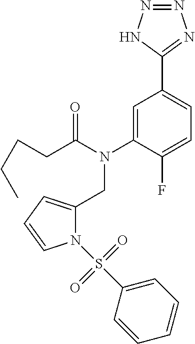 |
| 5 | 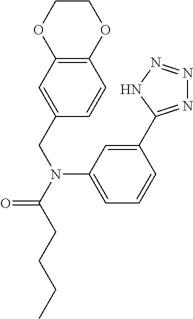 |
| 6 | 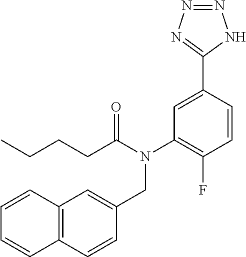 |
| 7 | 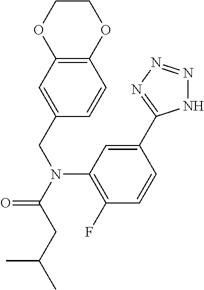 |
| 8 | 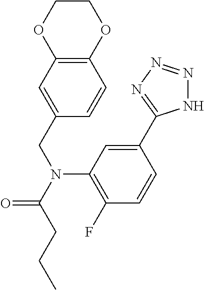 |
| 9 | 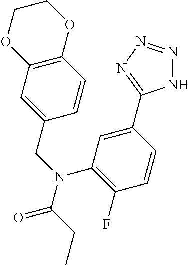 |
| 10 | 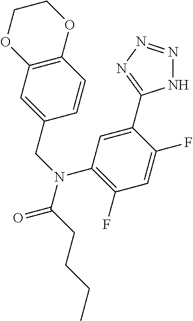 |
| 11 | 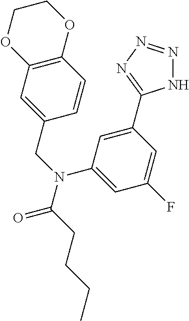 |
| 12 | 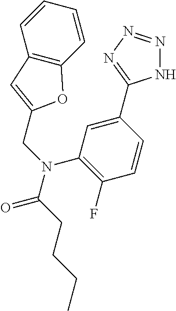 |
| 13 | 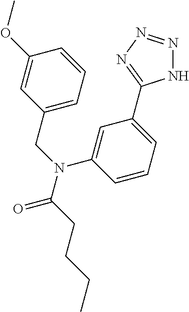 |
| 14 | 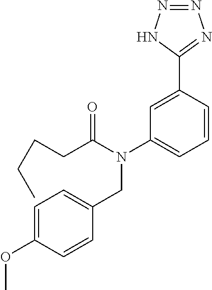 |
| 15 | 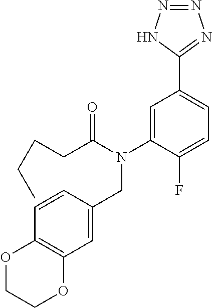 |
| 16 | 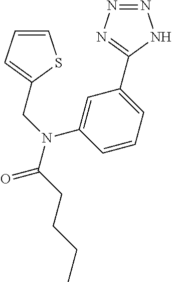 |
| 17 | 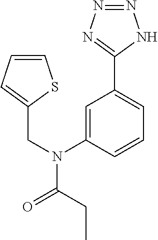 |
| 18 | 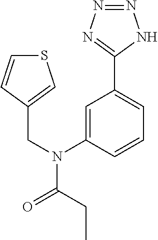 |
| 19 | 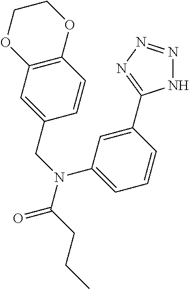 |
| 20 | 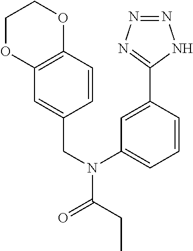 |
| 21 | 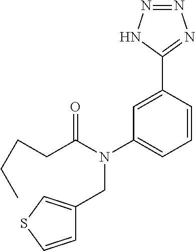 |
| 22 | 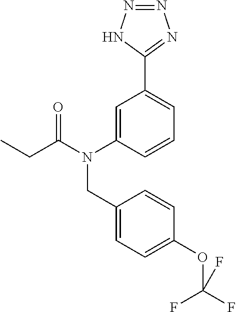 |
| 23 | 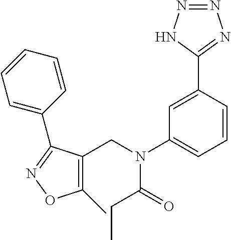 |
| 24 | 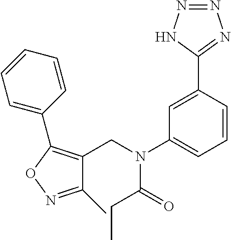 |
| 25 | 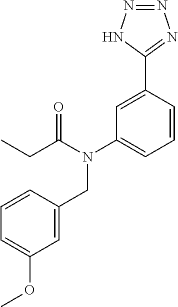 |
| 26 | 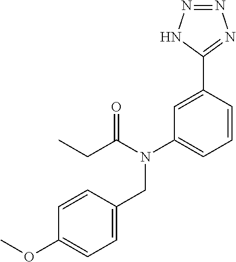 |
| 27 | 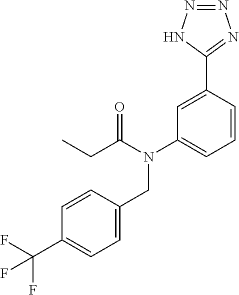 |
| 28 | 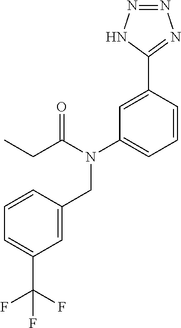 |
| 29 | 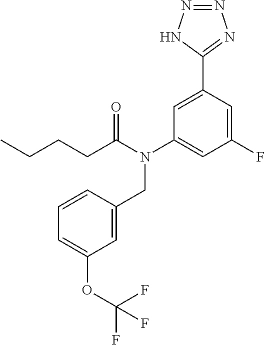 |
| 30 | 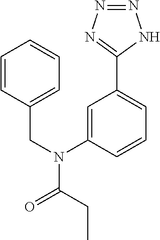 |
| 31 | 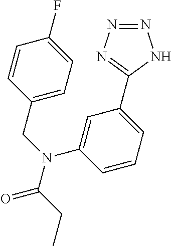 |
| 32 | 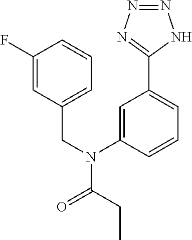 |
| 33 | 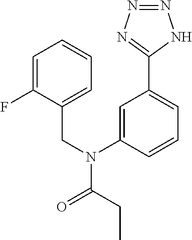 |
| 34 | 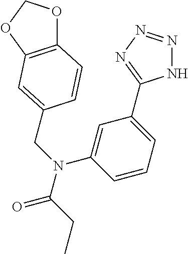 |
| 35 | 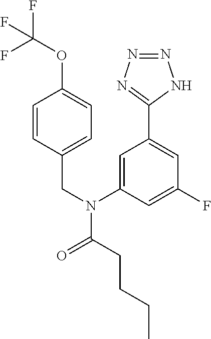 |
| 36 | 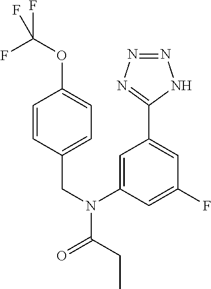 |
| 37 | 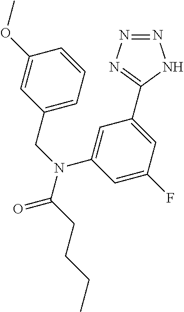 |
| 38 | 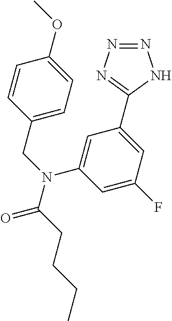 |
| 39 | 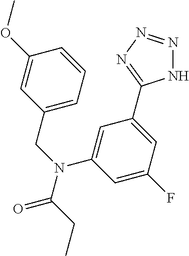 |
| 40 | 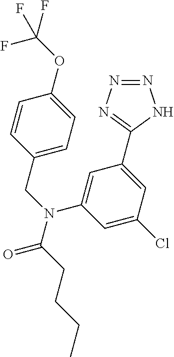 |
| 41 | 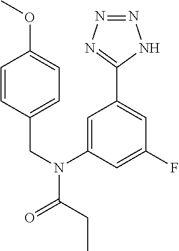 |
| 42 | 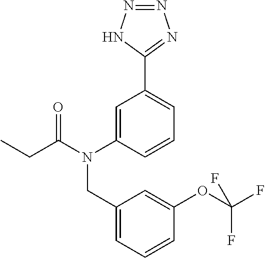 |
| 43 | 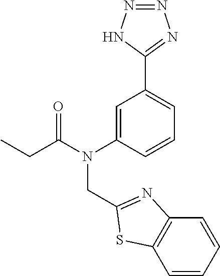 |
| 44 | 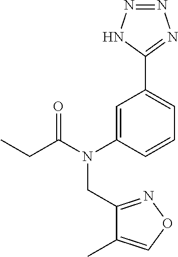 |
| 45 | 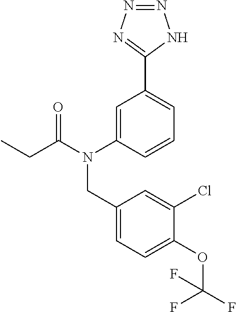 |
| 46 | 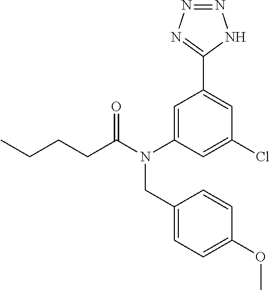 |
| 47 | 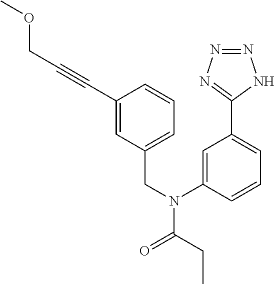 |
| 48 | 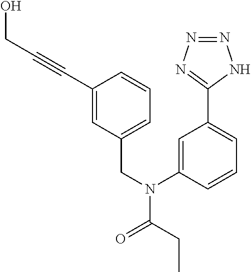 |
| 49 | 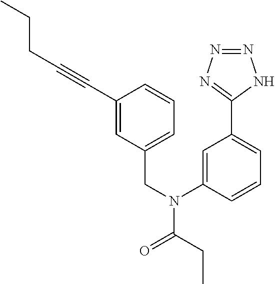 |
| 50 | 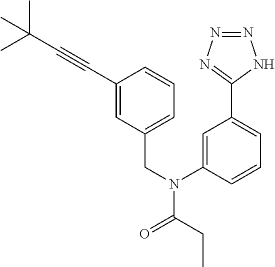 |
| 51 | 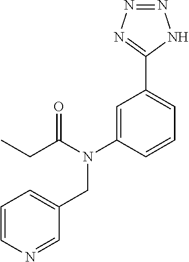 |
| 52 | 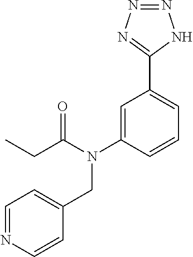 |
| 53 | 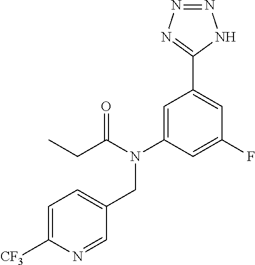 |
| 54 | 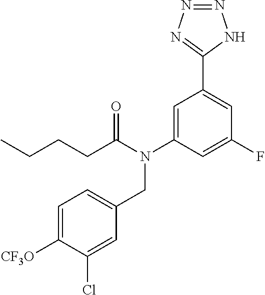 |
| 55 | 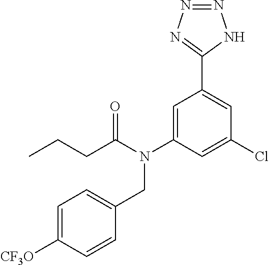 |
| 56 | 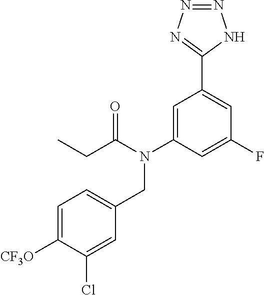 |
| 57 | 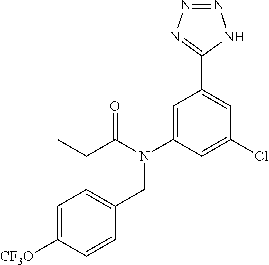 |
| 58 | 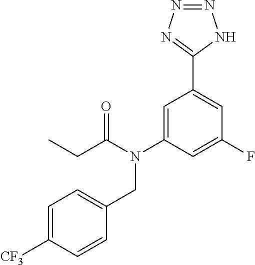 |
| 59 | 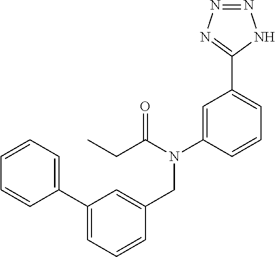 |
| 60 | 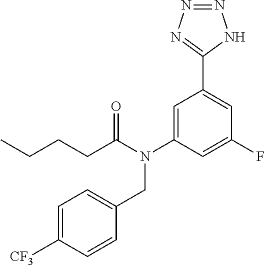 |
| 61 | 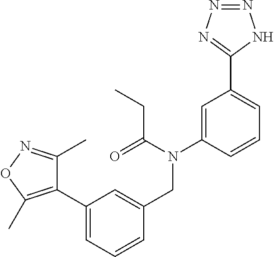 |
| 62 | 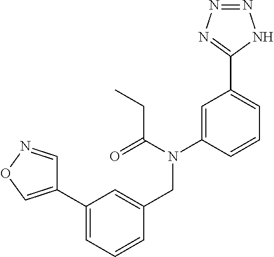 |
| 63 | 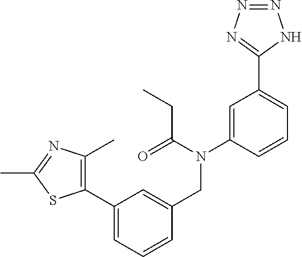 |
| 64 | 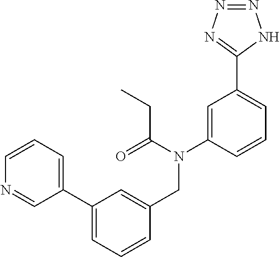 |
| 65 | 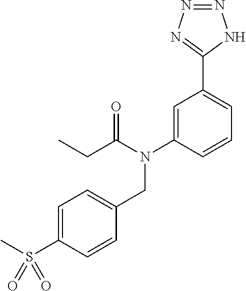 |
| 66 | 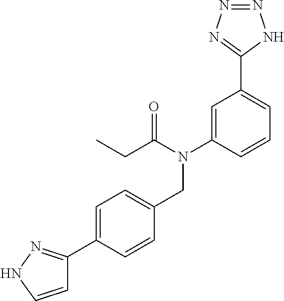 |
| 67 | 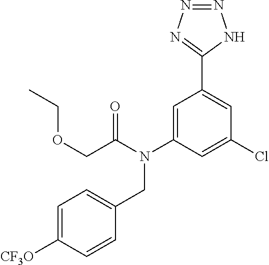 |
| 68 | 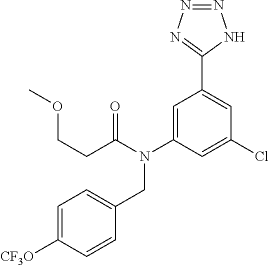 |
| 69 | 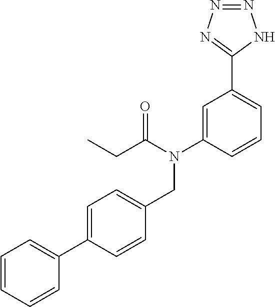 |
| 70 | 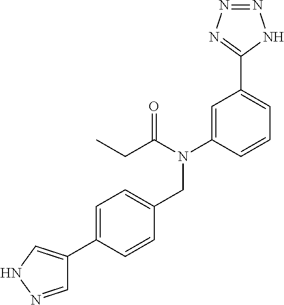 |
| 71 | 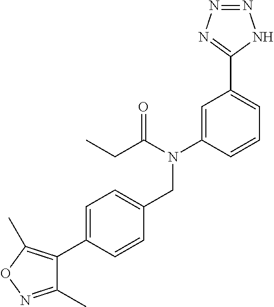 |
| 72 | 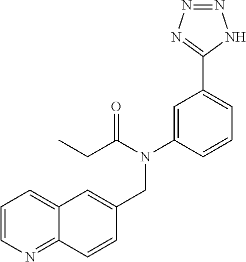 |
| 73 | 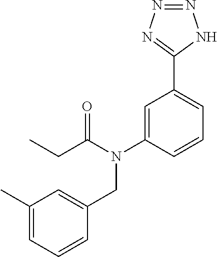 |
| 74 | 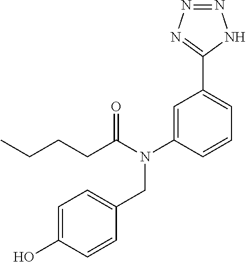 |
| 75 | 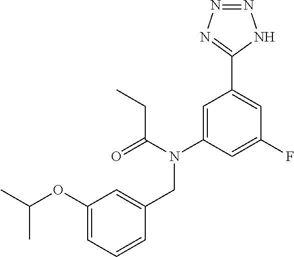 |
| 76 | 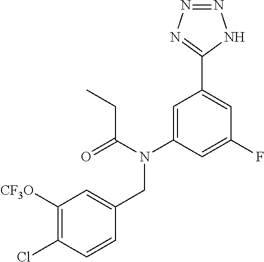 |
| 77 | 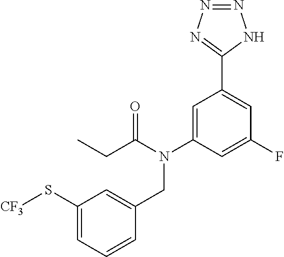 |
| 78 | 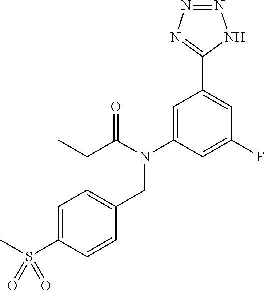 |
| 79 | 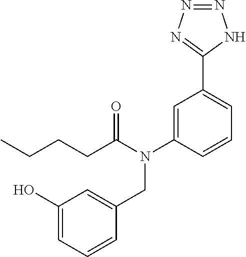 |
| 80 | 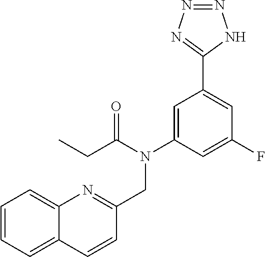 |
| 81 | 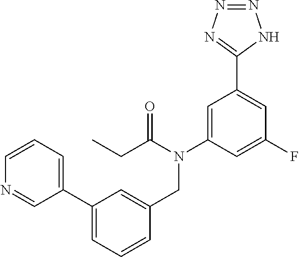 |
| 82 | 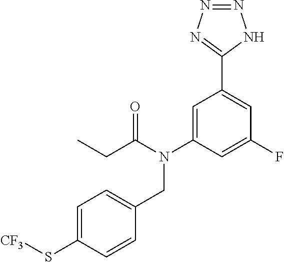 |
| 83 | 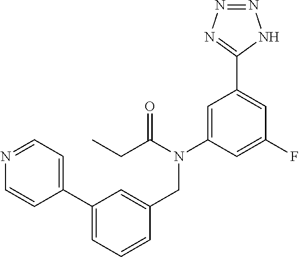 |
| 84 | 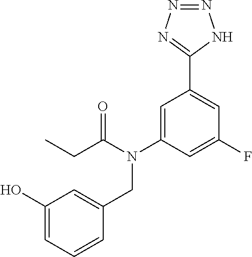 |
| 85 | 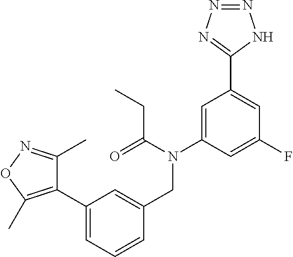 |
| 86 | 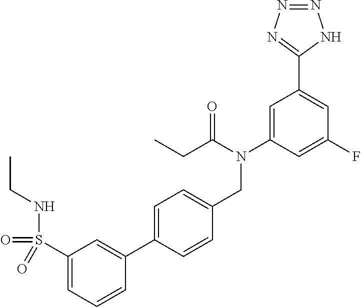 |
| 87 | 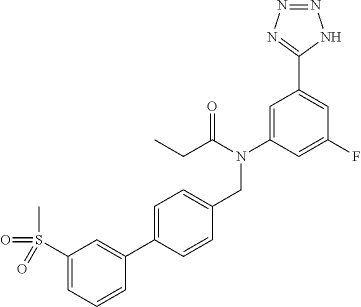 |
| 88 | 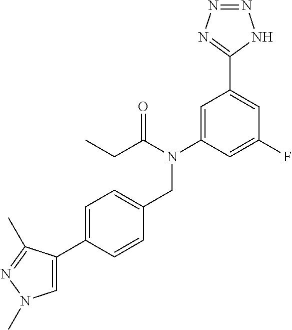 |
| 89 | 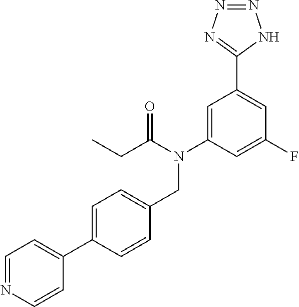 |
| 90 | 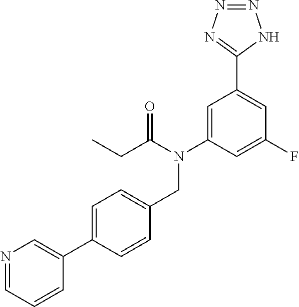 |
| 91 | 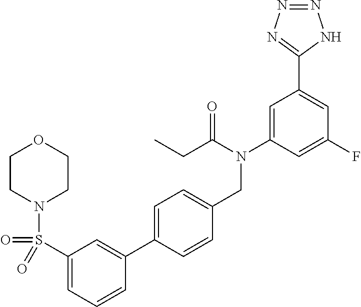 |
| 92 | 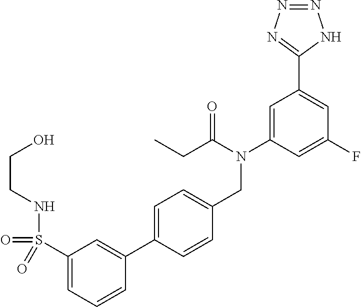 |
| 93 | 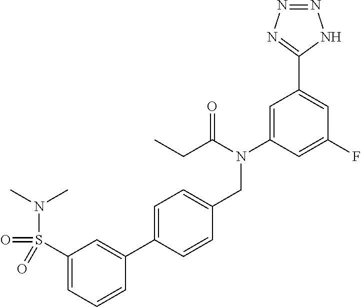 |
| 94 | 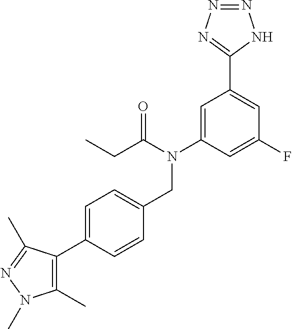 |
| 95 | 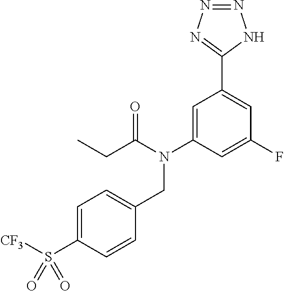 |
| 96 | 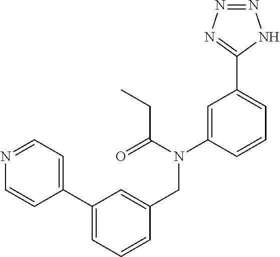 |
| 97 | 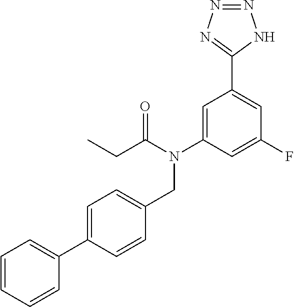 |
| 98 | 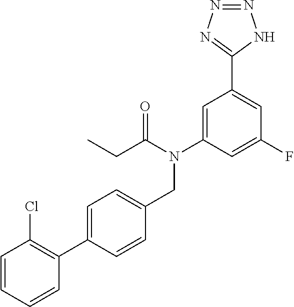 |
| 99 | 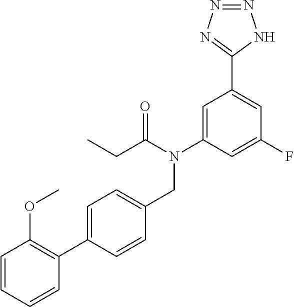 |
| 100 | 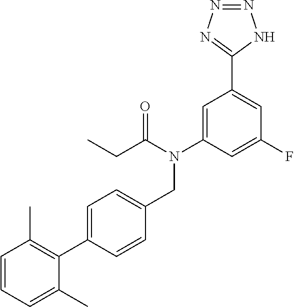 |
| 101 | 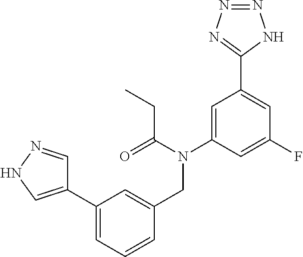 |
| 102 | 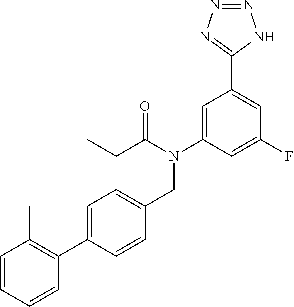 |
| 103 | 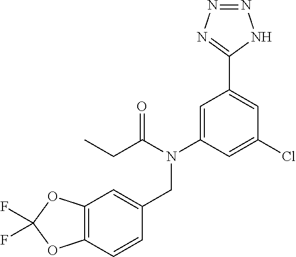 |
| 104 | 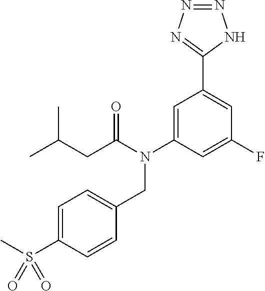 |
| 105 | 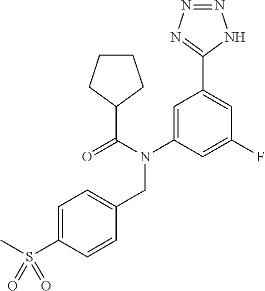 |
| 106 | 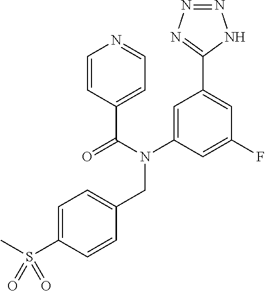 |
| 107 | 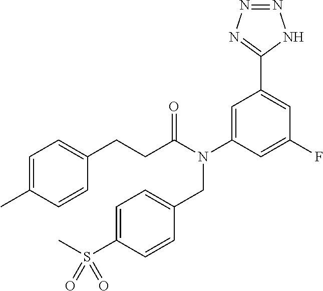 |
| 108 | 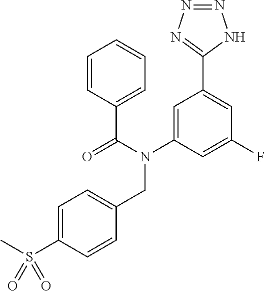 |
| 109 | 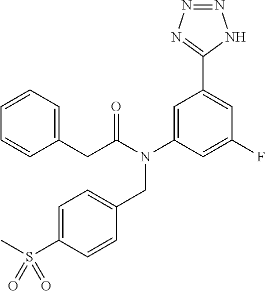 |
| 110 | 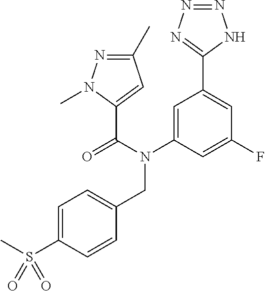 |
| 111 | 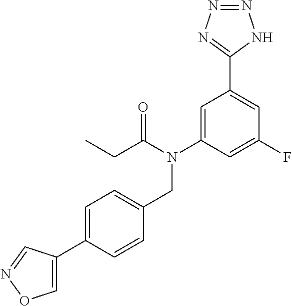 |
| 112 | 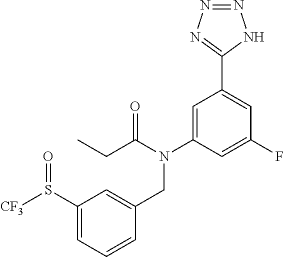 |
| 113 | 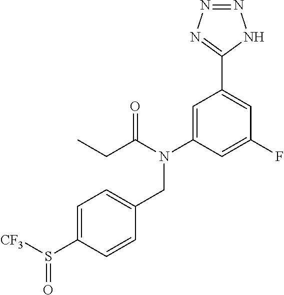 |
| 114 | 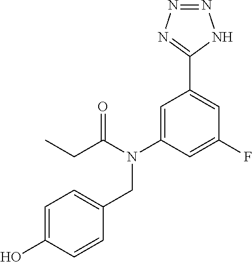 |
| 115 | 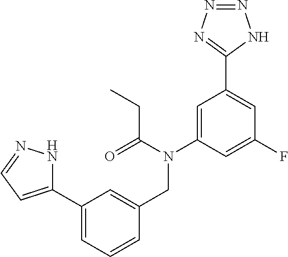 |
| 116 | 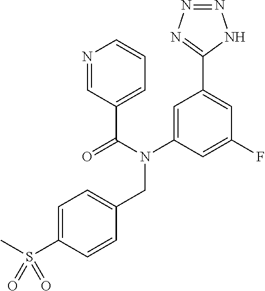 |
| 117 | 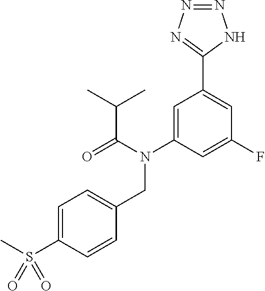 |
| 118 | 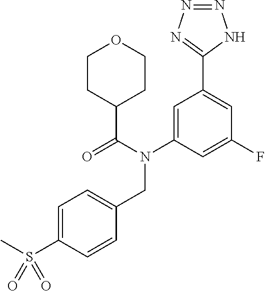 or |
| 119 | 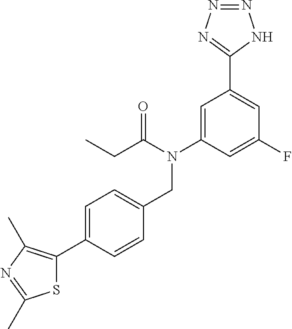 |

or derivatives, enantiomers, diastereoisomers, tautomers, salts, solvates and mixtures thereof.

22. A method of treating CRTH2 related diseases comprising the administration of a compound according to claim 16 to an individual having a CRTH2 related disease in an amount effective to treat said disease.

23. A method of treating allergic diseases or inflammatory dermatoses comprising the administration of a compound according to claim 16 to an individual having an allergic disease or inflammatory dermatoses in an amount effective to treat said disease.

24. The method according to claim 23, wherein the disease is selected from allergic asthma, allergic rhinitis, or allergic conjunctivitis.

25. The method according to claim 23, wherein the disease is selected from atopic dermatitis, contact hypersensitivity, allergic contact dermatitis, chronic urticaria/chronic idiopathic/autoimmune urticaria, drug-induced exanthems, photodermatosis or polymorphous light eruption, myositis neurodegenerative disorders, rheumatoid arthritis, multiple sclerosis, osteoarthritis, or inflammatory bowel disease (IBD).

26. A pharmaceutical composition comprising at least one compound according to claim 16 and an excipient and/or adjuvant.

27. The pharmaceutical composition according to claim 26, said composition further comprising at least one additional active ingredient.

28. A kit or a set consisting of separate packs of:
(a) an effective amount of a compound according to claim 16, and
(b) an effective amount of an additional active ingredient.

29. A process for the preparation of a compound of claim 16 comprising the step of reacting a compound of formula (III) with R²COCl in the presence of a base, 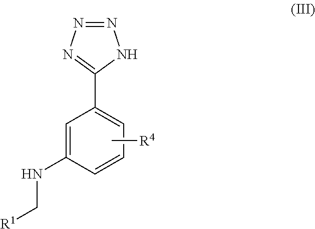
or comprising the step of reacting a compound of formula (VI) with TMS-N₃, in the presence of a catalyst selected from Bu₂SnO or Cu₂O, 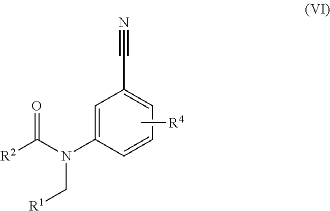
wherein R¹, R², and R⁴ are as defined in claim 16.
